(19) United States
(12) Patent Application Publication
(10) Pub. No.: US 2026/0272978 A1
(43) Pub. Date: Sep. 17, 2026

(54) METHODS OF MANUFACTURING T CELL THERAPIES

(71) Applicant: Celgene Corporation, Summit, NJ (US)

(72) Inventors: David R. MERTZ, Morristown, NJ (US); Jaymes FULLER, Westfield, NJ (US); Erica GRAF, Summit, NJ (US); Jessica MARFO, Summit, NJ (US)

(73) Assignee: Celgene Corporation, Summit, NJ (US)

(21) Appl. No.: 18/868,671

(22) PCT Filed: May 25, 2023
(86) PCT No.: PCT/US2023/067511

§ 371 Date: Nov. 22, 2024

Related U.S. Application Data
(60) Provisional application No. 63/345,813, filed on May 25, 2022.
(60) Provisional application No. 63/420,458, filed on Oct. 28, 2022.
(60) Provisional application No. 63/441,758, filed on Jan. 27, 2023.

(51) Int. Cl. A61K 35/17; A61K 40/11; A61K 40/31; A61K 40/42; C12N 5/0783; G01N 33/50

(52) U.S. Cl. CPC A61K 35/17; A61K 40/11; A61K 40/31; A61K 40/4215; C12N 5/0636; G01N 33/505; A61K 2239/13; C12N 2501/515; C12N 2503/00

(57) ABSTRACT

The present disclosure provides cell populations enriched for CD28 positive T cells, and methods for stimulating, genetically engineering, and/or cultivating such cell populations. Also included are methods for generating, isolating, enriching, or selecting CD28 positive T cells or populations of cells enriched for CD28 positive T cells.

### CROSS-REFERENCE TO RELATED APPLICATIONS

This application claims priority from U.S. provisional No. 63/345,813, filed May 25, 2022, U.S. provisional application No. 63/420,458, filed Oct. 28, 2022, and U.S. provisional application No. 63/441,758, filed Jan. 27, 2023, all entitled “METHODS OF MANUFACTURING T CELL THERAPIES” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 683772002240SeqList.xml created on May 24, 2023, which is 398,108 bytes in size. The information in the electronic format of the Sequence Listing is incorporated by reference in its entirety.

### FIELD

The present disclosure relates in some aspects to cell populations enriched for CD28 positive T cells, including by the selection of cell populations based on the percentage of CD28 positive T cells, the selection of CD28 positive T cells, or both, and methods for stimulating, genetically engineering, and/or cultivating such cell populations. Also included are methods for generating, isolating, enriching, or selecting CD28 positive T cells.

### BACKGROUND

Various methods for manufacturing genetically engineered cells, including for adoptive cell therapy, are available. Among these are methods involving the genetic engineering of immune cells, such as T cells, to express a recombinant receptor, such as a chimeric antigen receptor. However, in some cases, existing manufacturing methods may not be satisfactory, and may yield insufficient numbers of engineered cells for providing a dose of a cell therapy. Patient-to-patient variability, inherent in the autologous cell starting material is a key contributor to the manufacturing outcome. Improved methods for manufacturing such cell therapies are therefore needed, including to identify cells that will sufficiently proliferate or expand, as well as to improve the proliferation or expansion of engineered cells.

### SUMMARY

Provided herein is a method of selecting cells for manufacture of a cell therapy, including: (1) determining the percentage of CD28+ T cells in a first biological sample obtained from a subject, wherein the first biological sample contains T cells; and (2) selecting the subject for manufacturing a cell therapy from a second biological sample obtained from the subject if the percentage of CD28+ T cells in the first biological sample is above a threshold value, wherein the second biological sample contains T cells.

In some embodiments, the method further includes genetically engineering cells of the second biological sample to express a recombinant receptor, thereby generating a cell therapy comprising the genetically engineered cells.

Also provided herein is a method of manufacturing a cell therapy, including: (1) selecting a subject for manufacturing a cell therapy if the percentage of CD28+ T cells in a first biological sample obtained from the subject is above a threshold value, wherein the first biological sample contains T cells; and (2) genetically engineering cells of a second biological sample obtained from the subject to express a recombinant receptor, thereby generating a cell therapy containing the genetically engineered cells, wherein the second biological sample contains T cells.

In some embodiments, the method further includes administering a dose of the cell therapy to a subject.

Also provided herein is a method of treating a subject with a cell therapy, including administering a dose of a cell therapy containing genetically engineered cells to a subject, wherein: (1) the percentage of CD28+ T cells in a first biological sample obtained from a subject is determined to be above a threshold value, wherein the first biological sample contains T cells; (2) the subject is selected for manufacture of the cell therapy based on the determining in step (1); and (3) cells of a second biological sample obtained from the subject are genetically engineered to express a recombinant receptor, thereby generating the cell therapy, wherein the second biological sample contains T cells.

In some embodiments, the method further includes selecting the subject for manufacturing the cell therapy from the second biological sample obtained from the subject if the percentage of CD28+ T cells in the first biological sample is above a threshold value. In some embodiments, the method further includes determining the percentage of CD28+ T cells in the first biological sample. In some embodiments, the first biological sample and the second biological sample are the same sample. In some embodiments, the first biological sample and the second biological sample are the same sample, which is an apheresis sample or a leukapheresis sample. In some embodiments, the first biological sample and the second biological sample are the same sample, which is an apheresis sample. In some embodiments, the first biological sample and the second biological sample are the same sample, which is a leukapheresis sample.

Also provided herein is a method of selecting cells for manufacture of a cell therapy, including: (1) determining the percentage of CD28+ T cells in a first biological sample obtained from a subject; and (2) selecting the subject for manufacturing a cell therapy from a second biological sample obtained from the subject if the percentage of CD28+ T cells in the first biological sample is above a threshold value, wherein the first biological sample and the second biological sample are the same sample, which is an apheresis sample.

In some embodiments, the method further includes genetically engineering cells of the second biological sample to express a recombinant receptor, thereby generating a cell therapy comprising the genetically engineered cells.

Also provided herein is a method of manufacturing a cell therapy, including: (1) selecting a subject for manufacturing a cell therapy if the percentage of CD28+ T cells in a first biological sample obtained from the subject is above a threshold value; and (2) genetically engineering cells of a second biological sample obtained from the subject to express a recombinant receptor, thereby generating a cell therapy comprising the genetically engineered cells, wherein the first biological sample and the second biological sample are the same sample, which is an apheresis sample.

In some embodiments, the method further includes administering a dose of the cell therapy to a subject.

Also provided herein is a method of treating a subject with a cell therapy, including administering a dose of a cell therapy comprising genetically engineered cells to a subject, wherein: (1) the percentage of CD28+ T cells in a first biological sample obtained from a subject is determined to be above a threshold value; (2) the subject is selected for manufacture of the cell therapy based on the determining in step (1); and (3) cells of a second biological sample obtained from the subject are engineered to express a recombinant receptor, thereby generating the cell therapy, wherein the first biological sample and the second biological sample are the same sample, which is an apheresis sample.

In some embodiments, the method further includes selecting the subject for manufacturing the cell therapy from the second biological sample obtained from the subject if the percentage of CD28+ T cells in the first biological sample is above a threshold value.

In some embodiments, the percentage of CD28+ T cells is the percentage of total T cells that are CD28+. In some embodiments, the method further includes determining the percentage of CD28+ T cells in the first biological sample. In some embodiments, the threshold value is between about 30% and about 50% CD28+ T cells, or between about 35% and about 45% CD28+ T cells. In some embodiments, the threshold value is between about 30% and about 50% CD28+ T cells. In some embodiments, the threshold value is between about 35% and about 45% CD28+ T cells. In some embodiments, the threshold value is about 40% CD28+ T cells.

In some embodiments, the first biological sample and the second biological sample are different samples. In some embodiments, the first biological sample is a whole blood sample, an apheresis sample, or a leukapheresis sample. In some embodiments, the first biological sample is a whole blood sample. In some embodiments, the first biological sample is an apheresis sample. In some embodiments, the first biological sample is a leukapheresis sample. In some embodiments, the second biological sample is a whole blood sample, an apheresis sample, or a leukapheresis sample. In some embodiments, the second biological sample is a whole blood sample. In some embodiments, the second biological sample is an apheresis sample. In some embodiments, the second biological sample is a leukapheresis sample.

In some embodiments, the first biological sample is a whole blood sample or an apheresis sample, and the second biological sample is an apheresis sample or a leukapheresis sample. In some embodiments, the first biological sample is a whole blood sample and the second biological sample is a leukapheresis sample.

In some embodiments, the first biological sample and the second biological sample are the same sample, which is obtained from the subject between about six weeks and about one week prior to administration of the cell therapy to a subject. In some embodiments, the first biological sample and the second biological sample are the same sample, which is obtained from the subject about three weeks prior to administration of the cell therapy to a subject.

In some embodiments, (a) the first biological sample and the second biological sample are different samples; (b) the first biological sample is obtained from the subject between about eight weeks prior and about four weeks prior to administration of the cell therapy to a subject; and (c) the second biological sample is obtained from the subject between about four weeks and about two weeks prior to administration of the cell therapy to a subject.

In some embodiments, the first biological sample and the second biological sample are obtained from the subject between about two weeks apart and about six weeks apart. In some embodiments, the first biological sample and the second biological sample are obtained from the subject about three weeks apart. In some embodiments, the second biological sample is obtained from the subject about three weeks prior to administration of the cell therapy to a subject.

In some embodiments, prior to genetic engineering, the cells of the second biological sample are incubated under stimulating conditions.

In some embodiments, the stimulating conditions include the presence of a stimulatory reagent capable of activating an intracellular signaling domains of a component of a T cell receptor (TCR) complex and an intracellular signaling domain of a costimulatory molecule. In some embodiments, the stimulatory reagent includes (i) a primary agent that binds to a member of a TCR complex; and (ii) a secondary agent that binds to a T cell costimulatory molecule. In some embodiments, the primary agent binds to CD3. In some embodiments, the costimulatory molecule is selected from CD28, CD137 (4-1BB), OX40 and ICOS. In some embodiments, the primary agent comprises an antibody or an antigen-binding fragment thereof. In some embodiments, the secondary agent comprises an antibody or an antigen-binding fragment thereof. In some embodiments, the primary agent and the secondary agent comprises an antibody or an antigen-binding fragment thereof. In some embodiments, the primary agent is an anti-CD3 antibody or an antigen-binding fragment thereof, and the secondary agent is an anti-CD28 antibody or an antigen-binding fragment thereof.

In some embodiments, the stimulating conditions include the presence of a recombinant cytokine. In some embodiments, the recombinant cytokine includes IL-2, IL-7, IL-15, or a combination thereof.

In some embodiments, following genetic engineering, the genetically engineered cells are cultivated under conditions to allow for expansion or proliferation of the engineered cells. In some embodiments, the cultivation results in at least about a 2-fold, 3-fold, 4-fold, 5-fold increase in the number of viable genetically engineered cells, compared to at the initiation of cultivation. In some embodiments, the cultivation results in at least about a 2-fold increase in the number of viable genetically engineered cells, compared to at the initiation of cultivation. In some embodiments, the cultivation results in at least about a 3-fold increase in the number of viable genetically engineered cells, compared to at the initiation of cultivation. In some embodiments, the cultivation results in at least about a 4-fold increase in the number of viable genetically engineered cells, compared to at the initiation of cultivation. In some embodiments, the cultivation results in at least about a 5-fold increase in the number of viable genetically engineered cells, compared to at the initiation of cultivation.

In some embodiments, the cell therapy is an allogeneic cell therapy. In some embodiments, the cell therapy is an allogeneic cell therapy, and the subject from whom the first and second biological samples are obtained is different than the subject to whom the cell therapy is administered. In some embodiments, the cell therapy is an autologous cell therapy. In some embodiments, the cell therapy is an autologous cell therapy, and the subject from whom the first and second biological samples are obtained is the same subject to whom the cell therapy is administered.

In some embodiments, the subject administered the cell therapy has a disease or condition. In some embodiments, the disease or condition is an infectious disease or disorder, an autoimmune disease, an inflammatory disease, or a cancer. In some embodiments, the disease or condition is an infectious disease or disorder. In some embodiments, the disease or condition is an autoimmune disease. In some embodiments, the disease or condition is an inflammatory disease. In some embodiments, the disease or condition is a cancer. In some embodiments, the disease or condition is a cancer. In some embodiments, the cancer is a leukemia, a lymphoma, or a myeloma. In some embodiments, the cancer is a leukemia. In some embodiments, the cancer is a lymphoma. In some embodiments, the cancer is a myeloma. In some embodiments, the cancer is a multiple myeloma (MM). In some embodiments, the cancer is a relapsed/refractory MM.

In some embodiments, the recombinant receptor binds to an antigen expressed by cells of the disease or condition. In some embodiments, the recombinant receptor is a T cell receptor (TCR) is a chimeric antigen receptor (CAR). In some embodiments, the recombinant receptor is a CAR.

In some embodiments, the CAR contains an extracellular antigen binding domain that binds to the antigen, a transmembrane domain, and an intracellular signaling region. In some embodiments, the intracellular signaling region contains an intracellular signaling domain of a CD3-zeta (CD3ζ) chain and a costimulatory signaling region. In some embodiments, the costimulatory signaling region contains an intracellular signaling domain of CD28, 4-1BB, or ICOS. In some embodiments, the costimulatory signaling region comprises an intracellular signaling domain of 4-1BB. In some embodiments, the transmembrane domain is or comprises a transmembrane domain from CD28 or CD8. In some embodiments, the transmembrane domain is or comprises a transmembrane domain from human CD28 or human CD8. In some embodiments, the CAR further contains an extracellular spacer between the extracellular antigen binding domain and the transmembrane domain. In some embodiments, the spacer is from CD8. In some embodiments, the spacer is a CD8-alpha hinge. In some embodiments, the transmembrane domain and the spacer are from CD8.

In some embodiments, the extracellular antigen binding domain binds to B cell maturation antigen (BCMA). In some embodiments, the extracellular antigen-binding domain comprises a variable heavy chain (VH) region. In some embodiments, the extracellular antigen-binding domain comprises a variable heavy chain (VH) region and a variable light chain (VL) region.

In some embodiments, the VH region comprises a CDR-H1, a CDR-H2, and a CDR-H3 comprising the amino acid sequences set forth in SEQ ID NOS: 189, 190, and 191, respectively; and the VL region comprises a CDR-L1, a CDR-L2, and a CDR-L3 comprising the amino acid sequences set forth in SEQ ID NOS: 192, 193, and 194, respectively; or the VH region comprises a CDR-H1, a CDR-H2, and a CDR-H3 comprising the amino acid sequences set forth in SEQ ID NOS: 173, 174 and 175, respectively; and the VL region comprises a CDR-L1, a CDR-L2, and a CDR-L3 comprising the amino acid sequences set forth in SEQ ID NOS: 183, 184 and 185, respectively. In some embodiments, the VH region comprises a CDR-H1, a CDR-H2, and a CDR-H3 comprising the amino acid sequences set forth in SEQ ID NOS: 189, 190, and 191, respectively; and the VL region comprises a CDR-L1, a CDR-L2, and a CDR-L3 comprising the amino acid sequences set forth in SEQ ID NOS: 192, 193, and 194, respectively. In some embodiments, the VH region comprises a CDR-H1, a CDR-H2, and a CDR-H3 comprising the amino acid sequences set forth in SEQ ID NOS: 173, 174 and 175, respectively; and the VL region comprises a CDR-L1, a CDR-L2, and a CDR-L3 comprising the amino acid sequences set forth in SEQ ID NOS: 183, 184 and 185, respectively.

In some embodiments, the VH region comprises an amino acid sequence set forth in SEQ ID NO: 18 and the VL region comprises the amino acid sequence set forth in SEQ ID NO: 19; or the VH region comprises an amino acid sequence set forth in SEQ ID NO: 24, and the VL region comprises the amino acid sequence set forth in SEQ ID NO: 25. In some embodiments, the VH region comprises an amino acid sequence set forth in SEQ ID NO: 18 and the VL region comprises the amino acid sequence set forth in SEQ ID NO: 19. In some embodiments, the VH region comprises an amino acid sequence set forth in SEQ ID NO: 24, and the VL region comprises the amino acid sequence set forth in SEQ ID NO: 25.

In some embodiments, the extracellular antigen-binding domain is a single chain variable fragment (scFv). In some embodiments, the scFv comprises the amino acid sequence set forth in SEQ ID NO: 213 or SEQ ID NO: 188. In some embodiments, the scFv comprises the amino acid sequence set forth in SEQ ID NO: 213. In some embodiments, the scFv comprises the amino acid sequence set forth in SEQ ID NO: 188. In some embodiments, the CAR comprises the amino acid sequence set forth in SEQ ID NO: 116 or SEQ ID NO: 124. In some embodiments, the CAR comprises the amino acid sequence set forth in SEQ ID NO: 116. In some embodiments, the CAR comprises the amino acid sequence set forth in SEQ ID NO: 124. In some embodiments, the CAR is encoded by the polynucleotide sequence set forth in SEQ ID NO: 214.

In some embodiments, the dose of the cell therapy comprises: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BClcCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells. In some embodiments, the dose of the cell therapy comprises idecabtagene vicleucel cells.

In some embodiments, the dose of the cell therapy comprises T cells expressing a chimeric antigen receptor (CAR) as present in: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells. In some embodiments, the dose of the cell therapy comprises T cells expressing a chimeric antigen receptor (CAR) as present in idecabtagene vicleucel cells.

In some embodiments, the extracellular antigen binding domain binds to CD19.

In some embodiments, the cell therapy contains CD4+ T cells and/or CD8+ T cells. In some embodiments, the dose of the cell therapy contains a defined ratio of CD4+ T cells to CD8+ T cells, which is between about 1:3 and about 3:1 or is about 1:1. In some embodiments, the dose of the cell therapy contains a defined ratio of CD4+ T cells to CD8+ T cells, which is between about 1:3 and about 3:1. In some embodiments, the dose of the cell therapy contains a defined ratio of CD4+ T cells to CD8+ T cells, which is about 1:1.

In some embodiments, the dose of the cell therapy contains between about 0.5×10⁶ and about 6×10⁸ CAR-positive T cells. In some embodiments, the dose of the cell therapy contains between about 1×10⁸ and about 6×10⁸ CAR-positive T cells. In some embodiments, the dose of the cell therapy contains between about 1.5×10⁸ and about 4.5×10⁸ CAR-positive T cells. In some embodiments, the dose of the cell therapy contains about 1.5×10⁸, 3×10⁸, or about 4.5×10⁸ CAR-positive T cells. In some embodiments, the dose of the cell therapy contains between about 0.5×10⁶ and about 10×10⁶ CAR-positive T cells.

In some embodiments, the percentage of CD28+ T cells is the percentage of total T cells that are CD28+.

Also provided herein is a composition of cells produced by any of the methods provided herein.

Also provided herein is a method of treating a subject having or suspected of having a disease or condition, the method including administering to the subject a dose of the cell therapy produced by any of the methods provided herein.

Also provided herein is use of a dose of the cell therapy produced by any of the methods provided herein for the treatment of a disease or condition in a subject. Also provided herein is use of any of the compositions provided herein for the treatment of a disease or condition in a subject. Also provided herein is a dose of the cell therapy produced by any of the methods provided herein for use in treating a disease or condition in a subject. Also provided herein is any of the compositions provided herein for use in treating a disease or condition in a subject. Also provided herein is a dose of the cell therapy produced by any of the methods provided herein for use in the manufacture of a medicament for treating a disease or condition in a subject. Also provided herein is any of the compositions provided herein for use in the manufacture of a medicament for treating a disease or condition in a subject.

Also provided herein is a method of treating a disease or condition in a human subject with a T cell therapy, the method including administering to a human subject having a disease or condition a therapeutically effective amount of a T cell therapy, wherein: (a) at least about 40% of T cells in the subject are CD28+; and (b) manufacture of the T cell therapy relies on CD28-mediated expansion of the T cells of the T cell therapy. In some embodiments, the disease or condition is a multiple myeloma. In some embodiments, T cells in the subject are peripheral T cells in the subject. In some embodiments, at least about 44%, at least about 45%, or at least about 50% of T cells in the subject are CD28+. In some embodiments, at least about 44% of T cells in the subject are CD28+. In some embodiments, at least about 44%, at least about 45%, or at least about 50% of peripheral T cells in the subject are CD28+. In some embodiments, at least about 44% of peripheral T cells in the subject are CD28+.

Also provided herein is a method of treating multiple myeloma in a human subject, the method comprising administering to a human subject having a multiple myeloma a therapeutically effective amount of a T cell therapy, wherein at least about 40% of T cells in the subject are CD28+. In some embodiments, manufacture of the T cell therapy relies on CD28-mediated expansion of the T cells of the T cell therapy. In some embodiments, T cells in the subject are peripheral T cells in the subject.

In some embodiments, T cells in the subject are peripheral T cells in the subject. In some embodiments, at least about 44%, at least about 45%, or at least about 50% of T cells in the subject are CD28+. In some embodiments, at least about 44% of T cells in the subject are CD28+. In some embodiments, at least about 44%, at least about 45%, or at least about 50% of peripheral T cells in the subject are CD28+. In some embodiments, at least about 44% of peripheral T cells in the subject are CD28+. In some embodiments, prior to administration of the T cell therapy to the subject, it has been determined that at least about 40% of T cells in the subject are CD28+. In some embodiments, prior to administration of the T cell therapy to the subject, it has been determined that at least about 40% of peripheral T cells in the subject are CD28+.

In some embodiments, the multiple myeloma is a relapsed/refractory multiple myeloma.

In some embodiments, the T cell therapy targets B cell maturation antigen (BCMA). In some embodiments, the T cell therapy is an autologous T cell therapy. In some embodiments, the T cell therapy is a chimeric antigen receptor (CAR) T cell therapy. In some embodiments, the CAR T cell therapy targets BCMA. In some embodiments, the CAR contains an extracellular antigen binding domain that binds to the antigen, a transmembrane domain, and an intracellular signaling region. In some embodiments, the intracellular signaling region contains an intracellular signaling domain of a CD3-zeta (CD3ζ) chain and a costimulatory signaling region. In some embodiments, the costimulatory signaling region contains an intracellular signaling domain of CD28, 4-1BB, or ICOS. In some embodiments, the costimulatory signaling region comprises an intracellular signaling domain of 4-1BB. In some embodiments, the transmembrane domain is or comprises a transmembrane domain from CD28 or CD8. In some embodiments, the transmembrane domain is or comprises a transmembrane domain from human CD28 or human CD8. In some embodiments, the CAR further contains an extracellular spacer between the extracellular antigen binding domain and the transmembrane domain. In some embodiments, the spacer is from CD8. In some embodiments, the spacer is a CD8-alpha hinge. In some embodiments, the transmembrane domain and the spacer are from CD8.

In some embodiments, the extracellular antigen-binding domain comprises a variable heavy chain (VH) region. In some embodiments, the extracellular antigen-binding domain comprises a variable heavy chain (VH) region and a variable light chain (VL) region.

In some embodiments, the VH region comprises a CDR-H1, a CDR-H2, and a CDR-H3 comprising the amino acid sequences set forth in SEQ ID NOS: 189, 190, and 191, respectively; and the VL region comprises a CDR-L1, a CDR-L2, and a CDR-L3 comprising the amino acid sequences set forth in SEQ ID NOS: 192, 193, and 194, respectively; or the VH region comprises a CDR-H1, a CDR-H2, and a CDR-H3 comprising the amino acid sequences set forth in SEQ ID NOS: 173, 174 and 175, respectively; and the VL region comprises a CDR-L1, a CDR-L2, and a CDR-L3 comprising the amino acid sequences set forth in SEQ ID NOS: 183, 184 and 185, respectively. In some embodiments, the VH region comprises a CDR-H1, a CDR-H2, and a CDR-H3 comprising the amino acid sequences set forth in SEQ ID NOS: 189, 190, and 191, respectively; and the VL region comprises a CDR-L1, a CDR-L2, and a CDR-L3 comprising the amino acid sequences set forth in SEQ ID NOS: 192, 193, and 194, respectively. In some embodiments, the VH region comprises a CDR-H1, a CDR-H2, and a CDR-H3 comprising the amino acid sequences set forth in SEQ ID NOS: 173, 174 and 175, respectively; and the VL region comprises a CDR-L1, a CDR-L2, and a CDR-L3 comprising the amino acid sequences set forth in SEQ ID NOS: 183, 184 and 185, respectively.

In some embodiments, the VH region comprises an amino acid sequence set forth in SEQ ID NO: 18 and the VL region comprises the amino acid sequence set forth in SEQ ID NO: 19; or the VH region comprises an amino acid sequence set forth in SEQ ID NO: 24, and the VL region comprises the amino acid sequence set forth in SEQ ID NO: 25. In some embodiments, the VH region comprises an amino acid sequence set forth in SEQ ID NO: 18 and the VL region comprises the amino acid sequence set forth in SEQ ID NO: 19. In some embodiments, the VH region comprises an amino acid sequence set forth in SEQ ID NO: 24, and the VL region comprises the amino acid sequence set forth in SEQ ID NO: 25.

In some embodiments, the extracellular antigen-binding domain is a single chain variable fragment (scFv). In some embodiments, the scFv comprises the amino acid sequence set forth in SEQ ID NO: 213 or SEQ ID NO: 188. In some embodiments, the scFv comprises the amino acid sequence set forth in SEQ ID NO: 213. In some embodiments, the scFv comprises the amino acid sequence set forth in SEQ ID NO: 188. In some embodiments, the CAR comprises the amino acid sequence set forth in SEQ ID NO: 116 or SEQ ID NO: 124. In some embodiments, the CAR comprises the amino acid sequence set forth in SEQ ID NO: 116. In some embodiments, the CAR comprises the amino acid sequence set forth in SEQ ID NO: 124. In some embodiments, the CAR is encoded by the polynucleotide sequence set forth in SEQ ID NO: 214.

In some embodiments, the dose of the cell therapy comprises: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BClcCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells. In some embodiments, the dose of the cell therapy comprises idecabtagene vicleucel cells.

In some embodiments, the dose of the cell therapy comprises T cells expressing a chimeric antigen receptor (CAR) as present in: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells. In some embodiments, the dose of the cell therapy comprises T cells expressing a chimeric antigen receptor (CAR) as present in idecabtagene vicleucel cells.

In some embodiments, the extracellular antigen binding domain binds to CD19.

Also provided herein is a method of enriching for CD28+ cells, the method including: (a) performing a first selection, the first selection comprising enriching for either of CD28+ or CD3+ cells from a biological sample comprising peripheral blood mononuclear cells (PBMCs) obtained from a subject, thereby generating an enriched cell population; and (b) performing a second selection on the cells of the enriched cell population, thereby generating a CD28+ enriched population, wherein (i) the first selection comprises enriching for CD28+ cells and the second selection comprises enriching for CD3+ cells from the enriched population; or (ii) the first selection comprises enriching for CD3+ cells and the second selection comprises enriching for CD28+ cells from the enriched cell population, wherein the CD28+ enriched population has a higher percentage of CD28+ cells than the biological sample and is enriched for CD3+ cells.

Also provided herein is a method of enriching for CD28+ cells, the method including: (a) performing a first selection, the first selection comprising enriching for CD28+ cells from a biological sample comprising peripheral blood mononuclear cells (PBMCs) obtained from a subject, thereby generating a first enriched population, the first enriched population having a higher percentage of CD28+ cells than the biological sample; (b) performing a second selection on the cells from the first enriched population, the second selection comprising enriching for one of (i) CD4+ cells and (ii) CD8+ cells from the first enriched population, the enrichment thereby generating a second enriched population enriched for the one of (i) CD4+ cells and (ii) CD8+ cells and a non-selected population; and (c) performing a third selection, the third selection comprising enriching for the other of (i) CD4+ cells and (ii) CD8+ cells from the non-selected population, the enrichment thereby generating a third enriched population enriched for the other of the (i) CD4+ cells and (ii) CD8+ cells.

In some embodiments, the method further includes combining the second enriched population and the third enriched population, thereby generating a CD28+ enriched population containing the second enriched population and the third enriched population. In some embodiments, the method further includes combining the second enriched population and the third enriched population at a ratio of between about 1:3 and about 3:1, thereby generating a CD28+ enriched population containing the second enriched population and the third enriched population. In some embodiments, the method further includes combining the second enriched population and the third enriched population at a ratio of about 1:1, thereby generating a CD28+ enriched population containing the second enriched population and the third enriched population.

In some embodiments, the second selection includes enriching for CD8+ cells. In some embodiments, the biological sample is a whole blood sample, an apheresis sample, or a leukapheresis sample. In some embodiments, the biological sample is a whole blood sample. In some embodiments, the biological sample is an apheresis sample. In some embodiments, the biological sample is a leukapheresis sample.

In some embodiments, the CD28+ enriched population contains: (i) less than about 5% CD28− T cells; (ii) CD4+ T cells, wherein at least about 95% of the CD4+ T cells are CD28+; or (iii) CD8+ T cells, wherein at least about 95% of the CD8+ T cells are CD28+. In some embodiments, at least about 95% of the CD4+ T cells of the CD28+ enriched population comprises CD28+CD4+ T cells. In some embodiments, at least about 95% of the CD8+ T cells of the CD28+ enriched population comprises CD28+CD8+ T cells. In some embodiments, at least about 95% of the CD4+ T cells and at least about 95% of the CD8+ T cells of the CD28+ enriched population comprises CD28+CD4+ T cells and CD28+CD8+ T cells, respectively. In some embodiments, at least about 95% of the CD3+ T cells of the CD28+ enriched population comprises CD28+CD3+ T cells. In some embodiments, the percentage of the CD28− cells in the CD28+ enriched population is less than about or about 35%, 30%, 20%, 10%, 5%, 1% or 0.1% of the percentage of CD28− cells in the biological sample. In some embodiments, the CD28+ enriched population comprises less than about 3%, less than about 2%, less than about 1%, less than about 0.1% or less than about 0.01% CD28− cells. In some embodiments, the CD28+ enriched population is free or is essentially free of CD28− cells. In some embodiments, the percentage of naïve-like T cells in the CD28+ enriched population is at least about 10%, 20%, 30%, 40% or 50% greater than the percentage of naïve-like T cells in the biological sample. In some embodiments, the naïve-like T cells are surface positive for one or more of markers selected from CD45RA, CD27, and CCR7.

In some embodiments, the enriching for CD28+ cells includes immunoaffinity-based selection. In some embodiments, the immunoaffinity-based selection includes contacting cells with an antibody capable of specifically binding to CD28 and recovering cells bound to the antibody. In some embodiments, the antibody is immobilized on a solid surface. In some embodiments, the solid surface is a magnetic particle. In some embodiments, the antibody is immobilized on or attached to an affinity chromatography matrix.

In some embodiments, the cells of the CD28+ enriched population are genetically engineered to express a recombinant receptor. In some embodiments, prior to genetic engineering, the cells of the CD28+ enriched population are incubated under stimulating conditions.

In some embodiments, the stimulating conditions include the presence of a stimulatory reagent capable of activating an intracellular signaling domains of a component of a T cell receptor (TCR) complex and an intracellular signaling domain of a costimulatory molecule. In some embodiments, the stimulatory reagent includes (i) a primary agent that binds to a member of a TCR complex; and (ii) a secondary agent that binds to a T cell costimulatory molecule. In some embodiments, the primary agent binds to CD3. In some embodiments, the costimulatory molecule is selected from CD28, CD137 (4-1BB), OX40 and ICOS. In some embodiments, the primary agent comprises an antibody or an antigen-binding fragment thereof. In some embodiments, the secondary agent comprises an antibody or an antigen-binding fragment thereof. In some embodiments, the primary agent and the secondary agent comprises an antibody or an antigen-binding fragment thereof. In some embodiments, the primary agent is an anti-CD3 antibody or an antigen-binding fragment thereof, and the secondary agent is an anti-CD28 antibody or an antigen-binding fragment thereof.

In some embodiments, the stimulating conditions include the presence of a recombinant cytokine. In some embodiments, the recombinant cytokine includes IL-2, IL-7, IL-15, or a combination thereof.

In some embodiments, following genetic engineering, the genetically engineered cells are cultivated under conditions to allow for expansion or proliferation of the engineered cells. In some embodiments, the cultivation results in at least about a 2-fold, 3-fold, 4-fold, 5-fold increase in the number of viable genetically engineered cells, compared to at the initiation of cultivation. In some embodiments, the cultivation results in at least about a 2-fold increase in the number of viable genetically engineered cells, compared to at the initiation of cultivation. In some embodiments, the cultivation results in at least about a 3-fold increase in the number of viable genetically engineered cells, compared to at the initiation of cultivation. In some embodiments, the cultivation results in at least about a 4-fold increase in the number of viable genetically engineered cells, compared to at the initiation of cultivation. In some embodiments, the cultivation results in at least about a 5-fold increase in the number of viable genetically engineered cells, compared to at the initiation of cultivation.

Also provided herein is a method of increasing proliferation of T cells comprising incubating a population of T cells under stimulating conditions, wherein the population of T cells comprises a percentage of CD28+ T cells above a threshold value.

Also provided herein is a method of increasing proliferation of T cells comprising: (1) selecting a biological sample comprising a population of T cells in which the percentage of CD28+ T cells in the population of T cells is above a threshold value; and (2) incubating the selected population of T cells under stimulating conditions.

In some embodiments, the population of T cells is obtained from a biological sample.

In some embodiments, the biological sample is obtained from a subject.

In some embodiments, the subject is human.

In some embodiments, the biological sample is a whole blood sample, an apheresis sample, or a leukapheresis sample.

In some embodiments, the stimulating conditions comprise the presence of a stimulatory reagent capable of activating an intracellular signaling domains of a component of a T cell receptor (TCR) complex and an intracellular signaling domain of a costimulatory molecule.

In some embodiments, the stimulatory reagent comprises (i) a primary agent that binds to a member of a TCR complex, optionally that binds to CD3; and (ii) a secondary agent that binds to a T cell costimulatory molecule, optionally wherein the costimulatory molecule is selected from CD28, CD137 (4-1BB), OX40 and ICOS.

In some embodiments, the primary agent and/or the secondary agent comprises an antibody or an antigen-binding fragment thereof.

In some embodiments, the primary agent is an anti-CD3 antibody or an antigen-binding fragment thereof, and the secondary agent is an anti-CD28 antibody or an antigen-binding fragment thereof.

In some embodiments, the stimulating conditions comprise the presence of a recombinant cytokine.

In some embodiments, the recombinant cytokine comprises IL-2, IL-7, IL-15, or a combination thereof.

In some embodiments, the method results in increased proliferation compared to a population of T cells comprising a percentage of CD28+ T cells that is not at or above the threshold value.

In some embodiments, the method results in increased proliferation compared to a population of CD28+ T cells comprising a percentage of CD28+ T cells that is below the threshold value.

In some embodiments, the threshold value is between about 30% and about 50% CD28+ T cells, or between about 35% and about 45% CD28+ T cells.

In some embodiments, the threshold value is about 40% CD28+ T cells.

In some embodiments, the increase in proliferation occurs on day 1, 2, 3, 4, or 5 after incubation.

In some embodiments, determining the percentage of CD28+ T cells comprises measuring CD28 protein.

In some embodiments, determining the percentage of CD28+ T cells comprises measuring CD28 RNA.

Also provided herein is a method of manufacturing a cell therapy, in which the method comprises (1) selecting a subject for manufacturing a cell therapy if the percentage of any of CD28+, CD45RA+, CD45RO+, CD27+, CD197+, CD4+, CD57+, CD8+, CD25+, PD1+, LAG3+, CD3+ and/or TIM3+ T cells in a first biological sample obtained from the subject is above a threshold value, wherein the first biological sample comprises T cells; and (2) genetically engineering cells of a second biological sample obtained from the subject to express a recombinant receptor, thereby generating a cell therapy comprising the genetically engineered cells, wherein the second biological sample comprises T cells.

In some embodiments, the method further comprises determining the percentage of any of CD28+, CD45RA+, CD45RO+, CD27+, CD197+, CD4+, CD57+, CD8+, CD25+, PD1+, LAG3+, CD3+ and/or TIM3+ T cells in the first biological sample.

In some embodiments, the threshold value is calculated as the percentage of T cells that are CD28+, CD45RA+, CD45RO+, CD27+, CD197+, CD4+, CD57+, CD8+, CD25+, PD1+, LAG3+, CD3+ and/or TIM3+ in the first biological sample.

In some embodiments, a subject is selected for manufacturing a cell therapy if the percentage of CD28+ T cells in a first biological sample comprising T cells obtained from the subject is above a threshold value. In some embodiments, the threshold value is between about 20% and about 40% CD28+ T cells, between about 30% and about 50% CD28+ T cells, or between about 35% and about 45% CD28+ T cells.

In some embodiments, the threshold value is about 40% CD28+ T cells.

In some embodiments, a subject is selected for manufacturing a cell therapy if the percentage of CD45RA+ T cells in a first biological sample comprising T cells obtained from the subject is above a threshold value. In some embodiments, the threshold value is between about 20% and about 40% CD45RA+ T cells, between about 30% and about 50% CD45RA+ T cells, or between about 35% and about 45% CD45RA+ T cells.

In some embodiments, the threshold value is about 40% CD45RA+ T cells.

In some embodiments, a subject is selected for manufacturing a cell therapy if the percentage of CD45RO+ T cells in a first biological sample comprising T cells obtained from the subject is above a threshold value. In some embodiments, the threshold value is between about 20% and about 40% CD45RO+ T cells, between about 30% and about 50% CD45RO+ T cells, or between about 35% and about 45% CD45RO+ T cells.

In some embodiments, the threshold value is about 40% CD45RO+ T cells.

In some embodiments, a subject is selected for manufacturing a cell therapy if the percentage of CD27+ T cells in a first biological sample comprising T cells obtained from the subject is above a threshold value. In some embodiments, the threshold value is between about 20% and about 40% CD27+ T cells, between about 30% and about 50% CD27+ T cells, or between about 35% and about 45% CD27+ T cells.

In some embodiments, the threshold value is about 40% CD27+ T cells.

In some embodiments, a subject is selected for manufacturing a cell therapy if the percentage of CD197+ T cells in a first biological sample comprising T cells obtained from the subject is above a threshold value. In some embodiments, the threshold value is between about 20% and about 40% CD197+ T cells, between about 30% and about 50% CD197+ T cells, or between about 35% and about 45% CD197+ T cells.

In some embodiments, the threshold value is about 40% CD197+ T cells.

In some embodiments, the method further comprises administering a dose of the cell therapy to the selected subject.

Also provided herein is a composition of cells that is produced by any of the methods provided herein

Also provided herein is a method of manufacturing a cell therapy in which the method comprises (1) selecting a subject for manufacturing a cell therapy if T cell expression of interleukin 2 receptor subunit alpha (IL2RA), interleukin 6 family cytokine (LIF), and/or oncostatin M (OSM) in a first biological sample obtained from the subject is above a threshold value, wherein the first biological sample comprises T cells; and (2) genetically engineering cells of a second biological sample obtained from the subject to express a recombinant receptor, thereby generating a cell therapy comprising the genetically engineered cells, wherein the second biological sample comprises T cells.

In some embodiments, the method further comprises determining T cell expression of IL2RA, LIF, and/or OSM in the first biological sample.

In some embodiments, determining expression of IL2RA, LIF, and/or OSM comprises measuring RNA expression of IL2RA, LIF, and/or OSM. In some embodiments, RNA expression is measured in a single cell of the biological sample. In some embodiments, RNA expression is measured in a plurality of cells of the biological sample.
In some embodiments, the threshold value is calculated as the average expression level of IL2RA, LIF, and/or OSM in a reference T cell or T cell population. In some embodiments, the reference T cell or T cell population is obtained from a subject that is not selected in a method of manufacturing the cell therapy. n some embodiments, the subject that is not selected in a method of manufacturing the cell therapy has slow growing T cells.

In some embodiments, the threshold value is between about 20% and about 40% IL2RA, LIF, and/or OSM expression, between about 30% and about 50% IL2RA, LIF, and/or OSM expression, or between about 35% and about 45% IL2RA, LIF, and/or OSM expression. In some embodiments, the threshold value is about 40% IL2RA, LIF, and/or OSM expression.

In some embodiments, the method further comprises administering a dose of the cell therapy to the subject.

In some embodiments, the first biological sample and the second biological sample is an apheresis sample or a leukapheresis sample.

Also provided herein is a method of manufacturing a cell therapy, in which the method comprises (1) selecting a subject for manufacturing a cell therapy if one or more genes associated with CD4 proliferating T cells, CD4 central memory T cells (TCM), CD8 naïve cells, CD4 naïve cells, CD8 TCM cells, T regulatory cells (Treg), mucosal-associated invariant T cells (MAIT), cDC2 cells, plasmablast cells, or NK proliferating cells in a first biological sample obtained from the subject is above a threshold value, wherein the first biological sample comprises T cells; and (2) genetically engineering cells of a second biological sample obtained from the subject to express a recombinant receptor, thereby generating a cell therapy comprising the genetically engineered cells, wherein the second biological sample comprises T cells. In some embodiments, the method of manufacturing a cell therapy further comprises selecting a subject for manufacturing a cell therapy if one or more genes associated with CD4 proliferating cells in the first biological sample obtained from the subject is above the threshold value. In some embodiments, the method of manufacturing a cell therapy further comprises selecting a subject for manufacturing a cell therapy if one or more genes associated with CD4 TCM in the first biological sample obtained from the subject is above the threshold value.

In some embodiments, the one or more genes associated with CD4 proliferating T cells is selected from the group consisting of MKI67, TOP2A, CD4, CCR7, NKG7, IL32, LAG3, HAVCR2, and CD3G.

In some embodiments, the one or more genes associated with CD4 TCM is selected from the group consisting of CD4, CCR7, TCF7, IL7R, IL32, and CD3G.

### BRIEF DESCRIPTION OF THE DRAWINGS

FIGS. 1A-1B show the cumulative population doubling levels (cPDL) of engineered cells. FIG. 1A shows cPDL of engineered cells in static culture derived from “average” donor peripheral blood mononuclear cell (PBMC) samples (average), as well as from two donor PBMC samples identified as “slow growing” cells (SG). FIG. 1B shows cumulative PDL for normal and slow growers in a scale down expansion model. Mean±SD for 6 lots per group are plotted. * is p<0.05 for Sidak's multiple comparisons test, **** is p<0.0001.

FIG. 2 shows the percentage of CD3+ cells among viable cells in donor PBMC samples that went on to exhibit “normal growing” rates (normal) or “slow growing” rates (SG). 3.6% was the minimum percentage of CD3+ cells in PBMC samples observed that resulted in successful manufacture of drug product.

FIG. 3 shows the distribution of CD3+CD28+ positive cells (top panel) and CD3+CD57+ cells (bottom panel) in PBMC samples from donors with “normal growing” cells (normal) and “slow growing” cells (SG).

FIGS. 4A-4B show the distribution of areas under the curve (AUC) for different cell population characteristics across PBMC samples on day 0, prior to engineering. AUC=1.0 is a perfectly predictive assay, AUC=0.5 performs no better than random guess.

FIG. 5 shows the receiver-operating character (ROC) curves for different cell population characteristics across PBMC samples on day 0, prior to engineering.

FIG. 6 shows cytokine secretion (IFN-γ, TNF, CSF2, IL-4, IL-15, IL-17A, IL-1B and OSM) in pg/10⁶ cells. Normal grower (NG) and slow grower (SG) cells were stimulated with anti-CD3 and anti-CD28 or unstimulated. Paired unstimulated and stimulated cells are connected by solid lines. * is p<0.05 for a Sidak's multiple comparisons test, ** is p<0.01, *** is p<0.001, **** is p<0.0001 and ‘ns’ is not significant.

FIG. 7 shows confluence of CD3+ stimulated cells across time (days). CD28+ and CD28− donor cells were cultured on anti-CD3 coated plates (CD3 stim CD29+ or CD3 stim CD28−) or on non-anti-CD3 coated plates (unstim CD28+ or unstim CD28−).

FIG. 8 shows confluence of CD3+/CD28+ stimulated cells across time (days). CD28+ and CD28− donor cells were cultured on anti-CD3 coated plates and CD28 coated dynabeads (CD3/CD28 stim CD28+ or CD3/CD28 stim CD28−) or were without CD3 and CD28 (unstim CD28+ or unstim CD28−).

FIG. 9 shows a multi-dimensional scaling plot in samples clustered by coordinate 1 (stimulation) and coordinate 2 (growth). Bulk RNA was analyzed.

FIGS. 10A-10B show gene expression in normal grower (NG) cells and slow grower (SG) cells when unstimulated (FIG. 10A) and when stimulated (FIG. 10B). Bulk RNA was analyzed.

FIG. 11 shows CD28 RNA expression in normal grower (NG) cells and slow grower (SG) cells. * is p<0.05 for unpaired t test. Bulk RNA was analyzed.

FIGS. 12A-12D show unbiased clustering of a combined population of normal grower (NG) cells and slow grower (SG) cells. FIG. 12A shows unbiased cell clustering. FIG. 12B shows cell atlas-driven cluster prediction. FIG. 12C shows a heatmap depicting gene signatures driving T cell subset prediction. FIG. 12D shows differential cell compositions of normal grower (“Normal”) cells and slow grower (“Slow”) cells in unstimulated and stimulated conditions by single cell RNA sequencing analysis.

FIGS. 13A-13B show predicted cell clusters between slow grower (SG) cells and normal grower (NG) cells that are unstimulated (FIG. 13A) or stimulated (FIG. 13B). Cell types include central memory T cell (TCM), mucosal-associated invariant T cell (MAIT), effector memory T cell (TEM), double-negative T cell (dnT), and hematopoietic stem/progenitor cell (HSPC).

FIG. 14 shows validation of T cell subsets by expression of identity-associated genes. Predicted cell clusters are listed along the y-axis from human cell atlas PBMC reference set. A list of genes encoding for proteins that identify immunophenotypes is listed along the x-axis. Treg=T regulatory, NK=natural killer, MAIT=Mucosal-associated invariant T cell, gdT=gamma delta T cell, dnT=double negative T cell, TEM=effector memory T cell, TCM=central memory T cell, CTL=cytotoxic lymphocyte, and 28 and 40 refer to outlier clusters 28 and 40.

### DETAILED DESCRIPTION

Provided herein are methods and compositions useful for, inter alia, selecting, isolating, enriching, stimulating, genetically engineering, and/or expanding samples, populations, or compositions of cells having a threshold percentage of CD28+ T cells.

In some aspects, provided herein are methods of selecting cells for manufacture of a cell therapy. Also provided herein are methods of manufacturing a cell therapy. In some embodiments, the methods comprise determining the percentage of CD28+ T cells in a first biological sample obtained from a subject, and selecting the subject for manufacturing a cell therapy from a second biological sample obtained from the subject if the percentage of CD28+ T cells in the first biological sample is above a threshold value. In some embodiments, the first and second biological samples comprise peripheral blood mononuclear cells (PBMCs). In some embodiments, the first and second biological samples comprise T cells. In some embodiments, the cells of the second biological sample are genetically engineered to express a recombinant receptor, thereby generating a cell therapy comprising the genetically engineered cells. In some embodiments, the cell therapy is a T cell therapy. In some embodiments, the cell therapy is an autologous cell therapy. In some embodiments, the cell therapy is an allogeneic cell therapy.

In some embodiments, the first and second biological samples are the same biological sample. In some aspects, the biological sample is an apheresis or a leukapheresis (e.g., PBMC) sample obtained from a subject. In some embodiments, if the percentage of CD28+ T cells in the first biological sample is above a threshold value (e.g., 40%), the second biological sample (i.e., the same sample) is selected for manufacture of a cell therapy, such as by genetically engineering the cells of the biological sample to express a recombinant receptor (e.g., a chimeric antigen receptor).

In some embodiments, the first and second biological samples are different biological samples. In some cases, the first biological sample is a blood or apheresis (e.g., a leukapheresis) sample obtained from the subject. In some embodiments, the second biological sample is an apheresis or a leukapheresis (e.g., PBMC) sample. In some embodiments, if the percentage of CD28+ T cells in the first biological sample is above a threshold value (e.g., 40%), a second biological sample is obtained from the subject. In some embodiments, if the percentage of CD28+ T cells in the first biological sample is above a threshold value (e.g., 40%), a second biological sample obtained from the subject is selected for genetic engineering. In some cases, if the percentage of CD28+ T cells in the first biological sample is above a threshold value (e.g., 40%), the subject is selected for manufacture of a cell therapy from cells of the second biological sample, such as by genetically engineering the cells of the second sample to express a recombinant receptor (e.g., a chimeric antigen receptor).

In some cases, the first biological sample and the second biological sample are obtained from the subject between about two weeks and about six weeks apart. In some cases, the first biological sample is obtained from the subject at a screening, and the second biological sample is obtained from the subject about three weeks prior to administration of the cell therapy to a subject. In some embodiments, the cell therapy is an autologous cell therapy. In some embodiments, the subject from whom the first and second biological samples are obtained is the same subject to whom the cell therapy is administered. In some embodiments, the cell therapy is an allogeneic cell therapy. In some embodiments, the subject from whom the first and second biological samples are obtained is different than the subject to whom the cell therapy is administered.

In some cases, if the percentage of CD28+ T cells in the first biological sample is below a threshold value (e.g., 40%), cells of the second biological sample are not predicted to yield a sufficient number of cells for a dose of a cell therapy. Thus, in some embodiments, if the percentage of CD28+ T cells in the first biological sample is below a threshold value (e.g., 40%), cells of the second biological sample are not genetically engineered to generate a cell therapy. In some embodiments, if the percentage of CD28+ T cells in the first biological sample is below a threshold value (e.g., 40%), the subject is not selected for manufacture of a cell therapy from cells of the second biological sample. In some embodiments, if the percentage of CD28+ T cells in the first biological sample is below a threshold value (e.g., 40%), a second biological sample is not obtained from the subject.

Also provided herein are methods for enriching T cells that are or include selecting, isolating, or enriching for CD28+ cells from a biological sample, such as to generate a population of enriched CD28+ cells, or a population depleted for CD28− cells. In some aspects, provided herein is a method for enriching T cells that is or includes selecting, isolating, or enriching for CD28+ T cells from a biological sample obtained from a subject, such as to generate a population of enriched CD28+ T cells, or a population depleted for CD28-T cells. In some embodiments, the enriched CD28+ cell population is genetically engineered to express a recombinant receptor (e.g., a chimeric antigen receptor).

In some embodiments, prior to genetic engineering, the cells are incubated under stimulating conditions. In some embodiments, following genetic engineering, the engineered cells are cultivated under conditions to allow for proliferation or expansion of the cells. In some embodiments, the cells proliferate or expand to exhibit a particular level of expansion, such as at least five population doublings within about 10 days of initiation of the incubating.

Particular embodiments contemplate that existing methods for generating engineered cells, e.g., engineered T cells expressing chimeric antigen receptors (CARs) may include steps, stages, or phases where populations or compositions of T cells proliferate or expand, such as to produce a dose of a cell therapy having a sufficient number of cells. However, some engineered populations or compositions may not display any proliferation or expansion, or may expand slowly, thereby requiring extra days to achieve a sufficient number of cells for a dose of an autologous cell therapy. In this way, generation of a cell therapy product by genetic engineering of a subject's cells can result in a non-conforming product because the cells fail to sufficiently proliferate or expand during manufacture. Such manufacturing failures not only deplete resources, but also fail to provide a cell therapy product for patients in need. For example, in some cases, engineered populations of T cells obtained from a subject (e.g., from a biological sample from a subject) fail to achieve at least about five population doublings within about 10 days from the initiation of incubation (i.e., activation). In some cases, engineered populations of T cells obtained from a subject (e.g., from a biological sample from a subject) fail to expand to a minimum number of cells, such as from about to about 150-540×10⁶ recombinant receptor-expressing T cells, within a certain number of days (e.g., 10 days from the initiation of incubation).

Therefore, in some cases, existing manufacturing methods are not satisfactory for producing a sufficient number of engineered cells from a biological sample obtained from a subject, thereby leading to manufacturing failures. It has been reported that manufacturing failure rates for CAR T cells products, including those currently approved, range from 2 percent to 14 percent (“Off to the CAR T Races: Bringing CAR T-Cell Therapies to Cancer Patients,” published Dec. 4, 2017, available at ashclinicalnews.org/spotlight/off-car-t-races-bringing-car-t-cell-therapies-cancer-patients). For instance, the manufacturing failure rate of KYMRIAH®, an FDA approved autologous anti-CD19 CAR T cell therapy, was reported to be approximately 9% (Seimetz et al., Cell Med. (2019) 11: 2155179018822781). Improved manufacturing methods are therefore needed to predict and select biological samples that will sufficiently expand or proliferate to produce a dose of a cell therapy having a sufficient number of cells.

In some cases, manufacturing processes for manufacturing certain CAR products also can lead to patient-to-patient variability. For instance, idecabtagene-vicleucel (ide-cel), a CAR-T cell therapy for treating refractory/relapsing multiple myeloma, utilizes ex vivo T cell expansion during lentivirus transduction to achieve the minimum cell dose for re-infusion. However, patient-to-patient variability, which carries through to the autologous cell material used to manufacture ide-cel, is believed to be a key contributor to the manufacturing outcome.

The provided methods and compositions address these issues. The provided methods and compositions are directed to, inter alia, populations of cells, e.g., populations of enriched CD28+ T cells that undergo improved or more rapid proliferation and expansion, such as during processes for manufacturing engineered T cells, as compared to compositions of cells not having a threshold percentage of CD28+ T cells or compositions of cells not enriched for CD28+ T cells. Such CD28+ T cell enriched populations can be obtained by selecting a biological sample having a percentage of CD28+ T cells above a threshold value (e.g., 40%) or by enriching a biological sample for CD28+ T cells.

CD28 (which is also known as T-cell-specific surface glycoprotein CD28 and TP44) is involved in T-cell activation, proliferation, cytokine production, and survival. In particular aspects, based on observations described herein, CD28 expression (e.g., the percentage of CD28+ cells or CD28+CD3+ cells) may identify cells with increased proliferative capacity. Particular embodiments contemplate that starting cellular material used in genetic engineering processes having a higher percentage of CD28+ T cells (e.g., above 40% CD28+ T cells) is more likely to exhibit increased proliferative capacity and expand to produce a dose of a cell therapy having a sufficient number of cells. For example, it was observed herein that the percentage of CD28+ T cells in donor samples may sensitively predict which samples will yield a sufficient number of cells for a dose of a cell therapy (150-540×10⁶ CAR+ T cells by day 10 of manufacture).

Thus, in some aspects, selection of patient samples (e.g., leukapheresis samples) in which, e.g., greater than about 40% of all T cells express CD28+, improves manufacturing success and drug product consistency by selecting for biological samples that are better poised to expand. Similarly, enriching patient samples for CD28+ T cells can improve manufacturing success and drug product consistency by selecting for cells that are better poised to expand.

In some embodiments, the methods are used in connection with a process that generates or produces genetically engineered cells that are suitable for cell therapy in a manner that may be faster and/or more efficient than alternative processes. In certain embodiments, the methods provided herein have a high rate of success for generating or producing compositions of engineered cells than what may be possible from alternative processes wherein samples and/or cells are not selected. Thus, in some aspects, the provided methods allow for the identification of which subjects will be able to provide biological samples (e.g., apheresis or leukapheresis samples) that will sufficiently expand in culture, thereby reducing the costs and time associated with manufacturing failure. Thus, in some embodiments, the provided methods can reduce manufacturing failures and/or decrease the process duration for generating a cell therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### I. POPULATIONS OF ENRICHED T CELLS (E.G. ENRICHED CD28+ T CELLS)

Provided herein are populations of enriched CD28+ T cells. In some embodiments, a population of enriched CD28+ T cells is obtained by enriching a biological sample (e.g. an apheresis or leukapheresis sample) for CD28+ T cells, such as by positive selection. In some embodiments, a population of enriched CD28+ T cells is obtained by selecting a biological sample (e.g. an apheresis or leukapheresis sample) in which the percentage of CD28+ T cells is above a threshold value. In some embodiments, the biological sample is obtained from a subject. In some embodiments, the biological sample is a second biological sample obtained from the subject. In some embodiments, the population of CD28+ enriched cells is subjected to genetic engineering, thereby generating a cell therapy. In some embodiments, the subject has a disease or condition and is a candidate for treatment with the cell therapy. In some embodiments, the T cell therapy is generated from the biological sample.

In some embodiments, the subject to whom the cell therapy is administered has a disease or condition (e.g., cancer). In some embodiments, the recombinant receptor binds to an antigen express by cells of the disease or condition.

In certain embodiments, provided herein are populations of enriched CD28+ cells, e.g. CD28+ T cells (also referred to herein as CD28+ cell populations, compositions of enriched CD28+ cells, or CD28+ cell compositions). In certain embodiments, the provided populations of enriched CD28+ cells are used in connection with methods for stimulating, activating, engineering, transducing, cultivating, or expanding T cells, e.g., T cells of or originating from a population of enriched CD28+ cells. In some embodiments, the populations of enriched CD28+ cells result from or are products of isolation, selection, or enrichment, e.g., of a biological sample or cells therein, such as a biological sample (e.g. a second biological sample) containing one or more immune cells. In certain embodiments, the population of enriched CD28+ cells is or includes viable cells, CD3+ cells, CD4+ cells, and/or CD8+ cells. In particular embodiments, the cells of the population of enriched CD28+ cells are or include viable cells, CD3+ cells, CD4+ cells, and/or CD8+ cells or a combination of any of the foregoing. In various embodiments, the cells of the population of enriched CD28+ cells are or include viable CD28+ cells, CD28+CD3+ T cells, CD28+CD4+ T cells, CD28+CD8+ T cells, or a combination of any of the foregoing.

Particular embodiments contemplate that the percentage of cells expressing CD28, e.g., percentage of CD28+ cells, in a sample, population, or composition containing cells (e.g. a PBMC sample) may be measured by any suitable known means. In some embodiments, CD28 expression is measured in a sample, population, or composition to measure, assess, or determine the amount, frequency, or percentage of CD28+ cells, e.g., CD28+ T cells in the sample, population, or composition.

In some embodiments, cell compositions (e.g., apheresis or leukapheresis samples) having a higher percentage of CD28+ cells among T cells can result in a higher percentage of cells capable of proliferative expansion, such as during methods of manufacturing genetically engineered cells. In some cases, a cell composition (e.g., apheresis or leukapheresis samples) with a high percentage of CD28− cells among T cells is associated with a reduced proliferative capacity and may result in prolonged process times, higher doublings to achieve threshold cell numbers, increased cellular differentiation and/or failure to meet a harvest criterion in a manufacturing process for producing an engineered T cell composition for cell therapy.

Also provided in some aspects are methods for identifying a population of cells capable of expansion, the method including measuring the percentage of CD28+ T cells in the population (e.g., population of T cells), wherein the population of cells is identified as capable of expansion if the percentage of CD28+ T cells is above a threshold value (i.e., a threshold percentage). In some of any such embodiments, the threshold value is a percentage that is greater than about 30%, 31%, 32%, 33%, 34%, 35%, 36%, 37%, 38%, 39%, 40%, 41%, 42%, 43%, 44%, 45%, 46%, 47%, 48%, 49%, or 50%. In some of any such embodiments, the threshold value is a percentage that is greater than between about 40% and about 50%, such as between about 40% and about 45%. In some of any such embodiments, a population that is capable of expansion expands at least about 2-fold, 4-fold, 8-fold, or 16-fold within 4, 5, 6, 7 or 8 days of cultivation under conditions that promote proliferation or expansion. In some of any such embodiments, a population that is capable of expansion expands at least about 3-fold, 4-fold, 5-fold, 6-fold, or 7-fold within about 10 days of cultivation under conditions that promote proliferation or expansion (e.g., static culture).

Also provided in some aspects are methods for determining the capacity of expansion of a population of cells, the method including measuring a value of a trait associated with CD28 expression in a population of cells, wherein the if a population of cells is determined as capable of expansion if the value of the trait is greater than about a threshold value of the trait.

In certain embodiments, negative expression, e.g., negative expression of CD28 or CD28−, is an expression equal to or less than the level of background expression, e.g., as detected using a standard technique, such as a technique involving antibody-staining. In certain embodiments, negative expression is equal to or less than the level of background expression as detected by suitable techniques for assessing protein or gene expression, such as but not limited to immunohistochemistry, immunofluorescence, or flow cytometry based techniques. In some embodiments, positive expression, e.g., of a particular protein, is or includes surface expression of the protein in an amount, level, or concentration above background. In particular embodiments, negative expression, e.g., of a particular protein, is or includes surface expression of the protein in an amount, level, or concentration at or below background.

In certain embodiments, the methods provided herein include one or more steps of assessing, measuring, determining, and/or quantifying the expression of one or more proteins or genes (e.g., CD28) in a sample, population, or composition, such as to quantify cells in the sample, composition, or population with positive or negative expression for the protein or gene (e.g., CD28). Such steps may include assessing, measuring, determining, and/or quantifying any suitable trait associated with expression, such as measuring levels of protein, surface protein, mRNA, or gene accessibility, e.g., epigenetic gene accessibility.

In some embodiments, the expression of a protein (e.g., CD28) is or includes assessing, measuring, determining, and/or quantifying a level, amount, or concentration of the protein, or a protein encoded by the gene, expressed on the surface of cells. In particular embodiments, the expression of a protein (e.g., CD28) is assessed by assessing, measuring, determining, and/or quantifying the surface expression of the protein, e.g., the level, amount, or concentration of the protein on the surface of the cells. In particular embodiments, the amount, frequency, or percentage of cells positive for surface expression of the protein, e.g., cells with surfaces having a greater amount, concentration, or density of proteins on the surface that is greater than the background signal of the technique used to measure the surface protein. In particular embodiments, the surface expression of a protein (e.g., CD28) is measured by immunohistochemistry, immunofluorescence, or flow cytometry based techniques. In some embodiments, the amount, frequency, or percentage of cells positive for surface expression of a protein is determined by a suitable known technique such as an immunohistochemistry, immunofluorescence, or flow cytometry based technique.

In particular embodiments, the amount, frequency, or percentage of cells that are negative or positive for protein expression, e.g., surface expression, in the sample, composition, or population is determined by flow cytometry. In some embodiments, the protein is CD3, CD4, CD8, CD25, CD27, CD28, CD57, CCR7, or CD45RA. In particular embodiments, the protein is CD28.

In particular embodiments, the expression of a protein (e.g., CD28) in a sample, population, or composition is or includes any suitable method for assessing, measuring, determining, and/or quantifying the level, amount, or concentration of protein. Such methods include, but are not limited to, detection with immunoassays, nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays). In some embodiments, the immunoassay is or includes methods or assays that detect proteins based on an immunological reaction, e.g., by detecting the binding of an antibody or antigen binding antibody fragment to a gene product. Immunoassays include, but are not limited to, quantitative immunocytochemistry or immunohistochemistry, ELISA (including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), enzyme immunoassay (EIA), RIA (radioimmunoassay), and SPR (surface plasmon resonance).

In certain embodiments, the expression of a protein or its corresponding gene is measured, assessed, or quantified by measuring an mRNA (or cDNA product derived from the mRNA) that encodes the protein (e.g., CD28). In particular embodiments, the amount or level of the mRNA (or corresponding cDNA) is assessed, measured, determined, and/or quantified by any suitable means (PCR), including reverse transcriptase (rt) PCR, droplet digital PCR, real-time and quantitative PCR methods (including, e.g., TAQMAN®, molecular beacon, LIGHTUP™ SCORPION™, SIMPLEPROBES®; see, e.g., U.S. Pat. Nos. 5,538,848; 5,925,517; 6,174,670; 6,329,144; 6,326,145 and 6,635,427); northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or any other known assay methods such as discussed in Shendure et al., Nat. Rev. Genet. 5:335-44 (2004) or Nowrousian, Euk. Cell 9(9): 1300-1310 (2010), including such specific platforms as HELICOS®, ROCHE® 454, ILLUMINA®/SOLEXA®, ABI SOLiD®, and POLONATOR® sequencing. In some embodiments, the expression of mRNA is determined by a next generation sequencing method such as RNA sequencing (RNA-Seq). RNA sequencing methods have been adapted for the most common DNA sequencing platforms (HiSeq systems (Illumina), 454 Genome Sequencer FLX System (Roche), Applied Biosystems SOLiD (Life Technologies), IonTorrent (Life Technologies)). These platforms require initial reverse transcription of RNA into cDNA. Conversely, the single molecule sequencer HeliScope (Helicos BioSciences) is able to use RNA as a template for sequencing.

In some embodiments, the population of enriched CD28+ cells contains, contains about, or contains greater than about 30%, 31%, 32%, 33%, 34%, 35%, 36%, 37%, 38%, 39%, 40%, 41%, 42%, 43%, 44%, 45%, 46%, 47%, 48%, 49%, or 50% CD28+ T cells. In certain embodiments, the population of enriched CD28+ cells is essentially free of CD28− cells. In particular embodiments, the population of enriched CD28+ cells contains greater than at or about 30% CD28+ cells. In particular embodiments, the population of enriched CD28+ cells contains greater than at or about 35% CD28+ cells. In particular embodiments, the population of enriched CD28+ cells contains greater than at or about 40% CD28+ cells. In particular embodiments, the population of enriched CD28+ cells contains greater than at or about 44% CD28+ cells. In particular embodiments, the population of enriched CD28+ cells contains greater than at or about 45% CD28+ cells. In particular embodiments, the population of enriched CD28+ cells contains greater than at or about 50% CD28+ cells. In some embodiments, the population of enriched CD28+ cells contains less than at or about 50% CD28− cells. In some embodiments, the population of enriched CD28+ cells contains less than at or about 55% CD28− cells. In some embodiments, the population of enriched CD28+ cells contains less than at or about 60% CD28− cells. In some embodiments, the population of enriched CD28+ cells contains less than at or about 65% CD28− cells.

In certain embodiments, the population of enriched CD28+ cells contains, contains about, or contains at least at or about 30%, 35%, 40%, 45%, 50%, 55%, 60%, 65%, 70%, 75%, 80%, 85%, 90%, 95%, 97%, 98%, 99%, 99.5%, 99.9%, 99.99%, or 100% or about 100% CD28+ cells. In certain embodiments, the population of enriched CD28+ cells contains at least at or about 30% CD28+ cells. In certain embodiments, the population of enriched CD28+ cells contains at least at or about 35% CD28+ cells. In certain embodiments, the population of enriched CD28+ cells contains at least at or about 40% CD28+ cells. In certain embodiments, the population of enriched CD28+ cells contains at least at or about 45% CD28+ cells. In certain embodiments, the population of enriched CD28+ cells contains at least at or about 50% CD28+ cells. In certain embodiments, the population of enriched CD28+ cells contains at least at or about 55% CD28+ cells. In certain embodiments, the population of enriched CD28+ cells contains at least at or about 60% CD28+ cells. In various embodiments, the population of enriched CD28+ cells contains at least at or about 70%, 80%, 90%, 95%, or 99% CD28+ cells. In particular embodiments, all or essentially all of the cells of the population of enriched CD28+ cells are CD28+ cells.

In some embodiments, the population of enriched CD28+ cells contains, contains about, or contains at least at or about 30%, 35%, 40%, 45%, 50%, 55%, 60%, 65%, 70%, 75%, 80%, 85%, 90%, 95%, 97%, 98%, 99%, 99.5%, 99.9%, 99.99%, or 100% or about 100% CD28+CD3+ cells. In certain embodiments, the population of enriched CD28+ cells contains at least at or about 40% CD28+CD3+ cells. In certain embodiments, the population of enriched CD28+ cells contains at least at or about 45% CD28+CD3+ cells. In certain embodiments, the population of enriched CD28+ cells contains at least at or about 50% CD28+CD3+ cells. In various embodiments, the population of enriched CD28+ cells contains at least at or about 70%, 80%, 90%, 95%, or 99% CD28+CD3+ cells. In particular embodiments, all or essentially all of the cells of the population of enriched CD28+ cells are CD28+CD3+ cells.

In particular embodiments, the cells of the population of enriched CD28+ cells are or include viable cells. In some embodiments, cell viability is assessed with an assay that may include, but is not limited to, dye uptake assays (e.g., calcein AM assays), XTT cell viability assays, and dye exclusion assays (e.g., trypan blue, Eosin, or propidium dye exclusion assays). In particular embodiments, a viable cell has negative expression of one or more apoptotic markers, e.g., Annexin V or active Caspase 3. In some embodiments, the viable cell is negative for the expression of one or more apoptosis marker that may include, but are not limited to, a caspase or an active caspase, e.g., caspase 2, caspase 3, caspase 6, caspase 7, caspase 8, caspase 9, or caspase 10, Bcl-2 family members, e.g., Bax, Bad, and Bid, Annexin V, or TUNEL staining. In particular embodiments, the viable cells are active caspase 3 negative. In certain embodiments, the viable cells are Annexin V negative. In certain embodiments, at least at or about 60%, at least at or about 65%, at least at or about 70%, at least at or about 75%, at least at or about 80%, at least at or about 85%, at least at or about 90%, at least at or about 95%, at least at or about 97%, at least at or about 99%, at least at or about 99.5%, at least at or about 99.9%, or 100% or about 100% of the cells of the population of enriched CD28+ cells are viable cells. In some embodiments, the viable cells are or include viable CD3+, viable CD4+, viable CD8+, viable CD28+, viable CD28+CD3+, viable CD28+CD4+, or viable CD28+CD8+ T cells, or a combination of any of the foregoing. In some embodiments, the viable cells are active caspase 3 negative. In particular embodiments, the viable cells are Annexin V negative.

In certain embodiments, the population of enriched CD28+ cells contains, contains about, or contains at least at or about 30%, 35%, 40%, 45%, 50%, 55%, 60%, 65%, 70%, 75%, 80%, 85%, 90%, 95%, 97%, 98%, 99%, 99.5%, 99.9%, 99.99%, or 100% or about 100% CD28+CD4+ cells. In certain embodiments, the population of enriched CD28+ cells contains at least at or about 30% CD28+CD4+ T cells. In certain embodiments, the population of enriched CD28+ cells contains at least at or about 40% CD28+CD4+ cells. In various embodiments, the population of enriched CD28+ cells contains at least at or about 45% CD28+CD4+ cells. In particular embodiments, all or essentially all of the cells of the population of enriched CD28+ cells are CD28+CD4+ cells. In some embodiments, the population of enriched CD28+ cells contains, contains about, or contains at least at or about 30%, 35%, 40%, 45%, 50%, 55%, 60%, 65%, 70%, 75%, 80%, 85%0, 90%, 95%, 97%, 98%, 99%, 99.5%, 99.9%, 99.99%, or 100% or about 100% CD28+CD8+ cells. In certain embodiments, the population of enriched CD28+ cells contains at least at or about 30% CD28+CD8+ cells. In certain embodiments, the population of enriched CD28+ cells contains at least at or about 40% CD28+CD8+ cells. In various embodiments, the population of enriched CD28+ cells contains at least at or about 45% CD28+CD8+ cells. In particular embodiments, all or essentially all of the cells of the population of enriched CD28+ cells are CD28+CD8+ cells. In some embodiments, the population of enriched CD28+ cells contains, contains about, or contains at least at or about 30%, 35%, 40%, 45%, 50%, 55%, 60%, 65%, 70%, 75%, 80%, 85%, 90%, 95%, 97%, 98%, 99%, 99.5%, 99.9%, 99.99%, or 100% or about 100% CD28+CD3+ cells. In certain embodiments, the population of enriched CD28+ cells contains at least at or about 30% CD28+CD3+ cells. In certain embodiments, the population of enriched CD28+ cells contains at least at or about 40% CD28+CD3+ cells. In various embodiments, the population of enriched CD28+ cells contains at least at or about 45% CD28+CD3+ cells. In particular embodiments, all or essentially all of the cells of the population of enriched CD28+ cells are CD28+CD3+ cells.

In particular embodiments, a percentage of the cells of the population of enriched CD28+ cells are naïve-like cells (e.g., naïve-like T cells). In some embodiments, a naïve-like T cell is a T cell that is positive for the expression of one or more markers that indicate that the cell is naïve and/or is a naïve-like cell. In certain embodiments, a naïve-like T cell is a cell that is positive for the expression of a marker that is associated with a naïve or naïve-like state in T cells. In particular embodiments, a naïve-like T cell is a T cell that is negative for the expression of one or more markers that indicates that the cell is not naïve and/or is a not a naïve-like cell. In certain embodiments, a non-naïve or non-naïve-like state in a T cells includes, for example but not limited to, effector T (T_{EFF}) cells, memory T cells, central memory T cells (T_{CM}), effector memory T (T_{EM}) cells, and combinations thereof.

In some embodiments, a naïve-like T cell is positive for the expression of at least one or more markers that indicate that the cell is naïve and/or is a naïve-like cell, and/or is associated with a naïve or naïve-like state in T cells. In some embodiments, the markers are expressed on the cell surface. In certain embodiments, the naïve-like T cell is negative for the expression of at least one or more markers that indicate that the cell is non-naïve and/or is a non-naïve-like cell, and/or is associated with a non-naïve or non-naïve-like state in T cells.

Markers that indicate that the T cell is naïve and/or is a naïve-like T cell, and/or are associated with a naïve or naïve-like state in T cells include, but are not limited to, CD27, CD45RA, CD62L, and/or CCR7. In some embodiments, the naïve-like T cell, e.g., the naïve-like CD4+ and/or CD8+ T cell, is positive for expression of CD27, CD45RA, and/or CCR7. In certain embodiments, the naïve-like T cell is positive for the surface expression of one or more of CD27, CD45RA, and/or CCR7. In some embodiments, the naïve-like T cell, e.g., the naïve-like CD4+ and/or CD8+ T cell, is negative for expression of CD62L. In some embodiments, the naïve-like T cell, e.g., the naïve-like CD3+ T cell, is negative for expression of CD62L. In some embodiments, the naïve-like T cell, e.g., the naïve-like CD3+, CD4+, and/or CD8+ T cell, is negative for expression of CD62L. Markers that indicate that the cell is a non-naïve and/or is a non-naïve-like T cell, and/or are associated with a non-naïve or non-naïve-like state in T cells include, but are not limited to, CD25, CD45RO, CD56, KLRG1, and/or CD95. In some embodiments, the naïve-like T cell, e.g., a naïve-like CD4+ and/or CD8+ T cell, is negative for expression of CD25, CD45RO, CD56, and/or KLRG1. In particular embodiments, the naïve-like T cell, e.g., a naïve-like CD4+ and/or CD8+ T cell, has low expression of a marker associated with non-naïve or non-naïve-like cells. In some embodiments, the naïve-like T cell, e.g., a naïve-like CD3+ T cell, is negative for expression of CD25, CD45RO, CD56, and/or KLRG1. In particular embodiments, the naïve-like T cell, e.g., a naïve-like CD3+ T cell, has low expression of a marker associated with non-naïve or non-naïve-like cells. In particular embodiments, the naïve-like T cell has low expression of CD95. In certain embodiments, the naïve-like T cell is negative for the surface expression of one or more of CD25, CD45RO, CD56, and/or KLRG1.

In some embodiments, low expression of a marker associated with non-naïve or non-naïve-like cells is or includes at least 10%, at least 20%, at least 30%, at least 40%, at least 50%, at least 60%, at least 70%, at least 80%, at least 90%, at least 95%, or at least 99% less expression than the expression of the marker in a cell that is a non-naïve-like cells, and/or a cell that is positive for one or more markers that indicate that the cell is a non-naïve and/or is a non-naïve-like T cell, and/or are associated with a non-naïve or non-naïve-like state in T cells. In certain embodiments, low expression of a marker associated with non-naïve or non-naïve-like cells is or includes at least 10%, at least 20%, at least 30%, at least 40%, at least 50%, at least 60%, at least 70%, at least 80%, at least 90%, at least 95%, or at least 99% less expression than the expression of the marker in an effector T (T_{EFF}) cell, a memory T cell, a central memory T cell (T_{CM}), and/or an effector memory T (T_{EM}) cell.

In some embodiments, markers that indicate that the cell is a non-naïve and/or is a non-naïve-like T cell, and/or are associated with a non-naïve or non-naïve-like state in T cells include one or more cytokines. For example, in certain embodiments, a non-naïve or non-naïve-like T cell is negative for the expression and/or the production of one or more of IL-2, IFN-γ, IL-4, and IL-10. In some embodiments, the one or more cytokines are secreted. In particular embodiments, the one or more cytokines are expressed internally by the non-naïve-like T cells, for example, during or after treatment with an agent that prevents, inhibits, or reduces secretion.

In certain embodiments, a naïve-like T cell, e.g., a naïve-like CD28+ T cell, is positive for the expression, e.g., surface expression, of CD45RA and CCR7. In particular embodiments, a naïve-like CD4+ T cell is positive for the expression, e.g., surface expression, of CD45RA and CCR7. In some embodiments, a naïve-like CD8+ T cell is positive for the expression, e.g., surface expression, of CD45RA and CCR7. In some embodiments, a naïve-like CD3+ T cell is positive for the expression, e.g., surface expression, of CD45RA and CCR7. In particular embodiments, a naïve-like T cell is positive for the expression, e.g., surface expression, of CD45RA, CD27, and CCR7 and is negative for the expression, e.g., surface expression of CD45RO. In particular embodiments, a naïve-like CD4+ T cell is positive for the expression, e.g., surface expression, of CD45RA, CD27, and CCR7 and is negative for the expression, e.g., surface expression of CD45RO. In some embodiments, a naïve-like CD8+ T cell is positive for the expression, e.g., surface expression, of CD45RA, CD27, and CCR7 and is negative for the expression, e.g., surface expression of CD45RO. In some embodiments, a naïve-like CD3+ T cell is positive for the expression, e.g., surface expression, of CD45RA, CD27, and CCR7 and is negative for the expression, e.g., surface expression of CD45RO.

In certain embodiments, the population of enriched CD28+ cells contains, contains about, or contains at least at or about 10%, 15%, 20%, 25%, 30%, 35%, 40%, 45%, or 50% T cells that are positive for CD25 expression. In various embodiments, the population of enriched CD28+ cells contains, contains about, or contains at least at or about 10%, 15%, 20%, 25%, 30%, 35%, 40%, 45%, or 50% CD28+CD25+ T cells. In various embodiments, the population of enriched CD28+ cells contains between or between about 10% and 60%, 20% and 50%, or 25% and 40% CD25+ T cells, each inclusive. In some embodiments, the population of enriched CD28+ cells contains between or between about 10% and 60%, 20% and 50%, or 25% and 40% CD28+CD25+ T cells, each inclusive.

In certain embodiments, the population of enriched CD28+ cells contains, contains about, or contains at least at or about 10%, 15%, 20%, 25%, 30%, 35%, 40%, 45%, or 50% T cells that are positive for CD27 expression. In various embodiments, the population of enriched CD28+ cells contains, contains about, or contains at least at or about 10%, 15%, 20%, 25%, 30%, 35%, 40%, 45%, or 50% CD28+CD27+ T cells. In various embodiments, the population of enriched CD28+ cells contains between or between about 10% and 60%, 20% and 50%, or 25% and 40% CD27+ T cells, each inclusive. In some embodiments, the population of enriched CD28+ cells contains between or between about 10% and 60%, 20% and 50%, or 25% and 40% CD28+CD27+ T cells, each inclusive. In certain embodiments, the population of enriched CD28+ T cells contains at least at or about 25% CD27+ T cells.

In some embodiments, the population of enriched CD28+ cells contains, contains about, or contains at least at or about 10%, 15%, 20%, 25%, 30%, 35%, 40%, 45%, or 50% T cells that are positive for CCR7 expression. In certain embodiments, the population of enriched CD28+ cells contains, contains about, or contains at least at or about 5%, 10%, 15%, 20%, 25%, 30%, 35%, 40%, 45%, or 50% CD28+ CCR7+ T cells. In come embodiments, the population of enriched CD28+ cells contains between or between about at or about 5% and at or about 50%, at or about 5% and at or about 35%, or at or about 10% and at or about 25% CCR7+ T cells, each inclusive. In particular embodiments, the population of enriched CD28+ cells contains between or between about at or about 5% and at or about 50%, at or about 5% and at or about 35%, or at or about 10% and at or about 25% CD28+ CCR7+ T cells, each inclusive. In some embodiments, the population of enriched CD28+ cells contains at least at or about 25% CCR7+ T cells.

In some embodiments, the population of enriched CD28+ cells contains, contains about, or contains at least at or about 10%, 15%, 20%, 25%, 30%, 35%, 40%, 45%, or 50% T cells that are positive for CD45RA expression. In certain embodiments, the population of enriched CD28+ cells contains, contains about, or contains at least at or about 5%, 10%, 15%, 20%, 25%, 30%, 35%, 40%, 45%, or 50% CD28+CD45RA+ T cells. In come embodiments, the population of enriched CD28+ cells contains between or between about at or about 5% and at or about 50%, at or about 5% and at or about 35%, or at or about 10% and at or about 25% CD45RA+ T cells, each inclusive. In particular embodiments, the population of enriched CD28+ cells contains between or between about at or about 5% and at or about 50%, at or about 5% and at or about 35%, or at or about 10% and at or about 25% CD28+CD45RA+ T cells, each inclusive. In some embodiments, the population of enriched CD28+ T cells contains at least at or about 25% CD45RA+ T cells.

In some embodiments, the percentage of the naïve-like cells in the enriched CD28+ population is at least at or about 10%, 20%, 30%, 40%, or 50% greater than at or about the percentage of naïve-like cells in the biological sample. In some embodiments, the percentage of one or more of CD25+ T cells, CD27+ T cells, CCR7+ T cells, or CD45RA+ T cells in the enriched CD28+ population is at least at or about 10%, 20%, 30%, 40%, or 50% greater than at or about the percentage of the respective cells in the biological sample. In some embodiments, the CD28+ enriched population comprises at least at or about 15%, 20%, 25%, 30%, 35%, or 40% CD27+ T cells. In some embodiments, the CD28+ enriched population comprises at least at or about 10%, 15%, 20%, 25%, 30%, 35%, 40%, 50%, 60%, 70% or 80% CD27+CD28+ T cells. In some embodiments, the CD28+ enriched population comprises at least at or about 35% or 45% CD27+CD28+ T cells. In some embodiments, the enriched CD28+ population comprises at least at or about 10%, 15%, 20%, or 25% CCR7+ T cells.

In other aspects, provided herein are populations of cells enriched for non-CD28 markers. In some embodiments, provided herein are populations of cells enriched in CD45RA+, CD45RO+, CD27+, CD197+, CD4+, CD57+, CD8+, CD25+, PD1+, LAG3+, CD3+ and/or TIM3+ T cells. In some embodiments, a population of enriched CD45RA+, CD45RO+, CD27+, CD197+, CD4+, CD57+, CD8+, CD25+, PD1+, LAG3+, CD3+ and/or TIM3+ T cells is obtained by enriching a biological sample (e.g. an apheresis or leukapheresis sample) for CD45RA+, CD45RO+, CD27+, CD197+, CD4+, CD57+, CD8+, CD25+, PD1+, LAG3+, CD3+ and/or TIM3+ T cells, such as by positive selection. In some embodiments, a population of enriched CD45RA+, CD45RO+, CD27+, CD197+, CD4+, CD57+, CD8+, CD25+, PD1+, LAG3+, CD3+ and/or TIM3+ T cells is obtained by selecting a biological sample (e.g. an apheresis or leukapheresis sample) in which the percentage of CD45RA+, CD45RO+, CD27+, CD197+, CD4+, CD57+, CD8+, CD25+, PD1+, LAG3+, CD3+ and/or TIM3+ T cells is above a threshold value. In some embodiments, the biological sample is obtained from a subject. In some embodiments, the biological sample is a second biological sample obtained from the subject. In some embodiments, the population of CD45RA+, CD45RO+, CD27+, CD197+, CD4+, CD57+, CD8+, CD25+, PD1+, LAG3+, CD3+ and/or TIM3+ enriched cells is subjected to genetic engineering, thereby generating a cell therapy. In some embodiments, the subject has a disease or condition and is a candidate for treatment with the cell therapy. In some embodiments, the T cell therapy is generated from the biological sample.

In some embodiments, the subject to whom the cell therapy is administered has a disease or condition (e.g., cancer). In some embodiments, the recombinant receptor binds to an antigen express by cells of the disease or condition.

In certain embodiments, provided herein are populations of enriched CD45RA+, CD45RO+, CD27+, CD197+, CD4+, CD57+, CD8+, CD25+, PD1+, LAG3+, CD3+ and/or TIM3+ cells, e.g. CD45RA+, CD45RO+, CD27+, CD197+, CD4+, CD57+, CD8+, CD25+, PD1+, LAG3+, CD3+ and/or TIM3+ T cells (also referred to herein as CD45RA+, CD45RO+, CD27+, CD197+, CD4+, CD57+, CD8+, CD25+, PD1+, LAG3+, CD3+ and/or TIM3+ cell populations, compositions of enriched CD45RA+, CD45RO+, CD27+, CD197+, CD4+, CD57+, CD8+, CD25+, PD1+, LAG3+, CD3+ and/or TIM3+ cells, or CD45RA+, CD45RO+, CD27+, CD197+, CD4+, CD57+, CD8+, CD25+, PD1+, LAG3+, CD3+ and/or TIM3+ cell compositions). In certain embodiments, the provided populations of enriched CD45RA+, CD45RO+, CD27+, CD197+, CD4+, CD57+, CD8+, CD25+, PD1+, LAG3+, CD3+ and/or TIM3+ cells are used in connection with methods for stimulating, activating, engineering, transducing, cultivating, or expanding T cells, e.g., T cells of or originating from a population of enriched CD45RA+, CD45RO+, CD27+, CD197+, CD4+, CD57+, CD8+, CD25+, PD1+, LAG3+, CD3+ and/or TIM3+ cells. In some embodiments, the populations of enriched CD45RA+, CD45RO+, CD27+, CD197+, CD4+, CD57+, CD8+, CD25+, PD1+, LAG3+, CD3+ and/or TIM3+ cells result from or are products of isolation, selection, or enrichment, e.g., of a biological sample or cells therein, such as a biological sample (e.g. a second biological sample) containing one or more immune cells. In certain embodiments, the population of enriched CD45RA+, CD45RO+, CD27+, CD197+, CD4+, CD57+, CD8+, CD25+, PD1+, LAG3+, CD3+ and/or TIM3+ cells is or includes viable cells, CD3+ cells, CD4+ cells, and/or CD8+ cells. In particular embodiments, the cells of the population of enriched CD45RA+, CD45RO+, CD27+, CD197+, CD4+, CD57+, CD8+, CD25+, PD1+, LAG3+, CD3+ and/or TIM3+ cells are or include viable cells, CD3+ cells, CD4+ cells, and/or CD8+ cells or a combination of any of the foregoing. In various embodiments, the cells of the population of enriched CD45RA+, CD45RO+, CD27+, CD197+, CD4+, CD57+, CD8+, CD25+, PD1+, LAG3+, CD3+ and/or TIM3+ cells are or include: (i) viable CD45RA+, CD45RO+, CD27+, CD197+, CD4+, CD57+, CD8+, CD25+, PD1+, LAG3+, CD3+ and/or TIM3+ cells; (ii) viable CD45RA+CD3+ T cells, CD45RO+CD3+ T cells, CD27+CD3+ T cells, CD197+CD3+ T cells, CD4+CD3+ T cells, CD57+CD3+ T cells, CD8+CD3+ T cells, CD25+CD3+ T cells, PD1+CD3+ T cells, LAG3+CD3+ T cells, and/or TIM3+CD3+ T cells; (iii) viable CD45RA+CD4+ T cells, CD45RO+CD4+ T cells, CD27+CD4+ T cells, CD197+CD4+ T cells, CD57+CD4+ T cells, CD8+CD4+ T cells, CD25+CD4+ T cells, PD1+CD4+ T cells, LAG3+CD4+ T cells, CD3+CD4+ T cells and/or TIM3+CD4+ T cells; or (iv) viable CD45RA+CD8+ T cells, CD45RO+CD8+ T cells, CD27+CD8+ T cells, CD197+CD8+ T cells, CD4+CD8+ T cells, CD57+CD8+ T cells, CD25+CD8+ T cells, PD1+CD8+ T cells, LAG3+CD8+ T cells, CD3+CD8+ T cells and/or TIM3+CD8+ T cells, or a combination of any of the foregoing.

In some embodiments, the non-CD28 markers comprise interleukin 2 receptor subunit alpha (IL2RA), interleukin 6 family cytokine (LIF), and/or oncostatin M (OSM). In some embodiments, provided herein are populations of enriched IL2RA, LIF, and/or OSM expressing T cells. In some embodiments, the population of enriched IL2RA, LIF, and/or OSM expressing T cells is obtained from a first biological sample. In some embodiments, RNA expression of IL2RA, LIF, and/or OSM is measured in a single cell or a plurality of cells in the first biological sample. In some embodiments, a population of enriched IL2RA, LIF, and/or OSM expressing T cells comprises a percentage of IL2RA, LIF, and/or OSM expressing T cells that is above a threshold value. In some embodiments, the biological sample is obtained from a subject. In some embodiments, the biological sample is a second biological sample obtained from the subject. In some embodiments, the population of populations of enriched IL2RA, LIF, and/or OSM expressing T cells is subjected to genetic engineering, thereby generating a cell therapy. In some embodiments, the subject has a disease or condition and is a candidate for treatment with the cell therapy. In some embodiments, the T cell therapy is generated from the biological sample. In some embodiments, the subject to whom the cell therapy is administered has a disease or condition (e.g., cancer). In some embodiments, the recombinant receptor binds to an antigen express by cells of the disease or condition.

In some embodiments, the non-CD28 markers comprise MKI67, TOP2A, CD4, CCR7, NKG7, IL32, LAG3, HAVCR2, CD3G, TCF7, and/or IL7R. In some embodiments, provided herein are populations of cells enriched in MKI67, TOP2A, CD4, CCR7, NKG7, IL32, LAG3, HAVCR2, CD3G, TCF7, and/or IL7R expressing T cells. In some embodiments, the population of enriched MKI67, TOP2A, CD4, CCR7, NKG7, IL32, LAG3, HAVCR2, CD3G, TCF7, and/or IL7R expressing T cells is obtained from a first biological sample. In some embodiments, RNA expression of MKI67, TOP2A, CD4, CCR7, NKG7, IL32, LAG3, HAVCR2, CD3G, TCF7, and/or IL7R is measured in a single cell or a plurality of cells in the first biological sample. In some embodiments, a population of enriched MKI67, TOP2A, CD4, CCR7, NKG7, IL32, LAG3, HAVCR2, CD3G, TCF7, and/or IL7R expressing T cells comprises a percentage of MKI67, TOP2A, CD4, CCR7, NKG7, IL32, LAG3, HAVCR2, CD3G, TCF7, and/or IL7R expressing T cells that is above a threshold value. In some embodiments, the biological sample is obtained from a subject. In some embodiments, the biological sample is a second biological sample obtained from the subject. In some embodiments, the population of populations of enriched MKI67, TOP2A, CD4, CCR7, NKG7, IL32, LAG3, HAVCR2, CD3G, TCF7, and/or IL7R expressing T cells is subjected to genetic engineering, thereby generating a cell therapy. In some embodiments, the subject has a disease or condition and is a candidate for treatment with the cell therapy. In some embodiments, the T cell therapy is generated from the biological sample. In some embodiments, the subject to whom the cell therapy is administered has a disease or condition (e.g., cancer). In some embodiments, the recombinant receptor binds to an antigen express by cells of the disease or condition.

In certain embodiments, provided herein are enriched populations of CD28+ T cells obtained by the selection methods described in Section II or Section III. In some embodiments, provided herein are enriched populations of non-CD28 or CD28− T cells obtained by the selection method described in Section II or Section III.

### II. SELECTION OF ENRICHED T CELL POPULATIONS (E.G. ENRICHED CD28+ T CELL POPULATIONS)

Provided herein are methods of selecting enriched CD28+ T cell populations for genetic engineering. In some embodiments, a population of enriched CD28+ T cells is obtained by selecting a biological sample (e.g. an apheresis sample or a leukapheresis sample) for genetic engineering. In some embodiments, the biological sample is obtained from a subject. In some embodiments, the biological sample is a second biological sample obtained from a subject. In some embodiments, the percentage of CD28+ T cells in the biological sample is above a threshold value. In some embodiments, the biological sample comprises PBMCs. In some embodiments, the biological sample comprises T cells. In some embodiments, the biological sample is an apheresis sample. In some embodiments, the biological sample is a leukapheresis sample.

In some embodiments, a first biological sample is obtained from a subject. In some embodiments, the first biological sample comprises T cells. In some embodiments, a second biological sample is obtained from the subject. In some embodiments, the second biological sample comprises T cells. In some embodiments, the first biological sample and the second biological sample are the same sample. In some embodiments, if the percentage of CD28+ T cells in the first biological sample is above a threshold value, the second biological sample is selected for genetic engineering (a selected enriched CD28+ cell population). In some embodiments, the percentage of CD28+ T cells is the percentage of T cells that are CD28+.

In some embodiments, a first biological sample is obtained from a subject. In some embodiments, a second biological sample is obtained from the subject. In some embodiments, the first biological sample and the second biological sample are different samples. In some embodiments, the first and second biological sample are obtained from the subject between about two weeks apart and about eight weeks apart. In some embodiments, the first and second biological sample are obtained from the subject about three weeks apart. In some embodiments, the first biological sample is a whole blood sample or an apheresis sample. In some embodiments, the second biological sample is an apheresis sample or a leukapheresis sample. In some embodiments, if the percentage of CD28+ T cells among T cells in the first biological sample is above a threshold value, the second biological sample is selected for genetic engineering (a selected enriched CD28+ cell population).

In some embodiments, a selected enriched CD28+ cell population is subjected to one or more steps to achieve genetic engineering, such as to produce cells expressing a recombinant receptor (e.g., a chimeric antigen receptor). In some embodiments, a selected population of enriched CD28+ cells is predicted to expand and/or proliferate sufficiently to achieve a threshold number of population doublings or number of cells within a particular time period. For example, in some cases, a selected population of enriched CD28+ cells is predicted to exhibit at least 3, at least 4, at least 5, at least 6, or at least 6 population doublings within about 7, about 8, about 9, about 10, about 11, or about 12 days of cultivation under conditions that promote proliferation or expansion. In some embodiments, a selected population of enriched CD28+ cells is predicted to exhibit at least 3, at least 4, at least 5, at least 6, or at least 6 population doublings within about 10 days of cultivation under conditions that promote proliferation or expansion. In some embodiments, a selected population of enriched CD28+ cells is predicted to exhibit at least about 5 population doublings within about 10 days of cultivation under conditions that promote proliferation or expansion.

In some embodiments, a CD28+ enriched cell population is obtained by selecting a biological sample (e.g., an apheresis sample or a leukapheresis sample) comprising at or above a threshold value (i.e., a number or percentage) of CD28+ T cells among all T cells in the biological sample. In some embodiments, a biological sample (e.g. an apheresis sample or a PBMC leukapheresis sample) is assessed for the number or percentage of CD28+ T cells among all T cells in the biological sample, and a sample having greater than or equal to the threshold number or threshold percentage of CD28+ T cells is selected for genetic engineering.

In some embodiments, a CD28+ enriched cell population is obtained by selecting a biological sample (e.g., an apheresis sample or a leukapheresis sample) having at or above a threshold percentage of CD28+ T cells among all T cells in the biological sample. In some embodiments, a biological sample (e.g. an apheresis sample or a PBMC sample) is assessed for the percentage of CD28+ T cells among all T cells in the biological sample, and a sample having greater than or equal to the threshold percentage of CD28+ T cells is selected for genetic engineering.

In some embodiments, the threshold value (i.e., percentage) is about 30%, 31%, 32%, 33%, 34%, 35%, about 36%, about 37%, about 38%, about 39%, about 40%, about 41%, about 42%, about 43%, about 44%, about 45%, about 46%, about 47%, about 48%, about 49%, or about 50%. In some embodiments, the threshold percentage is between about 35% and about 50%. In some embodiments, the threshold percentage is between about 40% and about 45%. In some embodiments, the threshold percentage is about 40%. In some embodiments, the threshold percentage is about 41%. In some embodiments, the threshold percentage is about 42%. In some embodiments, the threshold percentage is about 43%. In some embodiments, the threshold percentage is about 44%. In some embodiments, the threshold percentage is about 45%.

Thus, in some embodiments, if a biological sample (e.g. an apheresis sample or a leukapheresis sample) has a percentage of CD28+ T cells among all T cells in the biological sample of between about 35% and about 50%, such as between about 40% and about 45%, the biological sample is selected for genetic engineering. Thus, in some embodiments, if a biological sample (e.g. an apheresis sample or a leukapheresis sample) has a percentage of CD28+ T cells among all T cells in the biological sample of between about 40% and about 50%, such as between about 42% and about 48%, the biological sample is selected for genetic engineering.

In some embodiments, a CD28+ enriched cell population is obtained by selecting a biological sample (e.g., an apheresis sample or a leukapheresis sample) having at or above a threshold value (i.e., a number or percentage) of CD3+CD28+ cells, e.g. among all T cells in the biological sample. In some embodiments, a biological sample (e.g. an apheresis sample or a leukapheresis sample) is assessed for the number or percentage of CD3+CD28+ cells, and a sample having greater than or equal to the threshold number or threshold percentage of CD3+CD28+ cells is selected for genetic engineering.

In some embodiments, a CD28+ enriched cell population is obtained by selecting a biological sample (e.g., an apheresis sample or a leukapheresis sample) comprising at or above a threshold percentage of CD3+CD28+ cells, e.g. among all T cells in the biological sample. In some embodiments, a biological sample (e.g. an apheresis sample or a leukapheresis sample) is assessed for the percentage of CD3+CD28+ cells, and a sample having greater than or equal to the threshold percentage of CD3+CD28+ cells is selected for genetic engineering.

In some embodiments, the threshold percentage is about 35%, about 36%, about 37%, about 38%, about 39%, about 40%, about 41%, about 42%, about 43%, about 44%, about 45%, about 46%, about 47%, about 48%, about 49%, or about 50%. In some embodiments, the threshold percentage is between about 35% and about 50%. In some embodiments, the threshold percentage is between about 40% and about 45%. In some embodiments, the threshold percentage is about 40%. In some embodiments, the threshold percentage is about 41%. In some embodiments, the threshold percentage is about 42%. In some embodiments, the threshold percentage is about 43%. In some embodiments, the threshold percentage is about 44%. In some embodiments, the threshold percentage is about 45%. In some embodiments, the threshold percentage is about 46%. In some embodiments, the threshold percentage is about 47%. In some embodiments, the threshold percentage is about 48%. In some embodiments, the threshold percentage is about 49%. In some embodiments, the threshold percentage is about 50%.

Thus, in some embodiments, if a biological sample (e.g. an apheresis sample or a leukapheresis sample) has a percentage of CD3+CD28+ cells, e.g. among all T cells in the biological sample, of between about 35% and about 50%, such as between about 40% and about 45%, the biological sample is selected for genetic engineering.

In some embodiments, a CD28+ enriched cell population is obtained by selecting a biological sample (e.g., an apheresis sample or a leukapheresis sample) having at or above a threshold value (i.e. a number or percentage) of CD4+CD28+ cells, e.g. among all CD4+ T cells in the biological sample. In some embodiments, a biological sample (e.g. an apheresis sample or a leukapheresis sample) is assessed for the number or percentage of CD4+CD28+ cells, e.g. among all CD4+ T cells in the biological sample, and a sample having greater than or equal to the threshold number or threshold percentage of CD4+CD28+ cells is selected for genetic engineering.

In some embodiments, a CD28+ enriched cell population is obtained by selecting a biological sample (e.g., an apheresis sample or a leukapheresis sample) comprising at or above a threshold percentage of CD4+CD28+ cells, e.g. among all CD4+ T cells in the biological sample. In some embodiments, a biological sample (e.g. an apheresis sample or a leukapheresis sample) is assessed for the percentage of CD4+CD28+ cells, e.g. among all CD4+ T cells in the biological sample, and a sample having greater than or equal to the threshold percentage of CD4+CD28+ cells is selected for genetic engineering.

In some embodiments, the threshold percentage is about 35%, about 36%, about 37%, about 38%, about 39%, about 40%, about 41%, about 42%, about 43%, about 44%, about 45%, about 46%, about 47%, about 48%, about 49%, or about 50%. In some embodiments, the threshold percentage is between about 35% and about 50%. In some embodiments, the threshold percentage is between about 40% and about 45%. In some embodiments, the threshold percentage is about 40%. In some embodiments, the threshold percentage is about 41%. In some embodiments, the threshold percentage is about 42%. In some embodiments, the threshold percentage is about 43%. In some embodiments, the threshold percentage is about 44%. In some embodiments, the threshold percentage is about 45%. In some embodiments, the threshold percentage is about 46%. In some embodiments, the threshold percentage is about 47%. In some embodiments, the threshold percentage is about 48%. In some embodiments, the threshold percentage is about 49%. In some embodiments, the threshold percentage is about 50%.

Thus, in some embodiments, if a biological sample (e.g. an apheresis sample or a leukapheresis sample) has a percentage of CD4+CD28+ cells, e.g. among all CD4+ T cells in the biological sample, of between about 35% and about 50%, such as between about 40% and about 45%, the biological sample is selected for genetic engineering.

In some embodiments, a CD28+ enriched cell population is obtained by selecting a biological sample (e.g., an apheresis sample or a leukapheresis sample) having at or above a threshold value (i.e. a number or percentage) of CD8+CD28+ cells, e.g. among all CD8+ T cells in the biological sample. In some embodiments, a biological sample (e.g. an apheresis sample or a leukapheresis sample) is assessed for the number or percentage of CD8+CD28+ cells, e.g. among all CD8+ T cells in the biological sample, and a sample having greater than or equal to the threshold number or threshold percentage of CD8+CD28+ cells is selected for genetic engineering.

In some embodiments, a CD28+ enriched cell population is obtained by selecting a biological sample (e.g., an apheresis sample or a leukapheresis sample) comprising at or above a threshold percentage of CD8+CD28+ cells, e.g. among all CD8+ T cells in the biological sample. In some embodiments, a biological sample (e.g. an apheresis sample or a leukapheresis sample) is assessed for the percentage of CD8+CD28+ cells, e.g. among all CD8+ T cells in the biological sample, and a sample having greater than or equal to the threshold percentage of CD8+CD28+ cells is selected for genetic engineering.

In some embodiments, the threshold percentage is about 35%, about 36%, about 37%, about 38%, about 39%, about 40%, about 41%, about 42%, about 43%, about 44%, about 45%, about 46%, about 47%, about 48%, about 49%, or about 50%. In some embodiments, the threshold percentage is between about 35% and about 50%. In some embodiments, the threshold percentage is between about 40% and about 45%. In some embodiments, the threshold percentage is about 40%. In some embodiments, the threshold percentage is about 41%. In some embodiments, the threshold percentage is about 42%. In some embodiments, the threshold percentage is about 43%. In some embodiments, the threshold percentage is about 44%. In some embodiments, the threshold percentage is about 45%. In some embodiments, the threshold percentage is about 46%. In some embodiments, the threshold percentage is about 47%. In some embodiments, the threshold percentage is about 48%. In some embodiments, the threshold percentage is about 49%. In some embodiments, the threshold percentage is about 50%.

Thus, in some embodiments, if a biological sample (e.g. an apheresis sample or a leukapheresis sample) has a percentage of CD8+CD28+ cells, e.g. among all CD8+ T cells in the biological sample of between about 35% and about 50%, such as between about 40% and about 45%, the biological sample is selected for genetic engineering.

In some embodiments, the first biological sample is obtained from the subject between about 8 weeks and about 3 weeks prior to treatment of a subject with a cell therapy. In some embodiments, the first biological sample is obtained from the subject about 8 weeks prior to treatment of the subject with a cell therapy. In some embodiments, the first biological sample is obtained from the subject about 7 weeks prior to treatment of a subject with a cell therapy. In some embodiments, the first biological sample is obtained from the subject about 6 weeks prior to treatment of a subject with a cell therapy. In some embodiments, the first biological sample is obtained from the subject about 5 weeks prior to treatment of a subject with a cell therapy. In some embodiments, the first biological sample is obtained from the subject about 4 weeks prior to treatment of a subject with a cell therapy. In some embodiments, the first biological sample is obtained from a subject about 3 weeks prior to treatment of the subject with a cell therapy. In some embodiments, the cell therapy is an autologous cell therapy. In some embodiments, the subject from whom the first and second biological samples are obtained is the same subject that is administered the cell therapy. In some embodiments, the cell therapy is an allogeneic cell therapy. In some embodiments, the subject from whom the first and second biological samples are obtained is different than the subject that is administered the cell therapy.

In some embodiments, the second biological sample is obtained from the subject between about 6 weeks and about 1 week prior to treatment of a subject with a cell therapy. In some embodiments, the second biological sample is obtained from the subject about 6 weeks prior to treatment of a subject with a cell therapy. In some embodiments, the second biological sample is obtained from the subject about 5 weeks prior to treatment of a subject with a cell therapy. In some embodiments, the second biological sample is obtained from a subject about 6 weeks prior to treatment of the subject with a cell therapy. In some embodiments, the second biological sample is obtained from the subject about 3 weeks prior to treatment of a subject with a cell therapy. In some embodiments, the second biological sample is obtained from the subject about 2 weeks prior to treatment of a subject with a cell therapy. In some embodiments, the second biological sample is obtained from the subject about 1 week prior to treatment of a subject with a cell therapy. In some embodiments, the cell therapy is an autologous cell therapy. In some embodiments, the subject from whom the first and second biological samples are obtained is the same subject that is administered the cell therapy. In some embodiments, the cell therapy is an allogeneic cell therapy. In some embodiments, the subject from whom the first and second biological samples are obtained is different than the subject that is administered the cell therapy.

In some embodiments, the first and second biological samples are obtained from the subject between about six weeks apart and about two weeks apart. In some embodiments, the first and second biological samples are obtained from the subject about three weeks apart.

In some embodiments, the T cell therapy is generated from the second biological sample. In some embodiments, cells of the second biological sample are genetically engineered to express a recombinant receptor, thereby generating the cell therapy.

In other aspects, provided herein are methods of selecting enriched cells for non-CD28 markers. Provided herein are methods of selecting enriched CD45RA+, CD45RO+, CD27+, CD197+, CD4+, CD57+, CD8+, CD25+, PD1+, LAG3+, CD3+ and/or TIM3+ T cell populations for genetic engineering. In some embodiments, a population of enriched CD45RA+, CD45RO+, CD27+, CD197+, CD4+, CD57+, CD8+, CD25+, PD1+, LAG3+, CD3+ and/or TIM3+ T cells is obtained by selecting a biological sample (e.g. an apheresis sample or a leukapheresis sample) for genetic engineering. In some embodiments, the biological sample is obtained from a subject. In some embodiments, the biological sample is a second biological sample obtained from a subject. In some embodiments, the percentage of CD45RA+, CD45RO+, CD27+, CD197+, CD4+, CD57+, CD8+, CD25+, PD1+, LAG3+, CD3+ and/or TIM3+ T cells in the biological sample is above a threshold value. In some embodiments, the biological sample comprises PBMCs. In some embodiments, the biological sample comprises T cells. In some embodiments, the biological sample is an apheresis sample. In some embodiments, the biological sample is a leukapheresis sample.

Provided herein are methods of selecting enriched IL2RA, LIF, and/or OSM expressing T cell populations for genetic engineering. In some embodiments, a population of enriched IL2RA, LIF, and/or OSM expressing T cells is obtained by selecting a biological sample (e.g. an apheresis sample or a leukapheresis sample) for genetic engineering. In some embodiments, the biological sample is obtained from a subject. In some embodiments, the biological sample is a second biological sample obtained from a subject. In some embodiments, the percentage of IL2RA, LIF, and/or OSM expressing T cells in the biological sample is above a threshold value. In some embodiments, the biological sample comprises PBMCs. In some embodiments, the biological sample comprises T cells. In some embodiments, the biological sample is an apheresis sample. In some embodiments, the biological sample is a leukapheresis sample.

Provided herein are methods of selecting enriched MKI67, TOP2A, CD4, CCR7, NKG7, IL32, LAG3, HAVCR2, CD3G, TCF7, and/or IL7R expressing T cell populations for genetic engineering. In some embodiments, a population of enriched MKI67, TOP2A, CD4, CCR7, NKG7, IL32, LAG3, HAVCR2, CD3G, TCF7, and/or IL7R expressing T cells is obtained by selecting a biological sample (e.g. an apheresis sample or a leukapheresis sample) for genetic engineering. In some embodiments, the biological sample is obtained from a subject. In some embodiments, the biological sample is a second biological sample obtained from a subject. In some embodiments, the percentage of MKI67, TOP2A, CD4, CCR7, NKG7, IL32, LAG3, HAVCR2, CD3G, TCF7, and/or IL7R expressing T cells in the biological sample is above a threshold value. In some embodiments, the biological sample comprises PBMCs. In some embodiments, the biological sample comprises T cells. In some embodiments, the biological sample is an apheresis sample. In some embodiments, the biological sample is a leukapheresis sample.

In certain embodiments, provided herein are methods of selecting and enriching the CD28+ T cells described in Section III. In certain embodiments, provided herein are methods of selecting and enriching the non-CD28 or CD28− T cells described in Section III.

### III. SELECTION OF T CELLS POSITIVE FOR A MARKER (E.G. CD28+) AND/OR DEPLETION OF T CELLS NEGATIVE FOR A MARKER (E.G. CD28−)

Provided herein are methods of selecting for CD28+ T cells and/or depleting CD28− T cells from a biological sample (e.g. an apheresis or a leukapheresis sample), thereby generating an enriched CD28+ cell population. In some embodiments, the biological sample is a second biological sample obtained from a subject. In some embodiments, cells of the enriched CD28+ cell population are genetically engineered, such as to produce cells expressing a recombinant receptor (e.g. a chimeric antigen receptor). In some embodiments, the genetically engineered cells are a cell therapy. In some embodiments, the cell therapy is an autologous cell therapy. In some embodiments, the cell therapy is an allogeneic cell therapy.

In some embodiments, the methods comprise selecting for CD28+ T cells from a biological sample (e.g. an apheresis or a leukapheresis sample). In some embodiments, the methods comprise depleting CD28− T cells from a biological sample (e.g. an apheresis or a leukapheresis sample). In some embodiments, an enriched CD28+ cell population is predicted to expand and/or proliferate sufficiently to achieve a threshold number of population doublings or number of cells within a particular time period. For example, in some cases, an enriched CD28 cell population cells is predicted to exhibit at least 3, at least 4, at least 5, at least 6, or at least 6 population doublings within about 7, about 8, about 9, about 10, about 11, or about 12 days of cultivation under conditions that promote proliferation or expansion. In some embodiments, an enriched CD28+ cell population is predicted to exhibit at least 3, at least 4, at least 5, at least 6, or at least 6 population doublings within about 10 days of cultivation under conditions that promote proliferation or expansion. In some embodiments, an enriched CD28+ cell population is predicted to exhibit at least about 5 population doublings within about 10 days of cultivation under conditions that promote proliferation or expansion.

In some embodiments, the population of enriched CD28+ cells is obtained from a biological sample. In particular embodiments, the population of enriched CD28+ cells is selected, isolated, or enriched from a biological sample. In particular embodiments, CD28− T cells are removed, separated, or depleted from a biological sample. In certain embodiments, at least 25%, 30%, 35%, 40%, 45%, 50%, 55%, 60%, 65%, 70%, 75%, 80%, 85%, 90%, 95%, 99%, or 99.9% CD28− cells are removed, separated, or depleted from the biological sample. In some embodiments, the biological sample is a leukapheresis sample.

In particular embodiments, subsets of cells, e.g., subsets of T cells, are selected, isolated, or enriched from the biological sample prior to selecting, isolating, or enriching CD28+ T cells from the biological sample. In some embodiments, subsets of cells, e.g., T cells are selected, isolated, or enriched from the population of enriched CD28+ T cells.

In some embodiments, the population of enriched CD28+ cells contains, contains about, or contains less than 50%, 45%, 40%, 35%, 30%, 25%, 20%, 15%, 10%, 5%, 1%, 0.1%, 0.01%, or 0.0010% of the CD28− cells of the biological sample, e.g., prior the selection, isolation, or enrichment. In particular embodiments, the population of enriched CD28+ cells contains, contains about, or contains less than 20% of the CD28− cells of the biological sample. In certain embodiments, the population of enriched CD28+ cells contains, contains about, or contains less than 5% of the CD28− cells of the biological sample. In some embodiments, the population of enriched CD28+ cells contains, contains about, or contains less than 1% of the CD28− cells of the biological sample e.g., prior the selection, isolation, or enrichment. In various embodiments, the population of enriched CD28+ cells contains, contains about, or contains less than 0.10% of the CD28− cells of the biological sample. In particular embodiments, the population of enriched CD28+ cells contains, contains about, or contains less than 0.010% of the CD28-cells of the biological sample. In some embodiments, the percentage of the CD28− cells in the depleted population is less than at or about 35%, 30%, 20%, 10%, 5%, 1%, or 0.1% of the percentage of CD28− cells in the biological sample. In some embodiments, the depleted population comprises less than at or about 3%, less than at or about 2%, less than at or about 1%, less than at or about 0.1%, or less than at or about 0.01% CD28− cells. In some embodiments, the depleted population is free or is essentially free of CD28− cells.

In particular embodiments, the cells of the population of enriched CD28+ cells are less differentiated than the cells of the biological sample, e.g., prior the selection, isolation, or enrichment. In certain embodiments, the population of enriched CD28+ cells contains a greater percentage of naïve-like cells than the biological sample. In certain embodiments, the population of enriched CD28+ cells includes, includes about, or includes at least at or about 25%, 30%, 35%, 40%, 45%, 50%, 55%, 60%, 65%, 70%, 75%, 80%, 85%, 90%, 95%, 100%, 125%, 150%, 1-fold, 2-fold, 3-fold, 4-fold, 5-fold, or 10 fold more naïve-like cells than the biological sample e.g., prior the selection, isolation, or enrichment.

In some embodiments, naïve-like cells include naïve T cells or central memory T cells. In some embodiments, naïve-like cells can include cells positive or expressing high levels of one or more surface markers, e.g., CD62L+, CCR7+, CD27+, CD127+, CD4+, CD8+, CD45RA+, and/or CD45RO+ T cells. In some aspects, the cells are CD27+. In some aspects, the cells are CCR7+. In particular aspects, CCR7 is expressed by naïve or naïve-like T cells (e.g. CCR7+CD45RA+ or CCR7+CD27+) and central memory T cells (CCR7+CD45RA−). In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+ CCR7+, where the cells are CD45RA+ or CD45RA−. In certain embodiments, naïve-like T cells or the T cells that are surface positive for a marker expressed on naïve-like T cells are CD62L-CCR7+. In certain embodiments, naïve-like cells include cells at an early stage of differentiation (e.g., cells that are CCR7+CD27+).

In certain embodiments, central memory T cells may include cells in various differentiation states and may be characterized by positive or high expression (e.g., surface expression) of certain cell markers and/or negative or low expression (e.g., surface expression) of other cell markers. In some aspects, less differentiated cells, e.g., central memory cells, are longer lived and exhaust less rapidly, thereby increasing persistence and durability. In some aspects, a responder to a cell therapy, such as a CAR-T cell therapy, has increased expression of central memory genes. See, e.g., Fraietta et al. (2018) Nat Med. 24(5):563-571. In some aspects, central memory T cells are characterized by positive or high expression of CD45RO, CD62L, CCR7, CD28, CD3, and/or CD127. In some aspects, central memory T cells are characterized by negative or low expression of CD45RA and/or granzyme B. In certain embodiments, central memory T cells or the T cells that are surface positive for a marker expressed on central memory T cells are CCR7+CD45RA−.

In particular embodiments, the population of enriched CD28+ cells includes, includes about, or includes at least at or about 25%, 30%, 35%, 40%, 45%, 50%, 55%, 60%, 65%, 70%, 75%, 80%, 85%, 90%, 95%, 100%, 125%, 150%, 1-fold, 2-fold, 3-fold, 4-fold, 5-fold, or 10 fold more CD27+ T cells than the biological sample e.g., prior the selection, isolation, or enrichment. In various embodiments, the population of enriched CD28+ cells includes, includes about, or includes at least at or about 25%, 30%, 35%, 40%, 45%, 50%, 55%, 60%, 65%, 70%, 75%, 80%, 85%, 90%, 95%, 100%, 125%, 150%, 1-fold, 2-fold, 3-fold, 4-fold, 5-fold, or 10 fold more CD25+ T cells than the biological sample e.g., prior the selection, isolation, or enrichment. In certain embodiments, the population of enriched CD28+ cells includes, includes about, or includes at least at or about 25%, 30%, 35%, 40%, 45%, 50%, 55%, 60%, 65%, 70%, 75%, 80%, 85%, 90%, 95%, 100%, 125%, 150%, 1-fold, 2-fold, 3-fold, 4-fold, 5-fold, or 10 fold more CCR7+ T cells than the biological sample e.g., prior the selection, isolation, or enrichment. In certain embodiments, the population of enriched CD28+ cells includes, includes about, or includes at least at or about 25%, 30%, 35%, 40%, 45%, 50%, 55%, 60%, 65%, 70%, 75%, 80%, 85%, 90%, 95%, 100%, 125%, 150%, 1-fold, 2-fold, 3-fold, 4-fold, 5-fold, or 10 fold more CD45RA+ cells than the biological sample e.g., prior the selection, isolation, or enrichment.

In certain embodiments, T cells, e.g., CD3+ T cells, are selected, isolated, or enriched from the biological sample prior to selecting, isolating, or enriching CD28+ T cells from the biological sample. In some embodiments, T cells, e.g., CD3+ T cells, are selected, isolated, or enriched from the population of enriched CD28+ T cells. In particular embodiments, selecting, isolating or enriching T cells, e.g. CD3+ T cells, involves positive selection of the cells from the sample.

In some embodiments, CD28− T cells are selected, isolated, or enriched from a sample, cell composition, or cell population, thereby producing isolated or selected CD28− T cells and a population of enriched CD28+ T cells. In certain embodiments, CD28− T cells are selected, isolated, or enriched from a biological sample, thereby producing isolated or selected CD28− T cells and a population of enriched CD28+ T cells. In certain embodiments, CD3+ T cells are enriched, selected, or isolated from the population of enriched CD28+ T cells, thereby generating a population of enriched CD28+CD3+ T cells

In certain embodiments, subsets of T cells, e.g., CD4+ or CD8+ T cells, are selected, isolated, or enriched from the biological sample prior to selecting, isolating, or enriching CD28+ T cells from the biological sample. In some embodiments, subsets of T cells, e.g., CD4+ or CD8+ T cells, are selected, isolated, or enriched from the population of enriched CD28+ T cells. In particular embodiments, the selecting, isolating or enriching a subset of T cells, e.g. CD4+ or CD8+ T cells, involves positive selection of the cells from the sample.

In some embodiments, CD28− T cells are selected, isolated, or enriched from a sample, cell composition, or cell population, thereby producing isolated or selected CD28− T cells and a population of enriched CD28+ cells, e.g., T cells. In certain embodiments, CD28− T cells are selected, isolated, or enriched from a biological sample, thereby producing isolated or selected CD28− T cells and a population of enriched CD28+ cells T cells. In particular embodiments, CD4+ T cells are enriched, selected, or isolated from the population of enriched CD28+ T cells, thereby generating a population of enriched CD28+CD4+ T cells and a non-selected population enriched for CD28+ cells. In certain embodiments, CD8+ T cells are enriched, selected, or isolated from the population of enriched CD28+ cells, thereby generating a population of enriched CD28+CD8+ T cells and a non-selected population of enriched CD28+ T cells. In certain embodiments, CD8+ T cells are enriched, selected, or isolated from the non-selected population of enriched CD28+ T cells, thereby generating a population of enriched CD28+CD8+ T cells. In particular embodiments, CD4+ T cells are enriched, selected, or isolated from the non-selected population of enriched CD28+ T cells, thereby generating a population of enriched CD28+CD4+ T cells.

In particular embodiments, CD4+ T cells are enriched, selected, or isolated from the population of enriched CD28+ T cells, thereby generating a population of enriched CD28+CD4+ T cells and a non-selected population enriched for CD28+ T cells, and then CD8+ T cells are enriched, selected, or isolated from the non-selected population of enriched CD28+ T cells, thereby generating a population of enriched CD28+CD8+ T cells. In various embodiments, CD4+ T cells are enriched, selected, or isolated from the population of enriched CD28+ T cells, thereby generating a population of enriched CD28+CD4+ T cells and a non-selected population enriched for CD28+ T cells, and then CD8+ T cells are enriched, selected, or isolated from the non-selected population of enriched CD28+ T cells, thereby generating a population of enriched CD28+CD8+ T cells.

In particular embodiments, (1) CD4+ T cells are enriched, selected, or isolated from a biological sample, thereby generating a population of enriched CD4+ T cells and a non-selected population enriched for CD4− cells; (2) CD8+ T cells are enriched, selected, or isolated from the non-selected population of enriched CD4− cells, thereby generating a population of enriched CD8+ T cells; and (3) CD28− T cells are depleted from the enriched CD4+ and CD8+ T cell populations, generating populations of enriched CD28+CD4+ and CD28+CD8+ T cells. In particular embodiments, (1) CD8+ T cells are enriched, selected, or isolated from a biological sample, thereby generating a population of enriched CD8+ T cells and a non-selected population enriched for CD8− cells; (2) CD4+ T cells are enriched, selected, or isolated from the non-selected population of enriched CD4− cells, thereby generating a population of enriched CD4+ T cells; and (3) CD28− T cells are depleted from the enriched CD4+ and CD8+ T cell populations, generating populations of enriched CD28+CD4+ and CD28+CD8+ T cells.

In particular embodiments, CD4+ T cells are enriched, selected, or isolated from a biological sample, thereby generating an enriched population of CD4+ T cells, and then CD28− T cells are removed from the enriched population of CD4+ T cells, thereby generating a population of enriched CD28+CD4+ T cells. In particular embodiments, CD8+ T cells are enriched, selected, or isolated from a biological sample, thereby generating an enriched population of CD8+ T cells, and then CD28− T cells are removed from the enriched population of CD8+ T cells, thereby generating a population of enriched CD28+CD8+ T cells.

In some embodiments, the one or more populations enriched CD28+ T cells are frozen, e.g., cryopreserved and/or cryoprotected, after isolation, selection and/or enrichment. In particular embodiments, a population of enriched CD28+CD4+ T cells are frozen, e.g., cryopreserved and/or cryoprotected, after isolation, selection and/or enrichment. In certain embodiments, a population of enriched CD28+CD8+ T cells are frozen, e.g., cryopreserved and/or cryoprotected, after isolation, selection and/or enrichment. In certain embodiments, a population of enriched CD28+CD3+ T cells are frozen, e.g., cryopreserved and/or cryoprotected, after isolation, selection and/or enrichment. In some embodiments, the one or more populations of enriched T cells are frozen e.g., cryopreserved and/or cryoprotected, prior to any steps of incubating, activating, stimulating, engineering, transducing, transfecting, cultivating, expanding, harvesting, and/or formulating the population of cells. In particular embodiments, a population of enriched CD28+CD4+ T cells are frozen e.g., cryopreserved and/or cryoprotected, prior to any steps of incubating, activating, stimulating, engineering, transducing, transfecting, cultivating, expanding, harvesting, and/or formulating the population of cells. In some embodiments, a population of enriched CD28+CD8+ T cells are frozen e.g., cryopreserved and/or cryoprotected, prior to any steps of incubating, activating, stimulating, engineering, transducing, transfecting, cultivating, expanding, harvesting, and/or formulating the population of cells. In some embodiments, a population of enriched CD28+CD3+ T cells are frozen e.g., cryopreserved and/or cryoprotected, prior to any steps of incubating, activating, stimulating, engineering, transducing, transfecting, cultivating, expanding, harvesting, and/or formulating the population of cells. In particular embodiments, the one or more cryoprotected input compositions are stored, e.g., at or at about −80° C., for between 12 hours and 7 days, between 24 hours and 120 hours, or between 2 days and 5 days. In particular embodiments, the one or more cryoprotected input compositions are stored at or at about −80° C., for an amount of time of less than 10 days, 9 days, 8 days, 7 days, 6 days, or 5 days, 4 days, 3 days, 2 days, or 1 day. In some embodiments, the one or more cryoprotected input compositions are stored at or at about −70° C. or −80° C. for less than 3 days, such as for about 2 days.

In some embodiments, “depleting” or “removing” when referring to one or more particular cell type or cell population, refers to decreasing the number or percentage of the cell type or population, e.g., compared to the total number of cells in or volume of the composition, or relative to other cell types, such as by negative selection based on markers expressed by the population or cell, or by positive selection based on a marker not present on the cell population or cell to be depleted. In general, the terms depleting or removing does not require complete removal of the cell, cell type, or population from the composition.

In some embodiments, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. In general, the term enriching does not require complete removal of other cells, cell type, or populations from the composition and does not require that the cells so enriched be present at or even near 100% in the enriched composition.

In some aspects, cell populations or cell compositions obtained from a subject, such as a human subject, for cell therapy, e.g., adoptive cell therapy, can exhibit low growth or slow growth, such that they do not reach (e.g., no growth) the threshold for harvesting cells (e.g., harvest criterion) for generating a therapeutic composition, or do not reach the threshold for harvesting cells (e.g., harvest criterion) for generating a therapeutic composition within a specific period of time (e.g., slow growth). In some aspects, some of such cell populations can contain a high percentage of CD28− T cells, such as a percentage of CD28− T cells above a threshold value. In other aspects, cell populations or cell compositions obtained from a subject, such as a human subject, for cell therapy, e.g., adoptive cell therapy, can exhibit improved growth compared to the populations exhibiting no growth or slow growth. In some aspects, such cell populations or cell compositions can contain a low percentage of CD28− T cells, such as a percentage of CD28− T cells less than a threshold value. In some aspects, cell populations or cell compositions that exhibit improved growth can exhibit phenotypes or express markers associated with naïve-like or central memory-like phenotypes, such as CD27+ and/or CCR7+. In some embodiments, the provided methods are based on observations that there is variability or heterogeneity in CD28+ T cell expression among T cells in a biological sample (e.g. leukapheresis or apheresis sample) from human subjects, which, in some aspects, can results in variability in the proliferation and/or expansion of engineered T cell compositions produced for use in adoptive cell therapy from a plurality of different subjects, even using the same manufacturing process. In particular embodiments, the provided methods control for or reduce such variability by selecting, isolating, or enriching CD28+ T cells from a biological sample, such as by removing, separating, or depleting CD28− T cells from the biological sample. Such cells can then be used in processes to engineer or manufacture cells for cell therapy to minimize variability among products, while also improving particular product attributes and features such as the ability to proliferate and/or expand, thereby resulting in a cell therapy product having a sufficient number of cells.

In some embodiments, the methods comprise selecting for CD45RA+, CD45RO+, CD27+, CD197+, CD4+, CD57+, CD8+, CD25+, PD1+, LAG3+, CD3+ and/or TIM3+ T cells from a biological sample (e.g. an apheresis or a leukapheresis sample). In some embodiments, the methods comprise depleting CD45RA−, CD45RO−, CD27−, CD197−, CD4−, CD57−, CD8−, CD25−, PD1−, LAG3−, CD3− and/or TIM3− T cells from a biological sample (e.g. an apheresis or a leukapheresis sample). In some embodiments, an enriched CD45RA+, CD45RO+, CD27+, CD197+, CD4+, CD57+, CD8+, CD25+, PD1+, LAG3+, CD3+ and/or TIM3+ cell population is predicted to expand and/or proliferate sufficiently to achieve a threshold number of population doublings or number of cells within a particular time period.

In some embodiments, provided herein are methods of selecting for IL2RA, LIF and/or OSM expressing T cells and/or depleting IL2RA, LIF and/or OSM non-expressing T cells from a biological sample (e.g. an apheresis or a leukapheresis sample), thereby generating an IL2RA, LIF and/or OSM expressing T cell population. In some embodiments, the biological sample is a second biological sample obtained from a subject. In some embodiments, cells of the IL2RA, LIF and/or OSM expressing T cell population are genetically engineered, such as to produce cells expressing a recombinant receptor (e.g. a chimeric antigen receptor). In some embodiments, the genetically engineered cells are a cell therapy. In some embodiments, the cell therapy is an autologous cell therapy. In some embodiments, the cell therapy is an allogeneic cell therapy.

In some embodiments, the methods comprise selecting for IL2RA, LIF and/or OSM expressing T cells from a biological sample (e.g. an apheresis or a leukapheresis sample). In some embodiments, the methods comprise depleting IL2RA, LIF and/or OSM non-expressing T cells from a biological sample (e.g. an apheresis or a leukapheresis sample). In some embodiments, an IL2RA, LIF and/or OSM expressing T cell population is predicted to expand and/or proliferate sufficiently to achieve a threshold number of population doublings or number of cells within a particular time period.

In some embodiments, provided herein are methods of selecting for MKI67, TOP2A, CD4, CCR7, NKG7, IL32, LAG3, HAVCR2, CD3G, TCF7, and/or IL7R expressing T cells and/or depleting MKI67, TOP2A, CD4, CCR7, NKG7, IL32, LAG3, HAVCR2, CD3G, TCF7, and/or IL7R non-expressing T cells from a biological sample (e.g. an apheresis or a leukapheresis sample), thereby generating an MKI67, TOP2A, CD4, CCR7, NKG7, IL32, LAG3, HAVCR2, CD3G, TCF7, and/or IL7R expressing T cell population. In some embodiments, the biological sample is a second biological sample obtained from a subject. In some embodiments, cells of the MKI67, TOP2A, CD4, CCR7, NKG7, IL32, LAG3, HAVCR2, CD3G, TCF7, and/or IL7R expressing T cell population are genetically engineered, such as to produce cells expressing a recombinant receptor (e.g. a chimeric antigen receptor). In some embodiments, the genetically engineered cells are a cell therapy. In some embodiments, the cell therapy is an autologous cell therapy. In some embodiments, the cell therapy is an allogeneic cell therapy.

In some embodiments, the methods comprise selecting for MKI67, TOP2A, CD4, CCR7, NKG7, IL32, LAG3, HAVCR2, CD3G, TCF7, and/or IL7R expressing T cells from a biological sample (e.g. an apheresis or a leukapheresis sample). In some embodiments, the methods comprise depleting MKI67, TOP2A, CD4, CCR7, NKG7, IL32, LAG3, HAVCR2, CD3G, TCF7, and/or IL7R non-expressing T cells from a biological sample (e.g. an apheresis or a leukapheresis sample). In some embodiments, an MKI67, TOP2A, CD4, CCR7, NKG7, IL32, LAG3, HAVCR2, CD3G, TCF7, and/or IL7R expressing T cell population is predicted to expand and/or proliferate sufficiently to achieve a threshold number of population doublings or number of cells within a particular time period.

In certain embodiments, provided herein are methods of selecting CD28+ T cells and/or depleting CD28− T cells for use in the selection method of Section II. In certain embodiments, provided herein are methods of selecting non-CD28 T cells and/or depleting non-CD28 T cells for use in the selection method of Section II.

### A. Samples and Cell Preparation

In particular embodiments, the provided methods are used in connection with isolating, selecting, or enriching cells from a biological sample (e.g., an apheresis or a leukapheresis sample) to generate one or more populations of enriched cells, e.g., CD28+ T cells. In some embodiments, the biological sample is a second biological sample obtained from a subject. In some embodiments, the provided methods include isolation of cells or populations thereof from biological samples, such as those obtained from or derived from a subject, such as one having a particular disease or condition or in need of a cell therapy or to which cell therapy will be administered. In some aspects, the subject is a human, such as a subject who is a patient in need of a particular therapeutic intervention, such as the adoptive cell therapy for which cells are being isolated, processed, and/or engineered. In some embodiments, the cells are engineered to generate an autologous cell therapy. In some embodiments, the provided methods include isolation of cells or populations thereof from biological samples, such as those obtained from or derived from a subject, such as one not identified as having a particular disease or condition or in need of a cell therapy or to which cell therapy will be administered. In some embodiments, the cells are engineered to generate an allogeneic cell therapy.

Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous sources. In some embodiments, the sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, the sample is or comprises a PBMC sample.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the sample containing cells (e.g., an apheresis product or a leukapheresis product) is washed in order to remove one or more anti-coagulants, such as heparin, added during apheresis or leukapheresis.

In some embodiments, the sample containing cells (e.g., a whole blood sample, a buffy coat sample, a peripheral blood mononuclear cells (PBMC) sample, an unfractionated T cell sample, a lymphocyte sample, a white blood cell sample, an apheresis product, or a leukapheresis product) is cryopreserved and/or cryoprotected (e.g., frozen) and then thawed and optionally washed prior to any steps for isolating, selecting, activating, stimulating, engineering, transducing, transfecting, incubating, culturing, harvesting, formulating a population of the cells, and/or administering the formulated cell population to a subject.

In some embodiments, a sample containing autologous peripheral blood mononuclear nells (PBMCs) from a subject is collected in a method suitable to ensure appropriate quality for manufacturing. In one aspect, the sample containing PBMCs is derived from fractionated whole blood. In some embodiments, whole blood from a subject is fractionated by leukapheresis using a centrifugal force and making use of the density differences between cellular phenotypes, when autologous mononuclear cells (MNCs) are preferentially enriched while other cellular phenotypes, such as red blood cells, are reduced in the collected cell composition. In some embodiments, autologous plasma is concurrently collected during the MNC collection, which in some aspects can allow for extended leukapheresis product stability. In one aspect, the autologous plasma is added to the leukapheresis product to improve the buffering capacity of the leukapheresis product matrix. In some aspects, a total volume of whole blood processed in order to generate the leukapheresis product is or is about 2 L, 4 L, 6 L, 8 L, 10 L, 12 L, 14 L, 16 L, 18 L, or 20 L, or is any value between any of the foregoing. In some embodiments, the volume of autologous plasma collected is or is about 10 mL, 50 mL, 100 mL, 150 mL, 200 mL, 250 mL, or 300 mL, or more, or is a volume between any of the foregoing. In some embodiments, the leukapheresis product is subjected to a procedure, e.g., washing and formulation for in-process cryopreservation, within about 48 hours of the leukapheresis collection completion. In some embodiments, the leukapheresis product is subjected to one or more wash steps, e.g., within about 2 hours, 6 hours, 12 hours, 18 hours, 24 hours, 36 hours, or 48 hours of the leukapheresis collection completion. In some aspects, the one or more wash step removes the anticoagulant during leukapheresis collection, cellular waste that may have accumulated in the leukapheresis product, residual platelets and/or cellular debris. In some embodiments, one or more buffer exchange is performed during the one or more wash step.

In particular embodiments, an apheresis product or a leukapheresis product is cryopreserved and/or cryoprotected (e.g., frozen) and then thawed before being subject to a cell enrichment, selection or isolation step (e.g., a T cell selection or isolation step) as described infra. In some embodiments, after a cryopreserved and/or cryoprotected apheresis product or leukapheresis product is subject to a T cell selection or isolation step, no additional cryopreservation and/or cryoprotection step is performed during or between any of the subsequent steps, such as the steps of activating, stimulating, engineering, transducing, transfecting, incubating, culturing, harvesting, formulating a population of the cells, and/or administering the formulated cell population to a subject. For example, T cells selected from a thawed cryopreserved and/or cryoprotected apheresis product or leukapheresis product are not again cryopreserved and/or cryoprotected before being thawed and optionally washed for a downstream process, such as T cell activation/stimulation or transduction.

In particular embodiments, an apheresis product or a leukapheresis product is cryopreserved and/or cryoprotected (e.g., frozen) at a density of, of about, or at least 5×10⁶ cells/mL, 10×10⁶ cells/mL, 20×10⁶ cells/mL, 30×10⁶ cells/mL, 40×10⁶ cells/mL, 50×10⁶ cells/mL, 60×10⁶ cells/mL, 70×10⁶ cells/mL, 80×10⁶ cells/mL, 90×10⁶ cells/mL, 100×10⁶ cells/mL, 110×10⁶ cells/mL, 120×10⁶ cells/mL, 130×10⁶ cells/mL, 140×10⁶ cells/mL, or 150×10⁶ cells/mL, or any value between any of the foregoing, in a cryopreservation solution or buffer. In some embodiments, the cryopreservation solution or buffer is or contains, for example, a DMSO solution optionally comprising human serum albumin (HSA), or other suitable cell freezing media.

In particular embodiments, the cryopreserved and/or cryoprotected apheresis product or leukapheresis product is banked (e.g., without T cell selection before freezing the sample), which, in some aspects, can allow more flexibility for subsequent manufacturing steps. In some aspects, the cryopreserved and/or cryoprotected apheresis product or leukapheresis product is aliquoted into multiple cryopreservation container such as bags, which can each individually or in combination be used in processing of the product. For example, when the total number of viable cells in the apheresis product or leukapheresis product is less than 15×10⁹ cells, the cryopreserved and/or cryoprotected apheresis product or leukapheresis product is aliquoted into four cryopreservation container such as bags. In some embodiments, when the total number of viable cells in the apheresis product or leukapheresis product is 15-30×10⁹ cells, the cryopreserved and/or cryoprotected apheresis product or leukapheresis product is aliquoted into eight cryopreservation container such as bags.

In one aspect, banking cells before selection increases cell yields for a downstream process, and banking cells earlier may mean they are healthier and may be easier to meet manufacturing success criteria. In another aspect, once thawed, the cryopreserved and/or cryoprotected apheresis product or leukapheresis product can be subject to one or more different selection methods. Advantages of this approach are, among other things, to enhance the availability, efficacy, and/or other aspects of cells of a cell therapy for treatment of a disease or condition of a subject, such as in the donor of the sample and/or another recipient.

In some embodiments, the sample (e.g. apheresis or leukapheresis sample) is collected and cryopreserved and/or cryoprotected prior to or without prior cell selection (e.g., without prior T cell selection, such as selection by chromatography), at a time after the donor is diagnosed with a disease or condition. In some aspects, the time of cryopreservation also is before the donor has received one or more of the following: any initial treatment for the disease or condition, any targeted treatment or any treatment labeled for treatment for the disease or condition, or any treatment other than radiation and/or chemotherapy. In some embodiments, the sample is collected after a first relapse of a disease following initial treatment for the disease, and before the donor or subject receives subsequent treatment for the disease. The initial and/or subsequent treatments may be a therapy other than a cell therapy. In some embodiments, the collected cells may be used in a cell therapy following initial and/or subsequent treatments. In one aspect, the cryopreserved and/or cryoprotected sample without prior cell selection may help reduce up-front costs, such as those associated with non-treatment patients in a randomized clinic trial who may crossover and require treatment later.

In some embodiments, the sample (e.g. apheresis or leukapheresis sample) is collected and cryopreserved and/or cryoprotected prior to or without prior cell selection (e.g., without prior T cell selection, such as selection by chromatography), at a time after a second relapse of a disease following a second line of treatment for the disease, and before the donor or subject receives subsequent treatment for the disease. In some embodiments, patients are identified as being likely to relapse after a second line of treatment, for example, by assessing certain risk factors. In some embodiments, the risk factors are based on disease type and/or genetics, such as relapsed/refractory multiple myeloma, double-hit lymphoma, primary refractory cancer, or activated B-cell lymphoma. In some embodiments, the risk factors are based on clinical presentation, such as early relapse after first-line treatment, or other poor prognostic indicators after treatment (e.g., IPI (International Prognostic Index)>2).

In some embodiments, the sample (e.g. apheresis or leukapheresis sample) is collected and cryopreserved and/or cryoprotected prior to or without prior cell selection (e.g., without prior T cell selection, such as selection by chromatography), at a time before the donor or subject is diagnosed with a disease. In some aspects, the donor or subject may be determined to be at risk for developing a disease. In some aspects, the donor or subject may be a healthy subject. In certain cases, the donor or subject may elect to bank or store cells without being deemed at risk for developing a disease or being diagnosed with a disease in the event that cell therapy is required at a later stage in life. In some embodiments, a donor or subject may be deemed at risk for developing a disease based on factors such as genetic mutations, genetic abnormalities, genetic disruptions, family history, protein abnormalities (such as deficiencies with protein production and/or processing), and lifestyle choices that may increase the risk of developing a disease. In some embodiments, the cells are collected as a prophylactic.

In some embodiments, the cryopreserved and/or cryoprotected sample of cells (e.g. apheresis or leukapheresis sample), such as a sample of cells that has not been subjected to a prior cell selection (e.g., without prior T cell selection, such as selection by chromatography) is stored, or banked, for a period of time greater than or equal to 12 hours, 24 hours, 36 hours, or 48 hours, or greater than or equal to 0.5 days, one day, 1.5 days, or two days. In some embodiments, the sample is stored or banked for a period of time greater than or equal to 1 week, 2 weeks, 3 weeks, or 4 weeks. In some embodiments, the sample is placed into long-term storage or long-term banking. In some aspects, the sample is stored for a period of time greater than or equal to 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, or more.

In some embodiments, an apheresis or leukapheresis sample taken from a donor is shipped in a cooled environment to a storage or processing facility, and/or cryogenically stored at the storage facility or processed at the processing facility. In some embodiments, before shipping, the sample is processed, for example, by selecting PBMCs, T cells, such as CD3+ T cells, CD4+ T cells, and/or CD8+ T cells. In some embodiments, such processing is performed after shipping and before cryogenically storing the sample. In some embodiments, the processing is performed after thawing the sample following cryogenically storage.

By allowing donors to store their cells at a stage when the donors, and thus their cells, have not undergone extensive treatment for a disease and/or prior to contracting of a disease or condition or diagnosis thereof, such cells may have certain advantages for use in cell therapy compared to cells harvested after one or after multiple rounds of treatment. For example, cells harvested before one or more rounds of treatment may be healthier, may exhibit higher levels of certain cellular activities, may grow more rapidly, and/or may be more receptive to genetic manipulation than cells that have undergone several rounds of treatment. Another example of an advantage according to embodiments described herein may include convenience. For example, by collecting, optionally processing, and storing a donor's cells before they are needed for cell therapy, the cells would be readily available if and when a recipient later needs them. This could increase apheresis lab capacity, providing technicians with greater flexibility for scheduling the apheresis collection process.

Exemplary methods and systems for cryogenic storage and processing of cells from a sample, such as an apheresis sample, can include those described in WO2018170188. In some embodiments, the method and systems involve collecting apheresis before the patient needs cell therapy, and then subjecting the apheresis sample to cryopreservation for later use in a process for engineering the cells, e.g. T cells, with a recombinant receptor (e.g. CAR). In some cases, such processes can include those described herein. In some embodiments, an apheresis sample is collected from a subject and cryopreserved prior to subsequent T cell selection, activation, stimulation, engineering, transduction, transfection, incubation, culturing, harvest, formulation of a population of the cells, and/or administration of the formulated cell population to a subject. In such examples, the cryopreserved apheresis sample is thawed prior to subjecting the sample to one or more selection steps, such as any as described herein.

In some embodiments, the cryopreserved and/or cryoprotected sample of cells (e.g. apheresis or leukapheresis sample), such as a sample of cells that has not been subject to a prior cell selection (e.g., without prior T cell selection, such as selection by chromatography) is thawed prior to its use for downstream processes for manufacture of a cell population for cell therapy, for example, a T cell population containing CAR+ T cells. In some embodiments, such a cryopreserved and/or cryoprotected sample of cells (e.g. apheresis or leukapheresis sample) is used in connection with the process provided herein for engineered a T cell therapy, such as a CAR+ T cell therapy. In particular examples, no further step of cryopreservation is carried out prior to or during the harvest/formulation steps.

In some embodiments, a cryopreserved and/or cryoprotected apheresis product or leukapheresis product is thawed. In some embodiments, the thawed cell composition is subjected to dilution (e.g., with a serum-free medium) and/or wash (e.g., with a serum-free medium), which in some cases can remove or reduce unwanted or undesired components. In some cases, the dilution and/or wash removes or reduces the presence of a cryoprotectant, e.g. DMSO, contained in the thawed sample, which otherwise may negatively impact cellular viability, yield, recovery upon extended room temperature exposure. In some embodiments, the dilution and/or wash allows media exchange of a thawed cryopreserved product into a serum-free medium, such as in PCT/US2018/064627, which is incorporated herein by reference.

In some embodiments, the serum-free medium comprises a basal medium (e.g. OpTmizer™ T-Cell Expansion Basal Medium (ThermoFisher), supplemented with one or more supplement. In some embodiments, the one or more supplement is serum-free. In some embodiments, the serum-free medium comprises a basal medium supplemented with one or more additional components for the maintenance, expansion, and/or activation of a cell (e.g., a T cell), such as provided by an additional supplement (e.g. OpTmizer™ T-Cell Expansion Supplement (ThermoFisher)). In some embodiments, the serum-free medium further comprises a free form of an amino acid such as L-glutamine. In some embodiments, the serum-free medium further comprises a dipeptide form of L-glutamine (e.g., L-alanyl-L-glutamine), such as the dipeptide in Glutamax™ (ThermoFisher). In some embodiments, the serum-free medium further comprises one or more recombinant cytokines, such as recombinant human IL-2, recombinant human IL-7, and/or recombinant human IL-15.

### B. Cell Selection

In some embodiments, selection, isolation, or enrichment of the cells, e.g., CD28+ or CD28-cells (e.g. CD28+ T cells), includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient. In certain embodiments, methods, techniques, and reagents for selection, isolation, and enrichment are described, for example, in PCT Application Nos. WO2013124474 and WO2015164675, which are hereby incorporated by reference in their entirety.

In certain embodiments, CD28+ T cells are isolated, enriched, or selected in a process or procedure that involves one or more selection steps. In some embodiments, the one or more selection steps are or involve negative selection. In certain embodiments, CD28+ T cells are isolated, enriched, or selected by separation or removal of CD28− T cells. In certain embodiments, a cell population enriched for CD28+ T cells results from negative selection of CD28− T cells from the population.

In certain embodiments, a bivalent antibody to link CD28+ T cells to a large density cell or bead. This technology has been used most prominently with red blood cells (e.g. RosetteSep™ STEMCELL Technologies), or any other similar or suitable technology to couple target cells, e.g., CD28+ T cells, to density gradients for removal.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In certain embodiments, such surface proteins may include CD28, CD4, or CD8. In certain embodiments, such surface proteins may include CD3. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. In some embodiments, the reagent or reagents for separation of cells is or include antibodies or antigen binding fragments thereof that bind to or recognize CD4, CD8, or CD28. In some embodiments, the reagent or reagents for separation of cells is or include antibodies or antigen binding fragments thereof that bind to or recognize CD3.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same system (e.g. a closed system) in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

Such separation steps can be based on positive selection, in which the cells having bound the reagents, e.g. antibody or binding partner, are retained for further use, and/or negative selection, in which the cells having not bound to the reagent, e.g., antibody or binding partner, are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, the process steps further include negative and/or positive selection of the incubated and cells, such as using a system or apparatus that can perform an affinity-based selection. In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker high) on the positively or negatively selected cells, respectively.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. In certain embodiments, separation steps are repeated and or performed more than once, where the positively or negatively selected fraction from one step is subjected to the same separation step, such as a repeated positive or negative selection. In some examples, a single separation step is repeated and/or performed more than once, for example to increase the purity of the selected cells and/or to further remove and/or deplete the negatively selected cells from the negatively selected fraction. In certain embodiments, one or more separation steps are performed two times, three times, four times, five times, six times, seven times, eight times, nine times, ten times, or more than ten times. In certain embodiments, the one or more selection steps are performed and/or repeated between one and ten times, between one and five times, or between three and five times.

For example, in some aspects, specific subpopulations of T cells, such as cells positive or expressing high levels of one or more surface markers, e.g., CD3+, CD4+, CD8+, or CD28+ T cells, are isolated by positive or negative selection techniques. In some embodiments, such cells are selected by incubation with one or more antibody or binding partner that specifically binds to such markers. In some embodiments, the antibody or binding partner can be conjugated, such as directly or indirectly, to a solid support or matrix to effect selection, such as a magnetic bead or paramagnetic bead. For example, in some embodiments, CD3+, CD4+ T cells, CD8+ T cells, or CD28+ T cells may be selected, e.g., positively selected, with CD3 Microbeads, CD4 Microbeads, CD8 Microbeads, or CD28 Microbeads (Miltenyl Biotec).

In certain embodiments, CD28+ T cells are separated from a PBMC sample by negative selection of cells negative for CD28 expression. In various embodiments, T cells are separated from a PBMC sample by negative selection of markers expressed on non-T cells, such as B cells, monocytes, or other white blood cells, such as CD14. In some aspects, a CD3+ selection step is used to separate T cells from non-T cells. Such a CD3+ population can be further sorted into sub-populations by positive or negative selection for CD4+ or CD8+, and/or markers expressed or expressed to a relatively higher degree on one or more naïve-like, memory, and/or effector T cell subpopulations. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naïve-like, memory, and/or effector T cell subpopulations.

In some embodiments, CD8+ cells are further enriched for CD28+ or depleted of CD28− T cells, such as by positive or negative selection based on surface expression of CD28. In certain embodiments, CD4+ cells are further enriched for CD28+ or depleted of CD28− T cells, such as by positive or negative selection based on surface expression of CD28. In certain embodiments, CD3+ cells are further enriched for CD28+ or depleted of CD28− T cells, such as by positive or negative selection based on surface expression of CD28.

In some embodiments, CD8+ cells are further enriched for or depleted of naïve, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., (2012) Blood. 1:72-82; Wang et al. (2012) J Immunother. 35(9):689-701. In some embodiments, combining TCM-enriched CD8+ T cells and CD4+ T cells further enhances efficacy.

In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps. In some embodiments, the selection for the CD4+ cell population and the selection for the CD8+ cell population are carried out simultaneously. In some embodiments, the CD4+ cell population and the selection for the CD8+ cell population are carried out sequentially, in either order. In some embodiments, methods for selecting cells can include those as described in published U.S. App. No. US20170037369. In some embodiments, the selected CD4+ cell population and the selected CD8+ cell population may be combined subsequent to the selecting. In some aspects, the selected CD4+ cell population and the selected CD8+ cell population may be combined in a bioreactor bag as described herein.

In various embodiments, a biological sample, e.g., a sample of PBMCs or other white blood cells, are subjected to selection of CD28− T cells, wherein the negative fractions containing enriched CD28+ T cells are retained. In some embodiments, the negative fraction enriched with CD28+ T cells is subjected to selection of CD3+ T cells, where the positive fraction is retained. In certain embodiments, CD8+ T cells are selected from the negative fraction enriched with CD28+ cells. In some embodiments, the negative fraction enriched with CD28+ T cells is subjected to selection of CD8+ T cells, where both the negative and positive fractions are retained. In certain embodiments, CD4+ T cells are selected from the negative fraction. In particular embodiments, from the negative fraction enriched with CD28+ T cells, are subjected to selection of CD4+ T cells, where both the negative and positive fractions are retained. In certain embodiments, CD8+ T cells are selected from the negative fraction.

In some aspects, the incubated sample or population of cells to be separated is incubated with a selection reagent containing small, magnetizable or magnetically responsive material, such as magnetically responsive particles or microparticles, such as paramagnetic beads (e.g., such as Dynabeads or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select. In some aspects, the selection agent is or includes a paramagnetic bead and an attached antibody or antigen binding fragment thereof that binds to or recognizes CD3, CD4, CD8, or CD28. In some embodiments, the selection agent is a CD3, CD4, CD8, or CD28 MACS® microbead.

In some embodiments, the magnetic particle or bead comprises a magnetically responsive material bound to a specific binding member, such as an antibody or other binding partner. Many well-known magnetically responsive materials for use in magnetic separation methods are known, e.g., those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 also may be used.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, separation is achieved in a procedure in which the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the affinity-based selection is via magnetic-activated cell sorting (MACS) (Miltenyi Biotech, Auburn, CA). Magnetic Activated Cell Sorting (MACS), e.g., CliniMACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells. In various embodiments, the selection agent is a CD3, CD4, CD8, or CD28 MACS® microbead.

In some embodiments, the suboptimal yield concentration of the affinity reagent is a concentration below a concentration used or required to achieve an optimal or maximal yield of bound cells in a given selection or enrichment involving incubating cells with the reagent and recovering or separating cells having bound to the reagent (“yield,” for example, being the number of the cells so-recovered or selected compared to the total number of cells in the incubation that are targeted by the reagent or to which the reagent is specific or that have a marker for which the reagent is specific and capable of binding). The suboptimal yield concentration generally is a concentration or amount of the reagent that in such process or step achieves less than all, e.g., no more than 70% yield of bound cells, e.g., CD28+, CD3+, CD4+, or CD8+ T cells, upon recovery of the cells having bound to the reagent. In some embodiments, no more than at or about 50%, 45%, 40%, 30%, or 25% yield is achieved by the suboptimal concentration of the affinity reagent. The concentration may be expressed in terms of number or mass of particles or surfaces per cell and/or number of mass or molecules of agent (e.g., antibody, such as antibody fragment) per cell.

In some embodiments, e.g., when operating in a suboptimal yield concentration for each or one or more of two or more selection reagents with affinity to CD28+, CD3+, CD4+, or CD8+ T cells, one or more of such reagents is used at a concentration that is higher than one or more of the other such reagent(s), in order to bias the ratio of the cell type recognized by that reagent as compared to the cell type(s) recognized by the other(s). For example, the reagent specifically binding to the marker for which it is desired to bias the ratio may be included at a concentration (e.g., agent or mass per cells) that is increased by half, 1-fold, 2-fold, 3-fold, 4-fold, 5-fold, 10-fold, or more, compared to other(s), depending on how much it is desired to increase the ratio. In some embodiments, when operating in the suboptimal range and/or with enough cells to achieve saturation of reagents, the amount of immunoaffinity reagent is proportional to the approximate yield of enriched cells. In certain embodiments, an appropriate amount or concentration of immunoaffinity reagents that depend on the desired ratio of the generated population containing the enriched or selected cells, e.g., CD28+, CD3+, CD4+, or CD8+ T cells, can be determined as a matter of routine.

In some embodiments, the separation and/or isolation steps are carried out using magnetic beads in which immunoaffinity reagents are reversibly bound, such as via a peptide ligand interaction with a streptavidin mutein as described in WO 2015/164675. Exemplary of such magnetic beads are Streptamers®. In some embodiments, the separation and/or steps is carried out using magnetic beads, such as those commercially available from Miltenyi Biotec.

In some embodiments, the magnetically responsive particles are left attached to the cells that are to be subsequently incubated, cultured and/or engineered. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the isolation and/or selection results in one or more populations of enriched T cells, e.g., CD28+ T cells, CD3+ T cells, CD4+ T cells, and/or CD8+ T cells. In some embodiments, two or more separate population of enriched T cells are isolated, selected, enriched, or obtained from a single biological sample. In some embodiments, separate populations are isolated, selected, enriched, and/or obtained from separate biological samples collected, taken, and/or obtained from the same subject.

In certain embodiments, the isolation and/or selection results in one or more populations of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD28+CD3+ T cells. In particular embodiment, the population of enriched T cells consists essentially of CD28+CD3+ T cells.

In certain embodiments, the isolation and/or enrichment results in a populations of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD28+CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the population of enriched T cells consists essentially of CD28+CD4+ T cells.

In certain embodiments, the isolation and/or enrichment results in a populations of enriched CD28+CD8+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD28+CD8+ T cells. In certain embodiments, the population of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the population of enriched T cells consists essentially of CD28+CD8+ T cells.

In aspects of the methods provided herein, cells of a sample, e.g., T cells, are selected by chromatographic isolation, such as by column chromatography including affinity chromatography or gel permeation chromatography. In some embodiments, cells, e.g., CD28+ T cells, are isolated, selected, or enriched by chromatographic isolation, such as by column chromatography including affinity chromatography or gel permeations chromatography. In some embodiments, the method employs a receptor binding reagent that binds to a receptor molecule (e.g., CD28) that is located on the surface of a target cell, such as the cell to be isolated, selected, or enriched (e.g., CD28+ cells). Such methods may be described as (traceless) cell affinity chromatography technology (CATCH) and may include any of the methods or techniques described in PCT Application Nos. WO2013124474 and WO2015164675, which are hereby incorporated by reference in its entirety.

In some embodiments, the target cells (e.g., CD28+ T cells), have or express a receptor molecule on the cell surface, such that the cells to be isolated, selected, or enriched are defined by the presence of at least one common specific receptor molecule (e.g., CD28). In some embodiments, the sample containing the target cell may also contain additional cells that are devoid of the receptor molecule. For example, in some embodiments, T cells are isolated, enriched, and or elected from a sample containing multiple cells types, e.g., red blood cells or B cells. In certain embodiments, CD28+ T cells are isolated, enriched, and or selected from a sample containing multiple cells types, e.g., red blood cells or B cells, thereby providing isolated CD28+ T cells and a non-selected population of cells, e.g., a population of enriched CD28− T cells.

In some embodiments, the receptor binding reagent is comprised in a chromatography column, e.g., bound directly or indirectly to the chromatography matrix (e.g., stationary phase). In some embodiments, the receptor binding reagent is present on the chromatography matrix (e.g., stationary phase) at the time the sample is added to the column. In some embodiments, the receptor binding reagent is capable of being bound indirectly to the chromatography matrix (e.g., stationary phase) through a reagent, e.g., an affinity reagent as described herein. In some embodiments, the affinity reagent is bound covalently or non-covalently to the stationary phase of the column. In some embodiments, the affinity reagent is reversibly immobilized on the chromatography matrix (e.g., stationary phase). In some cases, the affinity reagent is immobilized on the chromatography matrix (e.g., stationary phase) via covalent bonds. In some aspects, the affinity reagent is reversibly immobilized on the chromatography matrix (e.g., stationary phase) non-covalently.

In some embodiments, the chromatography matrix is used to remove or separate target cells from a sample, e.g., by negative selection. For example, in certain embodiments, a sample containing CD28+ cells and CD28− cells is contacted or incubated with a receptor binding reagent that binds to and or recognizes CD28. In certain embodiments, the sample and the receptor binding reagents are loaded onto the matrix, where, in some aspects, a complex is formed by the immobilized or attached affinity reagent, the receptor binding reagent, and a CD28+ T cell. In some embodiments, unbound cells are removed or rinsed from the chromatography matrix, thereby removing the bound CD28+ cells and providing a sample, e.g., a population, enriched for CD28+ cells.

In certain embodiments, the chromatography matrix is used to isolate, select, or enrich target cells from a sample, e.g., by positive selection. For example, in some embodiments, a sample containing CD4+ or CD8+ T cells and other cells, e.g., non-T cell immune cells, is contacted or incubated with a receptor binding reagent that binds to and or recognizes CD4 or CD8. In certain embodiments, the sample and the receptor binding reagents are loaded onto the matrix, where, in some aspects, a complex is formed by the immobilized or attached affinity reagent, the receptor binding reagent, and CD4+ or CD8+ T cell. In certain embodiments, unbound cells are removed or rinsed from the chromatography matrix. In particular embodiments, the immobilized CD4+ or CD8+ cells may be removed or released by the addition of the competition reagent, such as by disrupting the complex. In some aspects, the separated, released, or eluted CD4+ or CD8+ T cells are thus a sample, composition, or population of cells enriched for CD4+ or CD8+ T cells.

In certain embodiments, the chromatography matrix is used to isolate, select, or enrich target cells from a sample, e.g., by positive selection. For example, in some embodiments, a sample containing CD3+ T cells and other cells, e.g., non-T cell immune cells, is contacted or incubated with a receptor binding reagent that binds to and or recognizes CD3. In certain embodiments, the sample and the receptor binding reagents are loaded onto the matrix, where, in some aspects, a complex is formed by the immobilized or attached affinity reagent, the receptor binding reagent, and CD3+ T cell. In certain embodiments, unbound cells are removed or rinsed from the chromatography matrix. In particular embodiments, the immobilized CD3+ cells may be removed or released by the addition of the competition reagent, such as by disrupting the complex. In some aspects, the separated, released, or eluted CD3+ T cells are thus a sample, composition, or population of cells enriched for CD3+ T cells.

In some embodiments, multiple rounds of cell selection steps are carried out, where the positively or negatively selected fraction from one step is subjected to another selection step, such as a subsequent positive or negative selection. In certain embodiments, methods, techniques, and reagents for selection, isolation, and enrichment are described, for example, in PCT Application No. WO2015164675, which is hereby incorporated by reference in its entirety.

In some embodiments, a single selection step can be used to isolate target cells (e.g., CD28+ cells) from a sample. In some embodiments, the single selection step can be performed on a single chromatography column. In some examples, a single selection step can deplete cells expressing multiple markers simultaneously. Likewise, multiple cell types can simultaneously be positively selected. In certain embodiments, selection steps are repeated and or performed more than once, where the positively or negatively selected fraction from one step is subjected to the same selection step, such as a repeated positive or negative selection. In some examples, a single selection step is repeated and/or performed more than once, for example to increase the purity of the selected cells and/or to further remove and/or deplete the negatively selected cells from the negatively selected fraction. In certain embodiments, one or more selection steps are performed two times, three times, four times, five times, six times, seven times, eight times, nine times, ten times, or more than ten times. In certain embodiments, the one or more selection steps are performed and/or repeated between one and ten times, between one and five times, or between three and five times. In some embodiments, two selection steps are performed.

Cell selection may be performed using one or more chromatography columns. In some embodiments, the one or more chromatography columns are included in a closed system. In some embodiments, the closed system is an automated closed system, for example requiring minimal or no user (e.g., human) input. In some embodiments, cell selection is performed sequentially (e.g., a sequential selection technique). In some embodiments, the one or more chromatography columns are arranged sequentially. For example, a first column may be oriented such that the output of the column (e.g., eluent) can be fed, e.g., via connected tubing, to a second chromatography column. In some embodiments, a plurality of chromatography columns may be arranged sequentially. In some embodiments, cell selection may be achieved by carrying out sequential positive and negative selection steps, the subsequent step subjecting the negative and/or positive fraction from the previous step to further selection, where the entire process is carried out in the same tube or tubing set.

In some embodiments, a sample containing target cells is subjected to a sequential selection in which a first selection is effected to enrich for a CD28+ cell population, and the selected cells are used as the source of cells for a second selection to enrich for CD3+ populations. In some embodiments, a further selection or selections can be effected to enrich for sub-populations of the CD28+ population, for example, central memory T (T_{CM}) cells, naïve T cells, and/or cells positive for or expressing high levels of one or more surface markers, e.g., CD62L+, CCR7+, CD27+, CD127+, CD4+, CD8+, CD45RA+, and/or CD45RO+. In some embodiments, a sample containing target cells is subjected to a sequential selection in which a first selection is effected to enrich for a CD28+ population, and the selected cells are used as the source of cells for a second selection to enrich for CD3+ populations. In some embodiments, a further selection or selections can be effected to enrich for sub-populations of the CD28+CD3+ population, for example, central memory T (T_{CM}) cells, naïve T cells, and/or cells positive for or expressing high levels of one or more surface markers, e.g., CD62L+, CCR7+, CD27+, CD127+, CD4+, CD8+, CD45RA+, and/or CD45RO+. It is contemplated that in some aspects, specific subpopulations of T cells (e.g., CD28+ cells), such as cells positive or expressing high levels of one or more surface markers, e.g., CD62L+, CCR7+, CD27+, CD127+, CD4+, CD8+, CD45RA+, and/or CD45RO+ T cells, are selected by positive or negative sequential selection techniques.

In some embodiments, a sample containing target cells is subjected to a sequential selection in which a first selection is effected to enrich for a CD3+ population, and the selected cells are used as the source of cells for a second selection to enrich for CD28+ populations. In some embodiments, a sample containing target cells is subjected to a sequential selection in which a first selection is effected to enrich for a CD3+ population, and the selected cells are used as the source of cells for a second selection to enrich for CD28+ populations. In some embodiments, a further selection or selections can be effected to enrich for sub-populations of the CD3+CD28+ population, for example, central memory T (T_{CM}) cells, naïve T cells, and/or cells positive for or expressing high levels of one or more surface markers, e.g., CD62L+, CCR7+, CD27+, CD127+, CD4+, CD8+, CD45RA+, and/or CD45RO+. It is contemplated that in some aspects, specific subpopulations of T cells (e.g., CD28+ cells), such as cells positive or expressing high levels of one or more surface markers, e.g., CD62L+, CCR7+, CD27+, CD127+, CD4+, CD8+, CD45RA+, and/or CD45RO+ T cells, are selected by positive or negative sequential selection techniques.

In some embodiments, cell selection is performed in parallel (e.g., parallel selection technique). In some embodiments, the one or more chromatography columns are arranged in parallel. For example, two or more columns may be arranged such that a sample is loaded onto two or more columns at the same time via tubing that allows for the sample to be added to each column, for example, without the need for the sample to traverse through a first column. For example, using a parallel selection technique, cell selection may be achieved by carrying out positive and/or negative selection steps simultaneously, for example in a closed system where the entire process is carried out in the same tube or tubing set. In some embodiments, a sample containing target cells is subjected to a parallel selection in which the sample is load onto two or more chromatography columns, where each column effects selection of a cell population. In some embodiments, the two or more chromatography columns effect selection of CD28+, CD3+, CD4+, or CD8+ populations individually. In some embodiments, the two or more chromatography columns, including affinity chromatography or gel permeation chromatography, independently effect selection of the same cell population. For example, the two or more chromatography columns may effect selection of CD28+ cells. In some embodiments, the two or more chromatography columns, including affinity chromatography or gel permeation chromatography, independently effect selection of different cell populations. For example, the two or more chromatography columns independently may effect selection of CD28+ cells, CD4+ cells, CD3+ and/or CD8+ cells. In some embodiments, a further selection or selections, for example using sequential selection techniques, can be effected to enrich for sub-populations of one or all cell populations selected via parallel selection. For example, selected cells may be further selected for central memory T (T_{CM}) cells, naïve T cells, and/or cells positive for or expressing high levels of one or more surface markers, e.g., CD62L+, CCR7+, CD27+, CD127+, CD4+, CD8+, CD45RA+, and/or CD45RO+. In some embodiments, a sample containing target cells is subjected to a parallel selection in which parallel selection is effected to enrich for a CD28+ population on the two or more columns. In some embodiments, a further selection or selections can be effected to enrich for sub-populations of the CD28+ population, for example, central memory T (T_{CM}) cells, naïve T cells, and/or cells positive for or expressing high levels of one or more surface markers, e.g., CD62L+, CCR7+, CD27+, CD127+, CD4+, CD8+, CD45RA+, and/or CD45RO+. In some embodiments, a sample containing target cells is subjected to a parallel selection in which a selection is effected to enrich for a CD28+ population and a CD3+ population on the two or more columns, independently. In some embodiments, a further selection or selections can be effected to enrich for sub-populations of the CD28+ and CD3+ populations, for example, central memory T (T_{CM}) cells, naïve T cells, and/or cells positive for or expressing high levels of one or more surface markers, e.g., CD62L+, CCR7+, CD27+, CD127+, CD4+, CD8+, CD45RA+, and/or CD45RO+. In some embodiments, a sample containing target cells is subjected to a parallel selection in which a selection is effected to enrich for a CD28+ population and a CD4+ population on the two or more columns, independently. In some embodiments, a further selection or selections can be effected to enrich for sub-populations of the CD28+ and CD4+ populations, for example, central memory T (T_{CM}) cells, naïve T cells, and/or cells positive for or expressing high levels of one or more surface markers, e.g., CD62L+, CCR7+, CD27+, CD127+, CD4+, CD8+, CD45RA+, and/or CD45RO+. In some embodiments, a sample containing target cells is subjected to a parallel selection in which parallel selection is effected to enrich for a CD28+ population and a CD8+ population. In some embodiments, a further selection or selections can be effected to enrich for sub-populations of the CD28+ and CD8+ populations, for example, central memory T (T_{CM}) cells, naïve T cells, and/or cells positive for or expressing high levels of one or more surface markers, e.g., CD62L+, CCR7+, CD27+, CD127+, CD4+, CD8+, CD45RA+, and/or CD45RO+. In some embodiments, sequential and parallel selection techniques can be used in combination.

In some embodiments, two columns are used for parallel selection. In some embodiments, the two columns select for the same cell type (e.g., same selection marker). In some embodiments, the two columns each select for CD28+ cells.

In general, binding capacity of a stationary phase (e.g., selection resin) affects how much stationary phase is needed in order to select a certain number of target moieties, e.g., target cells such as T cells. The binding capacity, e.g., the number of target cells that can be immobilized per mL of the stationary phase (e.g., selection resin), can be used to determine or control the number of captured target cells on one or more columns. One or more chromatography column can be used for the on-column cell selection and stimulation disclosed herein. When multiple columns are used, they can be arranged sequentially, in parallel, or in a suitable combination thereof. Thus, the binding capacity of a stationary phase (e.g., selection resin) can be used to standardize the reagent amount in a single-column approach or the reagent amount for each column in a multiple-column approach.

In some embodiments, the binding capacity of the stationary phase used herein is the maximum number of target cells bound to the stationary phase at given solvent and cell concentration conditions, when an excess of target cells are loaded onto the stationary phase. In some embodiments, the binding capacity is or is about 100 million±25 million target cells (e.g., T cells) per mL of stationary phase. In some embodiments, the static binding capacity of the stationary phase (e.g., selection resin) disclosed herein ranges between about 75 million and about 125 million target cells per mL of stationary phase. In one aspect, the binding capacity of the stationary phase used herein for on-column cell selection and stimulation is a static binding capacity. In some embodiments, the static binding capacity is the maximum amount of cells capable of being immobilized on the stationary phase, e.g., at certain solvent and cell concentration conditions. In some embodiments, the static binding capacity of the stationary phase (e.g., selection resin) disclosed herein ranges between about 50 million and about 100 million target cells per mL of stationary phase. In some embodiments, the static binding capacity is or is about 100 million±25 million target cells (e.g., T cells) per mL of stationary phase. In some embodiments, the static binding capacity of the stationary phase (e.g., selection resin) disclosed herein ranges between about 75 million and about 125 million target cells per mL of stationary phase. In some embodiments, the static binding capacity of the stationary phase (e.g., selection resin) is between about 10 million and about 20 million, between about 20 million and about 30 million, between about 30 million and about 40 million, between about 40 million and about 50 million, between about 50 million and about 60 million, between about 60 million and about 70 million, between about 70 million and about 80 million, between about 80 million and about 90 million, between about 90 million and about 100 million, between about 110 million and about 120 million, between about 120 million and about 130 million, between about 130 million and about 140 million, between about 140 million and about 150 million, between about 150 million and about 160 million, between about 160 million and about 170 million, between about 170 million and about 180 million, between about 180 million and about 190 million, or between about 190 million and about 200 million target cells per mL of stationary phase. In some embodiments, the stationary phase is 20 mL. In some embodiments, the stationary phase has a binding capacity of 2 billion±0.5 billion cells.

As described above, in certain aspects, the methods provided herein employ a receptor binding reagent. In some embodiments, the reagent, as described in this Section, is a receptor binding reagent. In some embodiments, the receptor binding reagent binds to a molecule on the surface of a cell, such as a cell surface molecule. In some instances, the cell surface molecule is a selection marker. In some embodiments, the receptor binding reagent is capable of specifically binding to a selection marker expressed by one or more of the cells in a sample. In some embodiments, reference to specific binding to a molecule, such as a cell surface molecule or cell surface receptor, throughout the disclosure does not necessarily mean that the agent binds only to such molecule. For example, a reagent that specifically binds to a molecule may bind to other molecules, generally with much lower affinity as determined by, e.g., immunoassays, BIAcore®, KinExA 3000 instrument (Sapidyne Instruments, Boise, ID), or other assays. In some cases, the ability of a reagent, under specific binding conditions, to bind to a target molecule such that its affinity or avidity is at least 5 times as great, such as at least 10, 20, 30, 40, 50, 100, 250 or 500 times as great, or even at least 1000 times as great as the average affinity or avidity of the same agent to a collection of random peptides or polypeptides of sufficient statistical size.

In some embodiments, the cells, e.g., target cells (e.g., T cells), have or express a molecule on the cell surface, e.g., a selection marker, such that the cells to be selected are defined by the presence of at least one common specific molecule (e.g., selection marker). In some embodiments, the sample containing the target cell may also contain additional cells that are devoid of the molecule (e.g., selection marker). For example, in some embodiments, T cells may be selected from a sample containing multiple cells types, e.g., red blood cells or B cells. Selection marker and receptor molecule may be used interchangeably herein to refer to a cell surface molecule.

In some embodiments, the receptor molecule that is located on the cell surface, e.g., the target cell surface may be any molecule as long as it remains covalently or non-covalently bonded to the cell surface during a chromatographic separation process in a method according to the invention. The receptor molecule is a molecule against which a receptor binding reagent may be directed. In some embodiments the receptor is a peptide or a protein, such as a membrane receptor protein. In some embodiments the receptor is a lipid, a polysaccharide or a nucleic acid. A receptor that is a protein may be a peripheral membrane protein or an integral membrane protein. It may in some embodiments have one or more domains that span the membrane. In certain embodiments, the receptor molecule is a surface protein of an immune cell, e.g., CD3, CD4, CD8, or CD28. In some cases, for T cells the receptor molecule is CD3. In some cases, for T cells the receptor molecule is CD4 or CD8. In some embodiments the receptor molecule may be an antigen defining a desired cell population or subpopulation, for instance a population or subpopulation of blood cells, e. g. lymphocytes (e.g. T cells, CD28+ T cells, CD3+ T cells, CD4+ T cells, or CD8+ T cells).

In certain embodiments, the isolation and/or selection by chromatographic isolation results in one or more populations of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD28+CD3+ T cells. In particular embodiment, the population of enriched T cells consists essentially of CD28+CD3+ T cells.

In certain embodiments, the isolation and/or enrichment by chromatographic isolation results in a populations of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD28+CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the population of enriched T cells consists essentially of CD28+CD4+ T cells.

In certain embodiments, the isolation and/or enrichment by chromatographic isolation results in a populations of enriched CD28+CD8+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD28+CD8+ T cells. In certain embodiments, the population of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the population of enriched T cells consists essentially of CD28+CD8+ T cells.

### C. Input Compositions

In certain embodiments, the provided methods are used in connection with producing or preparing an input population or composition of cells (input composition or input population are used herein interchangeably). In some embodiments, the input composition is cells generated following any of the provided methods as described, e.g. infra, for selecting T cells from a biological sample (e.g. sample containing peripheral blood mononuclear cells, such as a leukapheresis or apheresis sample). In certain embodiments, the input cell composition includes a population of cells for use in genetic engineering, e.g., cells that will be genetically engineered or that will undergo a process to produce genetically engineered cells. In certain embodiments, the cells will be treated with, contacted with, or incubated with a nucleic acid that encodes a recombinant receptor. In certain embodiments, the input composition contains T cells, viable T cells, CD28+ T cells, CD3+ T cells, CD4+ T cells, CD8+ T cells, and/or subpopulations thereof. In some embodiments, genetically engineering the cells generates an autologous cell therapy.

In some embodiments, cell viability is assessed with an assay that may include, but is not limited to, dye uptake assays (e.g., calcein AM assays), XTT cell viability assays, and dye exclusion assays (e.g., trypan blue, Eosin, or propidium dye exclusion assays). In particular embodiments, a viable cell has negative expression of one or more apoptotic markers, e.g., Annexin V or active Caspase 3. In some embodiments, the viable cell is negative for the expression of one or more apoptosis marker that may include, but are not limited to, a caspase or an active caspase, e.g., caspase 2, caspase 3, caspase 6, caspase 7, caspase 8, caspase 9, or caspase 10, Bcl-2 family members, e.g., Bax, Bad, and Bid, Annexin V, or TUNEL staining. In particular embodiments, the viable cells are active caspase 3 negative. In certain embodiments, the viable cells are Annexin V negative.

In some embodiments, the input composition comprises a population of enriched CD28+ cells, e.g., viable CD28+ T cells. In some embodiments, at least 60%, at least 65%, at least 70%, at least 75%, at least 80%, at least 85%, at least 90%, at least 95%, at least 98%, at least 99%, at least 99.5%, at least 99.9%, or at or at about 100% of the cells of the input population are CD28+ T cells, e.g., viable CD28+ T cells. In some embodiments, the input population consists essentially of CD28+ T cells, e.g., viable CD28+ T cells.

In certain embodiments, the input population is a population of cells enriched for enriched CD4+ T cells and CD8+ T cells, e.g., CD4+ T cells and CD8+ T cells. In particular embodiments, the input population is or includes at least 60%, at least 65%, at least 70%, at least 75%, at least 80%, at least 85%, at least 90%, at least 95%, at least 98%, at least 99%, at least 99.5%, at least 99.9%, or at or at about 100% cells that are CD3+ T cells. In particular embodiments, the input population is or includes at least 60%, at least 65%, at least 70%, at least 75%, at least 80%, at least 85%, at least 90%, at least 95%, at least 98%, at least 99%, at least 99.5%, at least 99.9%, or at or at about 100% cells that are CD4+ and CD8+ T cells. In some embodiments, the input population consists essentially of CD4+ and CD8+ T cells. In particular embodiments, the input population is or includes at least 60%, at least 65%, at least 70%, at least 75%, at least 80%, at least 85%, at least 90%, at least 95%, at least 98%, at least 99%, at least 99.5%, at least 99.9%, or at or at about 100% cells CD3+ T cells (CD4+ and CD8+ T cells) that are CD28+, e.g. viable CD28+ T cells.

In certain embodiments, the input population is a population of enriched CD4+ T cells. In particular embodiments, at least 60%, at least 65%, at least 70%, at least 75%, at least 80%, at least 85%, at least 90%, at least 95%, at least 98%, at least 99%, at least 99.5%, at least 99.9%, or at or at about 100% of the cells of the input population are CD4+ T cells. In some embodiments, the input population consists essentially of CD4+ T cells. In particular embodiments, the input population is or includes at least 60%, at least 65%, at least 70%, at least 75%, at least 80%, at least 85%, at least 90%, at least 95%, at least 98%, at least 99%, at least 99.5%, at least 99.9%, or at or at about 100% cells CD4+ T cells that are CD28+, e.g. viable CD28+ T cells.

In certain embodiments, the input population is a population of enriched CD8+ T cells. In particular embodiments, at least 60%, at least 65%, at least 70%, at least 75%, at least 80%, at least 85%, at least 90%, at least 95%, at least 98%, at least 99%, at least 99.5%, at least 99.9%, or at or at about 100% of the cells of the input population are CD8+ T cells. In some embodiments, the input population consists essentially of CD8+ T cells. In particular embodiments, the input population is or includes at least 60%, at least 65%, at least 70%, at least 75%, at least 80%, at least 85%, at least 90%, at least 95%, at least 98%, at least 99%, at least 99.5%, at least 99.9%, or at or at about 100% cells CD8+ T cells that are CD28+, e.g. viable CD28+ T cells.

In some embodiments, cells from a population of enriched CD28+CD4+ T cells and cells from a population of enriched CD28+CD8+ T cells are mixed, combined, and/or pooled to generate an input population containing CD28+CD4+ T cells and CD28+CD8+ T cells. In certain embodiments, the populations of enriched CD28+CD4+ T cells and CD28+CD8+ T cells are pooled, mixed, and/or combined prior to stimulating cells, e.g., culturing the cells under stimulating conditions. In particular embodiments, the populations of enriched CD28+CD4+ and CD28+CD8+ T cells are pooled, mixed, and/or combined subsequent to freezing, e.g., cryopreserving, and thawing the populations of enriched CD28+CD4+ and CD28+CD8+ T cells.

In certain embodiments, the input population is produced, generated, or made by mixing, pooling, and/or combining cells from a population of enriched CD28+CD4+ cells with cells from a population of enriched CD28+CD8+ cells. In certain embodiments, the population of enriched CD28+CD4+ T cells contains at least 60%, 65%, 70%, 75%, 80%, 85%, 90%, 95%, 98%, 99%, or 99.9% CD28+CD4+ T cells. In particular embodiments, the population of enriched CD28+CD4+ T cells contains 100% CD28+CD4+ T cells or contains about 100% CD28+CD4+ T cells. In certain embodiments, the population of enriched T cells includes or contains less than 20%, less than 10%, less than 5%, less than 1%, less than 0.1%, or less than 0.01% CD28+CD8+ T cells, and/or contains no CD28+CD8+ T cells, and/or is free or substantially free of CD28+CD8+ T cells. In some embodiments, the populations of cells consist essentially of CD28+CD4+ T cells. In certain embodiments, the population of enriched CD28+CD8+ T cells contains at least 75%, 80%, 85%, 90%, 95%, 98%, 99%, or 99.9% CD28+CD8+ T cells, or contains or contains about 100% CD28+CD8+ T cells. In certain embodiments, the population of enriched CD28+CD8+ T cells includes or contains less than 20%, less than 10%, less than 5%, less than 1%, less than 0.1%, or less than 0.01% CD28+CD4+ T cells, and/or contains no CD28+CD4+ T cells, and/or is free or substantially free of CCD28+CD4+ T cells. In some embodiments, the populations of cells consist essentially of CD28+−CD8+ T cells.

In certain embodiments, CD4+ T cells and CD8+ T cells are pooled, mixed, and/or combined at a ratio of between 1:10 and 10:1, between 1:5 and 5:1, between 4:1 and 1:4, between 1:3 and 3:1, between 2:1 and 1:2, between 1.5:1 and 1:1.5, between 1.25:1 and 1:1.25, between 1.2:1 and 1:1.2, between 1.1:1 and 1:1.1, or about 1:1 or 1:1 CD4+ T cells to CD8+ T cells. In particular embodiments, viable CD4+ T cells and viable CD8+ T cells are pooled, mixed, and/or combined at a ratio of between 1:10 and 10:1, between 1:5 and 5:1, between 4:1 and 1:4, between 1:3 and 3:1, between 2:1 and 1:2, between 1.5:1 and 1:1.5, between 1.25:1 and 1:1.25, between 1.2:1 and 1:1.2, between 1.1:1 and 1:1.1, or about 1:1 or 1:1 CD4+ T cells to CD8+ T cells.

In particular embodiments, the input composition has an amount of, of about, or of at least 50×10⁶, 100×10⁶, 150×10⁶, 200×10⁶, 250×10⁶, 300×10⁶, 350×10⁶, 400×10⁶, 450×10⁶, 500×10⁶, 550×10⁶, 600×10⁶, 700×10⁶, 800×10⁶, 900×10⁶, 1,000×10⁶, 1,100×10⁶, or 1,200×10⁶ T cells, such as viable T cells, viable CD3+ T cells, or viable mixed CD4+ and CD8+ T cells. In particular embodiments, the input composition has an amount of, of about, or of at least 50×10⁶, 100×10⁶, 150×10⁶, 200×10⁶, 250×10⁶, 300×10⁶, 350×10⁶, 400×10⁶, 450×10⁶, 500×10⁶, 550×10⁶, 600×10⁶ CD4+ T cells, e.g., viable CD4+ T cells. In certain embodiments, the input composition has an amount of, of about, or of at least 50×10⁶, 100×10⁶, 150×10⁶, 200×10⁶, 250×10⁶, 300×10⁶, 350×10⁶, 400×10⁶, 450×10⁶, 500×10⁶, 550×10⁶, 600×10⁶ CD8+ T cells, e.g., viable CD8+ T cells. In some embodiments, the amount of cells is an amount of viable CD4+ and CD8+ T cells pooled, mixed and/or combined together in the same composition. In such embodiments, the CD4+ and CD8+ T cell are present at a ratio of between 1:3 and 3:1, between 2:1 and 1:2, between 1.5:1 and 1:1.5, between 1.25:1 and 1:1.25, between 1.2:1 and 1:1.2, between 1.1:1 and 1:1.1, or about 1:1 or 1:1 CD4+ T cells to CD8+ T cells. In some embodiments, the amount of cells is an amount of viable CD4+ and CD8+ T cells pooled, mixed and/or combined together at a ratio of about 1:1 or 1:1 CD4+ T cells to CD8+ T cells.

In particular embodiments, the input composition has an amount of between or between about 300×10⁶ and 600×10⁶ T cells, e.g., viable CD3+ cells, or mixed viable CD4+ and viable CD8+ cells (e.g., mixed at or at about a 1:1 ratio). In some embodiments, the input population has an amount of or of about 300×10⁶, e.g., viable CD3+ cells, or mixed viable CD4+ and viable CD8+ cells (e.g., mixed at or at about a 1:1 ratio). In some embodiments, the input population has an amount of or of about 400×10⁶, e.g., viable CD3+ cells or mixed viable CD4+ and viable CD8+ cells (e.g., mixed at or at about a 1:1 ratio). In some embodiments, the input population has an amount of or of about 500×10⁶, e.g., viable CD3+ cells or mixed viable CD4+ and viable CD8+ cells (e.g., mixed at or at about a 1:1 ratio). In some embodiments, the input population has an amount of or of about 600×10⁶, e.g., viable CD3+ cells or mixed viable CD4+ and viable CD8+ cells (e.g., mixed at or at about a 1:1 ratio). In some embodiments, the input population has an amount of or of about 700×10⁶, e.g., viable CD3+ cells or mixed viable CD4+ and viable CD8+ cells (e.g., mixed at or at about a 1:1 ratio). In some embodiments, the input population has an amount of or of about 800×10⁶, e.g., viable CD3+ cells or mixed viable CD4+ and viable CD8+ cells (e.g., mixed at or at about a 1:1 ratio). In some embodiments, the input population has an amount of or of about 900×10⁶, e.g., viable CD3+ cells or mixed viable CD4+ and viable CD8+ cells (e.g., mixed at or at about a 1:1 ratio). In some embodiments, the input population has an amount of or of about 100×10⁷, e.g., viable CD3+ cells or mixed viable CD4+ and viable CD8+ cells (e.g., mixed at or at about a 1:1 ratio). In some embodiments, the input population has an amount of or of about 110×10⁷, e.g., viable CD3+ cells or mixed viable CD4+ and viable CD8+ cells (e.g., mixed at or at about a 1:1 ratio). In some embodiments, the input population has an amount of or of about 120×10⁷, e.g., viable CD3+ cells or mixed viable CD4+ and viable CD8+ cells (e.g., mixed at or at about a 1:1 ratio).

Although in the above embodiments, the cell selection, isolation, separation, enrichment, and/or purification processes are discussed in the context of preparing an input composition, it should be understood that the cell selection, isolation, separation, enrichment, and/or purification processes disclosed herein can be used during, prior to, or between any of the subsequent steps (e.g., activation, stimulation, engineering, transduction, transfection, incubation, culturing, harvest, formulation, and/or administering a formulated cell population to a subject), in any suitable combination and/or order. For example, a T cell selection, isolation, separation, enrichment, and/or purification step can be performed between T cell activation/stimulation and T cell transduction. In another example, a T cell selection, isolation, separation, enrichment, and/or purification step can be performed after T cell transduction, but prior to harvesting, prior to collecting, and/or prior to formulating the cells. In a particular example, a T cell selection, isolation, separation, enrichment, and/or purification step can be performed immediately prior to harvesting the cells as a refining or clarification step. In some embodiments, a T cell selection step by chromatography is performed between T cell activation/stimulation and T cell transduction. In some embodiments, a T cell selection step by chromatography is performed after T cell transduction, but prior to harvesting, prior to collecting, and/or prior to formulating the cells. In some embodiments, a T cell selection step by chromatography is performed immediately prior to harvesting the cells.

In some embodiments, the input composition is generated by mixing, combining, and/or pooling a population enriched in CD28+CD8+ T cells generated from a starting sample, such as PBMCs, with a population enriched in CD28+CD4+ T cells generated from the starting sample. In some embodiments, the population enriched in CD28+CD4+ T cells is generated from the CD8-negative fraction generated during the process of generating the population enriched in CD8+ T cells from the starting sample. In particular embodiments, the input composition has a ratio of or of about 1:1 CD4+ T cells to CD8+ T cells, and is subjected to one or more wash step, e.g., with a serum-free medium described in PCT/US2018/064627, prior to stimulating the cells, e.g., culturing the cells under stimulating conditions. In some embodiments, the one or more wash step allows media exchange from a PBS/EDTA buffer containing albumin into the serum-free medium, which is also used in cell stimulation.

In other aspects, provided herein are methods of selecting cells enriched for non-CD28 markers. In some embodiments, provided herein are methods of selecting for CD45RA+, CD45RO+, CD27+, CD197+, CD4+, CD57+, CD8+, CD25+, PD1+, LAG3+, CD3+ and/or TIM3+ T cells and/or depleting CD28− T cells from a biological sample (e.g. an apheresis or a leukapheresis sample), thereby generating an enriched CD45RA+, CD45RO+, CD27+, CD197+, CD4+, CD57+, CD8+, CD25+, PD1+, LAG3+, CD3+ and/or TIM3+ cell population. In some embodiments, the biological sample is a second biological sample obtained from a subject. In some embodiments, cells of the enriched CD45RA+, CD45RO+, CD27+, CD197+, CD4+, CD57+, CD8+, CD25+, PD1+, LAG3+, CD3+ and/or TIM3+ cell population are genetically engineered, such as to produce cells expressing a recombinant receptor (e.g. a chimeric antigen receptor). In some embodiments, the genetically engineered cells are a cell therapy. In some embodiments, the cell therapy is an autologous cell therapy. In some embodiments, the cell therapy is an allogeneic cell therapy.

In some embodiments, the methods comprise selecting for CD45RA+, CD45RO+, CD27+, CD197+, CD4+, CD57+, CD8+, CD25+, PD1+, LAG3+, CD3+ and/or TIM3+ T cells from a biological sample (e.g. an apheresis or a leukapheresis sample). In some embodiments, the methods comprise depleting CD45RA−, CD45RO−, CD27−, CD197−, CD4−, CD57−, CD8−, CD25−, PD1−, LAG3−, CD3− and/or TIM3− T cells from a biological sample (e.g. an apheresis or a leukapheresis sample). In some embodiments, an enriched CD45RA+, CD45RO+, CD27+, CD197+, CD4+, CD57+, CD8+, CD25+, PD1+, LAG3+, CD3+ and/or TIM3+ cell population is predicted to expand and/or proliferate sufficiently to achieve a threshold number of population doublings or number of cells within a particular time period.

In some embodiments, provided herein are methods of selecting for IL2RA, LIF and/or OSM expressing T cells and/or depleting IL2RA, LIF and/or OSM non-expressing T cells from a biological sample (e.g. an apheresis or a leukapheresis sample), thereby generating an IL2RA, LIF and/or OSM expressing T cell population. In some embodiments, the biological sample is a second biological sample obtained from a subject. In some embodiments, cells of the IL2RA, LIF and/or OSM expressing T cell population are genetically engineered, such as to produce cells expressing a recombinant receptor (e.g. a chimeric antigen receptor). In some embodiments, the genetically engineered cells are a cell therapy. In some embodiments, the cell therapy is an autologous cell therapy. In some embodiments, the cell therapy is an allogeneic cell therapy.

In some embodiments, the methods comprise selecting for IL2RA, LIF and/or OSM expressing T cells from a biological sample (e.g. an apheresis or a leukapheresis sample). In some embodiments, the methods comprise depleting IL2RA, LIF and/or OSM non-expressing T cells from a biological sample (e.g. an apheresis or a leukapheresis sample). In some embodiments, an IL2RA, LIF and/or OSM expressing T cell population is predicted to expand and/or proliferate sufficiently to achieve a threshold number of population doublings or number of cells within a particular time period.

In some embodiments, provided herein are methods of selecting for MKI67, TOP2A, CD4, CCR7, NKG7, IL32, LAG3, HAVCR2, CD3G, TCF7, and/or IL7R expressing T cells and/or depleting MKI67, TOP2A, CD4, CCR7, NKG7, IL32, LAG3, HAVCR2, CD3G, TCF7, and/or IL7R non-expressing T cells from a biological sample (e.g. an apheresis or a leukapheresis sample), thereby generating an MKI67, TOP2A, CD4, CCR7, NKG7, IL32, LAG3, HAVCR2, CD3G, TCF7, and/or IL7R expressing T cell population. In some embodiments, the biological sample is a second biological sample obtained from a subject. In some embodiments, cells of the MKI67, TOP2A, CD4, CCR7, NKG7, IL32, LAG3, HAVCR2, CD3G, TCF7, and/or IL7R expressing T cell population are genetically engineered, such as to produce cells expressing a recombinant receptor (e.g. a chimeric antigen receptor). In some embodiments, the genetically engineered cells are a cell therapy. In some embodiments, the cell therapy is an autologous cell therapy. In some embodiments, the cell therapy is an allogeneic cell therapy.

In some embodiments, the methods comprise selecting for MKI67, TOP2A, CD4, CCR7, NKG7, IL32, LAG3, HAVCR2, CD3G, TCF7, and/or IL7R expressing T cells from a biological sample (e.g. an apheresis or a leukapheresis sample). In some embodiments, the methods comprise depleting MKI67, TOP2A, CD4, CCR7, NKG7, IL32, LAG3, HAVCR2, CD3G, TCF7, and/or IL7R non-expressing T cells from a biological sample (e.g. an apheresis or a leukapheresis sample). In some embodiments, an MKI67, TOP2A, CD4, CCR7, NKG7, IL32, LAG3, HAVCR2, CD3G, TCF7, and/or IL7R expressing T cell population is predicted to expand and/or proliferate sufficiently to achieve a threshold number of population doublings or number of cells within a particular time period.

In certain embodiments, provided herein are methods of selecting CD28+ T cells and/or depleting CD28− T cells for use in the selection method of Section II. In certain embodiments, provided herein are methods of selecting non-CD28 T cells and/or depleting non-CD28 T cells for use in the selection method of Section II.

### IV. SELECTION OF SUBJECTS FOR METHODS OF TREATMENT

Provided herein are methods of selecting subjects for treatment with a cell therapy (e.g., a T cell therapy). In some embodiments, the subject is selected for treatment with a cell therapy if the percentage of CD28+ T cells in a biological sample (e.g., a first biological sample, such as an apheresis or PBMC sample) obtained from the subject is above a threshold value. In some embodiments, the percentage of CD28+ T cells is the percentage of T cells in the sample that are CD28+. In some embodiments, the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, the sample is or comprises an apheresis sample. In some embodiments, the sample is or comprises a leukapheresis sample. In some embodiments, the sample is or comprises a PBMC sample. In some embodiments, the biological sample is a whole blood sample. In some embodiments, the biological sample is a PBMC sample. In some embodiments, the biological sample is an unfractionated T cell sample.

In some embodiments, the methods comprise obtaining a biological sample from a subject and determining the percentage of CD28+ T cells in the biological sample, e.g. among all T cells in the biological sample. In some embodiments, if the percentage of CD28+ T cells in the biological sample (e.g., first biological sample) is above a threshold value, the subject is selected for treatment with the cell therapy. In some embodiments, the method further comprises administering the cell therapy to the selected subject. In some embodiments, if the percentage of CD28+ T cells in the biological sample (e.g., first biological sample) is below a threshold value, the subject is not selected for treatment with the cell therapy. In some embodiments, cells of the cell therapy are obtained from the subject. In some embodiments, cells of the T cell therapy are autologous to the subject. In some embodiments, cells of the cell therapy are obtained from a different subject. In some embodiments, cells of the T cell therapy are allogeneic to the subject.

In some embodiments, the threshold percentage is about 30%, 31%, 32%, 33%, 34%, 35%, about 36%, about 37%, about 38%, about 39%, about 40%, about 41%, about 42%, about 43%, about 44%, about 45%, about 46%, about 47%, about 48%, about 49%, or about 50%. In some embodiments, the threshold percentage is between about 35% and about 50%. In some embodiments, the threshold percentage is between about 40% and about 45%. In some embodiments, the threshold percentage is about 40%. In some embodiments, the threshold percentage is about 41%. In some embodiments, the threshold percentage is about 42%. In some embodiments, the threshold percentage is about 43%. In some embodiments, the threshold percentage is about 44%. In some embodiments, the threshold percentage is about 45%.

In some embodiments, the subject is selected for treatment with the cell therapy (e.g. T cell therapy) if the percentage of CD28+ T cells in the biological sample, e.g. among all T cells in the biological sample, is above about 35%. In some embodiments, the subject is selected for treatment with the cell therapy if the percentage of CD28+ T cells in the biological sample, e.g. among all T cells in the biological sample, is above about 40%. In some embodiments, the subject is selected for treatment with the cell therapy if the percentage of CD28+ T cells in the biological sample, e.g. among all T cells in the biological sample, is above about 44%. In some embodiments, the subject is selected for treatment with the cell therapy if the percentage of CD28+ T cells in the biological sample, e.g. among all T cells in the biological sample, is above about 45%. In some embodiments, the subject is selected for treatment with the cell therapy if the percentage of CD28+ T cells in the biological sample, e.g. among all T cells in the biological sample, is above about 50%. In some embodiments, the subject is selected for treatment with the cell therapy if the percentage of CD28+ T cells in the biological sample, e.g. among all T cells in the biological sample, is between about 35% and about 100%. In some embodiments, the subject is selected for treatment with the cell therapy if the percentage of CD28+ T cells, e.g. among all T cells in the biological sample, in the biological sample is between about 40% and about 100%. In some embodiments, the subject is selected for treatment with the cell therapy if the percentage of CD28+ T cells in the biological sample, e.g. among all T cells in the biological sample, is between about 44% and about 100%. In some embodiments, the subject is selected for treatment with the cell therapy if the percentage of CD28+ T cells in the biological sample, e.g. among all T cells in the biological sample, is between about 45% and about 100%. In some embodiments, the subject is selected for treatment with the cell therapy if the percentage of CD28+ T cells in the biological sample, e.g. among all T cells in the biological sample, is between about 50% and about 100%. In some embodiments, the biological sample is a second biological sample obtained from the subject.

In some embodiments, the second biological sample is obtained from the subject between about 6 weeks and about 1 week prior to treatment of a subject with a cell therapy. In some embodiments, the second biological sample is obtained from the subject about 6 weeks prior to treatment of a subject with a cell therapy. In some embodiments, the second biological sample is obtained from the subject about 5 weeks prior to treatment of a subject with a cell therapy. In some embodiments, the second biological sample is obtained from the subject about 6 weeks prior to treatment of a subject with cell therapy. In some embodiments, the second biological sample is obtained from the subject about 3 weeks prior to treatment of a subject with a cell therapy. In some embodiments, the second biological sample is obtained from the subject about 2 weeks prior to treatment of a subject with a cell therapy. In some embodiments, the second biological sample is obtained from the subject about 1 week prior to treatment of a subject with a cell therapy. In some embodiments, the cell therapy is an autologous cell therapy. In some embodiments, the subject from whom the second biological sample is obtained is the same subject to whom the cell therapy is administered. In some embodiments, the cell therapy is an allogeneic cell therapy. In some embodiments, the subject from whom the second biological sample is different than the same subject to whom the cell therapy is administered.

In some embodiments, the subject is not selected for treatment with the T cell therapy if the percentage of CD28+ T cells in the biological sample, e.g. among all T cells in the biological sample, is below about 35%. In some embodiments, the subject is not selected for treatment with the T cell therapy if the percentage of CD28+ T cells in the biological sample, e.g. among all T cells in the biological sample, is below about 40%. In some embodiments, the subject is not selected for treatment with the T cell therapy if the percentage of CD28+ T cells in the biological sample, e.g. among all T cells in the biological sample, is below about 44%. In some embodiments, the subject is not selected for treatment with the T cell therapy if the percentage of CD28+ T cells in the biological sample, e.g. among all T cells in the biological sample, is below about 45%. In some embodiments, the subject is not selected for treatment with the T cell therapy if the percentage of CD28+ T cells in the biological sample, e.g. among all T cells in the biological sample, is below about 50%. In some embodiments, the subject is not selected for treatment with the T cell therapy if the percentage of CD28+ T cells in the biological sample, e.g. among all T cells in the biological sample, is between about 30% and about 35%. In some embodiments, the subject is not selected for treatment with the T cell therapy if the percentage of CD28+ T cells in the biological sample, e.g. among all T cells in the biological sample, is between about 0% and about 40%. In some embodiments, the subject is not selected for treatment with the T cell therapy if the percentage of CD28+ T cells in the biological sample, e.g. among all T cells in the biological sample, is between about 0% and about 44%. In In some embodiments, the subject is not selected for treatment with the T cell therapy if the percentage of CD28+ T cells in the biological sample, e.g. among all T cells in the biological sample, is between about 0% and about 45%. In some embodiments, the subject is not selected for treatment with the T cell therapy if the percentage of CD28+ T cells in the biological sample, e.g. among all T cells in the biological sample, is between about 0% and about 50%.

In some embodiments, the method provided herein comprise selecting a subject for treatment with a cell therapy, if at least about 40% of T cells (e.g., peripheral T cells) in the subject are CD28+. Also provided herein is a method of treating a disease or condition in a human subject with a T cell therapy, the method comprising administering to a human subject having a disease or condition a therapeutically effective amount of a T cell therapy, wherein: (a) at least about 40% of T cells (e.g., peripheral T cells) in the subject are CD28+; and (b) manufacture of the T cell therapy relies on CD28-mediated expansion of the T cells of the T cell therapy. In some embodiments, the disease or condition is a multiple myeloma.

Also provided herein is a method of treating multiple myeloma in a human subject, the method comprising administering to a human subject having a multiple myeloma a therapeutically effective amount of a T cell therapy targeting the multiple myeloma, wherein at least about 40% of T cells (e.g., peripheral T cells) in the subject are CD28+. In some embodiments, manufacture of the T cell therapy relies on CD28-mediated expansion of the T cells of the T cell therapy

In some embodiments, prior to administration of the T cell therapy to the human subject, it has been determined that at least about 40% of T cells (e.g., peripheral T cells) in the subject are CD28+. In some embodiments, the T cell therapy targets B cell maturation antigen (BCMA). In some embodiments, the T cell therapy is an autologous T cell therapy. In some embodiments, the T cell therapy is a chimeric antigen receptor (CAR) T cell therapy. In some embodiments, the CAR T cell therapy targets BCMA. In some embodiments, at least about 44%, at least about 45%, or at least about 50% of T cells (e.g., peripheral T cells) in the subject are CD28+. In some embodiments, the percentage of CD28+ T cells is the percentage of T cells that are CD28+.

In some aspects, provided herein are methods of selecting subjects for treatment with a cell therapy based on the expression of non-CD28 markers. In some embodiments, the subject is selected for treatment with a cell therapy if the percentage of CD45RA+, CD45RO+, CD27+, CD197+, CD4+, CD57+, CD8+, CD25+, PD1+, LAG3+, CD3+ and/or TIM3+ T cells in a biological sample (e.g., a first biological sample, such as an apheresis or PBMC sample) obtained from the subject is above a threshold value. In some embodiments, the percentage of CD45RA+, CD45RO+, CD27+, CD197+, CD4+, CD57+, CD8+, CD25+, PD1+, LAG3+, CD3+ and/or TIM3+ T cells is the percentage of T cells in the sample that are CD45RA+, CD45RO+, CD27+, CD197+, CD4+, CD57+, CD8+, CD25+, PD1+, LAG3+, CD3+ and/or TIM3+. In some embodiments, the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, the sample is or comprises an apheresis sample. In some embodiments, the sample is or comprises a leukapheresis sample. In some embodiments, the sample is or comprises a PBMC sample. In some embodiments, the biological sample is a whole blood sample. In some embodiments, the biological sample is a PBMC sample. In some embodiments, the biological sample is an unfractionated T cell sample.

In some embodiments, the methods comprise obtaining a biological sample from a subject and determining the percentage of CD45RA+, CD45RO+, CD27+, CD197+, CD4+, CD57+, CD8+, CD25+, PD1+, LAG3+, CD3+ and/or TIM3+ T cells in the biological sample, e.g. among all T cells in the biological sample. In some embodiments, if the percentage of CD45RA+, CD45RO+, CD27+, CD197+, CD4+, CD57+, CD8+, CD25+, PD1+, LAG3+, CD3+ and/or TIM3+ T cells in the biological sample (e.g., first biological sample) is above a threshold value, the subject is selected for treatment with the cell therapy. In some embodiments, the method further comprises administering the cell therapy to the selected subject. In some embodiments, if the percentage of CD45RA+, CD45RO+, CD27+, CD197+, CD4+, CD57+, CD8+, CD25+, PD1+, LAG3+, CD3+ and/or TIM3+ T cells in the biological sample (e.g., first biological sample) is below a threshold value, the subject is not selected for treatment with the cell therapy. In some embodiments, cells of the cell therapy are obtained from the subject. In some embodiments, cells of the T cell therapy are autologous to the subject. In some embodiments, cells of the cell therapy are obtained from a different subject. In some embodiments, cells of the T cell therapy are allogeneic to the subject.

In some embodiments, the subject is selected for treatment with a cell therapy if the percentage of IL2RA, LIF, and/or OSM expressing T cells in a biological sample (e.g., a first biological sample, such as an apheresis or PBMC sample) obtained from the subject is above a threshold value. In some embodiments, the percentage of IL2RA, LIF, and/or OSM expressing T cells is the percentage of T cells in the sample that express IL2RA, LIF, and/or OSM. In some embodiments, the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, the sample is or comprises an apheresis sample. In some embodiments, the sample is or comprises a leukapheresis sample. In some embodiments, the sample is or comprises a PBMC sample. In some embodiments, the biological sample is a whole blood sample. In some embodiments, the biological sample is a PBMC sample. In some embodiments, the biological sample is an unfractionated T cell sample.

In some embodiments, the methods comprise obtaining a biological sample from a subject and determining the percentage of IL2RA, LIF, and/or OSM expressing T cells in the biological sample, e.g. among all T cells in the biological sample. In some embodiments, if the percentage of IL2RA, LIF, and/or OSM expressing T cells in the biological sample (e.g., first biological sample) is above a threshold value, the subject is selected for treatment with the cell therapy. In some embodiments, the method further comprises administering the cell therapy to the selected subject. In some embodiments, if the percentage of IL2RA, LIF, and/or OSM expressing T cells in the biological sample (e.g., first biological sample) is below a threshold value, the subject is not selected for treatment with the cell therapy. In some embodiments, cells of the cell therapy are obtained from the subject. In some embodiments, cells of the T cell therapy are autologous to the subject. In some embodiments, cells of the cell therapy are obtained from a different subject. In some embodiments, cells of the T cell therapy are allogeneic to the subject.

In some embodiments, the subject is selected for treatment with a cell therapy if the percentage of MKI67, TOP2A, CD4, CCR7, NKG7, IL32, LAG3, HAVCR2, CD3G, TCF7, and/or IL7R expressing T cells in a biological sample (e.g., a first biological sample, such as an apheresis or PBMC sample) obtained from the subject is above a threshold value. In some embodiments, the percentage of MKI67, TOP2A, CD4, CCR7, NKG7, IL32, LAG3, HAVCR2, CD3G, TCF7, and/or IL7R expressing T cells is the percentage of T cells in the sample that express MKI67, TOP2A, CD4, CCR7, NKG7, IL32, LAG3, HAVCR2, CD3G, TCF7, and/or IL7R. In some embodiments, the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, the sample is or comprises an apheresis sample. In some embodiments, the sample is or comprises a leukapheresis sample. In some embodiments, the sample is or comprises a PBMC sample. In some embodiments, the biological sample is a whole blood sample. In some embodiments, the biological sample is a PBMC sample. In some embodiments, the biological sample is an unfractionated T cell sample.

In some embodiments, the methods comprise obtaining a biological sample from a subject and determining the percentage of MKI67, TOP2A, CD4, CCR7, NKG7, IL32, LAG3, HAVCR2, CD3G, TCF7, and/or IL7R expressing T cells in the biological sample, e.g. among all T cells in the biological sample. In some embodiments, if the percentage of MKI67, TOP2A, CD4, CCR7, NKG7, IL32, LAG3, HAVCR2, CD3G, TCF7, and/or IL7R expressing T cells in the biological sample (e.g., first biological sample) is above a threshold value, the subject is selected for treatment with the cell therapy. In some embodiments, the method further comprises administering the cell therapy to the selected subject. In some embodiments, if the percentage of MKI67, TOP2A, CD4, CCR7, NKG7, IL32, LAG3, HAVCR2, CD3G, TCF7, and/or IL7R expressing T cells in the biological sample (e.g., first biological sample) is below a threshold value, the subject is not selected for treatment with the cell therapy. In some embodiments, cells of the cell therapy are obtained from the subject. In some embodiments, cells of the T cell therapy are autologous to the subject. In some embodiments, cells of the cell therapy are obtained from a different subject. In some embodiments, cells of the T cell therapy are allogeneic to the subject.

Provided herein are methods of selecting subject having or suspected of having a disease for treatment comprising the methods described in any of Sections II, III and IV, or the population of cells described in Section I.

### V. PROCESS FOR GENETICALLY ENGINEERING ENRICHED T CELL POPULATIONS

Among provided methods are methods for genetically engineering T cells with a recombinant receptor, such as a chimeric antigen receptor (CAR). In some embodiments, the T cells may be enriched for CD28. In some embodiments, the T cells may be enriched for non-CD28 markers. In some embodiments, the provided methods can include one or more steps of stimulating, activating, engineering, cultivating and/or expanding one or more populations of T cells (e.g., CD28+ cells or non-CD28+ cells).

In certain embodiments, the one or more populations are or include any population of CD28+ cells described herein. In some embodiments, the one or more populations are isolated, selected, or enriched from a biological sample by any method or process described herein. In some embodiments, the one or more populations of enriched CD28+ cells are stimulated or activated, such as by incubating the cells of the population under stimulating conditions, such as any stimulating condition described herein. In certain embodiments, the one or more populations of enriched CD28+ cells are genetically engineered, such as by introducing a heterologous polynucleotide to the cells of the one or more populations. In some embodiments, the introducing is performed by any method for generic engineering provided herein. In some aspects, the provided methods can include incubating transduced T cells under conditions to permit integration of the viral vector into the genome of the cells.

In certain embodiments, the one or more populations of enriched CD28+ cells are cultivated, e.g., cultivated under conditions that promote or allow for T cell division, growth, or expansion, such as for a fixed amount of time or until a threshold limit for expansion is achieved. In some aspects, the cultivation is performed by any method described herein.

In particular embodiments, provided herein are methods for generating genetically engineered T cell composition from one or more initial, e.g., input, populations of CD28+ cells. In some embodiments, a population of enriched CD28+ cells is incubated under stimulating conditions, thereby generating a stimulated population. In certain embodiments, the stimulating, e.g., culturing the cells under stimulating conditions, is performed for a set or fixed amount of time, such as an amount of time under 2 days or for an amount of time between 18 hours and 30 hours. In some aspects, the stimulating with the stimulatory reagent is carried out for about 20 hours, about 24 hours, or about 48 hours.

In certain embodiments, a heterologous polynucleotide is introduced to cells of the stimulated population, thereby generating a transformed population. In particular embodiments, the cells are incubated either during or after genetically engineering the cells, for example, for an amount of time sufficient to allow for integration of a heterologous or recombinant polynucleotide encoding a recombinant protein or to allow for the expression of the recombinant protein. In certain embodiments, the cells are incubated for a set or fixed amount of time, such as an amount of time greater than 18 hours or less than 4 days, e.g., 72 hours±6 hours. In any of the provided embodiments, the introducing can be carried out on cells after they have been stimulated with the stimulatory reagent. In some embodiments, the engineering step is started or initiated within a set amount of time from when the stimulating is started or initiated, such as within 30 hours from when the stimulatory reagent is added, cultured, or contacted to the cells. In particular embodiments, the engineering step is started or initiated between 18 hours and 30 hours, such as 20 hours±4 hours, after the stimulatory reagent is added, cultured, or contacted to the cells.

In certain embodiments, the transformed population is then expanded, such as for a set amount of time or until a threshold expansion is achieved, thereby resulting in an expanded population. In some embodiments, the transformed population is expanded until the population comprises between about 150 and 540×10⁶ cells. In particular embodiments, the transformed population or the expanded population is harvested or collected, and optionally formulated, such as for administration to a subject or for cryopreservation. In some embodiments, the population is or contains CD28+CD4+ T cells and CD28+CD8+ T cells. In some embodiments, the population is or contains CD28+CD3+ T cells.

In particular embodiments, the populations of enriched T cells may be collected, formulated for cryoprotection, frozen (e.g., cryoprotected), and/or stored below 0° C., below −20° C., or at or below −70 C or −80° C. prior to, during, or after any stage or step of the process for generating engineered populations of enriched T cells expressing recombinant receptors. In some embodiments, the cells may be stored for an amount of time under 1, 2, 3, 4, 5, 6, 7, 8, 9, or 10 days, or an amount of time under 1, 2, 3, 4, 5, 6, 7, 8 weeks, or for an amount of time at least 1, 2, 3, 4, 5, 6, 7, or 8 weeks, or for more than 8 weeks. After storage, the populations of enriched T cells may be thawed and the processing may be resumed from the same point in the process. In some embodiments, input populations of enriched T cells are cryoprotected and stored prior to further processing, e.g., incubation under stimulating conditions. In particular embodiments, cultivated and/or formulated populations of enriched T cells are cryoprotected and stored prior to being administered to as subject, e.g., as an autologous cell therapy.

In certain embodiments, the methods provided herein are used in connection with a process whereby engineered cells are generated by a process that includes steps for stimulating the cells and then introducing a polynucleotide encoding a recombinant receptor, e.g., a CAR, into the cells. In particular embodiments, the stimulating is performed for between 18 and 30 hours, such as for about 24 hours, and the introduction of the polynucleotide is subsequently performed. In particular embodiments, the stimulating is performed for about 48 hours, and the introduction of the polynucleotide is subsequently performed. In various embodiments, the cells are harvested or collected, such as to be formulated for cryopreservation or administered to a subject, within 8-12 days after the incubation under stimulatory conditions is initiated. In various embodiments, the cells are harvested or collected, such as to be formulated for cryopreservation or administered to a subject, within 10 days after the incubation under stimulatory conditions is initiated.

In certain embodiments, provided herein are methods for generating genetically engineered T cell composition from two initial, e.g., input, populations of CD28+ cells. In some embodiments, the two populations of enriched CD28+ cells are separately incubated under stimulating conditions, thereby generating two separate stimulated populations. In certain embodiments, a heterologous polynucleotide is introduced to cells of the two separate stimulated populations, thereby generating two separate transformed populations. In certain embodiments, the two separate transformed populations are then expanded, such as for a set amount of time or until a threshold expansion is achieved, thereby resulting in two separate expanded populations. In particular embodiments, the two separate transformed populations or the two separate expanded populations are harvested or collected, and optionally formulated, such as for administration to a subject or for cryopreservation. In particular embodiments, the two separate populations originate or are derived from the same biological sample or different biological samples from the same individual subject. In some embodiments, the two separate populations are or contain a population of enriched CD28+CD4+ T cells and a separate population of CD28+CD8+ T cells.

Also provided are methods for identifying a population of cells capable of expanding or proliferating, such as during an incubation or cultivation under conditions that promote T cell proliferation or expansion, such as any such conditions described herein. In some embodiments, such methods are or include measuring the percentage of CD28+ cells in the population, wherein if the percentage of CD28+ cells are above a threshold value (i.e. a threshold percentage), the population is capable of expanding. In some embodiments, the threshold percentage is about 30%, 35%, 40%, 45%, 50%, or 55%. In some embodiments, the threshold is or is about 30%. In some embodiments, the threshold is or is about 35%. In some embodiments, the threshold is or is about 40%. In some embodiments, the threshold is or is about 45%. In some embodiments, a population that is capable of expanding expands at least 2-fold, 3-fold, 4-fold, or 5-fold within 10, 11, 12, 13, or 14 days during a cultivation under conditions that promote proliferation or expansion. In certain embodiments, a population that is capable of expanding expands at least 3-fold within 10 days during a cultivation. In certain embodiments, a population that is capable of expanding expands at least 4-fold within 10 days during a cultivation. In certain embodiments, a population that is capable of expanding expands at least 5-fold within 10 days during a cultivation.

In some embodiments, the method is or includes measuring a value of a trait associated with CD28 expression of a population of T cells, wherein the population of T cells is capable of expansion if the value of the trait is more than a threshold value of the trait. In some embodiments, the trait is a level or amount of a polypeptide encoded by the CD28 gene present in the total T cells, CD3+ T cells, CD4+ T cells, or CD8+ T cells of the dose. In certain embodiments, the trait is a level or amount of a polypeptide encoded by the CD28 gene present on the surface of the total T cells, CD3+ T cells, CD4+ T cells, or CD8+ T cells of the dose, in particular embodiments, the trait is a frequency, percentage, or amount of T cells, CD4+ T cells, or CD8+ T cells present positive for expression of the CD28. In some embodiments, the trait is a level or amount of mRNA of the CD28 gene present in the T cells. In particular embodiments a level or amount of accessibility of the CD28 gene.

In certain embodiments, the threshold value is at, at about, or within 25%, within 20%, within 15%, within 10%, or within 5% above a mean or median measurement of the trait associated with CD28 expression, and/or is above one standard deviation more than the mean or median measurement, in a plurality of reference T cell populations. In certain embodiments, the threshold value is above a highest measurement of the trait associated with CD28 expression, optionally within 50%, within 25%, within 20%, within 15%, within 10%, or within 5% above the highest measurement, in a population from among a plurality of reference T cell populations. In some embodiments, the threshold is above a mean or median measurement of the trait associated with CD28 expression calculated from among more than 65%, 75%, 80%, 85% of samples from a plurality of reference T cell compositions. In particular embodiments, the plurality of reference T cell populations are a plurality of populations that did not expand when cultivated under conditions that promote proliferation or expansion of T cells, optionally wherein the cells did not expand by at least 3-fold, 4-fold, or 5 fold, within 10, 11, 12, 13, or 14 days of cultivation, e.g., a cultivation as described herein. In some embodiments, the reference T cell populations did not expand by at least 2-fold within about 7 days of cultivation. In some embodiments, the reference T cell populations did not expand by at least 3-fold within about 7 days of cultivation. In some embodiments, the reference T cell populations did not expand by at least 4-fold within about 7 days of cultivation. In some embodiments, the reference T cell populations did not expand by at least 5-fold within about 7 days of cultivation. In some embodiments, the reference T cell populations did not expand by at least 6-fold within about 7 days of cultivation. In some embodiments, the reference T cell populations did not expand by at least 7-fold within about 7 days of cultivation. In some embodiments, the reference T cell populations did not expand by at least 8-fold within about 7 days of cultivation. In some embodiments, the reference T cell populations did not expand by at least 9-fold within about 7 days of cultivation. In some embodiments, the reference T cell populations did not expand by at least 10-fold within about 7 days of cultivation.

In some embodiments, the harvesting is performed at or after the time in which the engineered population or the expanded population of T cells include a threshold number of T cells, viable T cells, engineered T cells or viable engineered T cells, or a threshold concentration of T cells, viable T cells, engineered T cells or viable engineered T cells. In some embodiments, the threshold number or concentration of T cells, viable T cells, engineered T cells or viable engineered T cells is reached within at or about 4, 5, 6, 7, 8, 9, or 10 days after the initiation of stimulation. In some embodiments, the threshold number or concentration of T cells, viable T cells, engineered T cells or viable engineered T cells is reached within at or about 7 days after the initiation of stimulation. In some embodiments, the threshold number or concentration of T cells, viable T cells, engineered T cells or viable engineered T cells is reached within at or about 8 days after the initiation of stimulation. In some embodiments, the threshold number or concentration of T cells, viable T cells, engineered T cells or viable engineered T cells is reached within at or about 9 days after the initiation of stimulation. In some embodiments, the threshold number or concentration of T cells, viable T cells, engineered T cells or viable engineered T cells is reached within at or about 10 days after the initiation of stimulation.

In some embodiments, among a plurality of populations of engineered T cells or populations of expanded T cells, the threshold number or concentration of T cells, viable T cells, engineered T cells or viable engineered T cells is reached within at or about 7, 8, 9, or 10 days after the initiation of stimulation in at least at or about or at least at or about 70%, 80%, 90% or 95% of the plurality. In some embodiments, among a plurality of populations of engineered T cells or populations of expanded T cells, the threshold number or concentration of T cells, viable T cells, engineered T cells or viable engineered T cells is reached within at or about 10 days after the initiation of stimulation in at least at or about or at least at or about 70%, 80%, 90% or 95% of the plurality. In some embodiments, the threshold number or concentration of T cells, viable T cells, engineered T cells or viable engineered T cells is reached within at or about 2, 3, 4 or 5 population doublings after the initiation of stimulation. In some embodiments, the threshold number or concentration of T cells, viable T cells, engineered T cells or viable engineered T cells is reached within at or about 5 population doublings after the initiation of stimulation.

Among provided methods are methods for genetically engineering T cells expressing non-CD28 markers with a recombinant receptor, such as a CAR. In some aspects, the provided methods can include one or more steps of stimulating, activating, engineering, cultivating, and/or expanding one or more populations of enriched CD45RA+, CD45RO+, CD27+, CD197+, CD4+, CD57+, CD8+, CD25+, PD1+, LAG3+, CD3+ and/or TIM3+ cells. In certain embodiments, the one or more populations are or include any population of CD45RA+, CD45RO+, CD27+, CD197+, CD4+, CD57+, CD8+, CD25+, PD1+, LAG3+, CD3+ and/or TIM3+ cells described herein. In some embodiments, the one or more populations are isolated, selected, or enriched from a biological sample by any method or process described herein. In some embodiments, the one or more populations of enriched CD45RA+, CD45RO+, CD27+, CD197+, CD4+, CD57+, CD8+, CD25+, PD1+, LAG3+, CD3+ and/or TIM3+ cells are stimulated or activated, such as by incubating the cells of the population under stimulating conditions, such as any stimulating condition described herein. In certain embodiments, the one or more populations of enriched CD45RA+, CD45RO+, CD27+, CD197+, CD4+, CD57+, CD8+, CD25+, PD1+, LAG3+, CD3+ and/or TIM3+ cells are genetically engineered, such as by introducing a heterologous polynucleotide to the cells of the one or more populations. In some embodiments, the introducing is performed by any method for generic engineering provided herein. In some aspects, the provided methods can include incubating transduced T cells under conditions to permit integration of the viral vector into the genome of the cells.

In some embodiments, provided herein is a method of manufacturing a cell therapy, comprises: (1) selecting a subject for manufacturing a cell therapy if the percentage of any of CD28+, CD45RA+, CD45RO+, CD27+, CD197+, CD4+, CD57+, CD8+, CD25+, PD1+, LAG3+, CD3+ and/or TIM3+ T cells in a first biological sample obtained from the subject is above a threshold value, wherein the first biological sample comprises T cells; and (2) genetically engineering cells of a second biological sample obtained from the subject to express a recombinant receptor, thereby generating a cell therapy comprising the genetically engineered cells, wherein the second biological sample comprises T cells.

In some embodiments, the method further comprises determining the percentage of any of CD28+, CD45RA+, CD45RO+, CD27+, CD197+, CD4+, CD57+, CD8+, CD25+, PD1+, LAG3+, CD3+ and/or TIM3+ T cells in the first biological sample.

In some embodiments, the threshold value is calculated as the percentage of T cells that are CD28+, CD45RA+, CD45RO+, CD27+, CD197+, CD4+, CD57+, CD8+, CD25+, PD1+, LAG3+, CD3+ and/or TIM3+ in the first biological sample.

In some embodiments, the threshold value is between about 20% and about 40% CD28+ T cells, between about 30% and about 50% CD28+ T cells, or between about 35% and about 45% CD28+ T cells. In some embodiments, the threshold value is about 40% CD28+ T cells. In some embodiments, the threshold value is between about 20% and about 40% CD45RA+ T cells, between about 30% and about 50% CD45RA+ T cells, or between about 35% and about 45% CD45RA+ T cells. In some embodiments, the threshold value is about 40% CD45RA+ T cells. In some embodiments, the threshold value is between about 20% and about 40% CD45RO+ T cells, between about 30% and about 50% CD45RO+ T cells, or between about 35% and about 45% CD45RO+ T cells. In some embodiments, the threshold value is about 40% CD45RO+ T cells. In some embodiments, the threshold value is between about 20% and about 40% CD27+ T cells, between about 30% and about 50% CD27+ T cells, or between about 35% and about 45% CD27+ T cells. In some embodiments, the threshold value is about 40% CD27+ T cells. In some embodiments, the threshold value is between about 20% and about 40% CD197+ T cells, between about 30% and about 50% CD197+ T cells, or between about 35% and about 45% CD197+ T cells. In some embodiments, the threshold value is about 40% CD197+ T cells.

In some embodiments, the method further comprises administering a dose of the cell therapy to a subject. In some embodiments, a composition of cells is produced by any of the methods provided herein.

In some aspects, the provided methods can include one or more steps of stimulating, activating, engineering, cultivating, and/or expanding one or more populations of enriched IL2RA, LIF, and/or OSM expressing cells. In certain embodiments, the one or more populations are or include any population of IL2RA, LIF, and/or OSM expressing cells described herein. In some embodiments, the one or more populations are isolated, selected, or enriched from a biological sample by any method or process described herein. In some embodiments, the one or more populations of enriched IL2RA, LIF, and/or OSM expressing cells are stimulated or activated, such as by incubating the cells of the population under stimulating conditions, such as any stimulating condition described herein. In certain embodiments, the one or more populations of enriched IL2RA, LIF, and/or OSM expressing cells are genetically engineered, such as by introducing a heterologous polynucleotide to the cells of the one or more populations. In some embodiments, the introducing is performed by any method for generic engineering provided herein. In some aspects, the provided methods can include incubating transduced T cells under conditions to permit integration of the viral vector into the genome of the cells.

In some embodiments, a method of manufacturing a cell therapy, comprises (1) selecting a subject for manufacturing a cell therapy if T cell expression of interleukin 2 receptor subunit alpha (IL2RA), interleukin 6 family cytokine (LIF), and/or oncostatin M (OSM) in a first biological sample obtained from the subject is above a threshold value, wherein the first biological sample comprises T cells; and (2) genetically engineering cells of a second biological sample obtained from the subject to express a recombinant receptor, thereby generating a cell therapy comprising the genetically engineered cells, wherein the second biological sample comprises T cells.

In some embodiments, the method further comprises determining T cell expression of IL2RA, LIF, and/or OSM in the first biological sample.

In some embodiments, determining expression of IL2RA, LIF, and/or OSM comprises measuring RNA expression of IL2RA, LIF, and/or OSM. In some embodiments, RNA expression is measured in a single cell of the biological sample. In some embodiments, RNA expression is measured in a plurality of cells of the biological sample.

In some embodiments, the threshold value is calculated as the average expression level of IL2RA, LIF, and/or OSM in a reference T cell or T cell population. In some embodiments, the reference T cell or T cell population is obtained from a subject that is not selected in a method of manufacturing the cell therapy. In some embodiments, the subject that is not selected in a method of manufacturing the cell therapy has slow growing T cells.

In some embodiments, the threshold value is between about 20% and about 40% IL2RA, LIF, and/or OSM expression, between about 30% and about 50% IL2RA, LIF, and/or OSM expression, or between about 35% and about 45% IL2RA, LIF, and/or OSM expression. In some embodiments, the threshold value is about 40% IL2RA, LIF, and/or OSM expression. In some embodiments, the method further comprises administering a dose of the cell therapy to a subject. In some embodiments, the biological sample first biological sample and the second biological sample is an apheresis sample or a leukapheresis sample.

In some aspects, the provided methods can include one or more steps of stimulating, activating, engineering, cultivating, and/or expanding one or more populations of enriched MKI67, TOP2A, CD4, CCR7, NKG7, IL32, LAG3, HAVCR2, CD3G, TCF7, and/or IL7R expressing cells. In certain embodiments, the one or more populations are or include any population of MKI67, TOP2A, CD4, CCR7, NKG7, IL32, LAG3, HAVCR2, CD3G, TCF7, and/or IL7R expressing cells described herein. In some embodiments, the one or more populations are isolated, selected, or enriched from a biological sample by any method or process described herein. In some embodiments, the one or more populations of enriched MKI67, TOP2A, CD4, CCR7, NKG7, IL32, LAG3, HAVCR2, CD3G, TCF7, and/or IL7R expressing cells are stimulated or activated, such as by incubating the cells of the population under stimulating conditions, such as any stimulating condition described herein. In certain embodiments, the one or more populations of enriched MKI67, TOP2A, CD4, CCR7, NKG7, IL32, LAG3, HAVCR2, CD3G, TCF7, and/or IL7R expressing cells are genetically engineered, such as by introducing a heterologous polynucleotide to the cells of the one or more populations. In some embodiments, the introducing is performed by any method for generic engineering provided herein. In some aspects, the provided methods can include incubating transduced T cells under conditions to permit integration of the viral vector into the genome of the cells.

In some embodiments, the expression of MKI67, TOP2A, CD4, CCR7, NKG7, IL32, LAG3, HAVCR2, CD3G, TCF7, and/or IL7R is associated with sub-types and subpopulations of T cells and/or of CD4+ and/or of CD8+ T cells. In some embodiments, these sub-types comprise naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In other embodiments, these subtypes comprise CD4 proliferating T cells, CD4 central memory T cells (TCM), CD8 naïve cells, CD4 naïve cells, CD8 TCM cells, T regulatory cells (Treg), mucosal-associated invariant T cells (MAIT), cDC2 cells, plasmablast cells, or NK proliferating cells.

In some embodiments, the provided methods can include one or more steps of stimulating, activating, engineering, cultivating, and/or expanding one or more sub-types or subpopulations of T cells.

In some embodiments, a method of manufacturing a cell therapy, comprises: (1) selecting a subject for manufacturing a cell therapy if one or more genes associated with CD4 proliferating T cells, CD4 central memory T cells (TCM), CD8 naïve cells, CD4 naïve cells, CD8 TCM cells, T regulatory cells (Treg), mucosal-associated invariant T cells (MAIT), cDC2 cells, plasmablast cells, or NK proliferating cells in a first biological sample obtained from the subject is above a threshold value, wherein the first biological sample comprises T cells; and (2) genetically engineering cells of a second biological sample obtained from the subject to express a recombinant receptor, thereby generating a cell therapy comprising the genetically engineered cells, wherein the second biological sample comprises T cells.

In some embodiments, the method further comprises selecting a subject for manufacturing a cell therapy if one or more genes associated with CD4 proliferating cells in the first biological sample obtained from the subject is above the threshold value. In some embodiments, the method further comprises selecting a subject for manufacturing a cell therapy if one or more genes associated with CD4 TCM in the first biological sample obtained from the subject is above the threshold value.

In some embodiments, the one or more genes associated with CD4 proliferating T cells is selected from the group consisting of MKI67, TOP2A, CD4, CCR7, NKG7, IL32, LAG3, HAVCR2, and CD3G. In some embodiments, the one or more genes associated with CD4 TCM is selected from the group consisting of CD4, CCR7, TCF7, IL7R, IL32, and CD3G.

In particular embodiments, processes for genetically engineering enriched T cells comprises using the T cells selected and/or enriched in Sections I-IV.

In certain embodiments, provided herein are methods of genetically engineering the selected and enriched CD28+ T cells described in Sections II and Section III, and the populations of enriched CD28+ T cells in Section I. In certain embodiments, provided herein are methods of genetically engineering the selected and enriched non-CD28 T cells described in Sections II and Section III, and the populations of enriched non-CD28 T cells in Section I.

### A. Cells and Preparation for Genetic Engineering

In some embodiments, any of the cells provided herein can be used to prepare genetically engineered cells. For example, populations of enriched T cells can be used to prepare genetically engineered cells. In some embodiments, and as described throughout the present disclosure, the T cells may be enriched for CD28 or the T cells may be enriched for non-CD28 markers. In further embodiments, the T cells enriched for CD28 or the T cells enriched for non-CD28 markers may be obtained from a subject undergoing a cell selection process. In some embodiments, the selection process comprises obtaining T cells enriched for CD28 or not enriched for CD28 to determine whether the subject's cells can be used in a cell therapy.

In some embodiments, genetic engineering comprises introducing one or more recombinant receptors encoded by a virus or vector provided herein

Cells expressing the receptors administered by the provided methods are engineered cells (e.g., engineered T cells). The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the sub-types and subpopulations of T cells are CD4 proliferating T cells, CD4 central memory T cells (TCM), CD8 naïve cells, CD4 naïve cells, CD8 TCM cells, T regulatory cells (Treg), mucosal-associated invariant T cells (MAIT), cDC2 cells, plasmablast cells, or NK proliferating cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound The antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker high) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naïve, memory, and/or effector T cell subpopulations.

In some embodiments, CD8+ cells are further enriched for or deplete naïve, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., Blood. 1:72-82 (2012); Wang et al., J Immunother. 35(9):689-701 (2012). In some embodiments, combining TCM-enriched CD8+ T cells and CD4+ T cells further enhances efficacy.

In embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+T helper cells are sorted into naïve, central memory, and effector cells by identifying cell populations that have cell surface antigens. CD4+ lymphocytes can be obtained by standard methods. In some embodiments, naïve CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher© Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in PCT Publication No. WO2009/072003 or US Publication No. US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al., J Immunother. 35(9): 651-660 (2012), Terakura et al., Blood. 1:72-82 (2012), and Wang et al., J Immunother. 35(9):689-701 (2012).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al., Lab Chip 10, 1567-1573 (2010); and Godin et al., J Biophoton. 1(5):355-376 (2008). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al., J Immunother. 35(9): 651-660 (2012), Terakura et al., Blood. 1:72-82 (2012), and/or Wang et al., J Immunother. 35(9):689-701 (2012).

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees Celsius, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naïve or antigen specific T lymphocytes with antigen. For example, antigen-specific T cell lines or clones can be generated to cytomegalovirus antigens by isolating T cells from infected subjects and stimulating the cells in vitro with the same antigen.

In particular embodiments, selected or enriched cells of Sections II-IV or the population of enriched cells of Section I can be genetically engineered to express one or more recombinant receptors. In some embodiments, the recombinant receptors comprises the recombinant receptors of VIA.

### B. Stimulation

In some embodiments, the provided methods are used in connection with incubating cells, e.g., CD28+ T cells, under stimulating conditions. In some embodiments, the stimulating conditions include conditions that activate or stimulate, and/or are capable of activating or stimulating a signal in the cell, e.g., a CD4+ or a CD8+ T cell, such as a signal generated from a TCR and/or a coreceptor. In some embodiments, the stimulating conditions include one or more steps of culturing, cultivating, incubating, activating, propagating the cells with and/or in the presence of a stimulatory reagent, e.g., a reagent that activates or stimulates, and/or is capable of activating or stimulating a signal in the cell. In some embodiments, the stimulatory reagent stimulates and/or activates a TCR and/or a coreceptor. In particular embodiments, the stimulatory reagent is a reagent described herein.

In certain embodiments, one or more populations of enriched CD28+ T cells are incubated under stimulating conditions prior to genetically engineering the cells, e.g., transfecting and/or transducing the cell such as by a technique provided herein. In particular embodiments, one or more populations of enriched CD28+ T cells are incubated under stimulating conditions after the one or more compositions have been isolated, selected, enriched, or obtained from a biological sample (e.g. a second biological sample, such as an apheresis or PBMC sample). In particular embodiments, the one or more populations of enriched CD28+ T cells have been previously cryopreserved and stored, and are thawed prior to the incubation.

In certain embodiments, the one or more populations of enriched CD28+ T cells are or include two separate populations of enriched CD28+ T cells. In particular embodiments, the two separate compositions of enriched T cells, e.g., two separate compositions of enriched T cells selected, isolated, and/or enriched from the same biological sample, are separately incubated under stimulating conditions. In certain embodiments, the two separate compositions include a composition of enriched CD28+CD4+ T cells. In particular embodiments, the two separate compositions include a composition of enriched CD28+CD8+ T cells. In some embodiments, two separate compositions of enriched CD28+CD4+ T cells and enriched CD28+CD8+ T cells are separately incubated under stimulating conditions. In some embodiments, a single composition of enriched T cells is incubated under stimulating conditions. In certain embodiments, the single composition is a composition of enriched CD28+CD4+ T cells. In certain embodiments, the single composition is a composition of enriched CD28+CD8+ T cells. In certain embodiments, the single composition is a composition of enriched CD28+CD3+ T cells. In some embodiments, the single composition is a composition of enriched CD28+CD4+ and CD28+CD8+ T cells that have been combined from separate compositions prior to the incubation.

In some embodiments, the population of enriched CD28+CD4+ T cells that is incubated under stimulating conditions includes at least at or about 60%, at least at or about 65%, at least at or about 70%, at least at or about 75%, at least at or about 80%, at least at or about 85%, at least at or about 90%, at least at or about 95%, at least at or about 98%, at least at or about 99%, at least at or about 99.5%, at least at or about 99.9%, or at or at about 100% CD28+CD4+ T cells. In certain embodiments, the composition of enriched CD28+CD4+ T cells that is incubated under stimulating conditions includes less than at or about 40%, less than at or about 35%, less than at or about 30%, less than at or about 25%, less than at or about 20%, less than at or about 15%, less than at or about 10%, less than at or about 5%, less than at or about 1%, less than at or about 0.1%, or less than at or about 0.01% CD28− T cells.

In certain embodiments, the population of enriched CD28+CD8+ T cells that is incubated under stimulating conditions includes at least at or about 60%, at least at or about 65%, at least at or about 70%, at least at or about 75%, at least at or about 80%, at least at or about 85%, at least at or about 90%, at least at or about 95%, at least at or about 98%, at least at or about 99%, at least at or about 99.5%, at least at or about 99.9%, or at or at about 100% CD28+CD8+ T cells. In certain embodiments, the composition of enriched CD28+CD4+ T cells that is incubated under stimulating conditions includes less than at or about 40%, less than at or about 35%, less than at or about 30%, less than at or about 25%, less than at or about 20%, less than at or about 15%, less than at or about 10%, less than at or about 5%, less than at or about 1%, less than at or about 0.1%, or less than at or about 0.01% CD28− T cells. In certain embodiments, the composition of enriched CD28+CD8+ T cells that is incubated under stimulating conditions includes less than at or about 40%, less than at or about 35%, less than at or about 30%, less than at or about 25%, less than at or about 20%, less than at or about 15%, less than at or about 10%, less than at or about 5%, less than at or about 1%, less than at or about 0.1%, or less than at or about 0.01% CD28− T cells.

In certain embodiments, the population of enriched CD28+CD3+ T cells that is incubated under stimulating conditions includes at least at or about 60%, at least at or about 65%, at least at or about 70%, at least at or about 75%, at least at or about 80%, at least at or about 85%, at least at or about 90%, at least at or about 95%, at least at or about 98%, at least at or about 99%, at least at or about 99.5%, at least at or about 99.9%, or at or at about 100% CD28+CD3+ T cells. In certain embodiments, the composition of enriched CD28+CD3+ T cells that is incubated under stimulating conditions includes less than at or about 40%, less than at or about 35%, less than at or about 30%, less than at or about 25%, less than at or about 20%, less than at or about 15%, less than at or about 10%, less than at or about 5%, less than at or about 1%, less than at or about 0.1%, or less than at or about 0.01% CD28− T cells.

In certain embodiments, separate compositions of enriched CD28+CD4+ and CD28+CD8+ T cells are combined into a single composition and are incubated under stimulating conditions. In certain embodiments, separate stimulated compositions of enriched CD28+CD4+ and enriched CD28+CD8+ T cells are combined into a single composition after the incubation has been performed and/or completed.

In some embodiments, the incubation under stimulating conditions can include culture, cultivation, stimulation, activation, propagation, including by incubation in the presence of stimulating conditions, for example, conditions designed to induce proliferation, expansion, activation, and/or survival of cells in the population, to mimic antigen exposure, and/or to prime the cells for genetic engineering, such as for the introduction of a recombinant antigen receptor. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, the stimulation and/or incubation under stimulating conditions is carried out in accordance with techniques such as those described in U.S. Pat. No. 6,040,177 to Riddell et al., Klebanoff et al. (2012) J Immunother. 35(9): 651-660, Terakuraet al. (2012) Blood. 1:72-82, and/or Wang et al. (2012) J Immunother. 35(9):689-701.

In some embodiments, the CD28+ T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25° C., generally at least about 30 degrees, and generally at or about 37° C. In some embodiments, a temperature shift is effected during culture, such as from 37° C. to 35° C. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In particular embodiments, the stimulating conditions include incubating, culturing, and/or cultivating the cells with a stimulatory reagent. In particular embodiments, the stimulatory reagent is a reagent described herein. In certain embodiments, the stimulatory reagent contains or includes a bead. In particular embodiments, the stimulatory reagent contains or includes an oligomeric reagent, e.g., an oligomeric streptavidin mutein reagent. In certain embodiments, the start and or initiation of the incubation, culturing, and/or cultivating cells under stimulating conditions occurs when the cells are come into contact with and/or are incubated with the stimulatory reagent. In particular embodiments, the cells are incubated prior to, during, and/or subsequent to genetically engineering the cells, e.g., introducing a recombinant polynucleotide into the cell such as by transduction or transfection. In some embodiments, the composition of enriched T cells are incubated at a ratio of stimulatory reagent and/or beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of stimulatory reagent and/or beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. Exemplary stimulatory reagents are described below.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the stimulation, e.g. culturing the cells under stimulating conditions, is performed for, for about, or for less than, 48 hours, 42 hours, 36 hours, 30 hours, 24 hours, 22 hours, 20 hours, 18 hours, 16 hours, or 12 hours. In some embodiments, the stimulation, e.g. culturing the cells under stimulating conditions, is performed for 20±4 hours or between or between about 16 hours and 24 hours. In particular embodiments, the stimulation, e.g. culturing the cells under stimulating conditions, is performed for between or between about 36 hours and 12 hours, 30 hours and 18 hours, or for or for about 24 hours, or 22 hours. In some embodiments, the stimulation, e.g. culturing the cells under stimulating conditions, is performed for, for about, or for less than, 2 days or one day.

In particular embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes IL-2.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength e.g., WHO 1st International Standard for Human IL-2, 86/504. International Units are the only recognized and standardized method to report biological activity units that are published and are derived from an international collaborative research effort. In particular embodiments, the IU for population, sample, or source of a cytokine may be obtained through product comparison testing with an analogous WHO standard product. For example, in some embodiments, the IU/mg of a population, sample, or source of human recombinant IL-2, IL-7, or IL-15 is compared to the WHO standard IL-2 product (NIBSC code: 86/500), the WHO standard IL-17 product (NIBSC code: 90/530) and the WHO standard IL-15 product (NIBSC code: 95/554), respectively.

In some embodiments, the cells, e.g., the input cells, are stimulated or subjected to stimulation in the presence of a cytokine, e.g., a recombinant human cytokine, at a concentration of between 1 IU/mL and 1,000 IU/mL, between 10 IU/mL and 50 IU/mL, between 50 IU/mL and 100 IU/mL, between 100 IU/mL and 200 IU/mL, between 100 IU/mL and 500 IU/mL, between 250 IU/mL and 500 IU/mL, or between 500 IU/mL and 1,000 IU/mL.

In some embodiments, the cells, e.g., the input cells, are stimulated or subjected to stimulation in the presence of recombinant IL-2, e.g., human recombinant IL-2, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, cells, e.g., cells of the input population, are stimulated or subjected to stimulation in the presence of recombinant IL-2 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 100 IU/mL. In some embodiments, the cells, e.g., the input cells, are stimulated or subjected to stimulation in the presence of or of about 100 IU/mL of recombinant IL-2, e.g., human recombinant IL-2.

In some embodiments, the cells, e.g., the input cells, are stimulated or subjected to stimulation in the presence of recombinant IL-7, e.g., human recombinant IL-7, at a concentration between 100 IU/mL and 2,000 IU/mL, between 500 IU/mL and 1,000 IU/mL, between 100 IU/mL and 500 IU/mL, between 500 IU/mL and 750 IU/mL, between 750 IU/mL and 1,000 IU/mL, or between 550 IU/mL and 650 IU/mL. In particular embodiments, the cells, e.g., the input cells, are stimulated or subjected to stimulation in the presence of IL-7 at a concentration at or at about 50 IU/mL, 100 IU/mL, 150 IU/mL, 200 IU/mL, 250 IU/mL, 300 IU/mL, 350 IU/mL, 400 IU/mL, 450 IU/mL, 500 IU/mL, 550 IU/mL, 600 IU/mL, 650 IU/mL, 700 IU/mL, 750 IU/mL, 800 IU/mL, 750 IU/mL, 750 IU/mL, 750 IU/mL, or 1,000 IU/mL. In particular embodiments, the cells, e.g., the input cells, are stimulated or subjected to stimulation in the presence of or of about 600 IU/mL of recombinant IL-7, e.g., human recombinant IL-7.

In some embodiments, the cells, e.g., the input cells, are stimulated or subjected to stimulation in the presence of recombinant IL-15, e.g., human recombinant IL-15, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, cells, e.g., a cell of the input population, are stimulated or subjected to stimulation in the presence of recombinant IL-15 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 200 IU/mL. In some embodiments, the cells, e.g., the input cells, are stimulated or subjected to stimulation in the presence of or of about 100 IU/mL of recombinant IL-15, e.g., human recombinant IL-15.

In particular embodiments, the cells, e.g., cells from the input population, are stimulated or subjected to stimulation under stimulating conditions in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15. In certain embodiments, the cells are stimulated or subjected to stimulation under stimulating conditions in the presence of recombinant IL-2, IL-7, and IL-15. In certain embodiments, the cells are stimulated or subjected to stimulation under stimulating conditions in the presence of recombinant IL-2 of or of about 100 IU/mL, recombinant IL-7 of or of about 600 IU/mL, and recombinant IL-15 of or of about 100 IU/mL.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, stimulation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In certain embodiments, the stimulation is performed under static conditions, such as conditions that do not involve centrifugation, shaking, rotating, rocking, or perfusion, e.g., continuous or semi-continuous perfusion of the media. In some embodiments, either prior to or shortly after, e.g., within 5, 15, or 30 minutes of the initiation, the cells are transferred (e.g., transferred under sterile conditions) to a container such as a bag or vial, and placed in an incubator. In particular embodiments, incubator is set at, at about, or at least 16° C., 24° C., or 35° C. In some embodiments, the incubator is set at 37° C., at about at 37° C., or at 37° C.±2° C., ±1° C., ±0.5° C., or 0.1° C. In particular embodiments, the stimulation under static condition is performed in a cell culture bag placed in an incubator.

In some embodiments, the stimulation results in activation and/or proliferation of the cells, for example, prior to transduction.

### 1. Stimulatory Reagents

In particular embodiments, the stimulating conditions include incubating, culturing, and/or cultivating the cells with a stimulatory reagent. In certain embodiments, the stimulatory reagent contains or includes a bead. In certain embodiments, the initiation of the stimulation occurs when the cells are incubated or contacted with the stimulatory reagent. In particular embodiments, the stimulatory reagent contains or includes an oligomeric reagent, e.g., a streptavidin mutein oligomer. In particular embodiments, the stimulatory reagent activates and/or is capable of activating one or more intracellular signaling domains of one or more components of a TCR complex and/or one or more intracellular signaling domains of one or more costimulatory molecules.

In some embodiments, the stimulating conditions or stimulatory reagents include one or more agent, e.g., ligand, which is capable of activating an intracellular signaling domain of a TCR complex. In some embodiments, an agent as contemplated herein can include, but is not limited to, RNA, DNA, proteins (e.g., enzymes), antigens, polyclonal antibodies, monoclonal antibodies, antibody fragments, carbohydrates, lipids lectins, or any other biomolecule with an affinity for a desired target. In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS. The one or more agents may be attached directly or indirectly to the bead by a variety of known methods. The attachment may be covalent, noncovalent, electrostatic, or hydrophobic and may be accomplished by a variety of attachment means, including for example, a chemical means, a mechanical means, or an enzymatic means. In some embodiments, the agent is an antibody or antigen binding fragment thereof, such as a Fab. In some embodiments, a biomolecule (e.g., a biotinylated anti-CD3 antibody) may be attached indirectly to the bead via another biomolecule (e.g., anti-biotin antibody) that is directly attached to the bead.

In some embodiments, the stimulatory reagent contains one or more agents (e.g. antibody) that is attached to a bead (e.g., a paramagnetic bead) and specifically binds to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD30L, LIGHT, IL-2R, IL-12R, IL-1R, IL-15R; IFN-gammaR, TNF-alphaR, IL-4R, IL-10R, CD18/CD11a (LFA-1), CD62L (L-selectin), CD29/CD49d (VLA-4), Notch ligand (e.g. Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, an agent (e.g. antibody) attached to the bead specifically binds to one or more of the following macromolecules on a cell (e.g. a T cell): CD28, CD62L, CCR7, CD27, CD127, CD3, CD4, CD8, CD45RA, and/or CD45RO.

In some embodiments, one or more of the agents attached to the bead is an antibody. The antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the stimulatory reagent is an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species).

In some embodiments, the agent is an antibody that binds to and/or recognizes one or more components of a T cell receptor. In particular embodiments, the agent is an anti-CD3 antibody. In certain embodiments, the agent is an antibody that binds to and/or recognizes a co-receptor. In some embodiments, the stimulatory reagent comprises an anti-CD28 antibody. In some embodiments, the stimulatory reagent comprises an anti-CD28 antibody and an anti-CD3 antibody. In some embodiments, the stimulatory reagent comprises one or more stimulatory agents. In some embodiments, the stimulatory reagent comprises a primary and a secondary stimulatory agent. In some embodiments, the first stimulatory agent is an anti-CD3 antibody or antigen-binding fragment thereof, for example as described herein, and the second stimulatory agent is an anti-CD28 antibody or antigen-binding fragment thereof, for example as described herein. In some embodiments, the first stimulatory agent is an anti-CD3 Fab, for example as described herein, and the second stimulatory agent is an anti-CD28 Fab, for example as described herein.

In some embodiments, the stimulatory reagent binds to a molecule on the surface of a cell, which binding between the stimulatory reagent and the molecule is capable of inducing, delivering, or modulating a stimulatory signal in the cells. In some instances, the cell surface molecule (e.g. receptor) is a signaling molecule. In some such cases, the stimulatory reagent is capable of specifically binding to a signaling molecule expressed by one or more target cells (e.g., T cells). In some instances, the stimulatory reagent is any agent that is capable of inducing or delivering a stimulatory signal in a cell (e.g., a T cell) upon binding to a cell surface molecule, such as a receptor. In some embodiments, the stimulatory signal can be immunostimulatory, in which case the stimulatory agent is capable of inducing, delivering, or modulating a signal that is involved in or that does stimulate an immune response by the cell (e.g. T cell), e.g., increase immune cell proliferation or expansion, immune cell activation, immune cell differentiation, cytokine secretion, cytotoxic activity or one or more other functional activities of an immune cell. In some embodiments, the stimulatory signal can be inhibitory, in which case the stimulatory reagent is capable of inducing, delivering, or modulating a stimulatory signal in the cell (e.g. T cell) that is involved in or that does inhibit an immune response, e.g. inhibits or decreases immune cell proliferation or expansion, immune cell activation, immune cell differentiation, cytokine secretion, cytotoxic activity or one or more other functional activities of an immune cell.

In some embodiments, the stimulatory reagent comprises a primary stimulatory agent. In some embodiments, the primary stimulatory agent binds to a receptor molecule on the surface of the selected cells of the sample. Thus, in some cases, the primary stimulatory agent delivers, induces, or modulates a stimulatory signal. In some aspects, the delivering, inducing, or modulating of a stimulatory signal by the primary stimulatory agent effects the stimulation of the cells. Thus, in some cases, the primary stimulatory agent delivers a stimulatory signal or provides a primary activation signal to the cells, thereby stimulating and/or activating the cells. In some embodiments, the primary stimulatory agent further induces downregulation of a selection marker. As used herein, downregulation may encompass a reduction in expression, e.g., cell surface expression, of a selection marker compared to an earlier time point.

In some embodiments, the target cells (e.g., T cells) comprise TCR/CD3 complexes and costimulatory molecules, such as CD28. In this case, the primary stimulatory agent binds to a TCR/CD3 complex, thereby delivering a stimulatory signal (e.g., a primary signal, e.g., primary activation signal) in the T cells, and the secondary stimulatory agent binds to a costimulatory CD28 molecule. In particular aspects, the primary stimulatory agent and/or the secondary stimulatory agent further induce downregulation of a selection marker (e.g., a selection marker used to immobilize the target cells (e.g., T cells)).

In some embodiments, the primary stimulatory agent delivers a TCR/CD3 complex-associated stimulatory signal (e.g., primary signal) in the cells, e.g., T cells. In some embodiments, the primary stimulatory agent specifically binds to a molecule containing an immunoreceptor tyrosine-based activation motif or ITAM. In some aspects, the primary stimulatory agent specifically binds CD3. In some cases, a primary stimulatory agent that specifically binds CD3 may be selected from the group consisting of an anti-CD3-antibody, a divalent antibody fragment of an anti-CD3 antibody, a monovalent antibody fragment of an anti-CD3-antibody, and a proteinaceous CD3 binding molecule with antibody-like binding properties. The divalent antibody fragment may be a F(ab′)2-fragment, or a divalent single-chain Fv fragment while the monovalent antibody fragment may be selected from the group consisting of a Fab fragment, an Fv fragment, and a single-chain Fv fragment (scFv). In some cases, a proteinaceous CD3 binding molecule with antibody-like binding properties may be an aptamer, a mutein based on a polypeptide of the lipocalin family, a glubody, a protein based on the ankyrin scaffold, a protein based on the crystalline scaffold, an adnectin, or an avimer.

In some embodiments, an anti-CD3 Fab fragment can be derived from the CD3 binding monoclonal antibody produced by the hybridoma cell line OKT3 (ATCC® CRL-8001™; see also U.S. Pat. No. 4,361,549). The variable domain of the heavy chain and the variable domain of the light chain of the anti-CD3 antibody OKT3 are described in Arakawa et al J. Biochem. 120, 657-662 (1996).

In some embodiments, the stimulatory agent comprises a secondary stimulatory agent. In some embodiments, the secondary stimulatory agent binds to a molecule on the surface of the cells, such as a cell surface molecule, e.g., receptor molecule. In some embodiments, the secondary stimulatory agent is capable of enhancing, dampening, or modifying a stimulatory signal delivered through the molecule bound by the first stimulatory agent. In some embodiments, the secondary stimulatory agent delivers, induces, or modulates a stimulatory signal, e.g., a second or an additional stimulatory signal. In some aspects, the secondary stimulatory agent enhances or potentiates a stimulatory signal induced by the primary stimulatory agent. In some embodiments, the secondary stimulatory agent binds to an accessory molecule and/or can stimulate or induce an accessory or secondary stimulatory signal in the cell. In some aspects, the secondary stimulatory agent binds to a costimulatory molecule and/or provides a costimulatory signal.

In some embodiments, the stimulatory agent, which can comprise the secondary stimulatory agent, binds, e.g. specifically binds, to a second molecule that can be a costimulatory molecule, an accessory molecule, a cytokine receptor, a chemokine receptor, an immune checkpoint molecule, or a member of the TNF family or the TNF receptor family.

In some embodiments, the molecule on the cell, e.g., T cell, may be CD28 and the secondary stimulatory agent) specifically binds CD28. In some aspects, the secondary stimulatory agent that specifically binds CD28 may be selected from the group consisting of an anti-CD28− antibody, a divalent antibody fragment of an anti-CD28 antibody, a monovalent antibody fragment of an anti-CD28-antibody, and a proteinaceous CD28 binding molecule with antibody-like binding properties. The divalent antibody fragment may be an F(ab′)2-fragment, or a divalent single-chain Fv fragment while the monovalent antibody fragment may be selected from the group consisting of a Fab fragment, an Fv fragment, and a single-chain Fv fragment (scFv). A proteinaceous CD28 binding molecule with antibody-like binding properties may be an aptamer, a mutein based on a polypeptide of the lipocalin family, a glubody, a protein based on the ankyrin scaffold, a protein based on the crystalline scaffold, an adnectin, and an avimer.

In some embodiments, an anti-CD28 Fab fragment can be derived from antibody CD28.3 (deposited as a synthetic single chain Fv construct under GenBank Accession No. AF451974.1; see also Vanhove et al, BLOOD, 15 Jul. 2003, Vol. 102, No. 2, pages 564-570).

In any of the above examples, the divalent antibody fragment may be a (Fab)2′-fragment, or a divalent single-chain Fv fragment while the monovalent antibody fragment may be selected from the group consisting of a Fab fragment, an Fv fragment, and a single-chain Fv fragment (scFv). In any of the above examples, the proteinaceous binding molecule with antibody-like binding properties may be an aptamer, a mutein based on a polypeptide of the lipocalin family, a glubody, a protein based on the ankyrin scaffold, a protein based on the crystalline scaffold, an adnectin, and an avimer.

In some aspects, the stimulatory agent specifically targets a molecule expressed on the surface of the target cells in which the molecule is a TCR, a chimeric antigen receptor, or a molecule comprising an immunoreceptor tyrosine-based activation motif or ITAM. For example, the molecule expressed on the surface of the target cell is selected from a T cell or B cell antigen receptor complex, a CD3 chain, a CD3 zeta, an antigen-binding portion of a T cell receptor or a B cell receptor, or a chimeric antigen receptor. In some cases, the stimulatory agent targets peptide:MHC class I complexes.

In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS.

In some embodiments, for example when the stimulatory agent is not bound to a stimulatory reagent or a receptor-binding agent reagent, the stimulatory agent is an antibody, a divalent antibody fragment, a F(ab)₂, or a divalent single-chain Fv fragment.

In certain embodiments, the stimulatory reagent contains a particle, e.g., a bead, that is conjugated or linked to one or more agents, e.g., biomolecules, that are capable of activating and/or expanding cells, e.g., T cells. In some embodiments, the one or more agents are bound to a bead. In some embodiments, the bead is biocompatible, i.e., composed of a material that is suitable for biological use. In some embodiments, the beads are non-toxic to cultured cells, e.g., cultured T cells. In some embodiments, the beads may be any particles which are capable of attaching agents in a manner that permits an interaction between the agent and a cell.

In some embodiments, the stimulatory reagent contains a bead and one or more agents that directly interact with a macromolecule on the surface of a cell. In certain embodiments, the bead (e.g., a paramagnetic bead) interacts with a cell via one or more agents (e.g., an antibody) specific for one or more macromolecules on the cell (e.g., one or more cell surface proteins). In certain embodiments, the bead (e.g., a paramagnetic bead) is labeled with a first agent described herein, such as a primary antibody (e.g., an anti-biotin antibody) or other biomolecule, and then a second agent, such as a secondary antibody (e.g., a biotinylated anti-CD3 antibody) or other second biomolecule (e.g., streptavidin), is added, whereby the secondary antibody or other second biomolecule specifically binds to such primary antibodies or other biomolecule on the particle.

In some embodiments, the bead reacts in a magnetic field. In some embodiments, the bead is a magnetic bead. In some embodiments, the magnetic bead is paramagnetic. In particular embodiments, the magnetic bead is superparamagnetic. In certain embodiments, the beads do not display any magnetic properties unless they are exposed to a magnetic field.

In particular embodiments, the bead comprises a magnetic core, a paramagnetic core, or a superparamagnetic core.

In certain embodiments, the bead contains a magnetic, paramagnetic, and/or superparamagnetic core that is covered by a surface functionalized coat or coating. In some embodiments, the coat can contain a material that can include, but is not limited to, a polymer, a polysaccharide, a silica, a fatty acid, a protein, a carbon, agarose, sepharose, or a combination thereof. In some embodiments, the polymer can be a polyethylene glycol, poly (lactic-co-glycolic acid), polyglutaraldehyde, polyurethane, polystyrene, or a polyvinyl alcohol. In certain embodiments, the outer coat or coating comprises polystyrene. In particular embodiments, the outer coating is surface functionalized.

In particular embodiments, the stimulatory reagent contains an oligomeric reagent, e.g., a streptavidin mutein reagent, that is conjugated, linked, or attached to one or more agent, e.g., ligand, which is capable of activating an intracellular signaling domain of a TCR complex. In some embodiments, the one or more agents have an attached binding domain or binding partner that is capable of binding to oligomeric reagent at a particular binding sites. In some embodiments, a plurality of the agent is reversibly bound to the oligomeric reagent. In various embodiments, the oligomeric reagent has a plurality of the particular binding sites which, in certain embodiments, are reversibly bound to a plurality of agents at the binding domain. In some embodiments, the amount of bound agents are reduced or decreased in the presence of a competition reagent, e.g., a reagent that is also capable of binding to the particular binding sites.

In some embodiments, the oligomeric stimulatory reagent is or includes a reversible system in which at least one agent (e.g., an agent that is capable of producing a signal in a cell such as a T cell) is associated, e.g., reversibly associated, with the oligomeric reagent. Non-limiting examples of oligomeric stimulatory reagents may be found, for example, in International published PCT Appl. No. WO 2018/197949, the contents of which are incorporated herein by reference in their entirety. In some embodiments, the reagent contains a plurality of binding sites capable of binding, e.g., reversibly binding, to the agent. In some cases, the reagent is an oligomeric particle reagent having at least one attached agent capable of producing a signal in a cell such as a T cell. Substances that may be used as oligomeric reagents in such reversible systems are known, see e.g., U.S. Pat. Nos. 5,168,049; 5,506,121; 6,103,493; 7,776,562; 7,981,632; 8,298,782; 8,735,540; 9,023,604; and International published PCT Appl. Nos. WO2013/124474 and WO2014/076277. Non-limiting examples of reagents and binding partners capable of forming a reversible interaction, as well as substances (e.g. competition reagents) capable of reversing such binding, are described below.

In some embodiments, the oligomeric reagent is an oligomer of streptavidin, streptavidin mutein or analog, avidin, an avidin mutein or analog (such as neutravidin) or a mixture thereof, in which such oligomeric reagent contains one or more binding sites for reversible association with the binding domain of the agent. In some embodiments, the binding domain of the agent can be a biotin, a biotin derivative or analog, or a streptavidin-binding peptide or other molecule that is able to specifically bind to streptavidin, a streptavidin mutein or analog, avidin or an avidin mutein or analog.

In certain embodiments, one or more agents (e.g., agents that are capable of producing a signal in a cell such as a T cell) associate with, such as are reversibly bound to, the oligomeric reagent, such as via the plurality of the particular binding sites present on the oligomeric reagent. In some cases, this results in the agents being closely arranged to each other such that an avidity effect can take place if a target cell having (at least two copies of) a cell surface molecule that is bound by or recognized by the agent is brought into contact with the agent.

In some embodiments, the oligomeric reagent is a streptavidin oligomer, a streptavidin mutein oligomer, a streptavidin analog oligomer, an avidin oligomer, an oligomer composed of avidin mutein or avidin analog (such as neutravidin) or a mixture thereof. In particular embodiments, the oligomeric reagents contain particular binding sites that are capable of binding to a binding domain of an agent. In some embodiments, the binding domain can be a biotin, a biotin derivative or analog, or a streptavidin-binding peptide or other molecule that is able to specifically bind to streptavidin, a streptavidin mutein or analog, avidin or an avidin mutein or analog. The methods provided herein further contemplate that the oligomeric reagent may comprise a molecule capable of binding to an oligohistidine affinity tag, a glutathione-S-transferase, calmodulin or an analog thereof, calmodulin binding peptide (CBP), a FLAG-peptide, an HA-tag, maltose binding protein (MBP), an HSV epitope, a myc epitope, and/or a biotinylated carrier protein.

In some embodiments, the streptavidin can be wild-type streptavidin, streptavidin muteins or analogs, such as streptavidin-like polypeptides. Likewise, avidin, in some aspects, includes wild-type avidin or muteins or analogs of avidin such as neutravidin, a deglycosylated avidin with modified arginines that typically exhibits a more neutral pi and is available as an alternative to native avidin. Generally, deglycosylated, neutral forms of avidin include those commercially available forms such as “Extravidin”, available through Sigma Aldrich, or “NeutrAvidin” available from Thermo Scientific or Invitrogen, for example.

Examples of streptavidins or streptavidin muteins are mentioned, for example, in WO 86/02077, DE 19641876 A1, U.S. Pat. No. 6,022,951, WO 98/40396 or WO 96/24606. Examples of streptavidin muteins are known, see e.g., U.S. Pat. Nos. 5,168,049; 5,506,121; 6,022,951; 6,156,493; 6,165,750; 6,103,493; or 6,368,813; or International published PCT App. No. WO2014/076277.

In some embodiments, the stimulatory reagent is removed or separated from the cells or cell populations prior to collecting, harvesting, or formulating the cells. In some embodiments, the stimulatory reagents are removed or separated from the cells or cell populations after or during the incubation, e.g., an incubation described herein. In certain embodiments, the cells or cell population undergoes a process, procedure, step, or technique to remove the stimulatory reagent after the incubation but prior to steps for collecting, harvesting, or formulating the cells. In particular embodiments, the cells or cell population undergoes a process, procedure, step, or technique to remove the stimulatory reagent after the incubation. In some aspects, when stimulatory reagent is separated or removed from the cells during the incubation, the cells are returned to the same incubation conditions as prior to the separation or removal for the remaining duration of the incubation.

In certain embodiments, the stimulatory reagent is removed and/or separated from the cells. Without wishing to be bound by theory, particular embodiments contemplate that the binding and/or association between a stimulatory reagent and cells may, in some circumstances, be reduced over time during the incubation. In certain embodiments, one or more agents may be added to reduce the binding and/or association between the stimulatory reagent and the cells. In particular embodiments, a change in cell culture conditions, e.g., the addition of an agent, may reduce the binding and/or association between the stimulatory reagent and the cells. Thus, in some embodiments, the stimulatory reagent may be removed from an incubation, cell culture system, and/or a solution separately from the cells, e.g., without removing the cells from the incubation, cell culture system, and/or a solution as well.

In certain embodiments, the stimulatory reagent is separated and/or removed from the cells after an amount of time. In particular embodiments, the amount of time is an amount of time from the initiation of the stimulation. In particular embodiments the start of the incubation is considered at or at about the time the cells are contacted with the stimulatory reagent and/or a media or solution containing the stimulatory reagent. In particular embodiments, the stimulatory reagent is removed or separated from the cells within or within about 120 hours, 108 hours, 96 hours, 84 hours, 72 hours, 60 hours, 48 hours, 36 hours, 24 hours, or 12 hours, inclusive, of the initiation of the stimulation. In particular embodiments, the stimulatory reagent is removed or separated from the cells at or at about 48 hours after the stimulation is initiated. In certain embodiments, the stimulatory reagent is removed or separated from the cells at or at about 72 hours after the stimulation is initiated. In some embodiments, the stimulatory reagent is removed or separated from the cells at or at about 96 hours after the stimulation is initiated.

### C. Genetic Engineering

In some embodiments, the provided methods include genetically engineering the cells, e.g., cells of or derived from a population of enriched CD28+ T cells, such as by introducing a heterologous polynucleotide encoding a recombinant protein. Such recombinant proteins may include recombinant receptors, such as any described herein. Introduction of the polynucleotides, e.g., heterologous or recombinant polynucleotides, encoding the recombinant protein into the cell may be carried out using any of a number of known vectors. Such vectors include viral, including lentiviral and gammaretroviral, systems. Exemplary methods include those for transfer of heterologous polynucleotides encoding the receptors, including via viral, e.g., retroviral or lentiviral, transduction. In some embodiments, a population of stimulated cells is genetically engineered, such as to introduce a heterologous or recombinant polynucleotide encoding a recombinant receptor, thereby generating a population of transformed cells (also referred to herein as a transformed population of cells).

In particular embodiments, the cells are genetically engineered, transformed, or transduced after the cells have been stimulated, activated, and/or incubated under stimulating conditions, such as by any of the methods provided herein. In particular embodiments, the one or more stimulated populations have been previously enriched for CD28+ T cells.

In certain embodiments, methods for genetic engineering are carried out by contacting or introducing one or more cells of a population with a polynucleotide encoding a recombinant protein, e.g. a recombinant receptor. In certain embodiments, the nucleic acid molecule or polynucleotide is heterologous to the cells. In particular embodiments, the heterologous polynucleotide is not native to the cells. In certain embodiments, the heterologous polynucleotide is not native to any vector, e.g., viral vector, from which it is delivered. In certain embodiments, the heterologous polynucleotide encodes a protein, e.g., a recombinant protein, that is not natively expressed by the cell. In particular embodiments, the heterologous nucleic polynucleotide is or contains a nucleic acid sequence that is not found in the cell prior to the introduction.

In some embodiments, the cells, e.g., stimulated cells, are engineered, e.g., transduced or in the presence of a transduction adjuvant. Exemplary transduction adjuvants include, but are not limited to, polycations, fibronectin or fibronectin-derived fragments or variants, and RetroNectin. In certain embodiments, the cells are engineered in the presence of polycations, fibronectin or fibronectin-derived fragments or variants, and/or RetroNectin. In particular embodiments, the cells are engineered in the presence of a polycation that is polybrene, DEAE-dextran, protamine sulfate, poly-L-lysine, or a cationic liposome. In particular embodiments, the cells are engineered in the presence of protamine sulfate.

In some embodiments, the genetic engineering, e.g., transduction, is carried out in serum free media. In some embodiments, the serum free media is a defined or well-defined cell culture media. In certain embodiments, the serum free media is a controlled culture media that has been processed, e.g., filtered to remove inhibitors and/or growth factors. In some embodiments, the serum free media contains proteins. In certain embodiments, the serum-free media may contain serum albumin, hydrolysates, growth factors, hormones, carrier proteins, and/or attachment factors.

In particular embodiments, the cells are engineered in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In some embodiments, the cells are genetically engineered, transformed, or transduced in the presence of the same or similar media as was present during the stimulation. In some embodiments, the cells are genetically engineered, transformed, or transduced in media having the same cytokines as the media present during stimulation. In certain embodiments, the cells are genetically engineered, transformed, or transduced, in media having the same cytokines at the same concentrations as the media present during stimulation.

In some embodiments, the cells are genetically engineered, transformed, or transduced in the presence of the same or similar media as was present during the stimulation. In some embodiments, the cells are genetically engineered, transformed, or transduced in media having the same cytokines as the media present during stimulation. In certain embodiments, the cells are genetically engineered, transformed, or transduced, in media having the same cytokines at the same concentrations as the media present during stimulation.

### 1. Transduction

In some embodiments, genetically engineering the cells is or includes introducing the polynucleotide, e.g., the heterologous polynucleotide, into the cells by transduction. In some embodiments, the cells are transduced with a viral vector. In some embodiments, the virus is a retroviral vector, such as a gammaretroviral vector or a lentiviral vector. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the transduction is carried out by contacting one or more cells of a population with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the provided methods are used in connection with transducing a viral vector containing a polynucleotide encoding a recombinant receptor into, into about, or into less than 300×10⁶ cells, e.g., viable T cells of a stimulated cell population. In certain embodiments, at or about 100×10⁶ cells, e.g., viable T cells of a stimulated cell population are transduced.

In some embodiments, the transduction is performed in serum free media. In some embodiments, the transduction is performed in the presence of IL-2, IL-7, and IL-15. In particular embodiments, the cells, e.g., the cells of the stimulated cell population contain at least 80%, at least 85%, at least 90%, or at least 95% cells that are CD3+ T cells. In particular embodiments, the cells, e.g., the cells of the stimulated cell population contain at least 80%, at least 85%, at least 90%, or at least 95% cells that are CD4+ T cells or CD8+ T cells. In some embodiments, the transduction is performed for between 24 and 48 hours, between 36 and 12 hours, between 18 and 30 hours, or for or for about 24 hours. In certain embodiments, the transduction step is initiated within two days, within 36 hours, or within 30 hours of the start or initiation of the incubation, e.g., the incubation under stimulating conditions.

### 2. Viral Vector Particles

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gammaretroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

The viral vector genome is typically constructed in a plasmid form that can be transfected into a packaging or producer cell line. In any of such examples, the nucleic acid encoding a recombinant protein, such as a recombinant receptor, is inserted or located in a region of the viral vector, such as generally in a non-essential region of the viral genome. In some embodiments, the nucleic acid is inserted into the viral genome in the place of certain viral sequences to produce a virus that is replication defective.

Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g. antigen receptor, such as CAR, can be detected.

3. Incubation with Viral Vector

In particular embodiments, transforming or transducing the cells is or includes one or more steps of incubating the cells, e.g., in the presence of the viral vector. In some embodiments, cells, e.g., cells of the transformed cell population, are incubated subsequent to genetically engineering, transforming, transducing, or transfecting the cells.

In certain embodiments, the incubation is performed under static conditions, such as conditions that do not involve centrifugation, shaking, rotating, rocking, or perfusion, e.g., continuous or semi-continuous perfusion of the media. In some embodiments, either prior to or shortly after, e.g., within 5, 15, or 30 minutes of the initiation of the incubation, the cells are transferred (e.g., transferred under sterile conditions) to a container such as a bag or vial, and placed in an incubator.

In some embodiments, the incubation is performed in serum free media. In some embodiments, the serum free media is a defined and/or well-defined cell culture media. In certain embodiments, the serum free media is a controlled culture media that has been processed, e.g., filtered to remove inhibitors and/or growth factors. In some embodiments, the serum free media contains proteins. In certain embodiments, the serum-free media may contain serum albumin, hydrolysates, growth factors, hormones, carrier proteins, and/or attachment factors.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In particular embodiments, the cells are incubated in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In some embodiments, the cells are incubated in the absence of recombinant cytokines.

In some embodiments, all or a portion of the incubation is performed in basal media. In some embodiments, the basal media is a balanced salt solution (e.g., PBS, DPBS, HBSS, EBSS). In some embodiments, the basal media is selected from Dulbecco's Modified Eagle's Medium (DMEM), Minimal Essential Medium (MEM), Basal Medium Eagle (BME), F-10, F-12, RPMI 1640, Glasgow's Minimal Essential Medium (GMEM), alpha Minimal Essential Medium (alpha MEM), Iscove's Modified Dulbecco's Medium, and M199. In some embodiments, the base media is a complex medium (e.g., RPMI-1640, IMDM). In some embodiments, the base medium is OpTmizer™ CTS™ T-Cell Expansion Basal Medium (ThermoFisher).

In some embodiments, cells are incubated with the heterologous polynucleotide, e.g., the viral vector. In certain embodiments, the cells are incubated the cells with the polynucleotide, e.g., viral vector, for, for about, or for at least 18 hours, 24 hours, 30 hours, 36 hours, 40 hours, 48 hours, 54 hours, 60 hours, 72 hours, 84 hours, 96 hours, or more than 96 hours. In certain embodiments, the total duration of the incubation is, is about, or is at least 12 hours, 18 hours, 24 hours, 30 hours, 36 hours, 42 hours, 48 hours, 54 hours, 60 hours, 72 hours, 84 hours, 96 hours, 108 hours, or 120 hours. In particular embodiments, the incubation is completed at, at about, or within 120 hours, 108 hours, 96 hours, 84 hours, 72 hours, 60 hours, 54 hours, 48 hours, 42 hours, 36 hours, 30 hours, 24 hours, 18 hours, or 12 hours. In some embodiments, the total duration of the incubation is between or between about 12 hour and 120 hours, 18 hour and 96 hours, 24 hours and 72 hours, or 24 hours and 48 hours, inclusive. In some embodiments, the total duration of the incubation is between or about between 1 hour and 48 hours, 4 hours and 36 hours, 8 hours and 30 hours or 12 hours and 24 hours, inclusive.

### D. Cultivation

In particular embodiments, processes for generating compositions of engineered T cells provided herein are performed in connection with an optional cultivation step or a step where cells undergo expansion or proliferation in vitro, such as subsequent to an introduction of a heterologous polynucleotide into the cell. In some embodiments, the provided methods include one or more steps for cultivating cells, e.g., cultivating cells under conditions that promote proliferation or expansion. In some embodiments, cells are cultivated under conditions that promote proliferation or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. Thus, in some aspects, cells of a transformed population of enriched T cells are cultivated. In particular embodiments, the one or more transformed populations have been previously enriched for CD28+ T cells.

In some embodiments, the process or method for generating or manufacturing engineered cell compositions do not include a step for cultivation, e.g., to expand the number of engineered cells in the therapeutic composition.

In certain embodiments, the one or more populations of engineered T cells are or include two separate populations of enriched T cells. In particular embodiments, two separate populations of enriched T cells, e.g., two separate populations of enriched T cells selected, isolated, and/or enriched from the same biological sample, are separately cultivated under stimulating conditions. In certain embodiments, the two separate populations include a population of enriched CD4+ T cells, e.g., enriched CD28+CD4+ T cells. In particular embodiments, the two separate populations include a population of enriched CD8+ T cells, e.g., enriched CD28+CD8+ T cells. In some embodiments, two separate populations of enriched CD4+ T cells and enriched CD8+ T cells, e.g., two separate populations of enriched CD28+CD4+ T cells and enriched CD28+CD8+ T cells, are separately cultivated, e.g., under conditions that promote proliferation and/or expansion. In some embodiments, a single population of enriched T cells is cultivated, e.g., a single population including or containing CD28+CD4+ T cells and CD28+CD8+ T cells. In certain embodiments, the single population is a population of enriched CD4+ T cells. In some embodiments, the single population is a population of enriched CD4+ and CD8+ T cells that have been combined from separate populations prior to the cultivation.

In certain embodiments, the populations of engineered T cells is populations of enriched T cells (e.g., a population of CD3+ T cells). In some embodiments, a population of enriched CD3+ T cells (e.g., CD28+CD3+ T cells) that is cultivated, e.g., under conditions that promote proliferation and/or expansion, includes at least at or about 60%, at least at or about 65%, at least at or about 70%, at least at or about 75%, at least at or about 80%, at least at or about 85%, at least at or about 90%, at least at or about 95%, at least at or about 98%, at least at or about 99%, at least at or about 99.5%, at least at or about 99.9%, or at or at about 100% CD3+ T cells (e.g., CD28+CD3+ T cells). In some embodiments, the population includes at least at or about 30%, at least at or about 40%, at least at or about 50%, at least at or about 60%, at least at or about 70%, at least at or about 80%, at least at or about 90%, at least at or about 95%, at least at or about 98%, at least at or about 99%, at least at or about 99.5%, at least at or about 99.9%, or at or at about 100% CD3+T (e.g., CD28+CD3+ T cells) cells that express the recombinant receptor and/or have been transduced or transfected with the recombinant polynucleotide.

In some embodiments, the cultivation is performed under conditions that generally include a temperature suitable for the growth of primary immune cells, such as human T lymphocytes, for example, at least at or about 25 degrees Celsius, generally at least at or about 30 degrees, and generally at or about 37 degrees Celsius. In some embodiments, the population of enriched T cells is incubated at a temperature of 25 to 38° C., such as 30 to 37° C., for example at or about 37° C.±2° C. In some embodiments, the incubation is carried out for a time period until the culture, e.g. cultivation or expansion, results in a desired or threshold density, number or dose of cells. In some embodiments, the cultivation is greater than or greater than about or is for about or 24 hours, 48 hours, 72 hours, 96 hours, 5 days, 6 days, 7 days, 8 days, 9 days or more. In some embodiments, the cultivation is for about 5 days, 6 days, 7 days, 8 days, or 9 days. In some embodiments, the cultivation is for about 7 days.

In some embodiments, the cells are cultivated to achieve a threshold expansion that is an amount, concentration, or density of cells that is least 50%, at least at or about 60%, at least at or about 70%, at least at or about 80%, at least at or about 90%, at least at or about 95%, at least at or about 100%, at least at or about 150%, at least at or about 1-fold, at least at or about 2-fold, at least at or about 3-fold, at least at or about 4-fold, at least at or about 5-fold, at least at or about 10-fold, at least at or about 20-fold, at least at or about 50-fold greater as compared to the amount, concentration, or density of cells at the beginning of the cultivation. In some embodiments, the cells are cultivated to achieve a threshold expansion that is at least about 5-fold or at least about 10-fold greater as compared to the amount, concentration, or density of cells at the beginning of the cultivation. In some embodiments, the cells are cultivated to achieve a threshold expansion that is at least about 5-fold greater as compared to the amount, concentration, or density of cells at the beginning of the cultivation.

In particular embodiments, a composition of enriched T cells is cultivated in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In some aspects, cultivation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

### E. Harvesting, Collecting, and Formulating Cells

In some embodiments, one or more process steps (e.g. carried out in the centrifugal chamber and/or closed system) for manufacturing, generating or producing a cell therapy and/or engineered cells may include formulation of cells, such as formulation of genetically engineered cells resulting from the provided transduction processing steps prior to or after the culturing, e.g. cultivation and expansion, and/or one or more other processing steps as described. In some embodiments, the provided methods associated with formulation of cells include processing transduced cells, such as cells transduced and/or expanded using the processing steps described above, in a closed system.

In some embodiments, the stimulatory reagent is removed and/or separated from the cells prior to the formulating. In particular embodiments, the stimulatory reagent is removed and/or separated from the cells after the cultivation. In certain embodiments, the stimulatory agent is removed and/or separated from the cells subsequent to the cultivation and prior to formulating the cultivated cells, e.g., under conditions that promote proliferation and/or expansion. In certain embodiments, the stimulatory reagent is a stimulatory reagent that is described in herein. In particular embodiments, the stimulatory reagent is removed and/or separated from the cells as described herein.

In some embodiments, the cells are formulated between 0 days and 10 days, between 0 and 5 days, between 2 days and 7 days, between 0.5 days, and 4 days, or between 1 day and 3 days after the cells after the threshold cell count, density, and/or expansion has been achieved during the cultivation. In certain embodiments, the cells are formulated at or at or about or within 12 hours, 18 hours, 24 hours, 1 day, 2 days, or 3 days after the threshold cell count, density, and/or expansion has been achieved during the cultivation. In some embodiments, the cells are formulated within or within about 1 day after the threshold cell count, density, and/or expansion has been achieved during the cultivation.

In certain embodiments, the amount of time from the initiation of the stimulation to collecting, harvesting, or formulating the cells is about 7 days, 8 days, 9 days, or 10 days. In certain embodiments, the amount of time from the initiation of the stimulation to collecting, harvesting, or formulating the cells is nor more than 10 days. In some embodiments, the amount of time from the initiation of the stimulation to collecting, harvesting, or formulating the cells for generating engineered cells, from the initiation of the stimulation to collecting, harvesting, or formulating the cells is between about 5 days and about 10 days.

In certain embodiments, the cells are harvested or collected at least when the integrated vector is detected in the genome. In some embodiments, the cells are harvested or collected prior to stable integrated vector copy number (iVCN) per diploid genome. In particular embodiments, the cells are harvested or collected after the integrated vector is detected in the genome but prior to when a stable iVCN per diploid genome is achieved.

In some embodiments, the provided methods for manufacturing, generating or producing a cell therapy and/or engineered cells may include formulation of cells, such as formulation of genetically engineered cells resulting from the provided processing steps prior to or after the incubating, engineering, and cultivating, and/or one or more other processing steps as described. In some embodiments, the dose of cells comprising cells engineered with a recombinant antigen receptor, e.g. CAR or TCR, is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods.

In some cases, the cells are processed in one or more steps for manufacturing, generating or producing a cell therapy and/or engineered cells may include formulation of cells, such as formulation of genetically engineered cells resulting from the provided transduction processing steps prior to or after the culturing, e.g. cultivation and expansion, and/or one or more other processing steps as described. In some cases, the cells can be formulated in an amount for dosage administration, such as for a single unit dosage administration or multiple dosage administration. In some embodiments, the provided methods associated with formulation of cells include processing transduced cells, such as cells transduced and/or expanded using the processing steps described above, in a closed system.

In certain embodiments, one or more compositions of enriched T cells are formulated. In particular embodiments, one or more compositions of enriched T cells are formulated after the one or more compositions have been engineered and/or cultivated. In particular embodiments, the one or more compositions are input compositions. In some embodiments, the one or more input compositions have been previously cryopreserved and stored, and are thawed prior to the incubation.

In certain embodiments, the formulated cells are output cells. In some embodiments, a formulated composition of enriched T cells is an output composition of enriched T cells.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cell are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryopreserved or cryoprotected, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryopreserved or cryoprotected, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the composition of enriched T cells, e.g., T cells that have been stimulated, engineered, and/or cultivated, are formulated, cryopreserved, and then stored for an amount of time. In certain embodiments, the formulated, cryopreserved cells are stored until the cells are released for infusion. In particular embodiments, the formulated cryopreserved cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryopreserved and stored for, for about, or for less than 1 days, 2 days, 3 days, 4 days, 5 days, 6 days, or 7 days. In certain embodiments, the cells are thawed and administered to a subject after the storage. In certain embodiments, the cells are stored for or for about 5 days.

In some embodiments, such cells produced by the method, or a composition comprising such cells, are administered to a subject for treating a disease or condition.

### F. Sequential Selection and Parallel Selection

The methods provided herein allow for multiple selection steps, for example by column chromatography, to isolate and/or enrich a target cell population (e.g., T cells, CD3+, CD4+, CD8+, CD28+ T cells). In some embodiments, one or more selection steps are carried out at one or more time points or following certain steps of the process for creating an output therapeutic cell composition. In some embodiments, a selection step includes multiple selection steps for, for example, further purifying the cell composition, selection of specific cell subtypes, selection of viable cells, selection of engineered cells, and/or adjusting the ratio, total number, or concentration of cells. In some embodiments, a selection step is performed prior to incubation. In some embodiments, a selection step is performed prior to harvesting and collection.

In some aspects, such methods (e.g., selection steps) are achieved by a single process stream, such as in a closed system, by employing sequential selections in which a plurality of different cell populations from a sample (e.g., output composition of stimulated and/or engineered cells), as provided herein, are enriched and/or isolated. In some aspects, carrying out the separation or isolation in the same vessel or set of vessels, e.g., tubing set, is achieved by carrying out sequential positive and negative selection steps, the subsequent step subjecting the negative and/or positive fraction from the previous step to further selection, where the entire process is carried out in the same tube or tubing set. In one embodiment, a sample (e.g., output composition of stimulated and/or engineered cells) containing target cells is subjected to a sequential selection in which a first selection is effected to enrich for CD3+ cells, and the selected cells from the first selection are used as the source of cells for a second selection to enrich for CD28+ cells. In one embodiment, a sample (e.g., output composition of stimulated and/or engineered cells) containing target cells is subjected to a sequential selection in which a first selection is effected to enrich for CD28+ cells, and the selected cells from the first selection are used as the source of cells for a second selection to enrich for CD3+ cells. In some embodiments, a cell population (e.g., output composition of stimulated and/or engineered cells) containing target cells is subjected to a sequential selection for viable cells. In some embodiments, the ratio or total number of cells in the cell population (e.g., output composition of stimulated and/or engineered cells) containing target cells is controlled or adjusted.

In some aspects, such methods (e.g., selection steps) are achieved by a single process stream, such as in a closed system, by employing sequential selections in which a plurality of different cell populations from a sample (e.g., output composition of stimulated and/or engineered cells), as provided herein, are enriched and/or isolated. In some aspects, carrying out the separation or isolation in the same vessel or set of vessels, e.g., tubing set, is achieved by carrying out sequential positive and negative selection steps, the subsequent step subjecting the negative and/or positive fraction from the previous step to further selection, where the entire process is carried out in the same tube or tubing set. In one embodiment, a sample (e.g., output composition of stimulated and/or engineered cells) containing target cells is subjected to a sequential selection in which a first selection is effected to enrich for one of the CD4+ or CD8+ populations, and the non-selected cells from the first selection are used as the source of cells for a second selection to enrich for the other of the CD4+ or CD8+ populations. In some embodiments, a further selection or selections can be effected to enrich for sub-populations of one or both of the CD4+ or CD8+ population, for example, CD28+ T cells. In some embodiments, a cell population (e.g., output composition of stimulated and/or engineered cells) containing target cells is subjected to a sequential selection for viable cells. In some embodiments, the ratio or total number of cells in the cell population (e.g., output composition of stimulated and/or engineered cells) containing target cells is controlled or adjusted.

In some aspects, such methods (e.g., selection steps) are achieved by a single process stream, such as in a closed system, by employing sequential selections in which a plurality of different cell populations from a sample (e.g., output composition of stimulated and/or engineered cells), as provided herein, are enriched and/or isolated. In some aspects, carrying out the separation or isolation in the same vessel or set of vessels, e.g., tubing set, is achieved by carrying out sequential negative and positive selection steps, the subsequent step subjecting the negative and/or positive fraction from the previous step to further selection, where the entire process is carried out in the same tube or tubing set. In one embodiment, a sample (e.g., output composition of stimulated and/or engineered cells) containing target cells is subjected to a sequential selection in which a first selection is effected to remove CD28-T cell populations. In some embodiments, a further selection or selections can be effected to enrich for one or both of CD4+ or CD8+ population, for example, CD28+CD4+ or CD28+CD8+ cells. In some embodiments, a cell population (e.g., output composition of stimulated and/or engineered cells) containing target cells is subjected to a sequential selection for viable cells. In some embodiments, the ratio or total number of cells in the cell population (e.g., output composition of stimulated and/or engineered cells) containing target cells is controlled or adjusted.

In one embodiment, a sample (e.g., output composition of stimulated and/or engineered cells) containing target cells is subjected to a sequential selection in which a first selection is effected to enrich for a CD3+ population. In some embodiments, a further selection or selections can be effected to enrich for sub-populations of the CD3+ population, for example, CD28+ T cells. In some embodiments, the further selection or selections can be effected to enrich for viable cells. In some embodiments, the further selection or selections can be effected to enrich subpopulations of CD28+CD3+ cells, for example CD3+CD28+CD4+ or CD3+CD28+CD8+ cells that are viable. In some embodiments, selecting viable cells includes or consists of removing dead cells from the cell population (e.g., output composition of stimulated and/or engineered cells or subpopulations thereof).

In one embodiment, a sample (e.g., output composition of stimulated and/or engineered cells) containing target cells is subjected to a sequential selection in which a first selection is effected to remove CD28− cells. In some embodiments, a further selection or selections can be effected to enrich for sub-populations of the CD28+ T cell population, for example, CD4+ and/or CD8+ cells. In some embodiments, the further selection or selections can be effected to enrich for viable cells. In some embodiments, selecting viable cells includes or consists of removing dead cells from the cell population (e.g., output composition of stimulated and/or engineered cells or subpopulations thereof).

In some embodiments, the methods (e.g., selection steps) disclosed in this Section do not need to be carried out using sequential selection techniques. In some embodiments, the methods (e.g., selection steps) disclosed in this Section can be carried out using sequential selection techniques in combination with parallel selection techniques. In some embodiments, the selection step does not employ sequential selection or may employ sequential selection that does not occur in a closed system or in a set of vessels using the same tubing. In some embodiments, the selection step is accomplished in a single step, for example using a single chromatography column. In some embodiments, the selection step is accomplished using a parallel selection technique. For example, the selection step is achieved by carrying out positive and/or negative selection steps simultaneously, for example in a closed system where the entire process is carried out in the same tube or tubing set. In some embodiments, a sample (e.g., output composition of stimulated and/or engineered cells) containing target cells is subjected to a parallel selection in which the sample (e.g., output composition of stimulated and/or engineered cells) is load onto two or more chromatography columns, where each column effects selection of a cell population. In some embodiments, the two or more chromatography columns effect selection of CD28+, CD3+, CD4+, or CD8+ populations individually. In some embodiments, the two or more chromatography columns effect selection of the same cell population. For example, the two or more chromatography columns may effect selection of CD28+ cells. In some embodiments, the two or more chromatography columns, including affinity chromatography or gel permeation chromatography, independently effect selection of the same cell population. In some embodiments, the two or more chromatography columns, including affinity chromatography or gel permeation chromatography, independently effect selection of different cell populations. In some embodiments, a further selection or selections can be effected to enrich for subpopulations of one or all cell populations selected via parallel selection. In some embodiments, a sample (e.g., output composition of stimulated and/or engineered cells) containing target cells (e.g., CD28+ cells) is subjected to a parallel selection in which parallel selection is effected to enrich for a CD3+ population.

In some embodiments, a selection step can be carried out using beads labeled with selection agents as described herein, and the positive and negative fractions from the first selection step can be retained, followed by further positive selection of the positive fraction to enrich for a second selection marker, such as by using beads labeled with a second selection agent or by subjecting the positive fraction to column chromatography as described above. In some embodiments, one or more selection steps are carried out using column chromatography as described herein. In some embodiments, selection steps are accomplished using one or more methods including bead separation and column chromatography. In some embodiments, the selection are accomplished using column chromatography.

In some aspects, isolating the plurality of populations in a single or in the same isolation or separation vessel or set of vessels, such as a single column or set of columns, and/or same tube, or tubing set or using the same separation matrix or media or reagents, such as the same magnetic matrix, affinity-labeled solid support, or antibodies or other binding partners, include features that streamline the isolation, for example, resulting in reduced cost, time, complexity, need for handling of samples, use of resources, reagents, or equipment. In some aspects, such features are advantageous in that they minimize cost, efficiency, time, and/or complexity associated with the methods, and/or avoid potential harm to the cell product, such as harm caused by infection, contamination, and/or changes in temperature. The methods provided herein allow for multiple selection steps to enrich target populations both prior to or following cell selection combined with on-column stimulation.

The methods provided herein further allow for the selection and enrichment of successfully stimulated and engineered cells. For example, in some embodiments, the sequential selection, parallel selection, or single selection procedures described above may be used to identify stimulated cells expressing recombinant receptors (e.g., CARs, TCRs). In some embodiments, cells expressing the recombinant receptor (e.g., CAR) can be further enriched for sub-population cells, e.g., CD3+ CAR+ T cells and/or viable cells. In some embodiments, the selection step allows control or adjustment of the ratio, concentration, or total number of cells expressing a recombinant receptor (e.g., CAR, TCR) and/or subpopulations thereof. In some embodiments, enriched populations can be formulated for use (e.g., administration) for cell therapy.

### G. Exemplary Features of the Process

In some aspects, the provided methods and compositions relate to populations of T cells enriched for CD28+ T cells. In particular embodiments are drawn to methods of generating populations of enriched CD28+ T cells, such as by negative selection of CD28− cells and, in some aspects, positive selection of T cells, such as CD4+ T cells and/or CD8+ T cells. In particular embodiments are drawn to methods of generating populations of enriched CD28+ T cells, such as by negative selection of CD28− cells and, in some aspects, positive selection of T cells, such as CD3+ T cells. Certain embodiments are drawn to methods of incubating, stimulating, activating, engineering, transducing, cultivating, and/or expanding populations of enriched CD28+ T cells. In certain embodiments, incubating, stimulating, activating, engineering, transducing, cultivating, and/or expanding populations of enriched CD28+ T cells provide advantages over such steps or processes with alternative populations of T cells, such as populations containing amounts or high amounts of CD28− T cells. Such advantages include, but are not limited to, improved proliferation or expansion and less differentiation, e.g., terminal differentiation.

In some embodiments, the provided methods are or include steps of enriching T cells, such as by selecting CD28+ T cells from a biological sample containing peripheral blood mononuclear cells (PBMCs) to generate a population of T cells depleted of CD28− T cells, e.g., a population of enriched CD28+ T cells. In some embodiments, the population contains less than or less than about 10%, 5%, 1%, or 0.1% CD28− T cells. In particular embodiments, the population contains less than or less than about 25%, 20%, 15%, 10%, or 5% of the percentage of CD28− T cells that were present the biological sample. In certain embodiments, at least 85%, 90%, 95%, or 99% of the CD4+ T cells of the population are CD28+CD4+ T cells. In particular embodiments, at least 85%, 90%, 95%, or 99% of the CD8+ T cells of the population are CD28+CD8+ T cells. In particular embodiments, at least 85%, 90%, 95%, or 99% of the CD3+ T cells of the population are CD28+CD3+ T cells.

In certain embodiments, enriching T cells includes selecting or removing CD28− cells from a biological sample, and then separately selecting for CD4+ T cells and CD8+ T cells from the population negatively selected for CD28, such as to generated a population of enriched CD28+CD4+ T cells and a population of enriched CD28+CD8+ T cells. In some embodiments, these populations remain separate, such as are subsequently separately cryoprotected and stored and/or are separately engineered to express a recombinant receptor. In particular embodiments, the separate populations are combined, such as at a ratio of 1:1 CD28+CD4+ T cells to CD28+CD8+ T cells.

In some embodiments, the provided methods include stimulating populations of enriched CD28+ T cells. In certain embodiments, the provided methods include one or more steps for stimulating populations of enriched CD28+CD4+ T cells. In particular embodiments, the provided methods include one or more steps for stimulating populations of enriched CD28+CD8+ T cells. In certain embodiments, the populations of enriched CD28+CD4+ T cells and populations of enriched CD28+CD8+ T cells are stimulated such as by incubating the cells under stimulating conditions, e.g., any stimulating conditions described herein. In particular embodiments, the stimulating conditions are or include the presence of a stimulatory reagent. In certain embodiments, separate populations of enriched CD28+CD4+ T cells and enriched CD28+CD8+ T cells are separately stimulated. In particular embodiments, separate populations of enriched CD28+CD4+ T cells and enriched CD28+CD8+ T cells are combined or mixed prior to being stimulated, such that a combined composition of enriched CD28+CD4+ T cells and CD28+CD8+ T cells is stimulated.

In some embodiments, the genetic engineering is performed by (a) incubating populations of enriched CD28+ T cells in the presence of a stimulatory reagent capable of activating one or more intracellular signaling domains of one or more components of a TCR complex and one or more intracellular signaling domains of one or more costimulatory molecules thereby generating stimulated T cells; (b) introducing a heterologous polynucleotide into the stimulated T cells of the first and second enriched populations by transducing the stimulated T cells with a viral vector containing a heterologous polynucleotide to generate transformed T cells; (c) cultivating the transformed T cells under conditions to promote proliferation or expansion of the transformed T cells; and (d) harvesting or collecting the expanded T cells.

In some embodiments, a method for generically engineering T cells is or includes steps selecting or removing CD28− cells from a biological sample, and then separately selecting for CD3+ T cells from the population negatively selected for CD28, such as to generated a population of enriched CD28+CD3+ T cells. In certain embodiments, the CD28+CD3+ T cells are incubated in the presence of a stimulatory reagent, e.g., an anti-CD3 and anti-CD28 antibody conjugated paramagnetic bead. In particular embodiments, the stimulatory reagent is or includes a streptavidin mutein oligomeric particle with reversibly bound anti-CD3 and anti-CD28 Fabs, to stimulate the T cells prior steps for introducing a heterologous polynucleotide encoding a recombinant receptor. In some embodiments, the stimulated CD28+CD3+ T cells are transduced with a virus carrying the heterologous polynucleotide, such as by steps including spinoculation and/or incubation in the presence of the virus, such as to generate transformed CD28+CD3+ T cells.

In some embodiments, the transformed T cells are cultivated under conditions to promote proliferation or expansion of the transformed T cells, e.g., in the presence of cytokines such as IL-2, IL-7, or IL-15. In some aspects, the cells are cultivated until the cells achieve an expansion of at least 3-fold, 4-fold, or 5-fold.

In certain embodiments, the expanded cells are collected or harvested, such as to be formulated for cryoprotection and storage, or for administration to a subject as a cell therapy. In certain embodiments, the transformed cells are collected or harvested.

In certain embodiments, the provided methods are used in connection with successfully generating or producing output compositions of engineered T cells that are suitable for use in cell therapy. In some embodiments, an output composition is successfully generated if the cells of the composition achieve the threshold cell count, density, and/or expansion during cultivation. In particular embodiments, the provided methods have an at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% probability or likelihood of successfully generating or producing an population of T cells suitable for a cell therapy, e.g., from an initial population of enriched T cells or from a biological sample.

In certain embodiments, the population of enriched T cells generated from the provided methods for use in a cell therapy are active and expand, and/or are capable of activation and expansion, in vivo, when administered to a subject. In particular embodiments, the cells display features and/or characteristics that indicate or are associated with in vivo efficacy, activity, and/or expansion. For example, in some embodiments, such features or characters may include the expression of a protein, such as a surface protein, that is associated with activation, proliferation, and/or expansion after administration to a subject in vivo.

In certain embodiments, the provided methods are used in connection with successfully generating or producing compositions of engineered T cells that are suitable for use in cell therapy. In some embodiments, a composition is successfully generated if the cells of the composition achieve a target cell count, density, and/or expansion during cultivation.

In particular embodiments, the provided methods have an at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% probability or likelihood of successfully generating or producing a population of enriched T cells suitable for a cell therapy. In certain embodiments, the probability or likelihood is between 85% and 100%, between 90% and 95%, or between 92% and 94%. In certain embodiments, the provided methods successfully generate or produce an a population of enriched T cells suitable for a cell therapy from at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the samples or populations of enriched CD28+ cells.

In some embodiments, the entire process is performed with a single population of enriched T cells, e.g., CD3+ cells. In some embodiments, the enriched T cells are or include engineered T cells, e.g., T cells transduced to express a recombinant receptor.

### VI. T CELL THERAPY

Provided herein are methods of treating a subject having a disease or condition, comprising administration of a cell therapy (e.g., a T cell therapy, such as CAR T cells), wherein the subject is selected from treatment with the cell therapy. In some embodiments, cell therapy is an autologous T cell therapy. In some embodiments, the methods comprise selecting the subject for treatment with the cell therapy if the percentage of CD28+ T cells in a biological sample obtained from the same subject is above a threshold value. In some embodiments, cell therapy is an allogeneic T cell therapy. In some embodiments, the percentage of CD28+ T cells is the percentage of T cells that are CD28+. In some embodiments, the methods comprise selecting a subject for treatment with the cell therapy if the percentage of CD28+ T cells in a biological sample obtained from a different subject is above a threshold value. In some embodiments, the biological sample is obtained from a subject between about 1 week and about 6 weeks prior to a subject receiving the cell therapy, such as about 3 weeks prior. In some embodiments, the methods further comprise, administering the cell therapy to the selected subject.

In some embodiments, the biological sample is an apheresis sample or a leukapheresis sample. In some embodiments, the biological sample is a PBMC sample. In some embodiments, cells of the biological sample are engineered to express a recombinant receptor, thereby generating the cell therapy (e.g. T cell therapy).

Also provided herein are methods of treating a subject having a disease or condition, comprising administration of a T cell therapy to the subject, wherein at least about 40% of the T cells in the subject are CD28+. In some embodiments, the disease or condition is a multiple myeloma.

In some embodiments, the cell therapy (i.e. T cell therapy) comprises engineered T cells expressing a recombinant receptor (e.g., a chimeric antigen receptor), such as one that contains an extracellular domain including an antigen binding moiety, such as an antibody or fragment as described herein. Also provided are populations of such cells, compositions containing such cells and/or enriched for such cells, such as in which cells expressing an antigen-binding moiety make up at least 50, 60, 70, 80, 90, 91, 92, 93, 94, 95, 96, 97, 98, 99, or more percent of the total cells in the composition or cells of a certain type such as PBMCs, T cells or CD3+, CD8+ or CD4+ cells.

Among the compositions are pharmaceutical compositions and formulations for administration, such as for adoptive cell therapy. Also provided are therapeutic methods for administering the cells and compositions to subjects, e.g., patients.

Thus, also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a recombinant receptor described herein.

In some embodiments, the T cell therapy for use in accord with the provided methods includes administering engineered T cells expressing recombinant receptors designed to recognize and/or specifically bind to molecules associated with a cancer. In some cases, the recombinant receptor binds to an antigen expressed by a cancer such as multiple myeloma, for example relapsed and refractory (R/R) multiple myeloma (MM) (e.g., BCMA). In some cases, the recombinant receptor binds to an antigen expressed by a cancer such as multiple myeloma, for example relapsed and refractory (R/R) multiple myeloma (MM) (e.g., GPRC5D). In some cases, the recombinant receptor binds to an antigen expressed by a cancer such as a leukemia or lymphoma, for example relapsed and refractory (R/R) leukemia or lymphoma (e.g., CD19). In some embodiments, binding to the antigen results in a response, such as an immune response against such molecules upon binding to such molecules. In some embodiments, the cells contain or are engineered to contain an engineered receptor, e.g., an engineered antigen receptor, such as a chimeric antigen receptor (CAR), or a T cell receptor (TCR). The recombinant receptor, such as a CAR, generally includes an extracellular antigen (or ligand) binding domain that is directed against an antigen (e.g., BCMA), linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Recombinant Receptors, e.g. Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

In some embodiments of the provided methods and uses, the engineered cells, such as T cells, express a chimeric receptor, such as a chimeric antigen receptor (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody, VHH) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IMGT or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (VH and VL, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Among the antigen binding domains included in the CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; heavy chain variable (VH) regions, single-chain antibody molecules such as scFvs and single-domain antibodies comprising only the VH region; and multispecific antibodies formed from antibody fragments. In particular embodiments, the antibodies are single-chain antibody fragments comprising a heavy chain variable (VH) region and/or a light chain variable (VL) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody. In certain embodiments, a single-domain antibody is a human single-domain antibody. In some embodiments, the CAR comprises an antibody heavy chain domain that specifically binds an antigen expressed by a cancer.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In some embodiments, the CAR includes an antigen-binding portion or portions of an antibody molecule, such as a single-chain antibody fragment (scFv) derived from the variable heavy (VH) and variable light (VL) chains of a monoclonal antibody (mAb), or a single domain antibody (sdAb), such as sdFv, nanobody, VHH and VNAR. In some embodiments, an antigen-binding fragment comprises antibody variable regions joined by a flexible linker.

In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the VH region. In some embodiments, the CAR comprises a sdAb. In some embodiments, the CAR comprises two sdAbs. In some embodiments, each of the two sdAbs is a VH domain. In some embodiments, the two sdAbs bind to different epitopes of an antigen (e.g., BCMA). In some embodiments, the two sdAbs bind to the same epitope of an antigen (e.g., BCMA). In some embodiments, the antibody or antigen binding fragment is an scFv comprising a heavy chain variable (VH) region and a light chain variable (VL) region.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the antibodies included in the provided CARs are murine antibodies. A “murine antibody” is an antibody with an amino acid sequence corresponding to that of an antibody produced by a murine or a murine cell, or non-murine source that utilizes murine antibody repertoires or other murine antibody-encoding sequences, including murine antibody libraries.

Also among the antibodies included in the provided CARs are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the antibodies included in the provided CARs are those that are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

Thus, in some embodiments, the chimeric antigen receptor, including TCR-like CARS, includes an extracellular portion containing an antibody or antibody fragment. In some embodiments, the antibody or fragment includes an scFv. In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the VH region. In some embodiments, the antibody or antigen binding fragment is an scFv comprising a heavy chain variable (VH) region and a light chain variable (VL) region.

In some embodiments, the antibody is an antigen-binding fragment, such as a scFv, that includes one or more linkers joining two antibody domains or regions, such as a heavy chain variable (VH) region and a light chain variable (VL) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline. In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO:26) or GGGS (3GS; SEQ ID NO:27), such as between 2, 3, 4, and 5 repeats of such a sequence. Exemplary linkers include those having or consisting of an sequence set forth in SEQ ID NO:28 (GGGGSGGGGSGGGGS), SEQ ID NO:29 (GSTSGSGKPGSGEGSTKG), SEQ ID NO: 30 (SRGGGGSGGGGSGGGGSLEMA), or SEQ ID NO:38 (ASGGGGSGGRASGGGGS). In some embodiments, the linker is or comprises the sequence set forth in SEQ ID NO:29.

In some embodiments of the provided methods, chimeric receptors, such as a chimeric antigen receptors, contain one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, WO2016/0046724, WO2016/014789, WO2016/090320, WO2016/094304, WO2017/025038, WO2017/173256, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, 8,479,118, and 9,765,342, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

Exemplary antigen receptors, e.g., CARs, also include any described in Marofi et al., Stem Cell Res Ther 12: 81 (2021); Townsend et al., J Exp Clin Cancer Res 37: 163 (2018); Ma et al., Int J Biol Sci 15(12): 2548-2560 (2019); Zhao and Cao, Front Immunol 10: 2250 (2019); Han et al., J Cancer 12(2): 326-334 (2021); Specht et al., Cancer Res 79: 4 Supplement, Abstract P2-09-13; Byers et al., Journal of Clinical Oncology 37, no. 15_suppl (2019); Panowski et al., Cancer Res 79 (13 Supplement) 2326 (2019); and Sauer et al., Blood 134 (Supplement 1): 1932 (2019); or can contain any of the antibodies or antigen-binding fragments described in U.S. Pat. Nos. 8,153,765; 8,603,477, 8,008,450; U.S. Pub. No. US20120189622 or US20100260748; and International PCT Publication Nos. WO2006099875, WO2009080829, WO2012092612, WO2014210064.

Further exemplary antigen receptors, e.g., CARs, such as anti-BCMA CARs, include the CARs of idecabtagene vicleucel, ABECMA©, BCMA02, JCARH125, JNJ-68284528 (LCAR-B38M; ciltacabtagene autoleucel; CARVYKTI™) (Janssen/Legend), P-BCMA-101 (Poseida), PBCAR269A (Poseida), P-BCMA-Allol (Poseida), Allo-715 (Pfizer/Allogene), CT053 (Carsgen), Descartes-08 (Cartesian), PHE885 (Novartis), ARI-002 (Hospital Clinic Barcelona, IDIBAPS), and CTX120 (CRISPR Therapeutics). In a particular embodiment, the CAR is the CAR of idecabtagene vicleucel cells. In a particular embodiment, the CAR is the CAR of ABECMA® cells (cells used in ABECMA® immunotherapy). In a particular embodiment, the CAR is the CAR of ciltacabtagene autoleucel cells. In a particular embodiment, the CAR is the CAR of CARVYKTI™ cells (cells used in CARVYKTI™ immunotherapy).

Exemplary antigen receptors, e.g., CARs, also include the CARs of FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel), ABECMA® (idecabtagene vicleucel), and CARVYKTI™ (ciltacabtagene autoleucel). In some of any of the provided embodiments, the CAR is the CAR of BREYANZI® (lisocabtagene maraleucel), TECARTUS™ (brexucabtagene autoleucel), KYMRIAH™ (tisagenlecleucel), YESCARTA™ (axicabtagene ciloleucel), ABECMA® (idecabtagene vicleucel), or CARVYKTI™ (ciltacabtagene autoleucel). In some of any of the provided embodiments, the CAR is the CAR of BREYANZI® (lisocabtagene maraleucel, see Sehgal et al., 2020, Journal of Clinical Oncology 38:15_suppl, 8040; Teoh et al., 2019, Blood 134(Supplement_1):593; and Abramson et al., 2020, The Lancet 396(10254): 839-852). In some of any of the provided embodiments, the CAR is the CAR of TECARTUS™ (brexucabtagene autoleucel, see Mian and Hill, 2021, Expert Opin Biol Ther; 21(4):435-441; and Wang et al., 2021, Blood 138(Supplement 1):744). In some of any of the provided embodiments, the CAR is the CAR of KYMRIAH™ (tisagenlecleucel, see Bishop et al., 2022, N Engl J Med 386:629:639; Schuster et al., 2019, N Engl J Med 380:45-56; Halford et al., 2021, Ann Pharmacother 55(4):466-479; Mueller et al., 2021, Blood Adv. 5(23):4980-4991; and Fowler et al., 2022, Nature Medicine 28:325-332). In some of any of the provided embodiments, the CAR is the CAR of YESCARTA™ (axicabtagene ciloleucel, see Neelapu et al., 2017, N Engl J Med 377(26):2531-2544; Jacobson et al., 2021, The Lancet 23(1):P91-103; and Locke et al., 2022, N Engl J Med 386:640-654). In some of any of the provided embodiments, the CAR is the CAR of ABECMA® (idecabtagene vicleucel, see Raje et al., 2019, N Engl J Med 380:1726-1737; and Munshi et al., 2021, N Engl J Med 384:705-716). In some of any of the provided embodiments, the CAR is the CAR of CARVYKTI™ (ciltacabtagene autoleucel, see Berdeja et al., Lancet. 2021 Jul. 24; 398(10297):314-324; and Martin, Abstract #549 [Oral], presented at 2021 American Society of Hematology (ASH) Annual Meeting & Exposition)).

In some embodiments, the antigen is BCMA. In some embodiments, the CAR includes a BCMA-binding portion or portions of the antibody molecule, such as a heavy chain variable (VH) region and/or light chain variable (VL) region of the antibody, e.g., an scFv antibody fragment. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the provided BCMA-binding CARs contain an antibody, such as an anti-BCMA antibody, or an antigen-binding fragment thereof that confers the BCMA-binding properties of the provided CAR. In some embodiments, the antibody or antigen-binding domain can be any anti-BCMA antibody described or derived from any anti-BCMA antibody described. See, e.g., Carpenter et al., Clin. Cancer Res., 2013, 19(8):2048-2060; Feng et al., Scand. J. Immunol. (2020) 92:e12910; U.S. Pat. Nos. 9,034,324 9,765,342; U.S. Patent Publication Nos. US2016/0046724, US20170183418; and International PCT Application Nos. WO 2016090320, WO2016090327, WO2016094304, WO2016014565, WO2016014789, WO2010104949, WO2017025038, WO2017173256, WO2018085690, or WO2021091978. Any of such anti-BCMA antibodies or antigen-binding fragments can be used in the provided CARs. In some embodiments, the anti-BCMA CAR contains one or more single-domain anti-BCMA antibodies. In some embodiments, the one or more single-domain anti-BCMA antibodies is derived from an antibody described in WO2017025038 or WO2018028647. In some embodiments, the anti-BCMA CAR contains two single-domain anti-BCMA antibodies. In some embodiments, the two single-domain anti-BCMA antibodies are derived from one or more antibodies described in WO2017025038 or WO2018028647. In some embodiments, the BCMA binding domain comprises or consists of A37353-G4S-A37917 (G4S being a linker between the two binding domains), described in WO2017025038 or WO2018028647, and provided, e.g., in SEQ ID NOs: 300, 301 and 302 of WO2017025038 or WO2018028647 (with or without signal peptide). In some embodiments, the anti-BCMA CAR contains an antigen-binding domain that is an scFv containing a variable heavy (VH) and/or a variable light (VL) region. In some embodiments, the scFv containing a variable heavy (VH) and/or a variable light (VL) region is derived from an antibody described in WO2016090320 or WO2016090327. In some embodiments, the scFv containing a variable heavy (VH) and/or a variable light (VL) region is derived from an antibody described in WO 2019/090003. In some embodiments, the scFv containing a variable heavy (VH) and/or a variable light (VL) region is derived from an antibody described in WO2016094304 or WO2021091978. In some embodiments, the scFv containing a variable heavy (VH) and/or a variable light (VL) region is derived from an antibody described in WO2018133877. In some embodiments, the scFv containing a variable heavy (VH) and/or a variable light (VL) region is derived from an antibody described in WO2019149269. In some embodiments, the anti-BCMA CAR is any as described in WO2019173636 or WO2020051374A. In some embodiments, the anti-BCMA CAR is any as described in WO2018102752. In some embodiments, the anti-BCMA CAR is any as described in WO2020112796 or WO2021173630.

In some embodiments, the antibody, e.g., the anti-BCMA antibody or antigen-binding fragment, contains a heavy and/or light chain variable (VH or VL) region sequence as described, or a sufficient antigen-binding portion thereof. In some embodiments, the anti-BCMA antibody, e.g., antigen-binding fragment, contains a VH region sequence or sufficient antigen-binding portion thereof that contains a CDR-H1, CDR-H2 and/or CDR-H3 as described. In some embodiments, the anti-BCMA antibody, e.g., antigen-binding fragment, contains a VL region sequence or sufficient antigen-binding portion that contains a CDR-L1, CDR-L2 and/or CDR-L3 as described. In some embodiments, the anti-BCMA antibody, e.g., antigen-binding fragment, contains a VH region sequence that contains a CDR-H1, CDR-H2 and/or CDR-H3 as described and contains a VL region sequence that contains a CDR-L1, CDR-L2 and/or CDR-L3 as described. Also among the antibodies are those having sequences at least at or about 90%, about 91%, about 92%, about 93%, about 94%, about 95%, about 96%, about 97%, about 98%, or about 99% identical to such a sequence.

In some embodiments, the antibody is a single domain antibody (sdAb) comprising only a VH region sequence or a sufficient antigen-binding portion thereof, such as any of the above described VH sequences (e.g., a CDR-H1, a CDR-H2, a CDR-H3 and/or a CDR-H4).

In some embodiments, an antibody provided herein (e.g., an anti-BCMA antibody) or antigen-binding fragment thereof comprising a VH region further comprises a light chain or a sufficient antigen binding portion thereof. For example, in some embodiments, the antibody or antigen-binding fragment thereof contains a VH region and a VL region, or a sufficient antigen-binding portion of a VH and VL region. In such embodiments, a VH region sequence can be any of the above described VH sequence. In some such embodiments, the antibody is an antigen-binding fragment, such as a Fab or an scFv. In some such embodiments, the antibody is a full-length antibody that also contains a constant region.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human BCMA antibodies, and cells expressing such chimeric receptors have been previously described. See Carpenter et al., Clin Cancer Res., 2013, 19(8):2048-2060, U.S. Pat. No. 9,765,342, WO 2016/090320, WO2016090327, WO2010104949A2, WO2016/0046724, WO2016/014789, WO2016/094304, WO2017/025038, and WO2017173256.

In some embodiments, the anti-BCMA CAR contains an antigen-binding domain, such as an scFv, containing a variable heavy (VH) and/or a variable light (VL) region derived from an antibody described in WO2016094304 or WO2021091978. In some embodiments, the antigen-binding domain is an antibody fragment containing a variable heavy chain (VH) and a variable light chain (VL) region. In some embodiments, the anti-BCMA CAR contains an antigen-binding domain, such as an scFv, containing a variable heavy (VH) and/or a variable light (VL) region derived from an antibody described in WO 2016/090320 or WO2016090327.

In some embodiments, the antigen-binding domain is an antibody fragment containing a variable heavy chain (VH) and a variable light chain (VL) region. In some aspects, the VH region is or includes an amino acid sequence having at least 90%, 91%, 92%, 93%, 94%, 95%, 96%, 97%, 98% or 99% sequence identity to the VH region amino acid sequence set forth in any of SEQ ID NOs: 18, 20, 22, 24, 32, 34, 36, 41, 43, 45, 47, 49, 51, 53, 55, 57, 59, 61, 63, 65, 67, 69, 71, 73, 75, 145, 147, 149 and 151; and/or the VL region is or includes an amino acid sequence having at least 90%, 91%, 92%, 93%, 94%, 95%, 96%, 97%, 98%, or 99% sequence identity to the VL region amino acid sequence set forth in any of SEQ ID NOs: 19, 21, 23, 25, 33, 35, 37, 42, 44, 46, 48, 50, 52, 54, 56, 58, 60, 62, 64, 66, 68, 70, 72, 74, 76, 146, 148, 150 and 152.

In some embodiments, the antigen-binding domain, such as an scFv, contains a VH set forth in SEQ ID NO: 18 and a VL set forth in SEQ ID NO: 19. In some embodiments, the antigen-binding domain, such as an scFv, contains a VH set forth in SEQ ID NO: 20 and a VL set forth in SEQ ID NO:21. In some embodiments, the antigen-binding domain, such as an scFv, contains a VH set forth in SEQ ID NO: 22 and a VL set forth in SEQ ID NO:23. In some embodiments, the antigen-binding domain, such as an scFv, contains a VH set forth in SEQ ID NO: 24 and a VL set forth in SEQ ID NO:25. In some embodiment the antigen-binding domain, such as an scFv, contains a VH set forth in SEQ ID NO: 32 and a VL set forth in SEQ ID NO:33. In some embodiments, the antigen-binding domain, such as an scFv, contains a VH set forth in SEQ ID NO:34 and a VL set forth in SEQ ID NO:35. In some embodiments, the antigen-binding domain, such as an scFv, contains a VH set forth in SEQ ID NO: 36 and a VL set forth in SEQ ID NO:37. In some embodiments, the antigen-binding domain, such as an scFv, contains a VH set forth in SEQ ID NO: 41 and a VL set forth in SEQ ID NO: 42. In some embodiments, the antigen-binding domain, such as an scFv, contains a VH set forth in SEQ ID NO: 43 and a VL set forth in SEQ ID NO: 44. In some embodiments, the antigen-binding domain, such as an scFv, contains a VH set forth in SEQ ID NO: 45 and a VL set forth in SEQ ID NO: 46. In some embodiments, the antigen-binding domain, such as an scFv, contains a VH set forth in SEQ ID NO: 47 and a VL set forth in SEQ ID NO: 48. In some embodiments, the antigen-binding domain, such as an scFv, contains a VH set forth in SEQ ID NO: 49 and a VL set forth in SEQ ID NO: 50. In some embodiments, the antigen-binding domain, such as an scFv, contains a VH set forth in SEQ ID NO: 51 and a VL set forth in SEQ ID NO: 52. In some embodiments, the antigen-binding domain, such as an scFv, contains a VH set forth in SEQ ID NO: 53 and a VL set forth in SEQ ID NO: 54. In some embodiments, the antigen-binding domain, such as an scFv, contains a VH set forth in SEQ ID NO: 55 and a VL set forth in SEQ ID NO: 56. In some embodiments, the antigen-binding domain, such as an scFv, contains a VH set forth in SEQ ID NO: 57 and a VL set forth in SEQ ID NO: 58. In some embodiments, the antigen-binding domain, such as an scFv, contains a VH set forth in SEQ ID NO: 59 and a VL set forth in SEQ ID NO: 60. In some embodiments, the antigen-binding domain, such as an scFv, contains a VH set forth in SEQ ID NO: 61 and a VL set forth in SEQ ID NO: 62. In some embodiments, the antigen-binding domain, such as an scFv, contains a VH set forth in SEQ ID NO: 63 and a VL set forth in SEQ ID NO: 64. In some embodiments, the antigen-binding domain, such as an scFv, contains a VH set forth in SEQ ID NO: 65 and a VL set forth in SEQ ID NO: 66. In some embodiments, the antigen-binding domain, such as an scFv, contains a VH set forth in SEQ ID NO: 67 and a VL set forth in SEQ ID NO: 68. In some embodiments, the antigen-binding domain, such as an scFv, contains a VH set forth in SEQ ID NO: 69 and a VL set forth in SEQ ID NO: 70. In some embodiments, the antigen-binding domain, such as an scFv, contains a VH set forth in SEQ ID NO: 71 and a VL set forth in SEQ ID NO: 72. In some embodiments, the antigen-binding domain, such as an scFv, contains a VH set forth in SEQ ID NO: 73 and a VL set forth in SEQ ID NO: 74. In some embodiments, the antigen-binding domain, such as an scFv, contains a VH set forth in SEQ ID NO: 75 and a VL set forth in SEQ ID NO: 76. In some embodiments, the antigen-binding domain, such as an scFv, contains a VH set forth in SEQ ID NO: 145 and a VL set forth in SEQ ID NO: 146. In some embodiments, the antigen-binding domain, such as an scFv, contains a VH set forth in SEQ ID NO: 147 and a VL set forth in SEQ ID NO: 148. In some embodiments, the antigen-binding domain, such as an scFv, contains a VH set forth in SEQ ID NO: 149 and a VL set forth in SEQ ID NO: 150. In some embodiments, the antigen-binding domain, such as an scFv, contains a VH set forth in SEQ ID NO: 151 and a VL set forth in SEQ ID NO: 152. In some embodiments, the VH or VL has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing VH or VL sequences, and retains binding to BCMA. In some embodiments, the VH region is amino-terminal to the VL region. In some embodiments, the VH region is carboxy-terminal to the VL region. In some embodiments, the variable heavy and variable light chains are connected by a linker. In some embodiments, the linker is set forth in SEQ ID NOs: 28, 29, 30, or 38.

Among a provided anti-BCMA CAR is a CAR in which the antibody or antigen-binding fragment contains a VH region comprising the sequence set forth in SEQ ID NO: 18 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 18; and contains a VL region comprising the sequence set forth in SEQ ID NO: 19 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 19. In some embodiments, the antibody or antigen-binding fragment of the provided CAR contains a VH region that has a CDRH1, a CDRH2 and a CDRH3 comprising the amino acid sequence of SEQ ID NOS: 189, 190, and 191, respectively and a VL region that has a CDRL1, a CDRL2 and a CDRL3 comprising the amino acid sequence of SEQ ID NOS: 192, 193, and 194, respectively. In some embodiments, the antibody or antigen-binding fragment of the provided CAR contains a VH region that has a CDRH1, a CDRH2 and a CDRH3 comprising the amino acid sequence of SEQ ID NOS: 195, 196, and 197, respectively and a VL region that has a CDRL1, a CDRL2 and a CDRL3 comprising the amino acid sequence of SEQ ID NOS: 198, 199, and 200, respectively. In some embodiments, the antibody or antigen-binding fragment of the provided CAR contains a VH region that has a CDRH1, a CDRH2 and a CDRH3 comprising the amino acid sequence of SEQ ID NOS: 201, 202, and 203, respectively and a VL region that has a CDRL1, a CDRL2 and a CDRL3 comprising the amino acid sequence of SEQ ID NOS: 204, 205, and 206, respectively. In some embodiments, the antibody or antigen-binding fragment of the provided CAR contains a VH region that has a CDRH1, a CDRH2 and a CDRH3 comprising the amino acid sequence of SEQ ID NOS: 207, 208, and 209, respectively and a VL region that has a CDRL1, a CDRL2 and a CDRL3 comprising the amino acid sequence of SEQ ID NOS: 210, 211, and 212, respectively. In some embodiments, the VH region comprises the sequence set forth in SEQ ID NO: 18 and the VL region comprises the sequence set forth in SEQ ID NO: 19. In some embodiments, the antibody or antigen-binding fragment is a single-chain antibody fragment, such as an scFv. In some embodiments, the scFv comprises the sequence of amino acids set forth in SEQ ID NO:213 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:213. In some embodiments, the anti-BCMA CAR has the sequence of amino acids set forth in SEQ NO: 116 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO: 116. In some embodiments, the anti-BCMA CAR is encoded by the polynucleotide sequence set forth in SEQ NO: 214 or a polynucleotide sequence of at least at or about 90%, at or about 91%, at or about 92%, at or about 93%, at or about 94%, at or about 95%, at or about 96%, at or about 97%, at or about 98%, or at or about 99% identity to SEQ ID NO:214.

Among a provided anti-BCMA CAR is a CAR in which the antibody or antigen-binding fragment contains a VH region comprising the sequence set forth in SEQ ID NO: 24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24; and contains a VL region comprising the sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25. In some embodiments, the antibody or antigen-binding fragment of the provided CAR contains a VH region that has a CDRH1, a CDRH2 and a CDRH3 comprising the amino acid sequence of SEQ ID NOS: 173, 174 and 175, respectively and a VL region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a VH region that has a CDRH1, a CDRH2 and a CDRH3 comprising the amino acid sequence of SEQ ID NOS: 176, 177 and 175, respectively and a VL region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a VH region that has a CDRH1, a CDRH2 and a CDRH3 comprising the amino acid sequence of SEQ ID NOS: 178, 179 and 175, respectively and a VL region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a VH region that has a CDRH1, a CDRH2 and a CDRH3 comprising the amino acid sequence of SEQ ID NOS: 180, 181 and 182, respectively and a VL region that has a CDRL1, a CDRL2 and a CDRL3 comprising the amino acid sequence of SEQ ID NOS: 186, 187 and 185, respectively. In some embodiments, the VH region comprises the sequence set forth in SEQ ID NO:24 and the VL region comprises the sequence set forth in SEQ ID NO:25. In some embodiments, the antibody or antigen-binding fragment is a single-chain antibody fragment, such as an scFv. In some embodiments, the scFv comprises the sequence of amino acids set forth in SEQ ID NO:188 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO: 188. In some embodiments, the anti-BCMA CAR has the sequence of amino acids set forth in SEQ NO: 124 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO: 124. In some embodiments, the anti-BCMA CAR has the sequence of amino acids set forth in SEQ NO: 125 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO: 125.

In some embodiments, the scFv comprises the amino acid sequence set forth in any one of SEQ ID NOS: 216-247, or an amino acid sequence having at least 90, 95, 96, 97, 98, 99, or 100% sequence identity to a sequence set forth in any one of SEQ ID NOS: 216-247.

In some embodiments, the antigen-binding domain comprises an sdAb. In some embodiments, the antigen-binding domain contains the sequence set forth by SEQ ID NO:77. In some embodiments, the antigen-binding domain comprises a sequence at least or about 50, 60, 70, 80, 85, 90, 95, 96, 97, 98, 99, or 100% identical to the sequence set forth by SEQ ID NO:77.

In some embodiments, the CAR comprises the amino acid sequence set forth in any one of SEQ ID NOS: 90-141, or an amino acid sequence having at least 90, 95, 96, 97, 98, or 99% sequence identity to a sequence set forth in any one of SEQ ID NOS: 90-141.

In some embodiments, the antigen targeted by the receptor is CD20, CD19, CD22, ROR1, CD45, CD21, CD5, CD33, Igkappa, Iglambda, CD79a, CD79b or CD30. In particular aspects, the antigen is CD19.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NOs: 251 and 252, respectively, and CDR-H3 set forth in SEQ ID NOs: 253 or 266 and CDR-L1 set forth in SEQ ID NO: 248 and CDR-L2 set forth in SEQ ID NOs: 249 or 267 and CDR-L3 sequences set forth in SEQ ID NOs: 250 or 268. In some embodiments, the FMC63 antibody comprises the heavy chain variable region (V_{H}) comprising the amino acid sequence of SEQ ID NO: 254 and the light chain variable region (V_{L}) comprising the amino acid sequence of SEQ ID NO: 255.

In some embodiments, the scFv comprises a variable light chain containing the CDR-L1 sequence of SEQ ID NO:248, a CDR-L2 sequence of SEQ ID NO:249, and a CDR-L3 sequence of SEQ ID NO:250 and/or a variable heavy chain containing a CDR-H1 sequence of SEQ ID NO:251, a CDR-H2 sequence of SEQ ID NO:252, and a CDR-H3 sequence of SEQ ID NO:253, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of FMC63 set forth in SEQ ID NO:254 and a variable light chain region of FMC63 set forth in SEQ ID NO:255, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:29. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a polynucleotide set forth in SEQ ID NO:269 or a sequence that exhibits at least 85%, 86%, 87%, 88%, 89%, 90%, 91%, 92%, 93%, 94%, 95%, 96%, 97%, 98%, or 99% sequence identity to SEQ ID NO:269. In some embodiments, the scFv comprises the sequence of amino acids set forth in SEQ ID NO:256 or a sequence that exhibits at least 85%, 86%, 87%, 88%, 89%, 90%, 91%, 92%, 93%, 94%, 95%, 96%, 97%, 98%, or 99% sequence identity to SEQ ID NO:256.

In some embodiments the antigen-binding domain includes a VH and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 260-262, respectively, and CDR-L1, CDR-L2 and CDR-L3 sequences set forth in SEQ ID NOS: 257-259, respectively. In some embodiments, the SJ25C1 antibody comprises the heavy chain variable region (V_{H}) comprising the amino acid sequence of SEQ ID NO: 263 and the light chain variable region (V_{L}) comprising the amino acid sequence of SEQ ID NO: 264. In some embodiments, the scFv comprises a variable light chain containing a CDR-L1 sequence of SEQ ID NO:257, a CDR-L2 sequence of SEQ ID NO: 258, and a CDR-L3 sequence of SEQ ID NO:259 and/or a variable heavy chain containing a CDR-H1 sequence of SEQ ID NO:260, a CDR-H2 sequence of SEQ ID NO:261, and a CDR-H3 sequence of SEQ ID NO:262, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:263 and a variable light chain region of SJ25C1 set forth in SEQ ID NO:264, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:28. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:265 or a sequence that exhibits at least 85%, 86%, 87%, 88%, 89%, 90%, 91%, 92%, 93%, 94%, 95%, 96%, 97%, 98%, or 99% sequence identity to SEQ ID NO:265.

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from rituximab, such as rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as m971 scFv.

In some embodiments, the antigen or antigen binding domain is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a V_{H} and a V_{L} from an antibody or antibody fragment set forth in International Publication Nos. WO 2016/090329 and WO 2016/090312.

In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a CH1/CL, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain.

In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a CH1/CL, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain.

The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is a spacer having at least a particular length, such as having a length that is at least 100 amino acids, such as at least 110, 125, 130, 135, 140, 145, 150, 160, 170, 180, 190, 200, 210, 220, 230, 240, or 250 amino acids in length. Exemplary spacers include IgG4 hinge alone, IgG4 hinge linked to CH2 and CH3 domains, or IgG4 hinge linked to the CH3 domain. Exemplary spacers include IgG4 hinge alone, IgG4 hinge linked to CH2 and CH3 domains, or IgG4 hinge linked to the CH3 domain. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al., Clin. Cancer Res., 19:3153 (2013), Hudecek et al. (2015) Cancer Immunol Res. 3(2): 125-135, international patent application publication number WO2014031687, U.S. Pat. No. 8,822,647 or published app. No. US2014/0271635. In some embodiments, the spacer includes a sequence of an immunoglobulin hinge region, a CH2 and CH3 region. In some embodiments, one of more of the hinge, CH2 and CH3 is derived all or in part from IgG4 or IgG2. In some cases, the hinge, CH2 and CH3 is derived from IgG4. In some aspects, one or more of the hinge, CH2 and CH3 is chimeric and contains sequence derived from IgG4 and IgG2. In some examples, the spacer contains an IgG4/2 chimeric hinge, an IgG2/4 CH2, and an IgG4 CH3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the CH2 region, of the full-length IgG4 Fc sequence or an N176Q at position 176, in the CH2 region, of the full-length IgG4 Fc sequence.

In some embodiments, the spacer has the sequence ESKYGPPCPPCP (set forth in SEQ ID NO: 1), and is encoded by the sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the encoded spacer is or contains the sequence set forth in SEQ ID NO: 31. In some embodiments, the constant region or portion is of IgD. In some embodiments, the spacer has the sequence set forth in SEQ ID NO: 5. In some embodiments, the spacer has the sequence set forth in SEQ ID NO: 89.

Other exemplary spacer regions include hinge regions derived from CD8a, CD28, CTLA4, PD-1, or FcγRIIIa. In some embodiments, the spacer contains a truncated extracellular domain or hinge region of a CD8a, CD28, CTLA4, PD-1, or FcγRIIIa. In some embodiments, the spacer is a truncated CD28 hinge region. In some embodiments, a short oligo- or polypeptide linker, for example, a linker of between 2 and 10 amino acids in length, such as one containing alanines or alanine and arginine, e.g., alanine triplet (AAA) or RAAA (SEQ ID NO: 144), is present and forms a linkage between the scFv and the spacer region of the CAR. In some embodiments, the spacer has the sequence set forth in SEQ ID NO: 78. In some embodiments, the spacer has the sequence set forth in SEQ ID NO: 80. In some embodiments, the spacer has the sequence set forth in any of SEQ ID NOs: 81-83, In some embodiments, the spacer has the sequence set forth in SEQ ID NO: 84. In some embodiments, the spacer has the sequence set forth in SEQ ID NO: 86. In some embodiments, the spacer has the sequence set forth in SEQ ID NO: 88.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4, 5, 31, 78, 80, 81, 82, 83, 84, 86, 88, or 89.

In some embodiments, the spacer has the sequence set forth in SEQ ID NOS: 157-165. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 157-165.

This antigen recognition domain generally is linked to one or more intracellular signaling components, such as signaling components that mimic stimulation and/or activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the chimeric antigen receptor includes a transmembrane domain linking the extracellular domain and the intracellular signaling domain. In some embodiments, the transmembrane domain is fused to the extracellular domain, such as linked or fused between the extracellular domain (e.g. scFv) and intracellular signaling domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD8a, CD9, CD16, CD22, CD33, CD37, CD64, CD80, CD86, CD134, CD137 (4-1BB), CD154, CTLA-4 or PD-1. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28. Exemplary sequences of transmembrane domains are or comprise the sequences set forth in SEQ ID NOs: 8, 79, 85, 87, 142, or 143. Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR CD3 chain that mediates T-cell stimulation and/or activation and cytotoxicity, e.g., CD3 zeta chain, CD3 gamma, CD3 delta, CD3 epsilon, FcR gamma, FcR beta, CDS, CD22, CD79a, CD79b and CD66d. In some examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell stimulation and/or activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25 or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell stimulation and/or activation is in some aspects described as being mediated by two classes of cytoplasmic signaling sequences: those that initiate antigen-dependent primary stimulation and/or activation through the TCR (primary cytoplasmic signaling regions, domains or sequences), and those that act in an antigen-independent manner to provide a secondary or co-stimulatory signal (secondary cytoplasmic signaling regions, domains or sequences). In some aspects, the CAR includes one or both of such signaling components.

In some embodiments, the CAR includes a signaling region and/or transmembrane portion of a costimulatory receptor, such as CD28, 4-1BB, OX40 (CD134), CD27, DAP10, DAP12, ICOS and/or other costimulatory receptors. In some aspects, the same CAR includes both the primary cytoplasmic signaling region and costimulatory signaling components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, one or more different recombinant receptors can contain one or more different intracellular signaling region(s) or domain(s). In some embodiments, the primary cytoplasmic signaling region is included within one CAR, whereas the costimulatory component is provided by another receptor, e.g., another CAR recognizing another antigen. In some embodiments, the CARs include activating or stimulatory CARs, and costimulatory CARs, both expressed on the same cell (see WO2014/055668).

In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12, NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P10747.1), or CD8a (Accession No. P01732.1), or variant thereof, such as a transmembrane domain that comprises the sequence of amino acids set forth in SEQ ID NOs: 8, 79, 142, or 143 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs: 8, 79, 142, or 143. In some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the transmembrane domain is a transmembrane domain from CD8a. In some embodiments, the transmembrane domain is any as described in Milone et al., Mol. Ther. (2009) 12(9):1453-64. In some embodiments, the transmembrane domain is or comprises the sequence set forth in SEQ ID NO: 143.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NOs: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs: 10 or 11. In some embodiments, the intracellular domain comprises an intracellular costimulatory signaling domain of 4-1BB (e.g. Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular domain comprises an intracellular costimulatory signaling domain of 4-1BB, In some embodiments, the 4-1BB co-stimulatory molecule is any as described in Milone et al., Mol. Ther. (2009) 12(9):1453-64. In some embodiments, the co-stimulatory molecular has the sequence set forth in SEQ ID NO: 12.

In some embodiments, the intracellular signaling domain of the recombinant receptor, e.g. the CAR, comprises a human CD3 zeta stimulatory signaling domain or functional variant thereof, such as a 112 AA cytoplasmic domain of isoform 3 of human CD3ζ (Accession No. P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NOs: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs: 13, 14 or 15. In some embodiments, the CD3-zeta domain is any as described in Milone et al., Mol. Ther. (2009) 12(9):1453-64. In some embodiments, the CD3-zeta is or comprises the sequence set forth in SEQ ID NO: 13.

In some aspects, the spacer contains only a hinge region of an IgG, such as only a hinge of IgG4 or IgG1, such as the hinge only spacer set forth in SEQ ID NO: 1 or SEQ ID NO: 89. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 4. In some embodiments, the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers. In some embodiments, the spacer is a CD8a hinge, such as set forth in any of SEQ ID NOs: 81-83, an FcγRIIIa hinge, such as set forth in SEQ ID NO: 88, a CTLA4 hinge, such as set forth in SEQ ID NO: 84, or a PD-1 hinge, such as set forth in SEQ ID NO: 86. In some embodiments the spacer is derived from CD8. In some embodiments, the spacer is a CD8a hinge sequence. In some embodiments, the hinge sequence is any as described in Milone et al., Mol. Ther. (2009) 12(9):1453-64. In some embodiments, the hinge is or comprises the sequence set forth in SEQ ID NO:82.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28− derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain. In some embodiments, the CAR includes an antibody or fragment, such as scFv, a spacer such as any of the Ig-hinge containing spacers, a CD8-derived transmembrane domain, a 4-1BB-derived intracellular signaling domain, and a CD3 zeta-derived signaling domain.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred. In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence. In some embodiments, such CAR constructs further includes a T2A ribosomal skip element and/or a tEGFR sequence, e.g., downstream of the CAR.

An exemplary polypeptide for a truncated EGFR (e.g. tEGFR) comprises the sequence of amino acids set forth in SEQ ID NOs: 7 or 166 or a sequence of amino acids that exhibits at least 85%, 86%, 87%, 88%, 89%, 90%, 91%, 92%, 93% 94% 95% 96%, 97% 98%, 99% or more sequence identity to SEQ ID NOs: 7 or 166. An exemplary T2A linker sequence comprises the sequence of amino acids set forth in SEQ ID NOs: 6 or 167 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs: 6 or 167.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NOs: 6 or 167, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs: 6 or 167. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NOs: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs: 7 or 166.

In some embodiments, the encoded CAR can sequence can further include a signal sequence or signal peptide that directs or delivers the CAR to the surface of the cell in which the CAR is expressed. In some embodiments, the signal peptide is derived from a transmembrane protein. In some examples the signal peptide is derived from CD8a, CD33, or an IgG. Exemplary signal peptides include the sequences set forth in SEQ ID NOs: 39, 40 and 153. In some examples the signal peptide is derived from CD8a. In some embodiments, the signal peptide is the sequence set forth in Accession No. NM_001768. In some embodiments, the signal peptide include the sequences set forth in SEQ ID NO: 39.

In some embodiments, the CAR includes an anti-BCMA antibody or fragment, such as any of the anti-human BCMA antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-BCMA antibody or fragment, such as any of the anti-human BCMA antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR.

In some embodiments, the CAR includes an anti-GPRC5D antibody or fragment, such as any of the anti-human GPRC5D antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-GPRC5D antibody or fragment, such as any of the anti-human GPRC5D antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR.

In some embodiments, the CAR includes an anti-CD19 antibody or fragment, such as any of the anti-human CD19 antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-CD19 antibody or fragment, such as any of the anti-human GPRC5D antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition. In some embodiments, the CAR specifically binds to BCMA, such as human BCMA, and includes an anti-human BCMA antibody or fragment as described. Non-limiting exemplary CAR sequences, including anti-BCMA CAR sequences, are set forth in SEQ ID NOs: 90-141. In some embodiments, an anti-BCMA CAR includes the amino acid sequence set forth in any of SEQ ID NOS: 90-141 or an amino acid sequence that exhibits at least at or about 90%, at or about 91%, at or about 92%, at or about 93%, at or about 94%, at or about 95%, at or about 96%, at or about 97%, at or about 96%, at or about 97%, at or about 98%, at or about 99% sequence identity to any one of SEQ ID NOS: 90-141, and wherein the CAR specifically binds BCMA, e.g. human BCMA.

In some embodiments, the dose of genetically engineered T cells comprises idecabtagene vicleucel cells (e.g., such as ABECMA® cells); bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells, or GC012F cells. In some embodiments, the dose of genetically engineered T cells comprises idecabtagene vicleucel cells (e.g., such as ABECMA® cells).

In some embodiments, among such antibodies or antigen-binding domains in the provided CARs are antibodies capable of binding a target protein, such as human BCMA protein, with at least a certain affinity, as measured by any of a number of known methods. In some embodiments, the affinity is represented by an equilibrium dissociation constant (KD); in some embodiments, the affinity is represented by EC50.

A variety of assays are known for assessing binding affinity and/or determining whether a binding molecule (e.g., an antibody or fragment thereof) specifically binds to a particular ligand (e.g., an antigen, such as a BCMA protein). It is within the level of a skilled artisan to determine the binding affinity of a binding molecule, e.g., an antibody, for a target protein, e.g., BCMA. For example, in some embodiments, a BIAcore® instrument can be used to determine the binding kinetics and constants of a complex between two proteins (e.g., an antibody or fragment thereof, and an antigen, such as a BCMA cell surface protein, soluble BCMA protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

In some embodiments, the binding molecule, e.g., antibody or fragment thereof or antigen-binding domain of a CAR, binds, such as specifically binds, to a target protein, e.g., a cell surface BCMA protein or soluble BCMA protein or an epitope therein, with an affinity or KA (i.e., an equilibrium association constant of a particular binding interaction with units of 1/M; equal to the ratio of the on-rate [kon or ka] to the off-rate [koff or kd] for this association reaction, assuming bimolecular interaction) equal to or greater than 105 M-1. In some embodiments, the antibody or fragment thereof or antigen-binding domain of a CAR exhibits a binding affinity for the peptide epitope with a KD (i.e., an equilibrium dissociation constant of a particular binding interaction with units of M; equal to the ratio of the off-rate [koff or kd] to the on-rate [kon or ka] for this association reaction, assuming bimolecular interaction) of equal to or less than 10-5 M. For example, the equilibrium dissociation constant KD ranges from 10-5 M to 10-13 M, such as 10-7 M to 10-11 M, 10-8 M to 10-10 M, or 10-9 M to 10-10 M. The on-rate (association rate constant; kon or ka; units of 1/Ms) and the off-rate (dissociation rate constant; koff or kd; units of 1/s) can be determined using any of the assay methods known in the art, for example, surface plasmon resonance (SPR).

In some embodiments, the binding affinity (EC50) and/or the dissociation constant of the antibody (e.g. antigen-binding fragment) or antigen-binding domain of a CAR to a target protein, such as human BCMA protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, is from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC50) and/or the equilibrium dissociation constant, KD, of the antibody to a target protein, such as human BCMA protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a target protein, such as human BCMA protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a KA of up to 107 M-1, up to 106 M-1, up to 105 M-1. In some cases, a binding molecule (e.g. antibody or fragment thereof) that exhibits high affinity binding to a particular epitope interacts with such epitope with a KA of at least 107 M-1, at least 108 M-1, at least 109 M-1, at least 1010 M-1, at least 1011 M-1, at least 1012 M-1, or at least 1013 M-1. In some embodiments, the binding affinity (EC50) and/or the equilibrium dissociation constant, KD, of the binding molecule, e.g., anti-BCMA antibody or fragment thereof or antigen-binding domain of a CAR, to a target (e.g., BCMA) protein, is from or from about 0.01 nM to about 1 μM, 0.1 nM to 1 μM, 1 nM to 1 μM, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC50) and/or the dissociation constant of the equilibrium dissociation constant, KD, of the binding molecule, e.g., anti-BCMA antibody or fragment thereof or antigen-binding domain of a CAR, to a target (e.g., BCMA) protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody (e.g., anti-BCMA reference antibody).

In some embodiments, the binding affinity of the antibody or antigen-binding domain of a CAR, for different form or topological type of antigens, e.g., soluble or shed target protein compared to the binding affinity to a membrane-bound target protein, to determine the preferential binding or relative affinity for a particular form or topological type. For example, in some aspects, an anti-BCMA antibodies or antigen-binding domain of a CAR can exhibit preferential binding to membrane-bound BCMA as compared to soluble or shed BCMA and/or exhibit greater binding affinity for, membrane-bound BCMA compared to soluble or shed BCMA. In some embodiments, the equilibrium dissociation constant, KD, for different form or topological type of BCMA proteins, can be compared to determine preferential binding or relative binding affinity. In some embodiments, the preferential binding or relative affinity to a membrane-bound BCMA compared to soluble or shed BCMA can be high. For example, in some cases, the ratio of KD for soluble or shed BCMA and the KD for membrane-bound BCMA is more than 10, 15, 20, 25, 30, 40, 50, 60, 70, 80, 90, 100, 200, 500, 1000, 2000 or more and the antibody or antigen-binding domain preferentially binds or has higher binding affinity for membrane-bound BCMA. In some cases, the ratio of KA for membrane-bound BCMA and the KA for soluble or shed BCMA is more than 10, 15, 20, 25, 30, 40, 50, 60, 70, 80, 90, 100, 200, 500, 1000, 2000 or more and the antibody or antigen-binding domain preferentially binds or has higher binding affinity for membrane-bound BCMA. In some cases, the antibody or antigen-binding domain of CAR binds soluble or shed BCMA and membrane-bound BCMA to a similar degree, e.g., the ratio of KD for soluble BCMA and KD for membrane-bound BCMA is or is about 1. In some cases, the antibody or antigen-binding domain of CAR binds soluble or shed BCMA and membrane-bound BCMA to a similar degree, e.g., the ratio of KA for soluble BCMA and KA for membrane-bound BCMA is or is about 1. The degree of preferential binding or relative affinity for membrane-bound BCMA or soluble or shed BCMA can be compared with that of a known antibody, such as a reference antibody (e.g., reference anti-BCMA CAR). In some embodiments, the reference antibody (e.g., reference anti-BCMA CAR) binds to membrane-bound and soluble or shed BCMA protein.

### B. Nucleic Acids and Vectors for Methods for Genetic Engineering

In some embodiments, the methods of genetically engineering cells to express one or more recombinant receptors comprises introduction of nucleic acids and vectors encoding the one or more recombinant receptors. In some embodiments, the cells, e.g., T cells, are genetically engineered to express a recombinant receptor. In some embodiments, the engineering is carried out by introducing nucleic acid molecules that encode the recombinant receptor. Also provided are nucleic acid molecules encoding a recombinant receptor, and vectors or constructs containing such nucleic acids and/or nucleic acid molecules.

In some cases, the nucleic acid sequence encoding the recombinant receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some embodiments, the signal peptide is derived from a transmembrane protein. In some examples the signal peptide is derived from CD8a, CD33, or an IgG. Non-limiting exemplary examples of signal peptides include, for example, the CD33 signal peptide set forth in SEQ ID NO: 153, CD8a signal peptide set forth in SEQ ID NO: 154, or the signal peptide set forth in SEQ ID NO:39 or modified variant thereof. In some embodiments, the signal peptide is the CD8a signal peptide set forth in Accession No. NM_001768.

In some embodiments, the nucleic acid molecule encoding the recombinant receptor contains at least one promoter that is operatively linked to control expression of the recombinant receptor. In some examples, the nucleic acid molecule contains two, three, or more promoters operatively linked to control expression of the recombinant receptor. In some embodiments, nucleic acid molecule can contain regulatory sequences, such as transcription and translation initiation and termination codons, which are specific to the type of host (e.g., bacterium, fungus, plant, or animal) into which the nucleic acid molecule is to be introduced, as appropriate and taking into consideration whether the nucleic acid molecule is DNA- or RNA-based. In some embodiments, the nucleic acid molecule can contain regulatory/control elements, such as a promoter, an enhancer, an intron, a polyadenylation signal, a Kozak consensus sequence, and splice acceptor or donor. In some embodiments, the nucleic acid molecule can contain a nonnative promoter operably linked to the nucleotide sequence encoding the recombinant receptor and/or one or more additional polypeptide(s). In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the nucleic acid molecule does not include a regulatory element, e.g. promoter.

In some embodiments, the nucleic acid molecule encoding the recombinant receptor, e.g., CAR or other antigen receptor, further includes nucleic acid sequences encoding a marker and/or cells expressing the CAR or other antigen receptor further includes a marker, e.g., a surrogate marker, such as a cell surface marker, which may be used to confirm transduction or engineering of the cell to express the receptor, such as a truncated version of a cell surface receptor, such as truncated EGFR (tEGFR). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the nucleic acid molecule, e.g., a nucleic acid molecule encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same nucleic acid molecule that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NOs:7 or 166) or a prostate-specific membrane antigen (PSMA) or modified form thereof tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some aspects, the marker, e.g. surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Publication No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NOs: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:7 or 166. An exemplary T2A linker sequence comprises the sequence of amino acids set forth in SEQ ID NOs:6 or 167 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:6 or 167.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NOs: 6 or 167, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs: 6 or 167. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NOs: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs: 7 or 166.

In some embodiments, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein, without limitation, 2A sequences from the foot-and-mouth disease virus (F2A; e.g. SEQ ID NO:171), equine rhinitis A virus (E2A; e.g. SEQ ID NO: 170), Thosea asigna virus (T2A, e.g., SEQ ID NOs: 6 or 167), and porcine teschovirus-1 (P2A; e.g. SEQ ID NOs:168 or 169) as described in U.S. Patent Publication No. 20070116690.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Introduction of the nucleic acid molecules encoding the recombinant receptor in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gammaretroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle. bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In certain embodiments, provided herein are nucleic acids and vectors for use in the method of genetic engineering described in Section V.C.

### C. Dosage and Administration of a Cell Therapy

In some embodiments of the methods, compositions, combinations, kits and uses provided herein, the treatment includes administering to a subject a cell therapy (e.g. a cell therapy, such as an autologous T cell therapy). For example, the cell therapy is an autologous T cell therapy, such as an anti-BCMA CAR T cell therapy.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

In some embodiments, the cell-based therapy is or comprises administration of cells, such as immune cells, for example T cell or NK cells, that target a molecule expressed on the surface of a lesion, such as a tumor or a cancer. In some embodiments, the cells express a recombinant receptor, e.g. CAR, that contains an extracellular ligand-binding domain that specifically binds to an antigen. In some embodiments, the recombinant receptor is a CAR that contains an extracellular antigen-recognition domain that specifically binds to BCMA. In some embodiments, the immune cells express a recombinant receptor, such as a chimeric antigen receptor (CAR). In some embodiments, the T cell therapy includes administering T cells engineered to express a chimeric antigen receptor (CAR). In particular embodiments, the cell therapy, e.g. anti-BCMA CAR T cell therapy, is for treating a multiple myeloma, such as a relapsed and refractory (R/R multiple myeloma). In some embodiments, the cells are autologous to the subject.

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4+vs CD8+ T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In some embodiments, the dose includes fewer than about 5×10⁸ total recombinant receptor (e.g., CAR)-expressing cells, T cells, or peripheral blood mononuclear cells (PBMCs), e.g., in the range of about 1×10⁸ to 5×10⁸ such cells, such as 1.5×10⁸, 3×10⁸, or 4.5×10⁸ or total such cells, or the range between any two of the foregoing values.

The cells can be administered by any suitable means. The cells are administered in a dosing regimen to achieve a therapeutic effect, such as a reduction in tumor burden. Various dosing schedules of the T cell therapy include but are not limited to single or multiple administrations over various time-points, bolus administration, and pulse infusion.

Preconditioning subjects with immunodepleting (e.g., lymphodepleting) therapies, including any of those described herein, in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is administered a preconditioning agent (lymphodepleting treatment) as described herein.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In some embodiments, a dose of cells is administered to subjects in accord with the provided T cell therapy methods. In some embodiments, the size or timing of the doses is determined as a function of the particular disease or condition in the subject. One may empirically determine the size or timing of the doses for a particular disease in view of the provided description.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 150 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the dose of cells is a flat dose of cells or fixed dose of cells such that the dose of cells is not tied to or based on the body surface area or weight of a subject.

In some embodiments, the cell therapy comprises administration of a dose comprising a number of cell from or from about 1×10⁵ to 2×10⁹ total recombinant receptor-expressing cells, total T cells, or total peripheral blood mononuclear cells (PBMCs), from or from about 5×10⁵ to 1×10⁹ total recombinant receptor-expressing cells, total T cells, or total peripheral blood mononuclear cells (PBMCs) or from or from about 1×10⁶ to 1×10⁹ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸, at least or about at least 1×10⁹ of such cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 1.0×10⁷ to at or about 1.2×10⁹ such cells, such as at or about 1.0×10⁷, 1.5×10⁷, 2.0×10⁷, 2.5×10⁷, 5×10⁷, 1.5×10⁸,3×10⁸, 4.5×10⁸, 6×10⁸, 8×10⁸ or 1.2×10⁹ total such cells, or the range between any two of the foregoing values. In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1.5×10⁸, 3×10⁸, 4.5×10⁸, 6×10⁸, 8×10⁸ or 1.2×10⁹ total such cells, or the range between any two of the foregoing values. In some embodiments, for example, where the subject is a human, the dose includes at or about 1.0×10⁷ total recombinant receptor (e.g., CAR)-expressing cells, T cells, or peripheral blood mononuclear cells (PBMCs). In some embodiments, for example, where the subject is a human, the dose includes at or about 1.5×10⁷ total recombinant receptor (e.g., CAR)-expressing cells, T cells, or peripheral blood mononuclear cells (PBMCs). In some embodiments, for example, where the subject is a human, the dose includes at or about 2.0×10⁷ total recombinant receptor (e.g., CAR)-expressing cells, T cells, or peripheral blood mononuclear cells (PBMCs). In some embodiments, for example, where the subject is a human, the dose includes at or about 2.5×10⁷ total recombinant receptor (e.g., CAR)-expressing cells, T cells, or peripheral blood mononuclear cells (PBMCs). In some embodiments, for example, where the subject is a human, the dose includes at or about 5×10⁷ total recombinant receptor (e.g., CAR)-expressing cells, T cells, or peripheral blood mononuclear cells (PBMCs). In some embodiments, for example, where the subject is a human, the dose includes at or about 1.5×10⁸ total recombinant receptor (e.g., CAR)-expressing cells, T cells, or peripheral blood mononuclear cells (PBMCs). In some embodiments, for example, where the subject is a human, the dose includes at or about 3×10⁸ total recombinant receptor (e.g., CAR)-expressing cells, T cells, or peripheral blood mononuclear cells (PBMCs). In some embodiments, for example, where the subject is a human, the dose includes at or about 4.5×10⁸ total recombinant receptor (e.g., CAR)-expressing cells, T cells, or peripheral blood mononuclear cells (PBMCs). In some embodiments, for example, where the subject is a human, the dose includes at or about 6×10⁸ total recombinant receptor (e.g., CAR)-expressing cells, T cells, or peripheral blood mononuclear cells (PBMCs). In some embodiments, for example, where the subject is a human, the dose includes at or about 8×10⁸ total recombinant receptor (e.g., CAR)-expressing cells, T cells, or peripheral blood mononuclear cells (PBMCs). In some embodiments, for example, where the subject is a human, the dose includes at or about 1.2×10⁹ total recombinant receptor (e.g., CAR)-expressing cells, T cells, or peripheral blood mononuclear cells (PBMCs).

In some embodiments, the dose of genetically engineered cells comprises from at or about 1×10⁵ to at or about 2×10⁹ total CAR-expressing (CAR+) T cells, from at or about 1×10⁵ to at or about 5×10⁸ total CAR-expressing T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells. In some embodiments, the dose of genetically engineered cells comprises from at or about 1.0×10⁷ to at or about 8×10⁸ total CAR-expressing (CAR+) T cells, from at or about 1.0×10⁷ to at or about 6.5×10⁸ total CAR+ T cells, from at or about 1.5×10⁷ to at or about 6.5×10⁸ total CAR+ T cells, from at or about 1.5×10⁷ to at or about 6.0×10⁸ total CAR+ T cells, from at or about 2.5×10⁷ to at or about 6.0×10⁸ total CAR+ T cells, or from at or about 5.0×10⁷ to at or about 6.0×10⁸ total CAR+ T cells.

In some embodiments, the dose of genetically engineered cells comprises between at or about 2.5×10⁷ CAR-expressing (CAR+) T cells, total T cells, or total peripheral blood mononuclear cells (PBMCs) and at or about 1.2×10⁹ CAR-expressing T cells, total T cells, or total PBMCs, between at or about 5.0×10⁷ CAR-expressing T cells, total T cells, or total peripheral blood mononuclear cells (PBMCs) and at or about 6.0×10⁸ CAR-expressing T cells, total T cells, or total PBMCs, between at or about 5.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 2.5×10⁷ to or to about 1.2×10⁹ CD3+ or CD8+ total T cells or CD3+ or CD8+ CAR-expressing cells, from or from about 5.0×10⁷ to or to about 6.0×10⁸ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+CAR-expressing cells, each inclusive.

In some embodiments, the dose is at or about 1.0×10⁷ CD3+ CAR-expressing cells. In some embodiments, the dose is at or about 1.5×10⁷ CD3+ CAR-expressing cells. In some embodiments, the dose is at or about 2.0×10⁷ CD3+ CAR-expressing cells. In some embodiments, the dose is at or about 2.5×10⁷ CD3+ CAR-expressing cells. In some embodiments, the dose is at or about 5×10⁷ CD3+ CAR-expressing cells. In some embodiments, the dose is at or about 1.5×10⁸ CD3+ CAR-expressing cells. In some embodiments, the dose is at or about 3×10⁸ CD3+ CAR-expressing cells. In some embodiments, the dose is at or about 4.5×10⁸ CD3+ CAR-expressing cells. In some embodiments, the dose is at or about 6×10⁸ CD3+ CAR-expressing cells. In some embodiments, the dose is at or about 8×10⁸ CD3+ CAR-expressing cells. In some embodiments, the dose is at or about 1.2×10⁹ CD3+ CAR-expressing cells.

In some embodiments, the dose of genetically engineered cells is with reference to the total number of CD3+ CAR-expressing (CAR+) or CD4+/CD8+ CAR-expressing (CAR+) cells. In some embodiments, the dose comprises a number of genetically engineered cells from or from about 1.0×10⁷ to or to about 1.2×10⁹ CD3+ or CD4+/CD8+ total T cells or CD3+ CAR-expressing or CD4+/CD8+ CAR-expressing cells, from or from about 1.5×10⁷ to or to about 1.2×10⁹ CD3+ or CD4+/CD8+ total T cells or CD3+ CAR-expressing or CD4+/CD8+ CAR-expressing cells, from or from about 2.0×10⁷ to or to about 1.2×10⁹ CD3+ or CD4+/CD8+ total T cells or CD3+ CAR-expressing or CD4+/CD8+ CAR-expressing cells, from or from about 2.5×10⁷ to or to about 1.2×10⁹ CD3+ or CD4+/CD8+ total T cells or CD3+ CAR-expressing or CD4+/CD8+ CAR-expressing cells, from or from about 5.0×10⁷ to or to about 6.0×10⁸ CD3+ or CD4+/CD8+ total T cells or CD3+ CAR-expressing or CD4+/CD8+ CAR-expressing cells, from or from about 5.0×10⁷ to or to about 4.5×10⁸ CD3+ or CD4+/CD8+ total T cells or CD3+CAR-expressing or CD4+/CD8+ CAR-expressing cells, or from or from about 1.5×10⁸ to or to about 3.0×10⁸ CD3+ or CD4+/CD8+ total T cells or CD3+ CAR-expressing or CD4+/CD8+CAR-expressing cells, each inclusive. In some embodiments, the dose comprises at or about 1.0×10⁷, 1.5×10⁷, 2.0×10⁷, 2.5×10⁷, 5×10⁷, 1.5×10⁸, 3×10⁸, 4.5×10⁸, 6×10⁸, 8×10⁸ or 1.2×10⁹ CD3+ or CD4+/CD8+ total T cells or CD3+ CAR-expressing or CD4+/CD8+CAR-expressing cells. In some embodiments, the dose comprises at or about 2.5×10⁷, 5×10⁷, 1.5×10⁸, 3×10⁸, 4.5×10⁸, 6×10⁸, 8×10⁸ or 1.2×10⁹CD3+ CAR-expressing cells. In some embodiments, the dose comprises at or about 1.0×10⁷, 1.5×10⁷, 2.0×10⁷, 2.5×10⁷, 5×10⁷, 1.5×10⁸, 3×10⁸, 4.5×10⁸, 6×10⁸, 8×10⁸ or 1.2×10⁹ CD4+/CD8+ CAR-expressing cells.

In some embodiments, the dose is at or about 1.0×10⁷ CD4+/CD8+ CAR-expressing cells. In some embodiments, the dose is at or about 1.5×10⁷ CD4+/CD8+ CAR-expressing cells. In some embodiments, the dose is at or about 2.0×10⁷ CD4+/CD8+ CAR-expressing cells. In some embodiments, the dose is at or about 2.5×10⁷ CD4+/CD8+ CAR-expressing cells. In some embodiments, the dose is at or about 5×10⁷ CD4+/CD8+ CAR-expressing cells. In some embodiments, the dose is at or about 1.5×10⁸ CD4+/CD8+ CAR-expressing cells. In some embodiments, the dose is at or about 3×10⁸ CD4+/CD8+ CAR-expressing cells. In some embodiments, the dose is at or about 4.5×10⁸ CD4+/CD8+ CAR-expressing cells. In some embodiments, the dose is at or about 6×10⁸ CD4+/CD8+ CAR-expressing cells. In some embodiments, the dose is at or about 8×10⁸ CD4+/CD8+ CAR-expressing cells. In some embodiments, the dose is at or about 1.2×10⁹ CD4+/CD8+ CAR-expressing cells. In some embodiments, the dose is at or about 2.5×10⁷ CD4+ or CD8+ CAR-expressing cells. In some embodiments, the dose is at or about 5×10⁷ CD4+ or CD8+ CAR-expressing cells. In some embodiments, the dose is at or about 1.5×10⁸ CD4+ or CD8+ CAR-expressing cells. In some embodiments, the dose is at or about 3×10⁸ CD4+ or CD8+ CAR-expressing cells. In some embodiments, the dose is at or about 4.5×10⁸ CD4+ or CD8+ CAR-expressing cells. In some embodiments, the dose is at or about 6×10⁸ CD4+ or CD8+ CAR-expressing cells. In some embodiments, the dose is at or about 6.5×10⁸ CD4+ or CD8+ CAR-expressing cells. In some embodiments, the dose is at or about 8×10⁸ CD4+ or CD8+ CAR-expressing cells. In some embodiments, the dose is at or about 1.2×10⁹ CD4+ or CD8+ CAR-expressing cells.

In some embodiments, the T cells of the dose include CD4+ T cells, CD8+ T cells or CD4+ and CD8+ T cells. In some embodiments, the T cells of the dose include CD4+ T cells. In some embodiments, the T cells of the dose include CD8+ T cells. In some embodiments, the T cells of the dose include CD4+ T cells or CD8+ T cells. In some embodiments, the T cells of the dose include CD4+ and CD8+ T cells.

In some embodiments, for example, where the subject is human, the total of CD4+ T cells and CD8+ T cells of the dose includes between at or about 1×10⁶ and at or about 2×10⁹ total CAR-expressing CD4+ cells and CAR-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 1.0×10⁷, at or about 2.5×10⁷, at or about 2.0×10⁷, at or about 2.5×10⁷, at or about 5×10⁷, at or about 1.5×10⁸, at or about 3×10⁸, at or about 4.5×10⁸, at or about 6×10⁸, at or about 6.5×10⁸, at or about 8×10⁸, or at or about 1.2×10⁹ total such cells, or the range between any two of the foregoing values. In some embodiments, for example, where the subject is human, the CD8+ T cells of the dose, including in a dose including CD4+ T cells and CD8+ T cells, includes between at or about 1×10⁶ and at or about 2×10⁹ total recombinant receptor (e.g., CAR)-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 2.5×10⁷, at or about 5×10⁷, at or about 1.5×10⁸, at or about 3×10⁸, at or about 4.5×10⁸, at or about 6×10⁸, at or about 8×10⁸, or at or about 1.2×10⁹ total such cells, or the range between any two of the foregoing values.

In some embodiments, the dose of cells, e.g., recombinant receptor-expressing T cells, is administered to the subject as a single dose or is administered only one time within a period of two weeks, one month, three months, six months, 1 year or more. In some embodiments, the patient is administered multiple doses, and each of the doses or the total dose can be within any of the foregoing values. In some embodiments, the engineered cells for administration or composition of engineered cells for administration, exhibits properties indicative of or consistent with cell health. In some embodiments, at or about or at least at or about 70, 75, 80, 85, or 90% CAR+ cells of such dose exhibit one or more properties or phenotypes indicative of cell health or biologically active CAR cell, such as absence expression of an apoptotic marker.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death, including blebbing, cell shrinkage, nuclear fragmentation, chromatin condensation, chromosomal DNA fragmentation, and global mRNA decay. In some aspects, early stages of apoptosis can be indicated by activation of certain caspases, e.g., 2, 8, 9, and 10. In some aspects, middle to late stages of apoptosis are characterized by further loss of membrane integrity, chromatin condensation and DNA fragmentation, include biochemical events such as activation of caspases 3, 6, and 7.

In particular embodiments, the phenotype is negative expression of one or more factors associated with programmed cell death, for example pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In certain embodiments, the phenotype is the absence of an indicator, e.g., an Annexin V molecule or by TUNEL staining, that will preferentially bind to cells undergoing apoptosis when incubated with or contacted to a cell composition. In some embodiments, the phenotype is or includes the expression of one or more markers that are indicative of an apoptotic state in the cell. In some embodiments, the phenotype is lack of expression and/or activation of a caspase, such as Caspase 3. In some aspects, activation of Caspase 3 is indicative of an increase or revival of apoptosis. In certain embodiments, caspase activation can be detected by known methods. In some embodiments, an antibody that binds specifically to an activated caspase (i.e., binds specifically to the cleaved polypeptide) can be used to detect caspase activation. In particular embodiments, the phenotype is or includes active Caspase 3. In some embodiments, the marker of apoptosis is a reagent that detects a feature in a cell that is associated with apoptosis. In certain embodiments, the reagent is an Annexin V molecule.

In some embodiments, the compositions containing the engineered cells for administration contain a certain number or amount of cells that exhibit phenotypes indicative of or consistent with cell health. In some of any embodiments, less than about 25%, 20%, 15%, 10%, 9%, 8%, 7%, 6%, 5%, 4%, 3%, 2% or 1% of the CAR-expressing T cells in the dose of engineered T cells express a marker of apoptosis, optionally Annexin V or active Caspase 3. In some of any embodiments, less than 5%, 4%, 3%, 2% or 1% of the CAR-expressing T cells in the dose of engineered T cells express Annexin V or active Caspase 3.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, the dose or composition of cells includes a defined or target ratio of CD4+ cells expressing a recombinant receptor to CD8+ cells expressing a recombinant receptor and/or of CD4+ cells to CD8+ cells that is approximately 1:1 or is between approximately 1:3 and approximately 3:1, such as approximately 1:1. In some embodiments, the dose or composition of cells includes a defined or target ratio of CD4+ cells expressing a recombinant receptor to CD8+ cells expressing a recombinant receptor and/or of CD4+ cells to CD8+ cells that is approximately 1:1. In some embodiments, the dose or composition of cells includes a defined or target ratio of CD4+ cells expressing a recombinant receptor to CD8+ cells expressing a recombinant receptor and/or of CD4+ cells to CD8+ cells that is between approximately 1:3 and approximately 3:1.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, for example, the dose contains between or between about 5.0×10⁶ and 2.25×10⁷, 5.0×10⁶ and 2.0×10⁷, 5.0×10⁶ and 1.5×10⁷, 5.0×10⁶ and 1.0×10⁷, 5.0×10⁶ and 7.5×10⁶, 7.5×10⁶ and 2.25×10⁷, 7.5×10⁶ and 2.0×10⁷, 7.5×10⁶ and 1.5×10⁷, 7.5×10⁶ and 1.0×10⁷, 1.0×10⁷ and 2.25×10⁷, 1.0×10⁷ and 2.0×10⁷, 1.0×10⁷ and 1.5×10⁷, 1.5×10⁷ and 2.25×10⁷, 1.5×10⁷ and 2.0×10⁷, 2.0×10⁷ and 2.25×10⁷ recombinant-receptor expressing cells. In some embodiments, the dose of cells contains a number of cells, that is about 1.5×10⁸ recombinant-receptor expressing cells, about 3.0×10⁸ recombinant-receptor expressing cells, or about 4.5×10⁸ recombinant-receptor expressing cells, such as recombinant-receptor expressing cells that are CD3+. In some embodiments, the dose of cells contains a number of cells, that is between at least or at least about 5×10⁶, 6×10⁶, 7×10⁶, 8×10⁶, 9×10⁶, 10×10⁶ and about 15×10⁶ recombinant-receptor expressing cells, such as recombinant-receptor expressing cells that are CD8+. In some embodiments, such dose, such as such target number of cells refers to the total recombinant-receptor expressing cells in the administered composition.

In some embodiments, for example, the lower dose contains less than about 5×10⁶ cells, recombinant receptor (e.g. CAR)-expressing cells, T cells, and/or PBMCs per kilogram body weight of the subject, such as less than about 4.5×10⁶, 4×10⁶, 3.5×10⁶, 3×10⁶, 2.5×10⁶, 2×10⁶, 1.5×10⁶, 1×10⁶, 5×10⁵, 2.5×10⁵, or 1×10⁵ such cells per kilogram body weight of the subject. In some embodiments, the lower dose contains less than about 1×10⁵, 2×10⁵, 5×10⁵, or 1×10⁶ of such cells per kilogram body weight of the subject, or a value within the range between any two of the foregoing values. In some embodiments, such values refer to numbers of recombinant receptor-expressing cells; in other embodiments, they refer to number of T cells or PBMCs or total cells administered.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses. In some embodiments, one or more subsequent dose of cells can be administered to the subject. In some embodiments, the subsequent dose of cells is administered greater than or greater than about 7 days, 14 days, 21 days, 28 days or 35 days after initiation of administration of the first dose of cells. The subsequent dose of cells can be more than, approximately the same as, or less than the first dose. In some embodiments, administration of the T cell therapy, such as administration of the first and/or second dose of cells, can be repeated.

Provided herein are exemplary T cell therapies for treating a subject having or suspected of having a disease comprising the population of cells described in Section I or Section V.A.

### VII. ARTICLES OF MANUFACTURE AND KITS

Provided herein are articles of manufacture containing a cell therapy, e.g. a T cell therapy. In some embodiments, the T cell therapy is an autologous cell therapy such as CAR T cells, or a composition thereof. In some embodiments, the T cell therapy is an allogeneic cell therapy such as CAR T cells, or a composition thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include a container with a composition contained therein, wherein the composition includes the cell therapy, such as CAR T cells. The article of manufacture may further include a package insert indicating that the composition can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VIII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In certain embodiments, “about X” refers to a value of 25%, ±10%, ±5%, ±2%, ±1%, ±0.1%, or ±0.01% of X.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New. Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, the term “enriched” with reference to a cell composition or population of cells refers to a composition or population of cells in which there is an increase in the number or percentage of the cell type or population as compared to the number or percentage of the cell type in a starting composition of the same volume, such as a starting composition directly obtained or isolated from a subject. The term does not require complete removal of other cells, cell type, or populations from the composition and does not require that the cells so enriched be present at or even near 100% in the enriched composition. In some embodiments, a cell composition or population of cells is enriched for cells positive for a given marker (e.g. CD28) if the sample contains at least 20%, at least 30%, at least 40%, at least 50% or more of cells in the sample that are positive for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, for instance, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### IX. EXEMPLARY EMBODIMENTS

Among the provided embodiments are:
1. A method of selecting cells for manufacture of a cell therapy, comprising:
   (1) determining the percentage of CD28+ T cells in a first biological sample obtained from a subject, wherein the first biological sample comprises T cells; and
   (2) selecting the subject for manufacturing a cell therapy from a second biological sample obtained from the subject if the percentage of CD28+ T cells in the first biological sample is above a threshold value, wherein the second biological sample comprises T cells.
2. The method of embodiment 1, further comprising genetically engineering cells of the second biological sample to express a recombinant receptor, thereby generating a cell therapy comprising the genetically engineered cells.
3. A method of manufacturing a cell therapy, comprising:
   (1) selecting a subject for manufacturing a cell therapy if the percentage of CD28+ T cells in a first biological sample obtained from the subject is above a threshold value, wherein the first biological sample comprises T cells; and
   (2) genetically engineering cells of a second biological sample obtained from the subject to express a recombinant receptor, thereby generating a cell therapy comprising the genetically engineered cells, wherein the second biological sample comprises T cells.
4. The method of embodiment 2 or embodiment 3, further comprising administering a dose of the cell therapy to a subject.
5. A method of treating a subject with a cell therapy, comprising administering a dose of a cell therapy comprising genetically engineered cells to a subject, wherein:
   (1) the percentage of CD28+ T cells in a first biological sample obtained from a subject is determined to be above a threshold value, wherein the first biological sample comprises T cells;
   (2) the subject is selected for manufacture of the cell therapy based on the determining in step (1); and
   (3) cells of a second biological sample obtained from the subject are genetically engineered to express a recombinant receptor, thereby generating the cell therapy, wherein the second biological sample comprises T cells.
6. The method of embodiment 5, further comprising selecting the subject for manufacturing the cell therapy from the second biological sample obtained from the subject if the percentage of CD28+ T cells in the first biological sample is above a threshold value.
7. The method of any of embodiments 3-6, further comprising determining the percentage of CD28+ T cells in the first biological sample.
8. The method of any of embodiments 1-7, wherein the first biological sample and the second biological sample are the same sample.
9. The method of any of embodiments 1-8, wherein the first biological sample and the second biological sample are the same sample, which is an apheresis sample or a leukapheresis sample.
10. A method of selecting cells for manufacture of a cell therapy, comprising:
   (1) determining the percentage of CD28+ T cells in a first biological sample obtained from a subject; and
   (2) selecting the subject for manufacturing a cell therapy from a second biological sample obtained from the subject if the percentage of CD28+ T cells in the first biological sample is above a threshold value,
   wherein the first biological sample and the second biological sample are the same sample, which is an apheresis sample.
11. The method of embodiment 10, further comprising genetically engineering cells of the second biological sample to express a recombinant receptor, thereby generating a cell therapy comprising the genetically engineered cells.
12. A method of manufacturing a cell therapy, comprising:
   (1) selecting a subject for manufacturing a cell therapy if the percentage of CD28+ T cells in a first biological sample obtained from the subject is above a threshold value; and
   (2) genetically engineering cells of a second biological sample obtained from the subject to express a recombinant receptor, thereby generating a cell therapy comprising the genetically engineered cells,
   wherein the first biological sample and the second biological sample are the same sample, which is an apheresis sample.
13. The method of embodiment 11 or embodiment 12, further comprising administering a dose of the cell therapy to a subject.
14. A method of treating a subject with a cell therapy, comprising administering a dose of a cell therapy comprising genetically engineered cells to a subject, wherein:
   (1) the percentage of CD28+ T cells in a first biological sample obtained from a subject is determined to be above a threshold value;
   (2) the subject is selected for manufacture of the cell therapy based on the determining in step (1); and
   (3) cells of a second biological sample obtained from the subject are engineered to express a recombinant receptor, thereby generating the cell therapy, wherein the first biological sample and the second biological sample are the same sample, which is an apheresis sample.
15. The method of embodiment 14, further comprising selecting the subject for manufacturing the cell therapy from the second biological sample obtained from the subject if the percentage of CD28+ T cells in the first biological sample is above a threshold value.
16. The method of any of embodiments 12-15, further comprising determining the percentage of CD28+ T cells in the first biological sample.
17. The method of any of embodiments 1-16, wherein the threshold value is between about 30% and about 50% CD28+ T cells, or between about 35% and about 45% CD28+ T cells.
18. The method of any of embodiments 1-17, wherein the threshold value is about 40% CD28+ T cells.
19. The method of any of embodiments 1-7, 17, and 18, wherein the first biological sample and the second biological sample are different samples.
20. The method of embodiment 19, wherein the first biological sample is a whole blood sample, an apheresis sample, or a leukapheresis sample.
21. The method of embodiment 19 or embodiment 20, wherein the second biological sample is a whole blood sample, an apheresis sample, or a leukapheresis sample.
22. The method of any of embodiments 1-7 and 17-21, wherein the first biological sample is a whole blood sample or an apheresis sample, and the second biological sample is an apheresis sample or a leukapheresis sample.
23. The method of any of embodiments 1-7 and 17-22, wherein the first biological sample is a whole blood sample and the second biological sample is a leukapheresis sample.
24. The method of any of embodiments 4-9 and 11-18, wherein the first biological sample and the second biological sample are the same sample, which is obtained from the subject between about six weeks and about one week prior to administration of the cell therapy to a subject.
25. The method of any of embodiments 4-9, 11-19, and 24, wherein the first biological sample and the second biological sample are the same sample, which is obtained from the subject about three weeks prior to administration of the cell therapy to a subject.
26. The method of any of embodiments 4-7 and 17-23, wherein:
   (a) the first biological sample and the second biological sample are different samples;
   (b) the first biological sample is obtained from the subject between about eight weeks prior and about four weeks prior to administration of the cell therapy to a subject; and
   (c) the second biological sample is obtained from the subject between about four weeks and about two weeks prior to administration of the cell therapy to a subject.
27. The method of any of embodiments 4-7, 17-23, and 26, wherein the first biological sample and the second biological sample are obtained from the subject between about two weeks apart and about six weeks apart.
28. The method of any of embodiments 4-7, 17-23, 26, and 27, wherein the first biological sample and the second biological sample are obtained from the subject about three weeks apart.
29. The method of any of embodiments 26-28, wherein the second biological sample is obtained from the subject about three weeks prior to administration of the cell therapy to a subject.
30. The method of any of embodiments 2-9 and 11-29, wherein, prior to genetic engineering, the cells of the second biological sample are incubated under stimulating conditions.
31. A method of enriching for CD28+ cells, the method comprising:
   (a) performing a first selection, the first selection comprising enriching for either of CD28+ or CD3+ cells from a biological sample comprising peripheral blood mononuclear cells (PBMCs) obtained from a subject, thereby generating an enriched cell population; and
   (b) performing a second selection on the cells of the enriched cell population, thereby generating a CD28+ enriched population, wherein (i) the first selection comprises enriching for CD28+ cells and the second selection comprises enriching for CD3+ cells from the enriched population; or (ii) the first selection comprises enriching for CD3+ cells and the second selection comprises enriching for CD28+ cells from the enriched cell population, wherein the CD28+ enriched population has a higher percentage of CD28+ cells than the biological sample and is enriched for CD3+ cells.
32. A method of enriching for CD28+ cells, the method comprising:
   (a) performing a first selection, the first selection comprising enriching for CD28+ cells from a biological sample comprising peripheral blood mononuclear cells (PBMCs) obtained from a subject, thereby generating a first enriched population, the first enriched population having a higher percentage of CD28+ cells than the biological sample;
   (b) performing a second selection on the cells from the first enriched population, the second selection comprising enriching for one of (i) CD4+ cells and (ii) CD8+ cells from the first enriched population, the enrichment thereby generating a second enriched population enriched for the one of (i) CD4+ cells and (ii) CD8+ cells and a non-selected population; and
   (c) performing a third selection, the third selection comprising enriching for the other of (i) CD4+ cells and (ii) CD8+ cells from the non-selected population, the enrichment thereby generating a third enriched population enriched for the other of the (i) CD4+ cells and (ii) CD8+ cells.
33. The method of embodiment 32, further comprising combining the second enriched population and the third enriched population, optionally at a ratio of between about 1:3 and about 3:1, optionally about 1:1, thereby generating a CD28+ enriched population comprising the second enriched population and the third enriched population.
34. The method of embodiment 32 or embodiment 33, wherein the second selection comprises enriching for CD8+ cells.
35. The method of any of embodiments 31-34 wherein the biological sample is a whole blood sample, an apheresis sample, or a leukapheresis sample.
36. The method of any of embodiments 31 and 33-35, wherein the CD28+ enriched population comprises:
   (i) less than about 5% CD28− T cells;
   (ii) CD4+ T cells, wherein at least about 95% of the CD4+ T cells are CD28+; or
   (iii) CD8+ T cells, wherein at least about 95% of the CD8+ T cells are CD28+.
37. The method of any of embodiments 31 and 33-36, wherein at least about 95% of the CD4+ T cells of the CD28+ enriched population comprises CD28+CD4+ T cells.
38. The method of any of embodiments 31 and 33-37, wherein at least about 95% of the CD8+ T cells of the CD28+ enriched population comprises CD28+CD8+ T cells.
39. The method of any of embodiments 31 and 33-38, wherein at least about 95% of the CD4+ T cells and at least about 95% of the CD8+ T cells of the CD28+ enriched population comprises CD28+CD4+ T cells and CD28+CD8+ T cells, respectively.
40. The method of any of embodiments 31 and 33-39, wherein at least about 95% of the CD3+ T cells of the CD28+ enriched population comprises CD28+CD3+ T cells.
41. The method of any of embodiments 31 and 33-40, wherein the percentage of the CD28− cells in the CD28+ enriched population is less than about or about 35%, 30%, 20%, 10%, 5%, 1% or 0.1% of the percentage of CD28− cells in the biological sample.
42. The method of any of embodiments 31 and 33-41, wherein CD28+ enriched population comprises less than about 3%, less than about 2%, less than about 1%, less than about 0.1% or less than about 0.01% CD28− cells.
43. The method of any of embodiments 31 and 33-42, wherein the CD28+ enriched population is free or is essentially free of CD28− cells.
44. The method of any of embodiments 31 and 33-43, wherein the percentage of naïve-like T cells in the CD28+ enriched population is at least about 10%, 20%, 30%, 40% or 50% greater than the percentage of naïve-like T cells in the biological sample.
45. The method of embodiment 44, wherein the naïve-like T cells are surface positive for one or more of markers selected from CD45RA, CD27, and CCR7.
46. The method of any of embodiments 31-45, wherein the enriching for CD28+ cells comprises immunoaffinity-based selection.
47. The method of embodiment 46, wherein the immunoaffinity-based selection comprises contacting cells with an antibody capable of specifically binding to CD28 and recovering cells bound to the antibody.
48. The method of embodiment 47, wherein the antibody is immobilized on a solid surface, optionally wherein the solid surface is a magnetic particle.
49. The method of embodiments 47 and embodiment 48, wherein the antibody is immobilized on or attached to an affinity chromatography matrix.
50. The method of any of embodiments 31 and 33-49, wherein the cells of the CD28+ enriched population are genetically engineered to express a recombinant receptor.
51. The method of embodiment 50, wherein, prior to genetic engineering, the cells of the CD28+ enriched population are incubated under stimulating conditions.
52. The method of any of embodiments 30 and 51, wherein the stimulating conditions comprise the presence of a stimulatory reagent capable of activating an intracellular signaling domains of a component of a T cell receptor (TCR) complex and an intracellular signaling domain of a costimulatory molecule.
53. The method of embodiment 52, wherein the stimulatory reagent comprises (i) a primary agent that binds to a member of a TCR complex, optionally that binds to CD3; and (ii) a secondary agent that binds to a T cell costimulatory molecule, optionally wherein the costimulatory molecule is selected from CD28, CD137 (4-1BB), OX40 and ICOS.
54. The method of embodiment 53, wherein the primary agent and/or the secondary agent comprises an antibody or an antigen-binding fragment thereof.
55. The method of embodiment 53 or embodiment 54, wherein the primary agent is an anti-CD3 antibody or an antigen-binding fragment thereof, and the secondary agent is an anti-CD28 antibody or an antigen-binding fragment thereof.
56. The method of any of embodiments 30 and 51-55, wherein the stimulating conditions comprise the presence of a recombinant cytokine.
57. The method of embodiment 56, wherein the recombinant cytokine comprises IL-2, IL-7, IL-15, or a combination thereof.
58. The method of any of embodiments 2-9, 11-30 and 50-57, wherein, following genetic engineering, the genetically engineered cells are cultivated under conditions to allow for expansion or proliferation of the engineered cells.
59. The method of embodiment 58, wherein the cultivation results in at least about a 2-fold, 3-fold, 4-fold, 5-fold increase in the number of viable genetically engineered cells, compared to at the initiation of cultivation.
60. The method of any one of embodiments 4-9, 13-30, and 52-59, wherein the cell therapy is an allogeneic cell therapy, and the subject from whom the first and second biological samples are obtained is different than the subject to whom the cell therapy is administered.
61. The method of any one of embodiments 4-9, 13-30, and 52-59, wherein the cell therapy is an autologous cell therapy, and the subject from whom the first and second biological samples are obtained is the same subject to whom the cell therapy is administered.
62. The method of any of embodiments 4-9, 13-30, and 52-61, wherein the subject administered the cell therapy has a disease or condition.
63. The method of embodiment 62, wherein the disease or condition is an infectious disease or disorder, an autoimmune disease, an inflammatory disease, or a cancer.
64. The method of embodiment 62 or embodiment 63, wherein the disease or condition is a cancer.
65. The method of embodiment 64, wherein the cancer is a leukemia, a lymphoma, or a myeloma.
66. The method of embodiment 64 or embodiment 65, wherein the cancer is a multiple myeloma (MM), optionally a relapsed/refractory MM.
67. The method of any of embodiments 62-66, wherein the recombinant receptor binds to an antigen expressed by cells of the disease or condition.
68. The method of any of embodiments 2-9, 11-30, and 50-67, wherein the recombinant receptor is a T cell receptor (TCR) is a chimeric antigen receptor (CAR).
69. The method of any of embodiments 2-9, 11-30, and 50-68, wherein the recombinant receptor is a CAR.
70. The method of embodiment 68 or embodiment 69, wherein the CAR comprises an extracellular antigen binding domain that binds to the antigen, a transmembrane domain, and an intracellular signaling region.
71. The method of embodiment 70, wherein the intracellular signaling region comprises an intracellular signaling domain of a CD3-zeta (CD3ζ) chain and a costimulatory signaling region.
72. The method of embodiment 71, wherein the costimulatory signaling region comprises an intracellular signaling domain of CD28, 4-1BB, or ICOS.
73. The method of embodiment 71 or embodiment 72, wherein the costimulatory signaling region comprises an intracellular signaling domain of 4-1BB.
74. The method of any one of embodiments 70-73, wherein the transmembrane domain is or comprises a transmembrane domain from CD28 or CD8, optionally human CD28 or CD8.
75. The method of any one of embodiments 70-74, wherein the CAR further comprises an extracellular spacer between the extracellular antigen binding domain and the transmembrane domain.
76. The method of embodiment 75, wherein the spacer is from CD8, optionally wherein the spacer is a CD8-alpha hinge.
77. The method of embodiment 75 or embodiment 76, wherein the transmembrane domain and the spacer are from CD8.
78. The method of any of embodiments 70-77, wherein the extracellular antigen binding domain binds to B cell maturation antigen (BCMA).
79. The method of any of embodiments 70-78, wherein the extracellular antigen-binding domain comprises a variable heavy chain (VH) region and, optionally, a variable light chain (VL) region.
80. The method of embodiment 79, wherein:
   the VH region comprises a CDR-H1, a CDR-H2, and a CDR-H3 comprising the amino acid sequences set forth in SEQ ID NOS: 189, 190, and 191, respectively; and the VL region comprises a CDR-L1, a CDR-L2, and a CDR-L3 comprising the amino acid sequences set forth in SEQ ID NOS: 192, 193, and 194, respectively; or
   the VH region comprises a CDR-H1, a CDR-H2, and a CDR-H3 comprising the amino acid sequences set forth in SEQ ID NOS: 173, 174 and 175, respectively; and the VL region comprises a CDR-L1, a CDR-L2, and a CDR-L3 comprising the amino acid sequences set forth in SEQ ID NOS: 183, 184 and 185, respectively.
81. The method of embodiment 79 or embodiment 80, wherein:
   the VH region comprises an amino acid sequence set forth in SEQ ID NO: 18 and the VL region comprises the amino acid sequence set forth in SEQ ID NO: 19;
   or the VH region comprises an amino acid sequence set forth in SEQ ID NO: 24, and the VL region comprises the amino acid sequence set forth in SEQ ID NO: 25.
82. The method of any one of embodiments 70-81, wherein the extracellular antigen-binding domain is a single chain variable fragment (scFv).
83. The method of embodiment 82, wherein the scFv comprises the amino acid sequence set forth in SEQ ID NO: 213 or SEQ ID NO: 188.
84. The method of any one of embodiments 68-83, wherein the CAR comprises the amino acid sequence set forth in SEQ ID NO: 116 or SEQ ID NO: 124.
85. The method of any one of embodiments 68-84, wherein the CAR is encoded by the polynucleotide sequence set forth in SEQ ID NO: 214.
86. The method of any one of embodiments 4-9, 13-30, and 52-85, wherein the dose of the cell therapy comprises T cells expressing a chimeric antigen receptor (CAR) as present in: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BClcCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells.
87. The method of any one of embodiments 4-9, 13-30, and 52-86, wherein the dose of the cell therapy comprises T cells expressing a chimeric antigen receptor (CAR) as present in idecabtagene vicleucel cells.
88. The method of any of embodiments 70-77, wherein the extracellular antigen binding domain binds to CD19.
89. The method of any one of embodiments 1-30 and 52-88, wherein the cell therapy comprises CD4+ T cells and/or CD8+ T cells.
90. The method of embodiment 89, wherein the dose of the cell therapy comprises a defined ratio of CD4+ T cells to CD8+ T cells, which is between about 1:3 and about 3:1 or is about 1:1.
91. The method of any one of embodiments 4-9, 13-30, and 52-90, wherein the dose of the cell therapy comprises between about 0.5×10⁶ and about 6×10⁸ CAR-positive T cells.
92. The method of any one of embodiments 4-9, 13-30, and 52-91, wherein the dose of the cell therapy comprises between about 1×10⁸ and about 6×10⁸ CAR-positive T cells.
93. The method of any one of embodiments 4-9, 13-30, and 52-92, wherein the dose of the cell therapy comprises between about 1.5×10⁸ and about 4.5×10⁹ CAR-positive T cells.
94. The method of any one of embodiments 4-9, 13-30, and 52-93, wherein the dose of the cell therapy comprises about 1.5×10⁸, 3×10⁸, or about 4.5×10⁹ CAR-positive T cells.
95. The method of any one of embodiments 4-9, 13-30, and 52-91, wherein the dose of the cell therapy comprises between about 0.5×10⁶ and about 10×10⁶ CAR-positive T cells.
96. The method of any one of embodiments 1-30 and 52-95, wherein the percentage of CD28+ T cells is the percentage of total T cells that are CD28+.
97. A composition of cells produced by the method of any of embodiments 1-4, 7-13, and 16-96.
98. A method of treating a subject having or suspected of having a disease or condition, comprising administering to the subject a dose of the cell therapy produced by the method of any of embodiments 2-4, 7-13, 16-30, and 52-96.
99. Use of a dose of the cell therapy produced by the method of any of embodiments 2-4, 7-13, 16-30, and 53-96 or the composition of embodiment 97 for the treatment of a disease or condition in a subject.
100. A dose of the cell therapy produced by the method of any of embodiments 2-4, 7-13, 16-30, and 53-96 or the composition of embodiment 97 for use in treating a disease or condition in a subject.
101. A dose of the cell therapy produced by the method of any of embodiments 2-4, 7-13, 16-30, and 53-96 or the composition of embodiment 97 for use in the manufacture of a medicament for treating a disease or condition in a subject.
102. A method of treating a disease or condition in a human subject with a T cell therapy, the method comprising administering to a human subject having a disease or condition a therapeutically effective amount of a T cell therapy, wherein:
   (a) at least about 40% of T cells in the subject are CD28+; and
   (b) manufacture of the T cell therapy relies on CD28-mediated expansion of the T cells of the T cell therapy.
103. The method of embodiment 102, wherein the disease or condition is a multiple myeloma.
104. A method of treating multiple myeloma in a human subject, the method comprising administering to a human subject having a multiple myeloma a therapeutically effective amount of a T cell therapy, wherein at least about 40% of T cells in the subject are CD28+.
105. The method of embodiment 104, wherein manufacture of the T cell therapy relies on CD28-mediated expansion of the T cells of the T cell therapy.
106. The method of any one of embodiments 102-105, wherein, prior to administration of the T cell therapy to the subject, it has been determined that at least about 40% of T cells, e.g., peripheral T cells, in the subject are CD28+.
107. The method of any one of embodiments 102-106, wherein the T cell therapy targets B cell maturation antigen (BCMA).
108. The method of any one of embodiments 102-107, wherein the T cell therapy is an autologous T cell therapy.
109. The method of any one of embodiments 102-108, wherein the T cell therapy is a chimeric antigen receptor (CAR) T cell therapy.
110. The method of embodiment 109, wherein the CAR T cell therapy targets BCMA.
111. The method of any one of embodiments 102-110, wherein at least about 44%, at least about 45%, or at least about 50% of T cells, e.g., peripheral T cells, in the subject are CD28+.
112. The method of any one of embodiments 103-111, wherein the multiple myeloma is a relapsed/refractory multiple myeloma.
113. A method of increasing proliferation of T cells comprising incubating a population of T cells under stimulating conditions, wherein the population of T cells comprises a percentage of CD28+ T cells above a threshold value.
114. A method of increasing proliferation of T cells comprising:
   (1) selecting a biological sample comprising a population of T cells in which the percentage of CD28+ T cells in the population of T cells is above a threshold value; and
   (2) incubating the selected population of T cells under stimulating conditions.
115. The method of embodiment 113, wherein the population of T cells is obtained from a biological sample.
116. The method of embodiment 114 or embodiment 115, wherein the biological sample is obtained from a subject.
117. The method of embodiment 116, wherein the subject is human.
118. The method of any of embodiments 114-117, wherein the biological sample is a whole blood sample, an apheresis sample, or a leukapheresis sample.
119. The method of any of embodiments 113-118, wherein the stimulating conditions comprise the presence of a stimulatory reagent capable of activating an intracellular signaling domains of a component of a T cell receptor (TCR) complex and an intracellular signaling domain of a costimulatory molecule.
120. The method of embodiment 114, wherein the stimulatory reagent comprises (i) a primary agent that binds to a member of a TCR complex, optionally that binds to CD3; and (ii) a secondary agent that binds to a T cell costimulatory molecule, optionally wherein the costimulatory molecule is selected from CD28, CD137 (4-1BB), OX40 and ICOS.
121. The method of embodiment 115, wherein the primary agent and/or the secondary agent comprises an antibody or an antigen-binding fragment thereof.
122. The method of embodiment 115 or embodiment 116, wherein the primary agent is an anti-CD3 antibody or an antigen-binding fragment thereof, and the secondary agent is an anti-CD28 antibody or an antigen-binding fragment thereof.
123. The method of any of embodiments 113-117, wherein the stimulating conditions comprise the presence of a recombinant cytokine.
124. The method of embodiment 118, wherein the recombinant cytokine comprises IL-2, IL-7, IL-15, or a combination thereof.
125. The method of any of embodiments 113-124, wherein the method results in increased proliferation compared to a population of T cells comprising a percentage of CD28+ T cells that is not at or above the threshold value.
126. The method of any of embodiments 113-125, wherein the method results in increased proliferation compared to a population of CD28+ T cells comprising a percentage of CD28+ T cells that is below the threshold value.
127. The method of any of embodiments 113-126, wherein the threshold value is between about 30% and about 50% CD28+ T cells, or between about 35% and about 45% CD28+ T cells.
128. The method of any of embodiments 113-127, wherein the threshold value is about 40% CD28+ T cells.
129. The method of any of embodiments 113-128, wherein the increase in proliferation occurs on day 1, 2, 3, 4, or 5 after incubation.
130. The method of any of embodiments 1-129, wherein determining the percentage of CD28+ T cells comprises measuring CD28 protein.
131. The method of any of embodiments 1-129, wherein determining the percentage of CD28+ T cells comprises measuring CD28 RNA.
132. A method of manufacturing a cell therapy, comprising:
   (1) selecting a subject for manufacturing a cell therapy if the percentage of any of CD28+, CD45RA+, CD45RO+, CD27+, CD197+, CD4+, CD57+, CD8+, CD25+, PD1+, LAG3+, CD3+ and/or TIM3+ T cells in a first biological sample obtained from the subject is above a threshold value, wherein the first biological sample comprises T cells; and
   (2) genetically engineering cells of a second biological sample obtained from the subject to express a recombinant receptor, thereby generating a cell therapy comprising the genetically engineered cells, wherein the second biological sample comprises T cells.
133. The method of embodiment 132, further comprising determining the percentage of any of CD28+, CD45RA+, CD45RO+, CD27+, CD197+, CD4+, CD57+, CD8+, CD25+, PD1+, LAG3+, CD3+ and/or TIM3+ T cells in the first biological sample.
134. The method of embodiment 132 or embodiment 133, wherein the threshold value is calculated as the percentage of T cells that are CD28+, CD45RA+, CD45RO+, CD27+, CD197+, CD4+, CD57+, CD8+, CD25+, PD1+, LAG3+, CD3+ and/or TIM3+ in the first biological sample.
135. The method of any of embodiments 132-134, wherein the threshold value is between about 20% and about 40% CD28+ T cells, between about 30% and about 50% CD28+ T cells, or between about 35% and about 45% CD28+ T cells.
136. The method of any of embodiments 132-135, wherein the threshold value is about 40% CD28+ T cells.
137. The method of any of embodiments 132-134, wherein the threshold value is between about 20% and about 40% CD45RA+ T cells, between about 30% and about 50% CD45RA+ T cells, or between about 35% and about 45% CD45RA+ T cells.
138. The method of any of embodiments 132-134 or 137, wherein the threshold value is about 40% CD45RA+ T cells.
139. The method of any of embodiments 132-134, wherein the threshold value is between about 20% and about 40% CD45RO+ T cells, between about 30% and about 50% CD45RO+ T cells, or between about 35% and about 45% CD45RO+ T cells.
140. The method of any of embodiments 132-134 or 139, wherein the threshold value is about 40% CD45RO+ T cells.
141. The method of any of embodiments 132-134, wherein the threshold value is between about 20% and about 40% CD27+ T cells, between about 30% and about 50% CD27+ T cells, or between about 35% and about 45% CD27+ T cells.
142. The method of any of embodiments 132-134 or 141, wherein the threshold value is about 40% CD27+ T cells.
143. The method of any of embodiments 132-134, wherein the threshold value is between about 20% and about 40% CD197+ T cells, between about 30% and about 50% CD197+ T cells, or between about 35% and about 45% CD197+ T cells.
144. The method of any of embodiments 132-134 or 143, wherein the threshold value is about 40% CD197+ T cells.
145. The method of any of embodiments 132-144, further comprising administering a dose of the cell therapy to the subject.
146. A composition of cells produced by the method of any of embodiments 132-145.
147. A method of manufacturing a cell therapy, comprising:
   (1) selecting a subject for manufacturing a cell therapy if T cell expression of interleukin 2 receptor subunit alpha (IL2RA), interleukin 6 family cytokine (LIF), and/or oncostatin M (OSM) in a first biological sample obtained from the subject is above a threshold value, wherein the first biological sample comprises T cells; and
   (2) genetically engineering cells of a second biological sample obtained from the subject to express a recombinant receptor, thereby generating a cell therapy comprising the genetically engineered cells, wherein the second biological sample comprises T cells.
148. The method of embodiment 147, further comprising determining T cell expression of IL2RA, LIF, and/or OSM in the first biological sample.
149. The method of embodiment 148, wherein determining expression of IL2RA, LIF, and/or OSM comprises measuring RNA expression of IL2RA, LIF, and/or OSM.
150. The method of embodiment 149, wherein RNA expression is measured in a single cell of the biological sample.
151. The method of embodiment 149, wherein RNA expression is measured in a plurality of cells of the biological sample.
152. The method of any of embodiments 147-151, wherein the threshold value is calculated as the average expression level of IL2RA, LIF, and/or OSM in a reference T cell or T cell population.
153. The method of embodiment 152, wherein the reference T cell or T cell population is obtained from a subject that is not selected in a method of manufacturing the cell therapy.
154. The method of embodiment 153, wherein the subject that is not selected in a method of manufacturing the cell therapy has slow growing T cells.
155. The method of any of embodiments 147-154, wherein the threshold value is between about 20% and about 40% IL2RA, LIF, and/or OSM expression, between about 30% and about 50% IL2RA, LIF, and/or OSM expression, or between about 35% and about 45% IL2RA, LIF, and/or OSM expression.
156. The method of any of embodiments 147-155, wherein the threshold value is about 40% IL2RA, LIF, and/or OSM expression.
157. The method of any of embodiments 147-156, further comprising administering a dose of the cell therapy to the subject.
158. The method of any of embodiments 147-157, wherein the first biological sample and the second biological sample is an apheresis sample or a leukapheresis sample.
159. A method of manufacturing a cell therapy, comprising:
   (1) selecting a subject for manufacturing a cell therapy if one or more genes associated with CD4 proliferating T cells, CD4 central memory T cells (TCM), CD8 naïve cells, CD4 naïve cells, CD8 TCM cells, T regulatory cells (Treg), mucosal-associated invariant T cells (MAIT), cDC2 cells, plasmablast cells, or NK proliferating cells in a first biological sample obtained from the subject is above a threshold value, wherein the first biological sample comprises T cells; and
   (2) genetically engineering cells of a second biological sample obtained from the subject to express a recombinant receptor, thereby generating a cell therapy comprising the genetically engineered cells, wherein the second biological sample comprises T cells.
160. The method of embodiment 159, comprising selecting a subject for manufacturing a cell therapy if one or more genes associated with CD4 proliferating cells in the first biological sample obtained from the subject is above the threshold value.
161. The method of embodiment 159, comprising selecting a subject for manufacturing a cell therapy if one or more genes associated with CD4 TCM in the first biological sample obtained from the subject is above the threshold value.
162. The method of any of embodiments 159-161, wherein the one or more genes associated with CD4 proliferating T cells is selected from the group consisting of MK167, TOP2A, CD4, CCR7, NKG7, IL32, LAG3, HAVCR2, and CD3G.
163. The method of any of embodiments 159-162, wherein the one or more genes associated with CD4 TCM is selected from the group consisting of CD4, CCR7, TCF7, IL7R, IL32, and CD3G.

### X. EXAMPLES

The following examples are included for illustrative purposes only and are not intended to limit the scope of the invention.

### Example 1: Assessment of CAR T Cell Population Doubling Levels

Peripheral blood mononuclear cells (PBMCs) were obtained from donors and were engineered to express a chimeric antigen receptor (CAR) by a manufacturing process involving stimulation, transduction with a lentiviral vector encoding a CAR construct and cultivation for expansion. The proliferation of cells during expansion was assessed for two donor samples identified to be “slow growing” (“SG”), in that they failed to yield a sufficient number of cells (e.g., 150-540×10⁶ CAR+ T cells) by day 10 of the manufacture process.

To generate the engineered cell compositions, PBMCs were obtained from human donor apheresis collections and cryopreserved. On day 0, cryopreserved cell compositions were thawed and cultured with anti-CD3 and anti-CD28 antibodies for about 24 hours. On day 1, PBMCs were transduced with a viral preparation containing nucleic acid encoding an anti-BCMA chimeric antigen receptor (CAR) and static culture maintenance was performed from days 3-9. The CAR contained a munne anti-BCMA scFv and a human CD8a hinge and transmembrane domain fused to the T cell cytoplasmic signaling domains of 4-1BB and CD3zeta, in tandem. Cells were harvested at day 10 or later. The cumulative population doubling levels (cPDLs) were calculated for two different SG donor samples at different time points during expansion and compared to that of the average donor samples that yield a sufficient number of cells (e.g., 150-540×10⁶ CAR+ T cells) by day 10 of the manufacturing process (“average donors”).

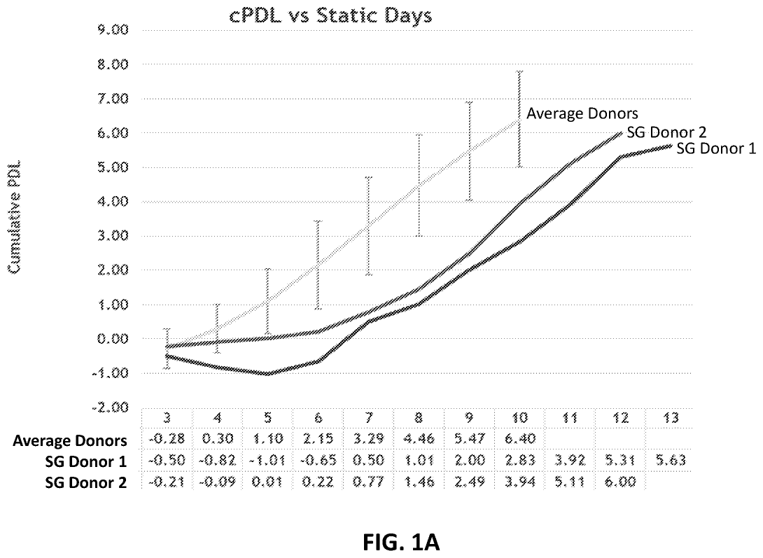

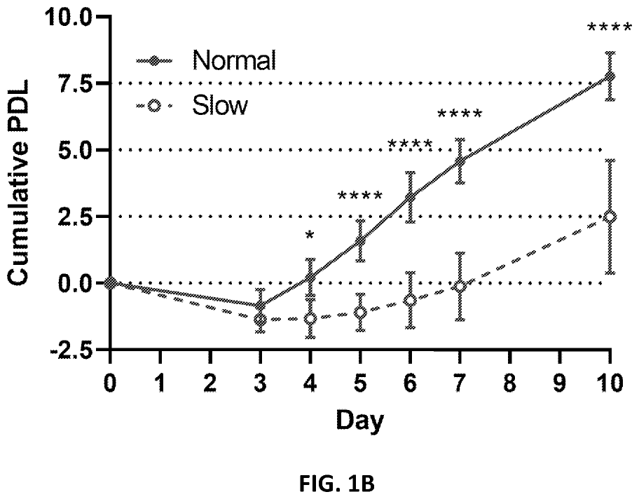

As shown in FIG. 1A, the average donor samples reached 5 PDLs by day 10 of incubation, whereas the SG donor samples did not reach a PDL of 2 until day 6 or 7 and did not yield a sufficient number of cells by day 10.

A scaled down expansion model was also performed in normal grower and slow grower cells. Briefly, six PBMC lots requiring extended culture to reach clinical dose (failing to make dose by day 10) and six with typical growth kinetics were selected from KarMMa-2/KarMMa-3 clinical trial patient material manufactured in Summit, NJ to confirm that expansion performance was intrinsic and not process-related. PBMCs were seeded at million cells/mL with soluble anti-CD3 and anti-CD28, and total cells were counted on days 3, 4, 5, 6, 7 and 10 with passaging on days 3-7 to normalize cell concentration to 3.0·10⁵/mL. As shown in FIG. 1B, slow growing lots show lower cumulative population doubling level (PDL) than normal growing lots in a 10 day scaled down expansion model.

### Example 2: Characterization of Slow Growing Donor Samples

Clinical trial patient lots for which anti-BCMA CAR-T cells were manufactured for therapy but failed to make dose were analyzed to assess in process kinetics of poorly expanding donor samples. Using the expansion method described in Example 1, clinical trial patient lots which failed to make dose by day 10 (D10) of expansion were compared with lots that performed within the normal bounds of successful manufacture. The study showed that the T cell compositions of the cultures changes over the culture period, particularly shifting from CD8+ lineage majority to CD4+ lineage majority. Phenotypic differences corresponding to the differential expansion performance of these two groups also was observed, as described in the following subsections.

### A. Percentage CD3+ Cells

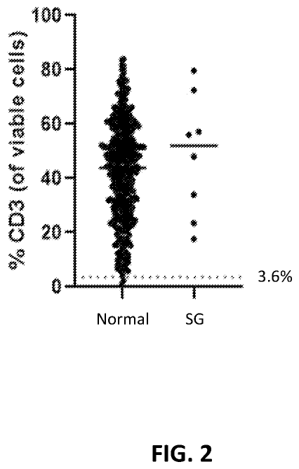

PBMCs containing a minimum of 3.6% CD3+ T cells were engineered to express an anti-BCMA CAR as described in Example 1 and harvested at day 10. The percentage of pre-culture CD3+ cells was calculated for donor samples that yielded a sufficient number of cells by day 10 (“normal growing” or “NG”) vs. slow growing donor samples that failed to yield a sufficient number of cells by day 10 (“SG”). As shown in FIG. 2, the SG samples exhibited a range of percentage of pre-culture CD3+ cells similar to the NG donor samples, indicating that the percentage of T cells entering the culture is not an indicator of manufacturing success.

### B. Percentage of CD3+CD28+ and CD3+CD57+ Cells

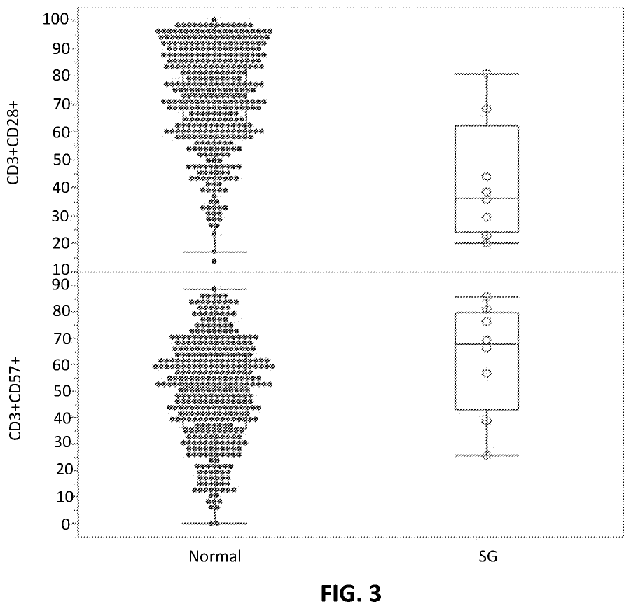

Expanded PBMCs from donors were harvested at different time points according to the manufacturing protocol described in Example 1, and the percentage of CD3+CD57+ and CD3+CD28+ cells was calculated for NG donor samples vs. SG donor samples. As shown in FIG. 3, in the NG donor samples, CD3+CD57+ cells (bottom panel) followed a normal distribution, which the SG donor samples fell within. By contrast, NG donor samples exhibited a skewed distribution of CD3+CD28+ cells (top panel), with the SG donor samples falling within the lower portion of the distribution. Without wishing to be bound by theory, the data suggest that CD28 expression may better resolve slow growing donor samples from normal growing donor samples.

### C. CD28 as a Differential Marker of Slow Growing Cells

371 donor samples were obtained from a large repository of day 0 PBMCs, manufactured as described in Example 1, and characterized using an extended T cell differentiation (eTDiff) phenotype assay and an activation and exhaustion (A&E) assay to evaluate expression markers that may be linked to the SG phenotype. The eTDiff phenotype assay was used to measure the frequencies of CD4+(helper T cells), CD8+(cytotoxic T cells), CD45RO+(memory T cell), and CD45RA+CD197+(T cell memory subsets), and CD27+ and CD28+(co stimulatory receptors, T cell memory markers) in PBMC donor samples by flow cytometry. The A&E assay was used to measure the percentage of CD25+(activation), LAG-3+, PD-1+, TIM-3+, 2B4+, CD57+CD3+, CD4+, CAR, (exhaustion, anergy and senescence), and CD3+, CD4+, CD8+, CAR, CD27+, CD45RO, CCR7, CD45RA, CD28 (T cell differentiation) cells within the viable CD3+, CD4+, and CD8+ T cell populations by flow cytometry.

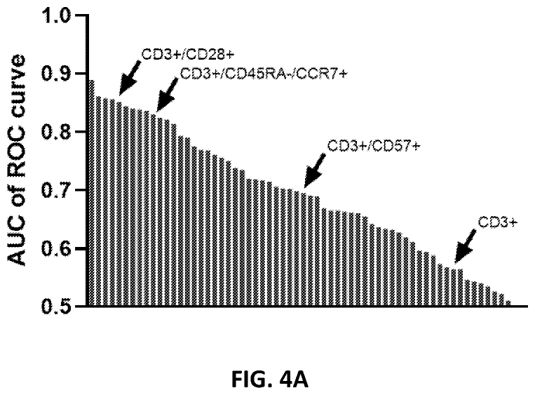

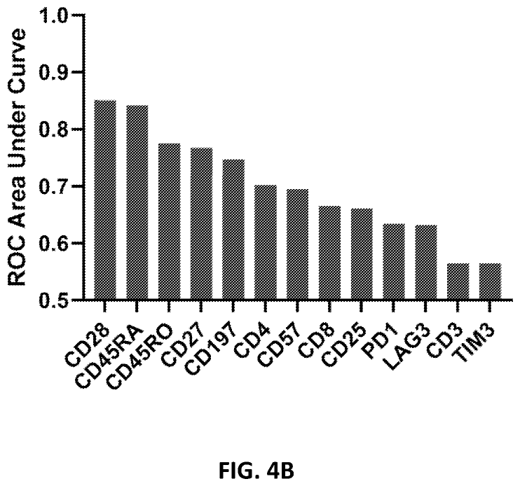

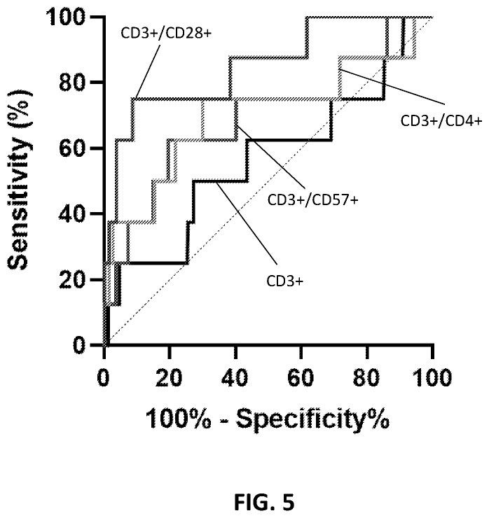

The donor samples were sorted between 8 SG lots and 363 NG lots, and Receiver-Operating Characteristic (ROC) analysis of the samples was performed to determine the power of 64 different population statistics across both characterization assays to sort between the SG and NG populations. FIG. 4 shows the distribution of AUCs across day 0 PBMC populations characterized using the eTDiff and A&E assays. As shown in FIGS. 4A, 4B and 5, the ROC curves indicated that the AUC of CD3+/CD28+ was 0.851, whereas the AUC of CD3+/CD57+ was 0.695. A threshold value for predicting which donor PBMC samples will yield a sufficient number of cells by day 10 of manufacture was determined by calculating the Youden Index, based on the sensitivity and specificity of the CD28 marker among T cells. An exemplary threshold value for predicting which donor PBMC samples will yield a sufficient number of cells by day 10 of manufacture was identified as greater than or equal to about 44% CD28+CD3+ cells among T cells from day 0 PBMC samples.

The data are consistent with a finding that CD28 exhibits predictive power for identifying donor samples that may fail to yield a sufficient number of cells during manufacture.

### D. Cytokine Secretion as a Measure of Biological Activity of Slow Growing Cells

4 slow grower and 4 normal grower lots selected from scale-down expansion model cohorts were used. Whole PBMCs were seeded into T25 flasks at 1 million live cells/mL for 48-hour culture with or without soluble anti-CD3 and anti CD28. At the end of culture, cells were collected and enriched for CD3, followed by dead cell removal through negative selections with Miltenyi MACS column separation. Supernatants also were collected and were assayed by OLINK Signature for cytokines that are associated with T cell activation.

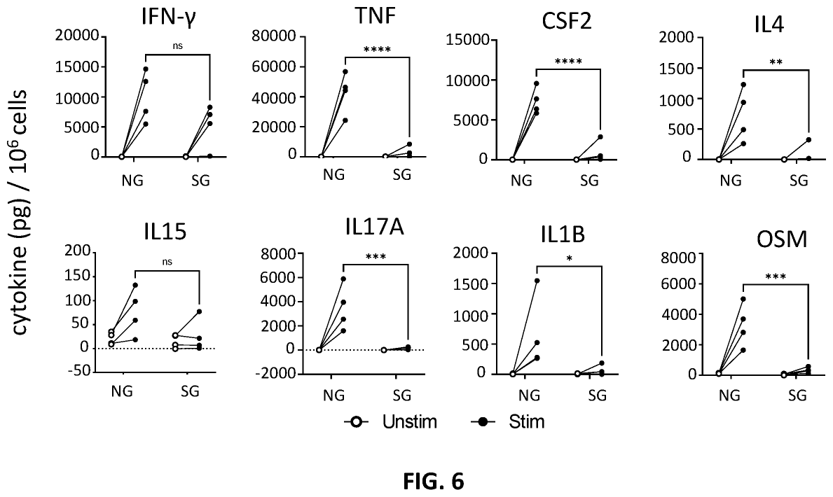

As shown in FIG. 6, several cytokines were reduced in slow-grower supernatants following 48 hour culture with T cell stimulation. The results show that slow growers fail to match output levels of numerous cytokines that are associated with T cell activation.

These data are consistent with a finding that the biological activity following T cell stimulation is impaired in slow growers.

### Example 3: Characterization of CD28+ Healthy Donor Cell Samples

In order to determine whether T cells with high initial CD28 surface expression expand more rapidly than T cells with initial low CD28 surface expression, T cells were sorted from the PBMC material of 3 healthy donors (PBMC samples had no record of poor growth) based on CD28 surface expression and then were stimulated by culturing with beads coated with anti-CD3 and anti-CD28 antibodies or by plating the cells on anti-CD3-coated well plates. Specifically, samples were stained with CD3 and CD28 fluorescent antibodies for sorting and gated on CD3+/CD28+ or CD28− cells. Sorted and recovered cell populations were plated at 30,000 cells per well and were either unstimulated, stimulated via anti-CD3, which was coated on plates, or stimulated with anti-CD3 and CD28 coated beads.

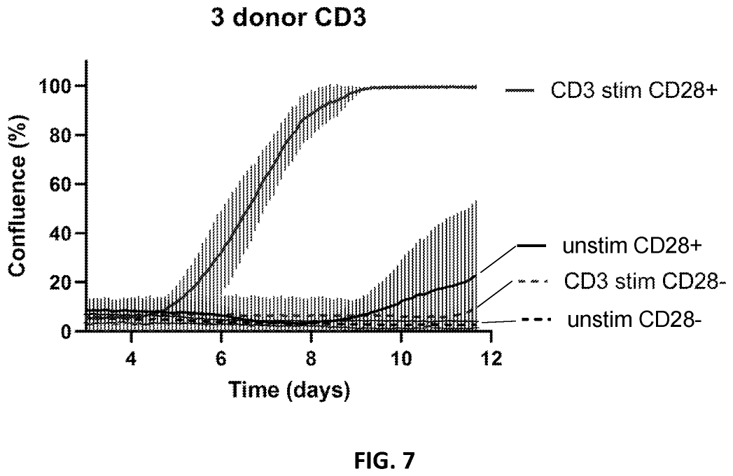

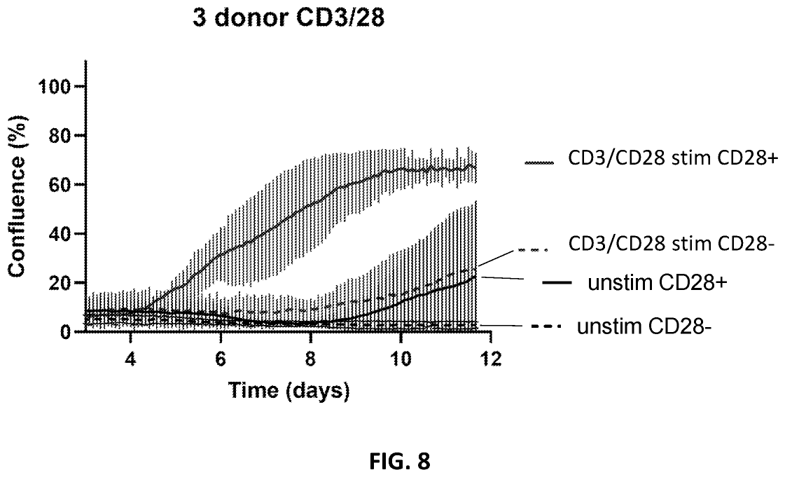

Measurement of plate confluence through continuous live cell imaging at 2-hour intervals over 12 days showed that CD28− cells did not proliferate regardless of stimulation condition over the 12 day period, CD3 stimulated CD28+ cells began rapid proliferation around day 5, and unstimulated CD28+ cells showed non-uniform proliferation beginning on day 9 (FIG. 7). Results also showed that CD3/CD28 stimulated CD28− cells showed proliferation variability beginning around days 8 and 9, and CD3/CD28 stimulated CD28+ cells began proliferation around day 5 (FIG. 8).

The results show that CD28+ T cells began doubling more quickly and reached maximum confluence more quickly than CD28− cells in a controlled proliferation assay regardless whether CD28 co-stimulation was present or absent, suggesting CD28 expression is a surrogate of intrinsic proliferative capacity. These results suggest that CD28 surface expression is a compelling surrogate marker for predicting intrinsic proliferation capability of T cells, yet CD28 co-stimulation is not necessary to initiate doubling of CD28+ cells. The data indicates that CD28 co-stimulation may only be a partial mechanism by which effective population doubling occurs after pan T cell stimulation.

### Example 4: Gene Expression Signatures of Slow Grower Cells and Normal Grower Cells

In order to determine whether gene expression is different between slow growers (SG) and normal growers (NG), 4 slow grower and 4 normal grower samples were assessed for RNA expression. Whole PBMCs were seeded into T25 flasks at 1 million live cells/mL for 48-hour culture with or without soluble anti-CD3 and anti CD28. At the end of culture, cells and supernatants were collected and enriched for CD3, followed by dead cell removal through negative selections with Miltenyi MACS column separation. Highly viable T cells were input into 10× Chromium 5′ workflow to generate GenEx and VDJ libraries. RNA was extracted from remaining enriched bulk cells using the RNEasy plus micro kit; RNA was run on NanoString nCounter assay with human Immune Exhaustion and Metabolic Pathways panels.

A. Gene Expression Changes Before and After Stimulation in NG and SG cells

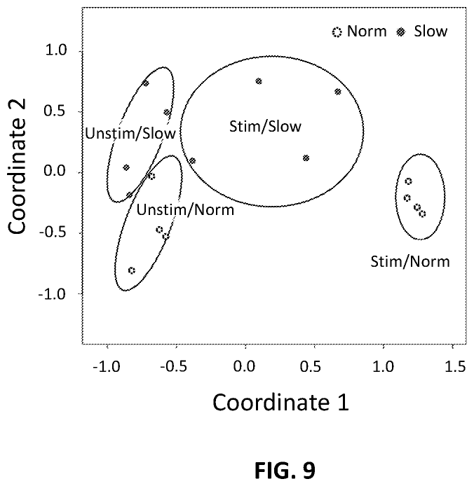

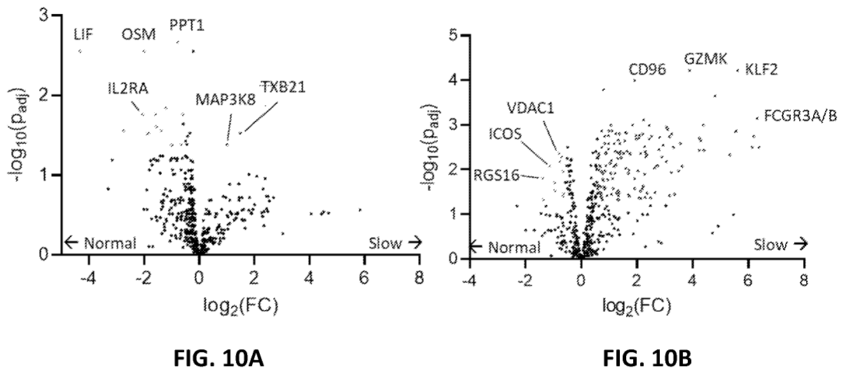

Gene expression signatures in bulk cells were measured to determine differences in gene expression between NG and SG. In a multi-dimensional scaling plot (FIG. 9), samples clustered by stimulation (coordinate 1) and by growth (coordinate 2). NG showed greater gene expression in the unstimulated condition (FIG. 10A) but there was a significant downregulation of gene expression compared to SG when stimulated (FIG. 10B), indicating that SG failed to downregulate numerous genes with stimulation.

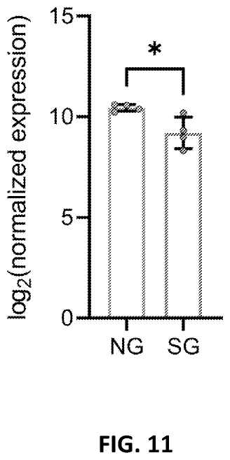

Further, and as shown in FIG. 11, CD28 RNA expression is significantly reduced in SG cells compared to NG cells. These data indicate that in addition to protein characterization of CD28+NG cells, RNA expression of CD28 in bulk cells can also be used as a marker to predict NG cells.

### B. T Cell Heterogeneity in a Combined Population of NG and SG Cells

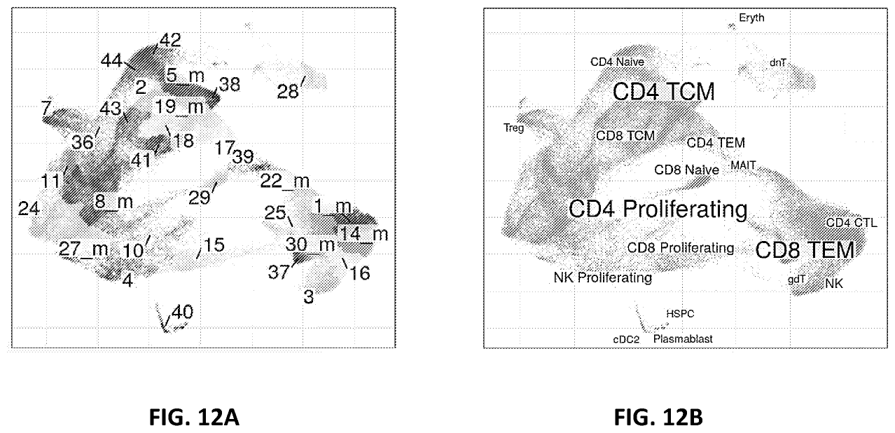

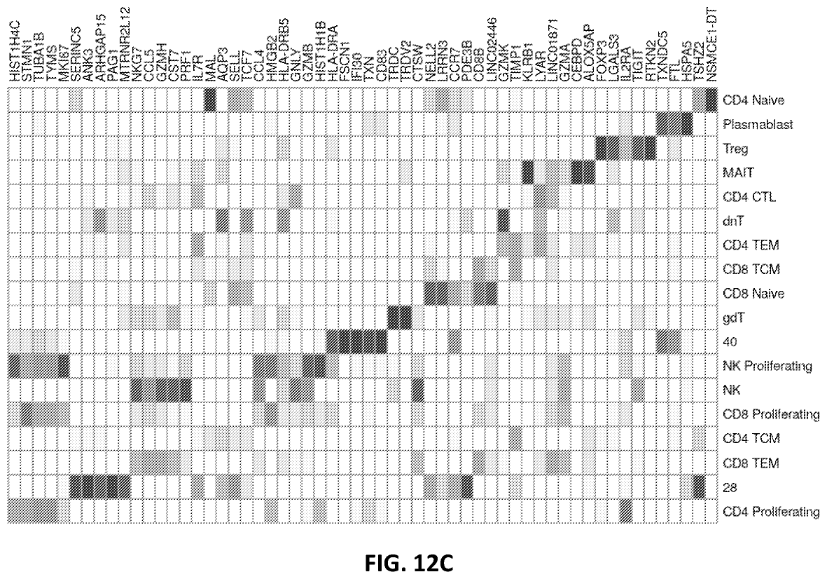

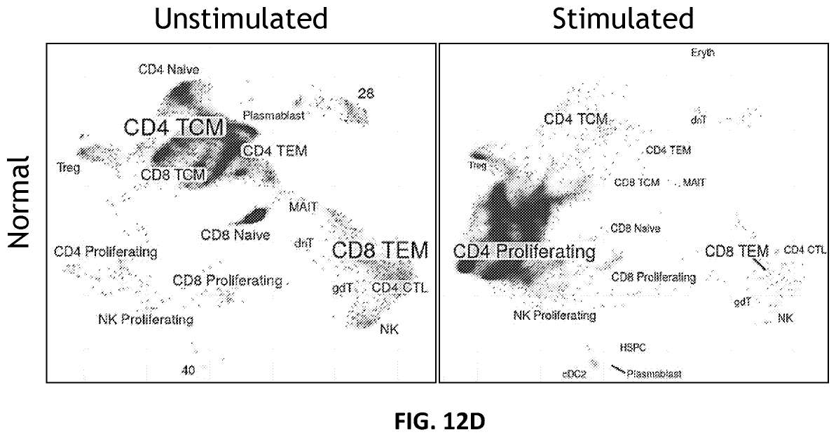

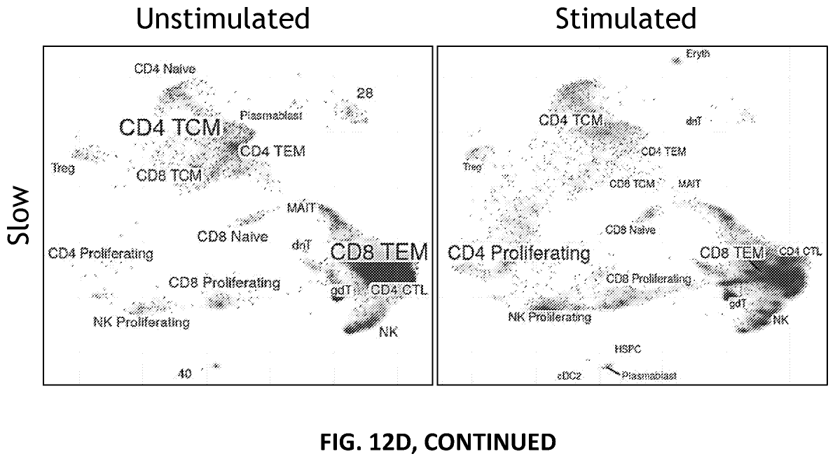

Cells from SG and NG samples were combined to generate a combined sample for unbiased clustering analysis. Cells of the combined sample were clustered based on single-cell RNA expression levels, which revealed heterogeneity within the T cell population of the combined sample (FIG. 12A). A predictive algorithm using cell atlas-driven cluster prediction was used to label clusters as predicted T cell subsets (FIG. 12B). The predictive algorithm labels the regions as T cell subsets based on the gene expression signature displayed in FIG. 12C.

Gene expression signatures of the predicted T cell subsets were determined. Predicted T cell subtypes differed between slow grower and normal grower samples, as well as between unstimulated and stimulated samples. The single cell RNA sequencing thus shows differential cell composition in NG and SG, which is dependent on stimulation. Stimulation created varying shifts in population between SG and NG. Unstimulated samples showed the greatest skew in central memory T (TCM) cells CD4+ cells, proliferating CD4+ cells, naïve cells (NG), and effector memory T (TEM) cells (SG) (FIG. 12D). Stimulated samples showed the greatest skew in proliferating CD4+ cells (NG), TEM CD8+ cells and proliferating CD8+ cells (SG) (FIG. 12D).

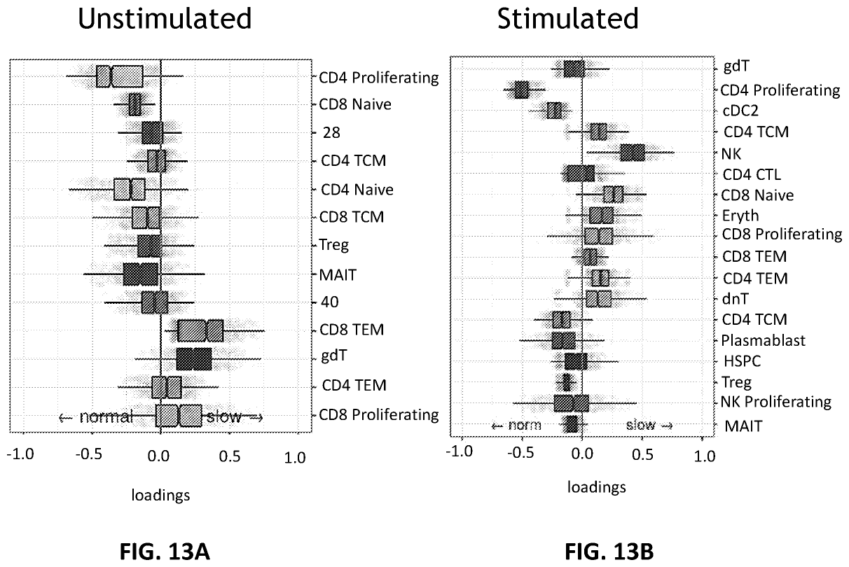

The loadings of selected predicted cell clusters between slow grower and normal grower samples is depicted for unstimulated cells (FIG. 13A) and stimulated cells (FIG. 13B). Unbiased statistical composition analysis showed that several cell types were significantly shifted toward NG or SG. The single cell analysis showed that normal growers displayed greater shift from central memory (CM) CD4 and effector memory (EM) CD8 subsets to proliferative signatures with stimulation. Unstimulated normal growers contained more prominent compartments of naïve and cycling CD4 cells.

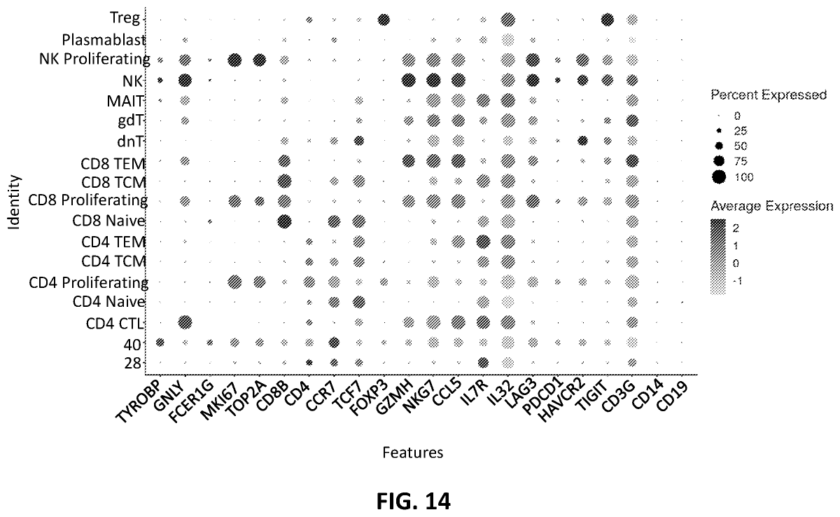

Validation of T cell subsets was determined by expression of identity-associated genes (FIG. 14). These expression patterns validated the predicted cell subsets shown in FIGS. 12A-12C. FIG. 14 indicates the percent of cells (0%, 25%, 50% or 100%) identified on the y-axis that express the genes listed on the x-axis and is referred to as “Percent Expressed.” FIG. 14 also indicates the “Average Expression” value for each gene. Average expression is calculated as log(counts of the gene in the cell/total counts). For example, MKI67 is expressed in 100% of CD4 proliferating cells in a population of CD4 proliferating cells with an average expression between 1 and 2. Additionally, CD3G is expressed in T cell clusters, CD8B is expressed in CD8 subsets, and FOXP3 is expressed in regulatory T cells. These high-dimensional data sets suggested that T cell differentiation state is a key driver of proliferative capacity. Because CD28 is a marker of naïve and CM T cells, CD28 could be a surrogate of the more-robust naïve and CM T cell populations found in normal-growing patient PBMCs.

In view of the results of Examples 1-4, future analyses include: (a) predicting signaling and metabolic pathways upregulated and downregulated in key cell clusters of slow growing cells to determine mechanisms causing lack of response to stimulation; (b) analyzing differences in differentiation states and proliferation capacities between normal growing and slow growing cells; (c) performing a gene-driven trajectory analysis of slow grower and normal grower cells through stimulation; and (d) defining and evaluating potential strategies to predict slow growing cells based on cell composition or cell state, while proposing process adjustments that will rescue poor expansion in identified slow grower cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ ID | | |
|---|---|---|
| NO. | SEQUENCE | DESCRIPTION |
| 1 | ESKYGPPCPPCP | spacer |
| | | (IgG4hinge) (aa) |
| | | Homo sapiens |
| 2 | GAATCTAAGTACGGACCGCCCTGCCCCCCTTGCCCT | spacer |
| | | (IgG4hinge) (nt) |
| | | Homo sapiens |
| 3 | ESKYGPPCPPCPGQPREPQVYTLPPSQEEMTKNQVSLTCLVKGFYP | Hinge-CH3 |
| | SDIAVEWESNGQPENNYKTTPPVLDSDGSFFLYSRLTVDKSRWQE | spacer |
| | GNVFSCSVMHEALHNHYTQKSLSLSLGK | Homo sapiens |
| 4 | ESKYGPPCPPCPAPEFLGGPSVFLFPPKPKDTLMISRTPEVTCVVVD | Hinge-CH2-CH3 |
| | VSQEDPEVQFNWYVDGVEVHNAKTKPREEQFNSTYRVVSVLTVL | spacer |
| | HQDWLNGKEYKCKVSNKGLPSSIEKTISKAKGQPREPQVYTLPPSQ | Homo sapiens |
| | EEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSL | |
| | GK | |
| 5 | RWPESPKAQASSVPTAQPQAEGSLAKATTAPATTRNTGRGGEEKK | IgD-hinge-Fc |
| | KEKEKEEQEERETKTPECPSHTQPLGVYLLTPAVQDLWLRDKATF | Homo sapiens |
| | TCFVVGSDLKDAHLTWEVAGKVPTGGVEEGLLERHSNGSQSQHS | |
| | RLTLPRSLWNAGTSVTCTLNHPSLPPQRLMALREPAAQAPVKLSL | |
| | NLLASSDPPEAASWLLCEVSGFSPPNILLMWLEDQREVNTSGFAPA | |
| | RPPPQPGSTTFWAWSVLRVPAPPSPQPATYTCVVSHEDSRTLLNAS | |
| | RSLEVSYVTDH | |
| 6 | LEGGGEGRGSLLTCGDVEENPGPR | T2A |
| | | artificial |
| 7 | RKVCNGIGIGEFKDSLSINATNIKHFKNCTSISGDLHILPVAFRGDSF | tEGFR |
| | THTPPLDPQELDILKTVKEITGFLLIQAWPENRTDLHAFENLEIIRGR | artificial |
| | TKQHGQFSLAVVSLNITSLGLRSLKEISDGDVIISGNKNLCYANTIN | |
| | WKKLFGTSGQKTKIISNRGENSCKATGQVCHALCSPEGCWGPEPR | |
| | DCVSCRNVSRGRECVDKCNLLEGEPREFVENSECIQCHPECLPQA | |
| | MNITCTGRGPDNCIQCAHYIDGPHCVKTCPAGVMGENNTLVWKY | |
| | ADAGHVCHLCHPNCTYGCTGPGLEGCPTNGPKIPSIATGMVGALL | |
| | LLLVVALGIGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKPFWVLVVV | CD28 (amino |
| | GGVLACYSLLVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to |
| | | GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 14 | RVKFSRSAEPPAYQQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 15 | RVKFSRSADAPAYKQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 16 | PGGG-(SGGGG)5-P- wherein P is proline, G is glycine | linker |
| | and S is serine | |
| 17 | GSADDAKKDAAKKDGKS | Linker |
| 18 | QIQLVQSGPELKKPGETVKISCKASGYTFTDYSINWVKRAPGKGLK | Variable heavy |
| | WMGWINTETREPAYAYDFRGRFAFSLETSASTAYLQINNLKYEDT | (VH) Anti- |
| | ATYFCALDYSYAMDYWGQGTSVTVSS | BCMA |
| 19 | DIVLTQSPPSLAMSLGKRATISCRASESVTILGSHLIHWYQQKPGQP | Variable light |
| | PTLLIQLASNVQTGVPARFSGSGSRTDFTLTIDPVEEDDVAVYYCL | (VL) Anti- |
| | QSRTIPRTFGGGTKLEIK | BCMA |
| 20 | QIQLVQSGPDLKKPGETVKLSCKASGYTFTNFGMNWVKQAPGKG | Variable heavy |
| | FKWMAWINTYTGESYFADDFKGRFAFSVETSATTAYLQINNLKTE | (VH) Anti- |
| | DTATYFCARGEIYYGYDGGFAYWGQGTLVTVSA | BCMA |
| 21 | DVVMTQSHRFMSTSVGDRVSITCRASQDVNTAVSWYQQKPGQSP | Variable light |
| | KLLIFSASYRYTGVPDRFTGSGSGADFTLTISSVQAEDLAVYYCQQ | (VL) Anti- |
| | HYSTPWTFGGGTKLDIK | BCMA |
| 22 | EVQLVQSGAEVKKPGESLKISCKGSGYSFTSYWIGWVRQMPGKGL | Variable heavy |
| | EWMGIIYPGDSDTRYSPSFQGHVTISADKSISTAYLQWSSLKASDT | (VH) Anti- |
| | AMYYCARYSGSFDNWGQGTLVTVSS | BCMA |
| 23 | SYELTQPPSASGTPGQRVTMSCSGTSSNIGSHSVNWYQQLPGTAPK | Variable light |
| | LLIYTNNQRPSGVPDRFSGSKSGTSASLAISGLQSEDEADYYCAAW | (VL) Anti- |
| | DGSLNGLVFGGGTKLTVLG | BCMA |
| 24 | EVQLVQSGAEMKKPGASLKLSCKASGYTFIDYYVYWMRQAPGQG | Variable heavy |
| | LESMGWINPNSGGTNYAQKFQGRVTMTRDTSISTAYMELSRLRSD | (VH) Anti- |
| | DTAMYYCARSQRDGYMDYWGQGTLVTVSS | BCMA |
| 25 | QSALTQPASVSASPGQSIAISCTGTSSDVGWYQQHPGKAPKLMIYE | Variable light |
| | DSKRPSGVSNRFSGSKSGNTASLTISGLQAEDEADYYCSSNTRSSTL | (VL) Anti- |
| | VFGGGTKLTVLG | BCMA |
| 26 | GGGGS | Linker |
| 27 | GGGS | Linker |
| 28 | GGGGSGGGGSGGGGS | Linker |
| 29 | GSTSGSGKPGSGEGSTKG | Linker |
| 30 | SRGGGGSGGGGSGGGGSLEMA | Linker |
| 31 | ESKYGPPCPPCPAPPVAGPSVFLFPPKPKDTLMISRTPEVTCVVVDV | Hinge-CH2-CH3 |
| | SQEDPEVQFNWYVDGVEVHNAKTKPREEQFQSTYRVVSVLTVLH | spacer |
| | QDWLNGKEYKCKVSNKGLPSSIEKTISKAKGQPREPQVYTLPPSQE | Homo sapiens |
| | EMTKNQVSLTCLVKGFYPSDIAVEWESNGQPENNYKTTPPVLDSD | |
| | GSFFLYSRLTVDKSRWQEGNVFSCSVMHEALHNHYTQKSLSLSLG | |
| | K | |
| 32 | EVQLVQSGAEVKKPGSSVKVSCKASGGTFSSYAISWVRQAPGQGL | Variable heavy |
| | EWMGRIIPILGIANYAQKFQGRVTMTEDTSTDTAYMELSSLRSEDT | (VH) Anti- |
| | AVYYCARSGYSKSIVSYMDYWGQGTLVTVSS | BCMA |
| 33 | LPVLTQPPSTSGTPGQRVTVSCSGSSSNIGSNVVFWYQQLPGTAPK | Variable light |
| | LVIYRNNQRPSGVPDRFSVSKSGTSASLAISGLRSEDEADYYCAAW | (VL) Anti- |
| | DDSLSGYVFGTGTKVTVLG | BCMA |
| 34 | QVQLVQSGAEVKKPGSSVKVSCKASGGTFSSYAISWVRQAPGQGL | Variable heavy |
| | EWMGRIIPILGTANYAQKFQGRVTITADESTSTAYMELSSLRSEDT | (VH) Anti- |
| | AVYYCARSGYGSYRWEDSWGQGTLVTVSS | BCMA |
| 35 | QAVLTQPPSASGTPGQRVTISCSGSSSNIGSNYVFWYQQLPGTAPK | Variable light |
| | LLIYSNNQRPSGVPDRFSGSKSGTSASLAISGLRSEDEADYYCAAW | (VL) Anti- |
| | DDSLSASYVFGTGTKVTVLG | BCMA |
| 36 | QVQLVQSGAEVKKPGASVKVSCKASGYTFTDYYMHWVRQAPGQ | Variable heavy |
| | RLEWMGWINPNSGGTNYAQKFQDRITVTRDTSSNTGYMELTRLRS | (VH) Anti- |
| | DDTAVYYCARSPYSGVLDKWGQGTLVTVSS | BCMA |
| 37 | QSVLTQPPSVSGAPGQRVTISCTGSSSNIGAGFDVHWYQQLPGTAP | Variable light |
| | KLLIYGNSNRPSGVPDRFSGSKSGTSASLAITGLQAEDEADYYCQS | (VL) Anti- |
| | YDSSLSGYVFGTGTKVTVLG | BCMA |
| 38 | ASGGGGSGGRASGGGGS | Linker |
| 39 | MALPVTALLLPLALLLHAARP | CD8a signal |
| | | peptide |
| 40 | METDTLLLWVLLLWVPGSTG | signal peptide |
| 41 | EVQLLESGGGLVQPGGSLRLSCAASGFTFSSYAMSWVRQAPGKGL | Variable heavy |
| | EWVSAISGSGGSTYYADSVKGRFTISRDNSKNTLYLQMNSLRAED | (VH) Anti- |
| | TAVYYCARAEMGAVFDIWGQGTMVTVSS | BCMA |
| 42 | EIVLTQSPATLSLSPGERATLSCRASQSVSRYLAWYQQKPGQAPRL | Variable light |
| | LIYDASNRATGIPARFSGSGSGTDFTLTISSLEPEDFAVYYCQQRIS | (VL) Anti- |
| | WPFTFGGGTKVEIK | BCMA |
| 43 | QVQLVESGGGVVQPGRSLRLSCAASGFTFSSYGMHWVRQAPGKG | Variable heavy |
| | LEWVAVISYDGSNKYYADSVKGRFTISRDNSKNTLYLQMNSLRAE | (VH) Anti- |
| | DTAVYYCARDGTYLGGLWYFDLWGRGTLVTVSS | BCMA |
| 44 | DIVMTQSPLSLPVTPGEPASISCRSSQSLLHSNGYNYLDWYLQKPG | Variable light |
| | QSPQLLIYLGSNRASGVPDRFSGSGSGTDFTLKISRVEAEDVGVYY | (VL) Anti- |
| | CMQGLGLPLTFGGGTKVEIK | BCMA |
| 45 | QVQLVQSGAEVKKPGASVKVSCKASGYTFTSYYMHWVRQAPGQ | Variable heavy |
| | GLEWMGIINPGGGSTSYAQKFQGRVTMTRDTSTSTVYMELSSLRS | (VH) Anti- |
| | EDTAVYYCARESWPMDVWGQGTTVTVSS | BCMA |
| 46 | EIVMTQSPATLSVSPGERATLSCRASQSVSSNLAWYQQKPGQAPRL | Variable light |
| | LIYGASTRATGIPARFSGSGSGTEFTLTISSLQSEDFAVYYCQQYAA | (VL) Anti- |
| | YPTFGGGTKVEIK | BCMA |
| 47 | QLQLQESGPGLVKPSETLSLTCTVSGGSISSSSYYWGWIRQPPGKG | Variable heavy |
| | LEWIGSISYSGSTYYNPSLKSRVTISVDTSKNQFSLKLSSVTAADTA | (VH) Anti- |
| | VYYCARGRGYATSLAFDIWGQGTMVTVSS | BCMA |
| 48 | EIVLTQSPATLSLSPGERATLSCRASQSVSSYLAWYQQKPGQAPRL | Variable light |
| | LIYDASNRATGIPARFSGSGSGTDFTLTISSLEPEDFAVYYCQQRHV | (VL) Anti- |
| | WPPTFGGGTKVEIK | BCMA |
| 49 | EVQLVESGGGLVQPGGSLRLSCAASGFTFSSYSMNWVRQAPGKGL | Variable heavy |
| | EWVSTISSSSSTIYYADSVKGRFTISRDNAKNSLYLQMNSLRAEDT | (VH) Anti- |
| | AVYYCARGSQEHLIFDYWGQGTLVTVSS | BCMA |
| 50 | EIVLTQSPATLSLSPGERATLSCRASQSVSRYLAWYQQKPGQAPRL | Variable light |
| | LIYDASNRATGIPARFSGSGSGTDFTLTISSLEPEDFAVYYCQQRFY | (VL) Anti- |
| | YPWTFGGGTKVEIK | BCMA |
| 51 | QVQLVESGGGVVQPGRSLRLSCAASGFTFSSYGMHWVRQAPGKG | Variable heavy |
| | LEWVAVISYDGSNKYYADSVKGRFTISRDNSKNTLYLQMNSLRAE | (VH) Anti- |
| | DTAVYYCARTDFWSGSPPGLDYWGQGTLVTVSS | BCMA |
| 52 | DIQLTQSPSSVSASVGDRVTITCRASQGISSWLAWYQQKPGKAPKL | Variable light |
| | LIYGASSLQSGVPSRFSGSGSGTDFTLTISSLQPEDFATYYCQQIYTF | (VL) Anti- |
| | PFTFGGGTKVEIK | BCMA |
| 53 | QVQLVQSGAEVKKPGSSVKVSCKASGGTFSSYAISWVRQAPGQGL | Variable heavy |
| | EWMGGIIPIFGTANYAQKFQGRVTITADESTSTAYMELSSLRSEDT | (VH) Anti- |
| | AVYYCARTPEYSSSIWHYYYGMDVWGQGTTVTVSS | BCMA |
| 54 | DIVMTQSPDSLAVSLGERATINCKSSQSVLYSSNNKNYLAWYQQK | Variable light |
| | PGQPPKLLIYWASTRESGVPDRFSGSGSGTDFTLTISSLQAEDVAVY | (VL) Anti- |
| | YCQQFAHTPFTFGGGTKVEIK | BCMA |
| 55 | QVQLVESGGGVVQPGRSLRLSCAASGFTFSSYGMHWVRQAPGKG | Variable heavy |
| | LEWVAVISYDGSNKYYADSVKGRFTISRDNSKNTLYLQMNSLRAE | (VH) Anti- |
| | DTAVYYCVKGPLQEPPYDYGMDVWGQGTTVTVSS | BCMA |
| 56 | EIVMTQSPATLSVSPGERATLSCRASQSVSSNLAWYQQKPGQAPRL | Variable light |
| | LIYSASTRATGIPARFSGSGSGTEFTLTISSLQSEDFAVYYCQQHHV | (VL) Anti- |
| | WPLTFGGGTKVEIK | BCMA |
| 57 | QVQLVQSGAEVKKPGSSVKVSCKASGGTFSSYAISWVRQAPGQGL | Variable heavy |
| | EWMGRIIPILGIANYAQKFQGRVTITADKSTSTAYMELSSLRSEDTA | (VH) Anti- |
| | VYYCARGGYYSHDMWSEDWGQGTLVTVSS | BCMA |
| 58 | LPVLTQPPSASGTPGQRVTISCSGRSSNIGSNSVNWYRQLPGAAPKL | Variable light |
| | LIYSNNQRPPGVPVRFSGSKSGTSASLAISGLQSEDEATYYCATWD | (VL) Anti- |
| | DNLNVHYVFGTGTKVTVLG | BCMA |
| 59 | QVQLVQSGSELKKPGASVKVSCKASGYTFTDYSINWVRQAPGQGL | Variable heavy |
| | EWMGWINTETREPAYAYDFRGRFVFSLDTSVSTAYLQISSLKAEDT | (VH) Anti- |
| | AVYYCARDYSYAMDYWGQGTLVTVSS | BCMA |
| 60 | DIVLTQSPASLAVSLGERATINCRASESVSVIGAHLIHWYQQKPGQP | Variable light |
| | PKLLIYLASNLETGVPARFSGSGSGTDFTLTISSLQAEDAAIYYCLQ | (VL) Anti- |
| | SRIFPRTFGQGTKLEIK | BCMA |
| 61 | EVQLVESGGGLVQPGGSLRLSCAVSGFALSNHGMSWVRRAPGKG | Variable heavy |
| | LEWVSGIVYSGSTYYAASVKGRFTISRDNSRNTLYLQMNSLRPEDT | (VH) Anti- |
| | AIYYCSAHGGESDVWGQGTTVTVSS | BCMA |
| 62 | DIQLTQSPSSLSASVGDRVTITCRASQSISSYLNWYQQKPGKAPKLL | Variable light |
| | IYAASSLQSGVPSRFSGSGSGTDFTLTISSLQPEDFATYYCQQSYSTP | (VL) Anti- |
| | YTFGQGTKVEIK | BCMA |
| 63 | QVQLVESGGGLVQPGRSLRLSCAASGFTFSNYAMSWVRQAPGKG | Variable heavy |
| | LGWVSGISRSGENTYYADSVKGRFTISRDNSKNTLYLQMNSLRDE | (VH) Anti- |
| | DTAVYYCARSPAHYYGGMDVWGQGTTVTVSS | BCMA |
| 64 | DIVLTQSPGTLSLSPGERATLSCRASQSISSSFLAWYQQKPGQAPRL | Variable light |
| | LIYGASRRATGIPDRFSGSGSGTDFTLTISRLEPEDSAVYYCQQYHS | (VL) Anti- |
| | SPSWTFGQGTKLEIK | BCMA |
| 65 | QVQLVESGGGLVQPGGSLRLSCAVSGFALSNHGMSWVRRAPGKG | Variable heavy |
| | LEWVSGIVYSGSTYYAASVKGRFTISRDNSRNTLYLQMNSLRPEDT | (VH) Anti- |
| | AIYYCSAHGGESDVWGQGTTVTVSS | BCMA |
| 66 | DIRLTQSPSPLSASVGDRVTITCQASEDINKFLNWYHQTPGKAPKLL | Variable light |
| | IYDASTLQTGVPSRFSGSGSGTDFTLTINSLQPEDIGTYYCQQYESLP | (VL) Anti- |
| | LTFGGGTKVEIK | BCMA |
| 67 | EVQLVESGGGLVQPGGSLRLSCAVSGFALSNHGMSWVRRAPGKG | Variable heavy |
| | LEWVSGIVYSGSTYYAASVKGRFTISRDNSRNTLYLQMNSLRPEDT | (VH) Anti- |
| | AIYYCSAHGGESDVWGQGTTVTVSS | BCMA |
| 68 | EIVLTQSPGTLSLSPGERATLSCRASQSIGSSSLAWYQQKPGQAPRL | Variable light |
| | LMYGASSRASGIPDRFSGSGSGTDFTLTISRLEPEDFAVYYCQQYA | (VL) Anti- |
| | GSPPFTFGQGTKVEIK | BCMA |
| 69 | QIQLVQSGPELKKPGETVKISCKASGYTFRHYSMNWVKQAPGKGL | Variable heavy |
| | KWMGRINTESGVPIYADDFKGRFAFSVETSASTAYLVINNLKDEDT | (VH) Anti- |
| | ASYFCSNDYLYSLDFWGQGTALTVSS | BCMA |
| 70 | DIVLTQSPPSLAMSLGKRATISCRASESVTILGSHLIYWYQQKPGQP | Variable light |
| | PTLLIQLASNVQTGVPARFSGSGSRTDFTLTIDPVEEDDVAVYYCL | (VL) Anti- |
| | QSRTIPRTFGGGTKLEIK | BCMA |
| 71 | QIQLVQSGPELKKPGETVKISCKASGYTFTHYSMNWVKQAPGKGL | Variable heavy |
| | KWMGRINTETGEPLYADDFKGRFAFSLETSASTAYLVINNLKNED | (VH) Anti- |
| | TATFFCSNDYLYSCDYWGQGTTLTVSS | BCMA |
| 72 | DIVLTQSPASLAMSLGKRATISCRASESVSVIGAHLIHWYQQKPGQ | Variable light |
| | PPKLLIYLASNLETGVPARFSGSGSGTDFTLTIDPVEEDDVAIYSCL | (VL) Anti- |
| | QSRIFPRTFGGGTKLEIK | BCMA |
| 73 | QVQLVQSGAEVKKPGASVKVSCKASGYSFPDYYINWVRQAPGQG | Variable heavy |
| | LEWMGWIYFASGNSEYNQKFTGRVTMTRDTSINTAYMELSSLTSE | (VH) Anti- |
| | DTAVYFCASLYDYDWYFDVWGQGTMVTVSS | BCMA |
| 74 | DIVMTQTPLSLSVTPGQPASISCKSSQSLVHSNGNTYLHWYLQKPG | Variable light |
| | QSPQLLIYKVSNRFSGVPDRFSGSGSGTDFTLKISRVEAEDVGIYYC | (VL) Anti- |
| | SQSSIYPWTFGQGTKLEIK | BCMA |
| 75 | QVQLVQSGAEVKKPGASVKVSCKASGYSFPDYYINWVRQAPGQG | Variable heavy |
| | LEWMGWIYFASGNSEYNQKFTGRVTMTRDTSSSTAYMELSSLRSE | (VH) Anti- |
| | DTAVYFCASLYDYDWYFDVWGQGTMVTVSS | BCMA |
| 76 | DIVMTQTPLSLSVTPGEPASISCKSSQSLVHSNGNTYLHWYLQKPG | Variable light |
| | QSPQLLIYKVSNRFSGVPDRFSGSGSGADFTLKISRVEAEDVGVYY | (VL) Anti- |
| | CAETSHVPWTFGQGTKLEIK | BCMA |
| 77 | QVQLVESGGGLVQPGGSLRLSCEASGFTLDYYAIGWFRQAPGKER | Anti-BCMA |
| | EGVICISRSDGSTYYADSVKGRFTISRDNAKKTVYLQMISLKPEDT | sdAb |
| | AAYYCAAGADCSGYLRDYEFRGQGTQVTVSS | |
| 78 | IEVMYPPPYLDNEKSNGTIIHVKGKHLCPSPLFPGPSKP | CD28 spacer |
| 79 | IYIWAPLAGTCGVLLLSLVITLYCN | CD8a TM |
| 80 | LDNEKSNGTIIHVKGKHLCPSPLFPGPSKP | CD28 spacer |
| | | (truncated) |
| 81 | PTTTPAPRPPTPAPTIASQPLSLRPEACRPAAGGAVHTRGLDFACD | CD8a hinge |
| 82 | TTTPAPRPPTPAPTIASQPLSLRPEACRPAAGGAVHTRGLDFACD | CD8a hinge |
| 83 | FVPVFLPAKPTTTPAPRPPTPAPTIASQPLSLRPEACRPAAGGA VHT | CD8a hinge |
| | RGLDFACD | |
| 84 | DTGLYICKVELMYPPPYYLGIGNGTQIYVIDPEPCPDSD | CTLA4 hinge |
| 85 | FLLWILAAVSSGLFFYSFLLTAVS | CTLA4 TM |
| 86 | QIKESLRAELRVTERRAEVPTAHPSPSPRPAGQFQTLV | PD-1 hinge |
| 87 | VGVVGGLLGSLVLLVWVLAVI | PD-1 TM |
| 88 | GLAVSTISSFFPPGYQ | Fc(gamma)RIIIa |
| | | hinge |
| 89 | EPKSPDKTHTCPPCPAPPVAGPSVFLFPPKPKDTLMIARTPEVTCVV | IgG1 hinge |
| | VDVSHEDPEVKFNWYVDGVEVHNAKTKPREEQYNSTYRVVSVLT | |
| | VLHQDWLNGKEYKCKVSNKALPAPIEKTISKAKGQPREPQVYTLP | |
| | PSRDELTKNQVSLTCLVKGFYPSDIAVEWESNGQPENNYKTTPPVL | |
| | DSDGSFFLYSKLTVDKSRWQQGNVFSCSVMHEALHNHYTQKSLSL | |
| | SPGK | |
| 90 | EVQLLESGGGLVQPGGSLRLSCAASGFTFSSYAMSWVRQAPGKGL | anti-BCMA CAR |
| | EWVSAISGSGGSTYYADSVKGRFTISRDNSKNTLYLQMNSLRAED | |
| | TAVYYCARAEMGAVFDIWGQGTMVTVSSGSTSGSGKPGSGEGST | |
| | KGEIVLTQSPATLSLSPGERATLSCRASQSVSRYLAWYQQKPGQAP | |
| | RLLIYDASNRATGIPARFSGSGSGTDFTLTISSLEPEDFAVYYCQQRI | |
| | SWPFTFGGGTKVEIKRAAALDNEKSNGTIIHVKGKHLCPSPLFPGPS | |
| | KPFWVLVVVGGVLACYSLLVTVAFIIFWVRSKRSRLLHSDYMNMT | |
| | PRRPGPTRKHYQPYAPPRDFAAYRSRVKFSRSADAPAYQQGQNQL | |
| | YNELNLGRREEYDVLDKRRGRDPEMGGKPRRKNPQEGLYNELQK | |
| | DKMAEAYSEIGMKGERRRGKGHDGLYQGLSTATKDTYDALHMQ | |
| | ALPPR | |
| 91 | EIVLTQSPATLSLSPGERATLSCRASQSVSRYLAWYQQKPGQAPRL | anti-BCMA CAR |
| | LIYDASNRATGIPARFSGSGSGTDFTLTISSLEPEDFAVYYCQQRIS | |
| | WPFTFGGGTKVEIKRGSTSGSGKPGSGEGSTKGEVQLLESGGGLV | |
| | QPGGSLRLSCAASGFTFSSYAMSWVRQAPGKGLEWVSAISGSGGS | |
| | TYYADSVKGRFTISRDNSKNTLYLQMNSLRAEDTAVYYCARAEM | |
| | GAVFDIWGQGTMVTVSSAAALDNEKSNGTIIHVKGKHLCPSPLFP | |
| | GPSKPFWVLVVVGGVLACYSLLVTVAFIIFWVRSKRSRLLHSDYM | |
| | NMTPRRPGPTRKHYQPYAPPRDFAAYRSRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 92 | QVQLVESGGGVVQPGRSLRLSCAASGFTFSSYGMHWVRQAPGKG | anti-BCMA CAR |
| | LEWVAVISYDGSNKYYADSVKGRFTISRDNSKNTLYLQMNSLRAE | |
| | DTAVYYCARDGTYLGGLWYFDLWGRGTLVTVSSGSTSGSGKPGS | |
| | GEGSTKGDIVMTQSPLSLPVTPGEPASISCRSSQSLLHSNGYNYLD | |
| | WYLQKPGQSPQLLIYLGSNRASGVPDRFSGSGSGTDFTLKISRVEA | |
| | EDVGVYYCMQGLGLPLTFGGGTKVEIKRAAALDNEKSNGTIIHVK | |
| | GKHLCPSPLFPGPSKPFWVLVVVGGVLACYSLLVTVAFIIFWVRSK | |
| | RSRLLHSDYMNMTPRRPGPTRKHYQPYAPPRDFAAYRSRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 93 | DIVMTQSPLSLPVTPGEPASISCRSSQSLLHSNGYNYLDWYLQKPG | anti-BCMA CAR |
| | QSPQLLIYLGSNRASGVPDRFSGSGSGTDFTLKISRVEAEDVGVYY | |
| | CMQGLGLPLTFGGGTKVEIKRGSTSGSGKPGSGEGSTKGQVQLVE | |
| | SGGGVVQPGRSLRLSCAASGFTFSSYGMHWVRQAPGKGLEWVAV | |
| | ISYDGSNKYYADSVKGRFTISRDNSKNTLYLQMNSLRAEDTAVYY | |
| | CARDGTYLGGLWYFDLWGRGTLVTVSSAAALDNEKSNGTIIHVK | |
| | GKHLCPSPLFPGPSKPFWVLVVVGGVLACYSLLVTVAFIIFWVRSK | |
| | RSRLLHSDYMNMTPRRPGPTRKHYQPYAPPRDFAAYRSRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 94 | QVQLVQSGAEVKKPGASVKVSCKASGYTFTSYYMHWVRQAPGQ | anti-BCMA CAR |
| | GLEWMGIINPGGGSTSYAQKFQGRVTMTRDTSTSTVYMELSSLRS | |
| | EDTAVYYCARESWPMDVWGQGTTVTVSSGSTSGSGKPGSGEGST | |
| | KGEIVMTQSPATLSVSPGERATLSCRASQSVSSNLAWYQQKPGQA | |
| | PRLLIYGASTRATGIPARFSGSGSGTEFTLTISSLQSEDFAVYYCQQY | |
| | AAYPTFGGGTKVEIKRAAALDNEKSNGTIIHVKGKHLCPSPLFPGP | |
| | SKPFWVLVVVGGVLACYSLLVTVAFIIFWVRSKRSRLLHSDYMNM | |
| | TPRRPGPTRKHYQPYAPPRDFAAYRSRVKFSRSADAPAYQQGQNQ | |
| | LYNELNLGRREEYDVLDKRRGRDPEMGGKPRRKNPQEGLYNELQ | |
| | KDKMAEAYSEIGMKGERRRGKGHDGLYQGLSTATKDTYDALHM | |
| | QALPPR | |
| 95 | EIVMTQSPATLSVSPGERATLSCRASQSVSSNLAWYQQKPGQAPRL | anti-BCMA CAR |
| | LIYGASTRATGIPARFSGSGSGTEFTLTISSLQSEDFAVYYCQQYAA | |
| | YPTFGGGTKVEIKRGSTSGSGKPGSGEGSTKGQVQLVQSGAEVKK | |
| | PGASVKVSCKASGYTFTSYYMHWVRQAPGQGLEWMGIINPGGGS | |
| | TSYAQKFQGRVTMTRDTSTSTVYMELSSLRSEDTAVYYCARESWP | |
| | MDVWGQGTTVTVSSAAALDNEKSNGTIIHVKGKHLCPSPLFPGPS | |
| | KPFWVLVVVGGVLACYSLLVTVAFIIFWVRSKRSRLLHSDYMNMT | |
| | PRRPGPTRKHYQPYAPPRDFAAYRSRVKFSRSADAPAYQQGQNQL | |
| | YNELNLGRREEYDVLDKRRGRDPEMGGKPRRKNPQEGLYNELQK | |
| | DKMAEAYSEIGMKGERRRGKGHDGLYQGLSTATKDTYDALHMQ | |
| | ALPPR | |
| 96 | QLQLQESGPGLVKPSETLSLTCTVSGGSISSSSYYWGWIRQPPGKG | anti-BCMA CAR |
| | LEWIGSISYSGSTYYNPSLKSRVTISVDTSKNQFSLKLSSVTAADTA | |
| | VYYCARGRGYATSLAFDIWGQGTMVTVSSGSTSGSGKPGSGEGST | |
| | KGEIVLTQSPATLSLSPGERATLSCRASQSVSSYLAWYQQKPGQAP | |
| | RLLIYDASNRATGIPARFSGSGSGTDFTLTISSLEPEDFAVYYCQQR | |
| | HVWPPTFGGGTKVEIKRAAALDNEKSNGTIIHVKGKHLCPSPLFPG | |
| | PSKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALH | |
| | MQALPPR | |
| 97 | EIVLTQSPATLSLSPGERATLSCRASQSVSSYLAWYQQKPGQAPRL | anti-BCMA CAR |
| | LIYDASNRATGIPARFSGSGSGTDFTLTISSLEPEDFAVYYCQQRHV | |
| | WPPTFGGGTKVEIKRGSTSGSGKPGSGEGSTKGQLQLQESGPGLVK | |
| | PSETLSLTCTVSGGSISSSSYYWGWIRQPPGKGLEWIGSISYSGSTY | |
| | YNPSLKSRVTISVDTSKNQFSLKLSSVTAADTAVYYCARGRGYATS | |
| | LAFDIWGQGTMVTVSSAAALDNEKSNGTIIHVKGKHLCPSPLFPGP | |
| | SKPFWVLVVVGGVLACYSLLVTVAFIIFWVRSKRSRLLHSDYMNM | |
| | TPRRPGPTRKHYQPYAPPRDFAAYRSRVKFSRSADAPAYQQGQNQ | |
| | LYNELNLGRREEYDVLDKRRGRDPEMGGKPRRKNPQEGLYNELQ | |
| | KDKMAEAYSEIGMKGERRRGKGHDGLYQGLSTATKDTYDALHM | |
| | QALPPR | |
| 98 | EVQLVESGGGLVQPGGSLRLSCAASGFTFSSYSMNWVRQAPGKGL | anti-BCMA CAR |
| | EWVSTISSSSSTIYYADSVKGRFTISRDNAKNSLYLQMNSLRAEDT | |
| | AVYYCARGSQEHLIFDYWGQGTLVTVSSGSTSGSGKPGSGEGSTK | |
| | GEIVLTQSPATLSLSPGERATLSCRASQSVSRYLAWYQQKPGQAPR | |
| | LLIYDASNRATGIPARFSGSGSGTDFTLTISSLEPEDFAVYYCQQRF | |
| | YYPWTFGGGTKVEIKRAAALDNEKSNGTIIHVKGKHLCPSPLFPGP | |
| | SKPFWVLVVVGGVLACYSLLVTVAFIIFWVRSKRSRLLHSDYMNM | |
| | TPRRPGPTRKHYQPYAPPRDFAAYRSRVKFSRSADAPAYQQGQNQ | |
| | LYNELNLGRREEYDVLDKRRGRDPEMGGKPRRKNPQEGLYNELQ | |
| | KDKMAEAYSEIGMKGERRRGKGHDGLYQGLSTATKDTYDALHM | |
| | QALPPR | |
| 99 | EIVLTQSPATLSLSPGERATLSCRASQSVSRYLAWYQQKPGQAPRL | anti-BCMA CAR |
| | LIYDASNRATGIPARFSGSGSGTDFTLTISSLEPEDFAVYYCQQRFY | |
| | YPWTFGGGTKVEIKRGSTSGSGKPGSGEGSTKGEVQLVESGGGLV | |
| | QPGGSLRLSCAASGFTFSSYSMNWVRQAPGKGLEWVSTISSSSSTI | |
| | YYADSVKGRFTISRDNAKNSLYLQMNSLRAEDTAVYYCARGSQE | |
| | HLIFDYWGQGTLVTVSSAAALDNEKSNGTIIHVKGKHLCPSPLFPG | |
| | PSKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALH | |
| | MQALPPR | |
| 100 | QVQLVESGGGVVQPGRSLRLSCAASGFTFSSYGMHWVRQAPGKG | anti-BCMA CAR |
| | LEWVAVISYDGSNKYYADSVKGRFTISRDNSKNTLYLQMNSLRAE | |
| | DTAVYYCARTDFWSGSPPGLDYWGQGTLVTVSSGSTSGSGKPGSG | |
| | EGSTKGDIQLTQSPSSVSASVGDRVTITCRASQGISSWLAWYQQKP | |
| | GKAPKLLIYGASSLQSGVPSRFSGSGSGTDFTLTISSLQPEDFATYY | |
| | CQQIYTFPFTFGGGTKVEIKRAAALDNEKSNGTIIHVKGKHLCPSPL | |
| | FPGPSKPFWVLVVVGGVLACYSLLVTVAFIIFWVRSKRSRLLHSDY | |
| | MNMTPRRPGPTRKHYQPYAPPRDFAAYRSRVKFSRSADAPAYQQ | |
| | GQNQLYNELNLGRREEYDVLDKRRGRDPEMGGKPRRKNPQEGLY | |
| | NELQKDKMAEAYSEIGMKGERRRGKGHDGLYQGLSTATKDTYD | |
| | ALHMQALPPR | |
| 101 | DIQLTQSPSSVSASVGDRVTITCRASQGISSWLAWYQQKPGKAPKL | anti-BCMA CAR |
| | LIYGASSLQSGVPSRFSGSGSGTDFTLTISSLQPEDFATYYCQQIYTF | |
| | PFTFGGGTKVEIKRGSTSGSGKPGSGEGSTKGQVQLVESGGGVVQP | |
| | GRSLRLSCAASGFTFSSYGMHWVRQAPGKGLEWVAVISYDGSNK | |
| | YYADSVKGRFTISRDNSKNTLYLQMNSLRAEDTAVYYCARTDFW | |
| | SGSPPGLDYWGQGTLVTVSSAAALDNEKSNGTIIHVKGKHLCPSPL | |
| | FPGPSKPFWVLVVVGGVLACYSLLVTVAFIIFWVRSKRSRLLHSDY | |
| | MNMTPRRPGPTRKHYQPYAPPRDFAAYRSRVKFSRSADAPAYQQ | |
| | GQNQLYNELNLGRREEYDVLDKRRGRDPEMGGKPRRKNPQEGLY | |
| | NELQKDKMAEAYSEIGMKGERRRGKGHDGLYQGLSTATKDTYD | |
| | ALHMQALPPR | |
| 102 | QVQLVQSGAEVKKPGSSVKVSCKASGGTFSSYAISWVRQAPGQGL | anti-BCMA CAR |
| | EWMGGIIPIFGTANYAQKFQGRVTITADESTSTAYMELSSLRSEDT | |
| | AVYYCARTPEYSSSIWHYYYGMDVWGQGTTVTVSSGSTSGSGKP | |
| | GSGEGSTKGDIVMTQSPDSLAVSLGERATINCKSSQSVLYSSNNKN | |
| | YLAWYQQKPGQPPKLLIYWASTRESGVPDRFSGSGSGTDFTLTISS | |
| | LQAEDVAVYYCQQFAHTPFTFGGGTKVEIKRAAALDNEKSNGTII | |
| | HVKGKHLCPSPLFPGPSKPFWVLVVVGGVLACYSLLVTVAFIIFWV | |
| | RSKRSRLLHSDYMNMTPRRPGPTRKHYQPYAPPRDFAAYRSRVKF | |
| | SRSADAPAYQQGQNQLYNELNLGRREEYDVLDKRRGRDPEMGGK | |
| | PRRKNPQEGLYNELQKDKMAEAYSEIGMKGERRRGKGHDGLYQG | |
| | LSTATKDTYDALHMQALPPR | |
| 103 | DIVMTQSPDSLAVSLGERATINCKSSQSVLYSSNNKNYLAWYQQK | anti-BCMA CAR |
| | PGQPPKLLIYWASTRESGVPDRFSGSGSGTDFTLTISSLQAEDVAVY | |
| | YCQQFAHTPFTFGGGTKVEIKRGSTSGSGKPGSGEGSTKGQVQLV | |
| | QSGAEVKKPGSSVKVSCKASGGTFSSYAISWVRQAPGQGLEWMG | |
| | GIIPIFGTANYAQKFQGRVTITADESTSTAYMELSSLRSEDTAVYYC | |
| | ARTPEYSSSIWHYYYGMDVWGQGTTVTVSSAAALDNEKSNGTIIH | |
| | VKGKHLCPSPLFPGPSKPFWVLVVVGGVLACYSLLVTVAFIIFWVR | |
| | SKRSRLLHSDYMNMTPRRPGPTRKHYQPYAPPRDFAAYRSRVKFS | |
| | RSADAPAYQQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 104 | QVQLVESGGGVVQPGRSLRLSCAASGFTFSSYGMHWVRQAPGKG | anti-BCMA CAR |
| | LEWVAVISYDGSNKYYADSVKGRFTISRDNSKNTLYLQMNSLRAE | |
| | DTAVYYCVKGPLQEPPYDYGMDVWGQGTTVTVSSGSTSGSGKPG | |
| | SGEGSTKGEIVMTQSPATLSVSPGERATLSCRASQSVSSNLAWYQQ | |
| | KPGQAPRLLIYSASTRATGIPARFSGSGSGTEFTLTISSLQSEDFAVY | |
| | YCQQHHVWPLTFGGGTKVEIKRAAALDNEKSNGTIIHVKGKHLCP | |
| | SPLFPGPSKPFWVLVVVGGVLACYSLLVTVAFIIFWVRSKRSRLLH | |
| | SDYMNMTPRRPGPTRKHYQPYAPPRDFAAYRSRVKFSRSADAPAY | |
| | QQGQNQLYNELNLGRREEYDVLDKRRGRDPEMGGKPRRKNPQEG | |
| | LYNELQKDKMAEAYSEIGMKGERRRGKGHDGLYQGLSTATKDTY | |
| | DALHMQALPPR | |
| 105 | EIVMTQSPATLSVSPGERATLSCRASQSVSSNLAWYQQKPGQAPRL | anti-BCMA CAR |
| | LIYSASTRATGIPARFSGSGSGTEFTLTISSLQSEDFAVYYCQQHHV | |
| | WPLTFGGGTKVEIKRGSTSGSGKPGSGEGSTKGQVQLVESGGGVV | |
| | QPGRSLRLSCAASGFTFSSYGMHWVRQAPGKGLEWVAVISYDGS | |
| | NKYYADSVKGRFTISRDNSKNTLYLQMNSLRAEDTAVYYCVKGP | |
| | LQEPPYDYGMDVWGQGTTVTVSSAAALDNEKSNGTIIHVKGKHL | |
| | CPSPLFPGPSKPFWVLVVVGGVLACYSLLVTVAFIIFWVRSKRSRL | |
| | LHSDYMNMTPRRPGPTRKHYQPYAPPRDFAAYRSRVKFSRSADAP | |
| | AYQQGQNQLYNELNLGRREEYDVLDKRRGRDPEMGGKPRRKNP | |
| | QEGLYNELQKDKMAEAYSEIGMKGERRRGKGHDGLYQGLSTATK | |
| | DTYDALHMQALPPR | |
| 106 | QSALTQPASVSASPGQSIAISCTGTSSDVGWYQQHPGKAPKLMIYE | anti-BCMA CAR |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSSAAAIEVMYPPPYLDNEKSNGTIIHVK | |
| | GKHLCPSPLFPGPSKPFWVLVVVGGVLACYSLLVTVAFIIFWVRSK | |
| | RSRLLHSDYMNMTPRRPGPTRKHYQPYAPPRDFAAYRSRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 107 | QSVLTQPPSVSGAPGQRVTISCTGSSSNIGAGFDVHWYQQLPGTAP | anti-BCMA CAR |
| | KLLIYGNSNRPSGVPDRFSGSKSGTSASLAITGLQAEDEADYYCQS | |
| | YDSSLSGYVFGTGTKVTVLGSRGGGGSGGGGSGGGGSLEMAQVQ | |
| | LVQSGAEVKKPGASVKVSCKASGYTFTDYYMHWVRQAPGQRLE | |
| | WMGWINPNSGGTNYAQKFQDRITVTRDTSSNTGYMELTRLRSDD | |
| | TAVYYCARSPYSGVLDKWGQGTLVTVSSAAAIEVMYPPPYLDNE | |
| | KSNGTIIHVKGKHLCPSPLFPGPSKPFWVLVVVGGVLACYSLLVTV | |
| | AFIIFWVRSKRSRLLHSDYMNMTPRRPGPTRKHYQPYAPPRDFAA | |
| | YRSRVKFSRSADAPAYQQGQNQLYNELNLGRREEYDVLDKRRGR | |
| | DPEMGGKPRRKNPQEGLYNELQKDKMAEAYSEIGMKGERRRGKG | |
| | HDGLYQGLSTATKDTYDALHMQALPPR | |
| 108 | SYELTQPPSASGTPGQRVTMSCSGTSSNIGSHSVNWYQQLPGTAPK | anti-BCMA CAR |
| | LLIYTNNQRPSGVPDRFSGSKSGTSASLAISGLQSEDEADYYCAAW | |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMYY | |
| | CARYSGSFDNWGQGTLVTVSSAAAIEVMYPPPYLDNEKSNGTIIHV | |
| | KGKHLCPSPLFPGPSKPFWVLVVVGGVLACYSLLVTVAFIIFWVRS | |
| | KRSRLLHSDYMNMTPRRPGPTRKHYQPYAPPRDFAAYRSRVKFSR | |
| | SADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 109 | LPVLTQPPSASGTPGQRVTISCSGRSSNIGSNSVNWYRQLPGAAPKL | anti-BCMA CAR |
| | LIYSNNQRPPGVPVRFSGSKSGTSASLAISGLQSEDEATYYCATWD | |
| | DNLNVHYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGIANYAQKFQGRVTITADKSTSTAYMELSSLRSEDTAVY | |
| | YCARGGYYSHDMWSEDWGQGTLVTVSSAAAIEVMYPPPYLDNE | |
| | KSNGTIIHVKGKHLCPSPLFPGPSKPFWVLVVVGGVLACYSLLVTV | |
| | AFIIFWVRSKRSRLLHSDYMNMTPRRPGPTRKHYQPYAPPRDFAA | |
| | YRSRVKFSRSADAPAYQQGQNQLYNELNLGRREEYDVLDKRRGR | |
| | DPEMGGKPRRKNPQEGLYNELQKDKMAEAYSEIGMKGERRRGKG | |
| | HDGLYQGLSTATKDTYDALHMQALPPR | |
| 110 | QAVLTQPPSASGTPGQRVTISCSGSSSNIGSNYVFWYQQLPGTAPK | anti-BCMA CAR |
| | LLIYSNNQRPSGVPDRFSGSKSGTSASLAISGLRSEDEADYYCAAW | |
| | DDSLSASYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGTANYAQKFQGRVTITADESTSTAYMELSSLRSEDTAVY | |
| | YCARSGYGSYRWEDSWGQGTLVTVSSAAAIEVMYPPPYLDNEKS | |
| | NGTIIHVKGKHLCPSPLFPGPSKPFWVLVVVGGVLACYSLLVTVAF | |
| | IIFWVRSKRSRLLHSDYMNMTPRRPGPTRKHYQPYAPPRDFAAYR | |
| | SRVKFSRSADAPAYQQGQNQLYNELNLGRREEYDVLDKRRGRDP | |
| | EMGGKPRRKNPQEGLYNELQKDKMAEAYSEIGMKGERRRGKGH | |
| | DGLYQGLSTATKDTYDALHMQALPPR | |
| 111 | LPVLTQPPSASGTPGQRVTISCSGRSSNIGSNSVNWYRQLPGAAPKL | anti-BCMA CAR |
| | LIYSNNQRPPGVPVRFSGSKSGTSASLAISGLQSEDEATYYCATWD | |
| | DNLNVHYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGIANYAQKFQGRVTITADKSTSTAYMELSSLRSEDTAVY | |
| | YCARGGYYSHDMWSEDWGQGTLVTVSSAAAPTTTPAPRPPTPAP | |
| | TIASQPLSLRPEACRPAAGGAVHTRGLDFACDIYIWAPLAGTCGVL | |
| | LLSLVITLYCNKRGRKKLLYIFKQPFMRPVQTTQEEDGCCRFPEEE | |
| | EGGCELRVKFSRSAEPPAYQQGQNQLYNELNLGRREEYDVLDKRR | |
| | GRDPEMGGKPRRKNPQEGLYNELQKDKMAEAYSEIGMKGERRRG | |
| | KGHDGLYQGLSTATKDTYDALHMQALPPR | |
| 112 | SYELTQPPSASGTPGQRVTMSCSGTSSNIGSHSVNWYQQLPGTAPK | anti-BCMA CAR |
| | LLIYTNNQRPSGVPDRFSGSKSGTSASLAISGLQSEDEADYYCAAW | |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMYY | |
| | CARYSGSFDNWGQGTLVTVSSAAAPTTTPAPRPPTPAPTIASQPLSL | |
| | RPEACRPAAGGAVHTRGLDFACDIYIWAPLAGTCGVLLLSLVITLY | |
| | CNKRGRKKLLYIFKQPFMRPVQTTQEEDGCSCRFPEEEEGGCELRV | |
| | KFSRSAEPPAYQQGQNQLYNELNLGRREEYDVLDKRRGRDPEMG | |
| | GKPRRKNPQEGLYNELQKDKMAEAYSEIGMKGERRRGKGHDGLY | |
| | QGLSTATKDTYDALHMQALPPR | |
| 113 | QAVLTQPPSASGTPGQRVTISCSGSSSNIGSNYVFWYQQLPGTAPK | anti-BCMA CAR |
| | LLIYSNNQRPSGVPDRFSGSKSGTSASLAISGLRSEDEADYYCAAW | |
| | DDSLSASYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGTANYAQKFQGRVTITADESTSTAYMELSSLRSEDTAVY | |
| | YCARSGYGSYRWEDSWGQGTLVTVSSAAAPTTTPAPRPPTPAPTIA | |
| | SQPLSLRPEACRPAAGGAVHTRGLDFACDIYIWAPLAGTCGVLLLS | |
| | LVITLYCNKRGRKKLLYIFKQPFMRPVQTTQEEDGCSCRFPEEEEG | |
| | GCELRVKFSRSAEPPAYQQGQNQLYNELNLGRREEYDVLDKRRG | |
| | RDPEMGGKPRRKNPQEGLYNELQKDKMAEAYSEIGMKGERRRGK | |
| | GHDGLYQGLSTATKDTYDALHMQALPPR | |
| 114 | QSVLTQPPSVSGAPGQRVTISCTGSSSNIGAGFDVHWYQQLPGTAP | anti-BCMA CAR |
| | KLLIYGNSNRPSGVPDRFSGSKSGTSASLAITGLQAEDEADYYCQS | |
| | YDSSLSGYVFGTGTKVTVLGSRGGGGSGGGGSGGGGSLEMAQVQ | |
| | LVQSGAEVKKPGASVKVSCKASGYTFTDYYMHWVRQAPGQRLE | |
| | WMGWINPNSGGTNYAQKFQDRITVTRDTSSNTGYMELTRLRSDD | |
| | TAVYYCARSPYSGVLDKWGQGTLVTVSSAAAPTTTPAPRPPTPAP | |
| | TIASQPLSLRPEACRPAAGGAVHTRGLDFACDIYIWAPLAGTCGVL | |
| | LLSLVITLYCNKRGRKKLLYIFKQPFMRPVQTTQEEDGCSCRFPEEE | |
| | EGGCELRVKFSRSAEPPAYQQGQNQLYNELNLGRREEYDVLDKRR | |
| | GRDPEMGGKPRRKNPQEGLYNELQKDKMAEAYSEIGMKGERRRG | |
| | KGHDGLYQGLSTATKDTYDALHMQALPPR | |
| 115 | QSALTQPASVSASPGQSIAISCTGTSSDVGWYQQHPGKAPKLMIYE | anti-BCMA CAR |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSSAAAPTTTPAPRPPTPAPTIASQPLSLR | |
| | PEACRPAAGGAVHTRGLDFACDIYIWAPLAGTCGVLLLSLVITLYC | |
| | NKRGRKKLLYIFKQPFMRPVQTTQEEDGCSCRFPEEEEGGCELRVK | |
| | FSRSAEPPAYQQGQNQLYNELNLGRREEYDVLDKRRGRDPEMGG | |
| | KPRRKNPQEGLYNELQKDKMAEAYSEIGMKGERRRGKGHDGLYQ | |
| | GLSTATKDTYDALHMQALPPR | |
| 116 | DIVLTQSPPSLAMSLGKRATISCRASESVTILGSHLIHWYQQKPGQP | anti-BCMA CAR |
| | PTLLIQLASNVQTGVPARFSGSGSRTDFTLTIDPVEEDDVAVYYCL | |
| | QSRTIPRTFGGGTKLEIKGSTSGSGKPGSGEGSTKGQIQLVQSGPEL | |
| | KKPGETVKISCKASGYTFTDYSINWVKRAPGKGLKWMGWINTET | |
| | REPAYAYDFRGRFAFSLETSASTAYLQINNLKYEDTATYFCALDYS | |
| | YAMDYWGQGTSVTVSSAAATTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 117 | DIVLTQSPASLAVSLGERATINCRASESVSVIGAHLIHWYQQKPGQP | anti-BCMA CAR |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSSAAATTTPAPRPPTPAPTIASQPLSLRPEACR | |
| | PAAGGAVHTRGLDFACDIYIWAPLAGTCGVLLLSLVITLYCKRGR | |
| | KKLLYIFKQPFMRPVQTTQEEDGCSCRFPEEEEGGCELRVKFSRSA | |
| | DAPAYQQGQNQLYNELNLGRREEYDVLDKRRGRDPEMGGKPRR | |
| | KNPQEGLYNELQKDKMAEAYSEIGMKGERRRGKGHDGLYQGLST | |
| | ATKDTYDALHMQALPPR | |
| 118 | DIVLTQSPASLAVSLGERATINCRASESVSVIGAHLIHWYQQKPGQP | anti-BCMA CAR |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSSAAADTGLYICKVELMYPPPYYLGIGNGTQI | |
| | YVIDPEPCPDSDFLLWILAAVSSGLFFYSFLLTAVSKRGRKKLLYIF | |
| | KQPFMRPVQTTQEEDGCSCRFPEEEEGGCELRVKFSRSADAPAYQ | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 119 | DIVLTQSPASLAVSLGERATINCRASESVSVIGAHLIHWYQQKPGQP | anti-BCMA CAR |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSSAAAQIKESLRAELRVTERRAEVPTAHPSPS | |
| | PRPAGQFQTLVVGVVGGLLGSLVLLVWVLAVICSKRGRKKLLYIF | |
| | KQPFMRPVQTTQEEDGCSCRFPEEEEGGCELRVKFSRSADAPAYQ | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 120 | EVQLVESGGGLVKPGGSLRLSCAASGFTFSDYYMSWIRQAPGKGL | anti-BCMA CAR |
| | EWVSYISSSGSTIYYADSVKGRFTISRDNAKNSLYLQMNSLRAEDT | |
| | AVYYCAKVDGDYTEDYWGQGTLVTVSSGGGGSGGGGSGGGGSQ | |
| | SALTQPASVSGSPGQSITISCTGSSSDVGKYNLVSWYQQPPGKAPK | |
| | LIIYDVNKRPSGVSNRFSGSKSGNTATLTISGLQGDDEADYYCSSY | |
| | GGSRSYVFGTGTKVTVLESKYGPPCPPCPAPPVAGPSVFLFPPKPK | |
| | DTLMISRTPEVTCVVVDVSQEDPEVQFNWYVDGVEVHNAKTKPR | |
| | EEQFQSTYRVVSVLTVLHQDWLNGKEYKCKVSNKGLPSSIEKTISK | |
| | AKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVM | |
| | HEALHNHYTQKSLSLSLGKMFWVLVVVGGVLACYSLLVTVAFIIF | |
| | WVKRGRKKLLYIFKQPFMRPVQTTQEEDGCSCRFPEEEEGGCELR | |
| | VKFSRSADAPAYQQGQNQLYNELNLGRREEYDVLDKRRGRDPEM | |
| | GGKPRRKNPQEGLYNELQKDKMAEAYSEIGMKGERRRGKGHDGL | |
| | YQGLSTATKDTYDALHMQALPPR | |
| 121 | EVQLVQSGGGLVQPGRSLRLSCTASGFTFGDYAMSWFKQAPGKG | anti-BCMA CAR |
| | LEWVGFIRSKAYGGTTEYAASVKGRFTISRDDSKSIAYLQMNSLKT | |
| | EDTAVYYCAAWSAPTDYWGQGTLVTVSSGGGGSGGGGSGGGGS | |
| | DIQMTQSPAFLSASVGDRVTVTCRASQGISNYLAWYQQKPGNAPR | |
| | LLIYSASTLQSGVPSRFRGTGYGTEFSLTIDSLQPEDFATYYCQQSY | |
| | TSRQTFGPGTRLDIKESKYGPPCPPCPAPPVAGPSVFLFPPKPKDTL | |
| | MISRTPEVTCVVVDVSQEDPEVQFNWYVDGVEVHNAKTKPREEQ | |
| | FQSTYRVVSVLTVLHQDWLNGKEYKCKVSNKGLPSSIEKTISKAK | |
| | GQPREPQVYTLPPSQEEMTKNQVSLTCLVKGFYPSDIAVEWESNG | |
| | QPENNYKTTPPVLDSDGSFFLYSRLTVDKSRWQEGNVFSCSVMHE | |
| | ALHNHYTQKSLSLSLGKMFWVLVVVGGVLACYSLLVTVAFIIFWV | |
| | KRGRKKLLYIFKQPFMRPVQTTQEEDGCSCRFPEEEEGGCELRVKF | |
| | SRSADAPAYQQGQNQLYNELNLGRREEYDVLDKRRGRDPEMGGK | |
| | PRRKNPQEGLYNELQKDKMAEAYSEIGMKGERRRGKGHDGLYQG | |
| | LSTATKDTYDALHMQALPPR | |
| 122 | EVQLVESGGGLVKPGGSLRLSCAASGFTFSDYYMSWIRQAPGKGL | anti-BCMA CAR |
| | EWVSYISSSGSTIYYADSVKGRFTISRDNAKNSLYLQMNSLRAEDT | |
| | AVYYCAKVDGPPSFDIWGQGTMVTVSSGGGGSGGGGSGGGGSSY | |
| | VLTQPPSVSVAPGQTARITCGANNIGSKSVHWYQQKPGQAPMLVV | |
| | YDDDDRPSGIPERFSGSNSGNTATLTISGVEAGDEADYFCHLWDRS | |
| | RDHYVFGTGTKLTVLESKYGPPCPPCPAPPVAGPSVFLFPPKPKDT | |
| | LMISRTPEVTCVVVDVSQEDPEVQFNWYVDGVEVHNAKTKPREE | |
| | QFQSTYRVVSVLTVLHQDWLNGKEYKCKVSNKGLPSSIEKTISKA | |
| | KGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMH | |
| | EALHNHYTQKSLSLSLGKMFWVLVVVGGVLACYSLLVTVAFIIFW | |
| | VKRGRKKLLYIFKQPFMRPVQTTQEEDGCSCRFPEEEEGGCELRVK | |
| | FSRSADAPAYQQGQNQLYNELNLGRREEYDVLDKRRGRDPEMGG | |
| | KPRRKNPQEGLYNELQKDKMAEAYSEIGMKGERRRGKGHDGLYQ | |
| | GLSTATKDTYDALHMQALPPR | |
| 123 | SYELTQPPSASGTPGQRVTMSCSGTSSNIGSHSVNWYQQLPGTAPK | anti-BCMA CAR |
| | LLIYTNNQRPSGVPDRFSGSKSGTSASLAISGLQSEDEADYYCAAW | |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMYY | |
| | CARYSGSFDNWGQGTLVTVSSESKYGPPCPPCPAPPVAGPSVFLFP | |
| | PKPKDTLMISRTPEVTCVVVDVSQEDPEVQFNWYVDGVEVHNAK | |
| | TKPREEQFQSTYRVVSVLTVLHQDWLNGKEYKCKVSNKGLPSSIE | |
| | KTISKAKGQPREPQVYTLPPSQEEMTKNQVSLTCLVKGFYPSDIAV | |
| | EWESNGQPENNYKTTPPVLDSDGSFFLYSRLTVDKSRWQEGNVFS | |
| | CSVMHEALHNHYTQKSLSLSLGKMFWVLVVVGGVLACYSLLVTV | |
| | AFIIFWVKRGRKKLLYIFKQPFMRPVQTTQEEDGCSCRFPEEEEGG | |
| | CELRVKFSRSADAPAYQQGQNQLYNELNLGRREEYDVLDKRRGR | |
| | DPEMGGKPRRKNPQEGLYNELQKDKMAEAYSEIGMKGERRRGKG | |
| | HDGLYQGLSTATKDTYDALHMQALPPR | |
| 124 | QSALTQPASVSASPGQSIAISCTGTSSDVGWYQQHPGKAPKLMIYE | anti-BCMA CAR |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSSESKYGPPCPPCPAPPVAGPSVFLFPP | |
| | KPKDTLMISRTPEVTCVVVDVSQEDPEVQFNWYVDGVEVHNAKT | |
| | KPREEQFQSTYRVVSVLTVLHQDWLNGKEYKCKVSNKGLPSSIEK | |
| | TISKAKGQPREPQVYTLPPSQEEMTKNQVSLTCLVKGFYPSDIAVE | |
| | WESNGQPENNYKTTPPVLDSDGSFFLYSRLTVDKSRWQEGNVFSC | |
| | SVMHEALHNHYTQKSLSLSLGKMFWVLVVVGGVLACYSLLVTVA | |
| | FIIFWVKRGRKKLLYIFKQPFMRPVQTTQEEDGCSCRFPEEEEGGCE | |
| | LRVKFSRSADAPAYQQGQNQLYNELNLGRREEYDVLDKRRGRDP | |
| | EMGGKPRRKNPQEGLYNELQKDKMAEAYSEIGMKGERRRGKGH | |
| | DGLYQGLSTATKDTYDALHMQALPPR | |
| 125 | QSALTQPASVSASPGQSIAISCTGTSSDVGWYQQHPGKAPKLMIYE | anti-BCMA CAR |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSSESKYGPPCPPCPAPPVAGPSVFLFPP | |
| | KPKDTLMISRTPEVTCVVVDVSQEDPEVQFNWYVDGVEVHNAKT | |
| | KPREEQFQSTYRVVSVLTVLHQDWLNGKEYKCKVSNKGLPSSIEK | |
| | TISKAKGQPREPQVYTLPPSQEEMTKNQVSLTCLVKGFYPSDIAVE | |
| | WESNGQPENNYKTTPPVLDSDGSFFLYSRLTVDKSRWQEGNVFSC | |
| | SVMHEALHNHYTQKSLSLSLGKMFWVLVVVGGVLACYSLLVTVA | |
| | FIIFWVRSKRSRLLHSDYMNMTPRRPGPTRKHYQPYAPPRDFAAY | |
| | RSRVKFSRSADAPAYQQGQNQLYNELNLGRREEYDVLDKRRGRD | |
| | PEMGGKPRRKNPQEGLYNELQKDKMAEAYSEIGMKGERRRGKGH | |
| | DGLYQGLSTATKDTYDALHMQALPPR | |
| 126 | EVQLVESGGGLVQPGGSLRLSCAVSGFALSNHGMSWVRRAPGKG | anti-BCMA CAR |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSDIQ | |
| | LTQSPSSLSASVGDRVTITCRASQSISSYLNWYQQKPGKAPKLLIYA | |
| | ASSLQSGVPSRFSGSGSGTDFTLTISSLQPEDFATYYCQQSYSTPYTF | |
| | GQGTKVEIKTTTPAPRPPTPAPTIASQPLSLRPEACRPAAGGAVHTR | |
| | GLDFACDIYIWAPLAGTCGVLLLSLVITLYCKRGRKKLLYIFKQPF | |
| | MRPVQTTQEEDGCSCRFPEEEEGGCELRVKFSRSADAPAYKQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALH | |
| | MQALPPR | |
| 127 | QVQLVESGGGLVQPGRSLRLSCAASGFTFSNYAMSWVRQAPGKG | anti-BCMA CAR |
| | LGWVSGISRSGENTYYADSVKGRFTISRDNSKNTLYLQMNSLRDE | |
| | DTAVYYCARSPAHYYGGMDVWGQGTTVTVSSASGGGGSGGRAS | |
| | GGGGSDIVLTQSPGTLSLSPGERATLSCRASQSISSSFLAWYQQKPG | |
| | QAPRLLIYGASRRATGIPDRFSGSGSGTDFTLTISRLEPEDSAVYYC | |
| | QQYHSSPSWTFGQGTKLEIKTTTPAPRPPTPAPTIASQPLSLRPEACR | |
| | PAAGGAVHTRGLDFACDIYIWAPLAGTCGVLLLSLVITLYCKRGR | |
| | KKLLYIFKQPFMRPVQTTQEEDGCSCRFPEEEEGGCELRVKFSRSA | |
| | DAPAYKQGQNQLYNELNLGRREEYDVLDKRRGRDPEMGGKPRR | |
| | KNPQEGLYNELQKDKMAEAYSEIGMKGERRRGKGHDGLYQGLST | |
| | ATKDTYDALHMQALPPR | |
| 128 | QVQLVESGGGLVQPGGSLRLSCAVSGFALSNHGMSWVRRAPGKG | anti-BCMA CAR |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSDIR | |
| | LTQSPSPLSASVGDRVTITCQASEDINKFLNWYHQTPGKAPKLLIY | |
| | DASTLQTGVPSRFSGSGSGTDFTLTINSLQPEDIGTYYCQQYESLPL | |
| | TFGGGTKVEIKTTTPAPRPPTPAPTIASQPLSLRPEACRPAAGGAVH | |
| | TRGLDFACDIYIWAPLAGTCGVLLLSLVITLYCKRGRKKLLYIFKQ | |
| | PFMRPVQTTQEEDGCSCRFPEEEEGGCELRVKFSRSADAPAYKQG | |
| | QNQLYNELNLGRREEYDVLDKRRGRDPEMGGKPRRKNPQEGLYN | |
| | ELQKDKMAEAYSEIGMKGERRRGKGHDGLYQGLSTATKDTYDAL | |
| | HMQALPPR | |
| 129 | EVQLVESGGGLVQPGGSLRLSCAVSGFALSNHGMSWVRRAPGKG | anti-BCMA CAR |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSEIV | |
| | LTQSPGTLSLSPGERATLSCRASQSIGSSSLAWYQQKPGQAPRLLM | |
| | YGASSRASGIPDRFSGSGSGTDFTLTISRLEPEDFAVYYCQQYAGSP | |
| | PFTFGQGTKVEIKTTTPAPRPPTPAPTIASQPLSLRPEACRPAAGGA | |
| | VHTRGLDFACDIYIWAPLAGTCGVLLLSLVITLYCKRGRKKLLYIF | |
| | KQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 130 | QIQLVQSGPDLKKPGETVKLSCKASGYTFTNFGMNWVKQAPGKG | anti-BCMA CAR |
| | FKWMAWINTYTGESYFADDFKGRFAFSVETSATTAYLQINNLKTE | |
| | DTATYFCARGEIYYGYDGGFAYWGQGTLVTVSAGGGGSGGGGSG | |
| | GGGSDVVMTQSHRFMSTSVGDRVSITCRASQDVNTAVSWYQQKP | |
| | GQSPKLLIFSASYRYTGVPDRFTGSGSGADFTLTISSVQAEDLAVYY | |
| | CQQHYSTPWTFGGGTKLDIKTTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRS | |
| | ADAPAYKQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 131 | QIQLVQSGPELKKPGETVKISCKASGYTFTDYSINWVKRAPGKGLK | anti-BCMA CAR |
| | WMGWINTETREPAYAYDFRGRFAFSLETSASTAYLQINNLKYEDT | |
| | ATYFCALDYSYAMDYWGQGTSVTVSSGGGGSGGGGSGGGGSQIQ | |
| | LVQSGPELKKPGETVKISCKASGYTFTDYSINWVKRAPGKGLKWM | |
| | GWINTETREPAYAYDFRGRFAFSLETSASTAYLQINNLKYEDTATY | |
| | FCALDYSYAMDYWGQGTSVTVSSTTTPAPRPPTPAPTIASQPLSLR | |
| | PEACRPAAGGAVHTRGLDFACDIYIWAPLAGTCGVLLLSLVITLYC | |
| | KRGRKKLLYIFKQPFMRPVQTTQEEDGCSCRFPEEEEGGCELRVKF | |
| | SRSADAPAYKQGQNQLYNELNLGRREEYDVLDKRRGRDPEMGGK | |
| | PRRKNPQEGLYNELQKDKMAEAYSEIGMKGERRRGKGHDGLYQG | |
| | LSTATKDTYDALHMQALPPR | |
| 132 | QIQLVQSGPELKKPGETVKISCKASGYTFTDYSINWVKRAPGKGLK | anti-BCMA CAR |
| | WMGWINTETREPAYAYDFRGRFAFSLETSASTAYLQINNLKYEDT | |
| | ATYFCALDYSYAMDYWGQGTSVTVSSGGGGSGGGGSGGGGSDIV | |
| | LTQSPASLAMSLGKRATISCRASESVSVIGAHLIHWYQQKPGQPPK | |
| | LLIYLASNLETGVPARFSGSGSGTDFTLTIDPVEEDDVAIYSCLQSRI | |
| | FPRTFGGGTKLEIKTTTPAPRPPTPAPTIASQPLSLRPEACRPAAGGA | |
| | VHTRGLDFACDIYIWAPLAGTCGVLLLSLVITLYCKRGRKKLLYIF | |
| | KQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 133 | QIQLVQSGPELKKPGETVKISCKASGYTFRHYSMNWVKQAPGKGL | anti-BCMA CAR |
| | KWMGRINTESGVPIYADDFKGRFAFSVETSASTAYLVINNLKDEDT | |
| | ASYFCSNDYLYSLDFWGQGTALTVSSGGGGSGGGGSGGGGSDIVL | |
| | TQSPPSLAMSLGKRATISCRASESVTILGSHLIYWYQQKPGQPPTLL | |
| | IQLASNVQTGVPARFSGSGSRTDFTLTIDPVEEDDVAVYYCLQSRTI | |
| | PRTFGGGTKLEIKTTTPAPRPPTPAPTIASQPLSLRPEACRPAAGGA | |
| | VHTRGLDFACDIYIWAPLAGTCGVLLLSLVITLYCKRGRKKLLYIF | |
| | KQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 134 | QIQLVQSGPELKKPGETVKISCKASGYTFTHYSMNWVKQAPGKGL | anti-BCMA CAR |
| | KWMGRINTETGEPLYADDFKGRFAFSLETSASTAYLVINNLKNED | |
| | TATFFCSNDYLYSCDYWGQGTTLTVSSGGGGSGGGGSGGGGSDIV | |
| | LTQSPPSLAMSLGKRATISCRASESVTILGSHLIYWYQQKPGQPPTL | |
| | LIQLASNVQTGVPARFSGSGSRTDFTLTIDPVEEDDVAVYYCLQSR | |
| | TIPRTFGGGTKLEIKTTTPAPRPPTPAPTIASQPLSLRPEACRPAAGG | |
| | AVHTRGLDFACDIYIWAPLAGTCGVLLLSLVITLYCKRGRKKLLYI | |
| | FKQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 135 | DIVLTQSPPSLAMSLGKRATISCRASESVTILGSHLIHWYQQKPGQP | anti-BCMA CAR |
| | PTLLIQLASNVQTGVPARFSGSGSRTDFTLTIDPVEEDDVAVYYCL | |
| | QSRTIPRTFGGGTKLEIKGSTSGSGKPGSGEGSTKGQIQLVQSGPEL | |
| | KKPGETVKISCKASGYTFTDYSINWVKRAPGKGLKWMGWINTET | |
| | REPAYAYDFRGRFAFSLETSASTAYLQINNLKYEDTATYFCALDYS | |
| | YAMDYWGQGTSVTVSSFVPVFLPAKPTTTPAPRPPTPAPTIASQPLS | |
| | LRPEACRPAAGGAVHTRGLDFACDIYIWAPLAGTCGVLLLSLVITL | |
| | YCNHRNRSKRSRLLHSDYMNMTPRRPGPTRKHYQPYAPPRDFAA | |
| | YRSRVKFSRSADAPAYQQGQNQLYNELNLGRREEYDVLDKRRGR | |
| | DPEMGGKPRRKNPQEGLYNELQKDKMAEAYSEIGMKGERRRGKG | |
| | HDGLYQGLSTATKDTYDALHMQALPPR | |
| 136 | QVQLVQSGAEVKKPGASVKVSCKASGYSFPDYYINWVRQAPGQG | anti-BCMA CAR |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIKGLAVSTISSFFPPGYQIYIWAPLAGTC | |
| | GVLLLSLVITLYCKRGRKKLLYIFKQPFMRPVQTTQEEDGCSCRFP | |
| | EEEEGGCELRVKFSRSADAPAYQQGQNQLYNELNLGRREEYDVL | |
| | DKRRGRDPEMGGKPRRKNPQEGLYNELQKDKMAEAYSEIGMKGE | |
| | RRRGKGHDGLYQGLSTATKDTYDALHMQALPPR | |
| 137 | QVQLVQSGAEVKKPGASVKVSCKASGYSFPDYYINWVRQAPGQG | anti-BCMA CAR |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIKTTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGLS | |
| | TATKDTYDALHMQALPPR | |
| 138 | QVQLVQSGAEVKKPGASVKVSCKASGYSFPDYYINWVRQAPGQG | anti-BCMA CAR |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIKEPKSPDKTHTCPPCPAPPVAGPSVFL | |
| | FPPKPKDTLMIARTPEVTCVVVDVSHEDPEVKFNWYVDGVEVHN | |
| | AKTKPREEQYNSTYRVVSVLTVLHQDWLNGKEYKCKVSNKALPA | |
| | PIEKTISKAKGQPREPQVYTLPPSRDELTKNQVSLTCLVKGFYPSDI | |
| | AVEWESNGQPENNYKTTPPVLDSDGSFFLYSKLTVDKSRWQQGN | |
| | VFSCSVMHEALHNHYTQKSLSLSPGKIYIWAPLAGTCGVLLLSLVI | |
| | TLYCKRGRKKLLYIFKQPFMRPVQTTQEEDGCSCRFPEEEEGGCEL | |
| | RVKFSRSADAPAYQQGQNQLYNELNLGRREEYDVLDKRRGRDPE | |
| | MGGKPRRKNPQEGLYNELQKDKMAEAYSEIGMKGERRRGKGHD | |
| | GLYQGLSTATKDTYDALHMQALPPR | |
| 139 | QVQLVQSGAEVKKPGASVKVSCKASGYSFPDYYINWVRQAPGQG | anti-BCMA CAR |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIKGLAVSTISSFFPPGYQIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMKG | |
| | ERRRGKGHDGLYQGLSTATKDTYDALHMQALPPR | |
| 140 | QVQLVQSGAEVKKPGASVKVSCKASGYSFPDYYINWVRQAPGQG | anti-BCMA CAR |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIKTTTPAPRPPTPAPTIASQPLSLRPE | |
| | ACRPAAGGAVHTRGLDFACDIYIWAPLAGTCGVLLLSLVITLYCK | |
| | RGRKKLLYIFKQPFMRPVQTTQEEDGCSCRFPEEEEGGCELRVKFS | |
| | RSADAPAYQQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 141 | QVQLVQSGAEVKKPGASVKVSCKASGYSFPDYYINWVRQAPGQG | anti-BCMA CAR |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIKEPKSPDKTHTCPPCPAPPVAGPSV | |
| | FLFPPKPKDTLMIARTPEVTCVVVDVSHEDPEVKFNWYVDGVEVH | |
| | NAKTKPREEQYNSTYRVVSVLTVLHQDWLNGKEYKCKVSNKALP | |
| | APIEKTISKAKGQPREPQVYTLPPSRDELTKNQVSLTCLVKGFYPSD | |
| | IAVEWESNGQPENNYKTTPPVLDSDGSFFLYSKLTVDKSRWQQGN | |
| | VFSCSVMHEALHNHYTQKSLSLSPGKIYIWAPLAGTCGVLLLSLVI | |
| | TLYCKRGRKKLLYIFKQPFMRPVQTTQEEDGCSCRFPEEEEGGCEL | |
| | RVKFSRSADAPAYQQGQNQLYNELNLGRREEYDVLDKRRGRDPE | |
| | MGGKPRRKNPQEGLYNELQKDKMAEAYSEIGMKGERRRGKGHD | |
| | GLYQGLSTATKDTYDALHMQALPPR | |
| 142 | IYIWAPLAGTCGVLLLSLVITLYCNHRN | CD8a TM |
| 143 | IYIWAPLAGTCGVLLLSLVIT | CD8a TM |
| 144 | RAAA | linking peptide |
| 145 | EVQLVESGGGLVKPGGSLRLSCAASGFTFSDYYMSWIRQAPGKGL | Variable heavy |
| | EWVSYISSSGSTIYYADSVKGRFTISRDNAKNSLYLQMNSLRAEDT | (VH) Anti- |
| | AVYYCAKVDGDYTEDYWGQGTLVTVSS | BCMA |
| 146 | QSALTQPASVSGSPGQSITISCTGSSSDVGKYNLVSWYQQPPGKAP | Variable light |
| | KLIIYDVNKRPSGVSNRFSGSKSGNTATLTISGLQGDDEADYYCSS | (VL) Anti- |
| | YGGSRSYVFGTGTKVTVL | BCMA |
| 147 | EVQLVQSGGGLVQPGRSLRLSCTASGFTFGDYAMSWFRQAPGKG | Variable heavy |
| | LEWVGFIRSKAYGGTTEYAASVKGRFTISRDDSKSIAYLQMNSLKT | (VH) Anti- |
| | EDTAVYYCAAWSAPTDYWGQGTLVTVSS | BCMA |
| 148 | DIQMTQSPAFLSASVGDRVTVTCRASQGISNYLAWYQQKPGNAPR | Variable light |
| | LLIYSASTLQSGVPSRFRGTGYGTEFSLTIDSLQPEDFATYYCQQSY | (VL) Anti- |
| | TSRQTFGPGTRLDIK | BCMA |
| 149 | EVQLVESGGGLVKPGGSLRLSCAASGFTFSDYYMSWIRQAPGKGL | Variable heavy |
| | EWVSYISSSGSTIYYADSVKGRFTISRDNAKNSLYLQMNSLRAEDT | (VH) Anti- |
| | AVYYCAKVDGPPSFDIWGQGTMVTVSS | BCMA |
| 150 | SYVLTQPPSVSVAPGQTARITCGANNIGSKSVHWYQQKPGQAPML | Variable light |
| | VVYDDDDRPSGIPERFSGSNSGNTATLTISGVEAGDEADYFCHLW | (VL) Anti- |
| | DRSRDHYVFGTGTKLTVL | BCMA |
| 151 | EVQLVQSGAEVKKPGSSVKVSCKASGGTFSSYAISWVRQAPGQGL | Variable heavy |
| | EWMGRIIPILGIANYAQKFQGRVTMTEDTSTDTAYMELSSLRSEDT | (VH) Anti- |
| | AVYYCARSGYSKSIVSYMDYWGQGTLVTVSS | BCMA |
| 152 | LPVLTQPPSTSGTPGQRVTVSCSGSSSNIGSNVVFWYQQLPGTAPK | Variable light |
| | LVIYRNNQRPSGVPDRFSVSKSGTSASLAISGLRSEDEADYYCAAW | (VL) Anti- |
| | DDSLSGYVFGTGTKVTVLG | BCMA |
| 153 | MPLLLLLPLLWAGALA | CD33 Signal |
| | | peptide |
| 154 | MALPVTALLLPLALLLHA | CD8 alpha signal |
| | | peptide |
| 155 | atgcttctcctggtgacaagccttctgctctgtgagttaccacacccagcattc | GMCSFR alpha |
| | ctcctgatccca | chain signal |
| | | sequence |
| 156 | MLLLVTSLLLCELPHPAFLLIP | GMCSFR alpha |
| | | chain signal |
| | | sequence |
| 157 | Glu Val Val Val Lys Tyr Gly Pro Pro Cys Pro Pro Cys | Exemplary IgG |
| | Pro | Hinge |
| 158 | X1PPX2P | Exemplary IgG |
| | X1 is glycine, cysteine or arginine | Hinge |
| | X2 is cysteine or threonine | |
| 159 | Glu Pro Lys Ser Cys Asp Lys Thr His Thr Cys Pro Pro | Exemplary IgG |
| | Cys Pro | Hinge |
| 160 | Glu Arg Lys Cys Cys Val Glu Cys Pro Pro Cys Pro | Exemplary IgG |
| | | Hinge |
| 161 | ELKTPLGDTHTCPRCPEPKSCDTPPPCPRCPEPKSCDTPPPCPRCPEP | Exemplary IgG |
| | KSCDTPPPCPRCP | Hinge |
| 162 | Glu Ser Lys Tyr Gly Pro Pro Cys Pro Ser Cys Pro | Exemplary IgG |
| | | Hinge |
| 163 | Glu Ser Lys Tyr Gly Pro Pro Cys Pro Pro Cys Pro | Exemplary IgG |
| | | Hinge |
| 164 | Tyr Gly Pro Pro Cys Pro Pro Cys Pro | Exemplary IgG |
| | | Hinge |
| 165 | Lys Tyr Gly Pro Pro Cys Pro Pro Cys Pro | Exemplary IgG |
| | | Hinge |
| 166 | MLLLVTSLLLCELPHPAFLLIPRKVCNGIGIGEFKDSLSINATNIKHF | tEGFR |
| | KNCTSISGDLHILPVAFRGDSFTHTPPLDPQELDILKTVKEITGFLLI | artificial |
| | QAWPENRTDLHAFENLEIIRGRTKQHGQFSLAVVSLNITSLGLRSL | |
| | KEISDGDVIISGNKNLCYANTINWKKLFGTSGQKTKIISNRGENSCK | |
| | ATGQVCHALCSPEGCWGPEPRDCVSCRNVSRGRECVDKCNLLEG | |
| | EPREFVENSECIQCHPECLPQAMNITCTGRGPDNCIQCAHYIDGPHC | |
| | VKTCPAGVMGENNTLVWKYADAGHVCHLCHPNCTYGCTGPGLE | |
| | GCPTNGPKIPSIATGMVGALLLLLVVALGIGLFM | |
| 167 | EGRGSLLTCGDVEENPGP | T2A artificial |
| 168 | GSGATNFSLLKQAGDVEENPGP | P2A |
| 169 | ATNFSLLKQAGDVEENPGP | P2A |
| 170 | QCTNYALLKLAGDVESNPGP | E2A |
| 171 | VKQTLNFDLLKLAGDVESNPGP | F2A |
| 172 | MLQMAGQCSQNEYFDSLLHACIPCQLRCSSNTPPLTCQRYCNASV | BCMA-Fc fusion |
| | TNSVKGTNAGGGGSPKSSDKTHTCPPCPAPEAEGAPSVFLFPPKPK | polypeptide |
| | DTLMISRTPEVTCVVVDVSHEDPEVKFNWYVDGVEVHNAKTKPR | |
| | EEQYNSTYRVVSVLTVLHQDWLNGKEYKCKVSNKALPSSIEKTIS | |
| | KAKGQPREPQVYTLPPSRDELTKNQVSLTCLVKGFYPSDIA VEWES | |
| | NGQPENNYKTTPPVLDSDGSFFLYSKLTVDKSRWQQGNVFSCSVM | |
| | HEALHNHYTQKSLSLSPGK | |
| 173 | DYYVY | CDR-H1 |
| 174 | WINPNSGGTNYAQKFQG | CDR-H2 |
| 175 | SQRDGYMDY | CDR-H3 |
| 176 | GYTFIDY | CDR-H1 |
| 177 | NPNSGG | CDR-H2 |
| 178 | GYTFIDYYVY | CDR-H1 |
| 179 | WINPNSGGTN | CDR-H2 |
| 180 | GYTFIDYY | CDR-H1 |
| 181 | INPNSGGT | CDR-H2 |
| 182 | ARSQRDGYMDY | CDR-H3 |
| 183 | TGTSSDVG | CDR-L1 |
| 184 | EDSKRPS | CDR-L2 |
| 185 | SSNTRSSTLV | CDR-L3 |
| 186 | ISCTGTSSD | CDR-L1 |
| 187 | EDS | CDR-L2 |
| 188 | QSALTQPASVSASPGQSIAISCTGTSSDVGWYQQHPGKAPKLMIYE | anti-BCMA scFv |
| | DSKRPSGVSNRFSGSKSGNTASLTISGLQAEDEADYYCSSNTRSSTL | |
| | VFGGGTKLTVLGSRGGGGSGGGGSGGGGSLEMAEVQLVQSGAEM | |
| | KKPGASLKLSCKASGYTFIDYYVYWMRQAPGQGLESMGWINPNS | |
| | GGTNYAQKFQGRVTMTRDTSISTAYMELSRLRSDDTAMYYCARS | |
| | QRDGYMDYWGQGTLVTVSS | |
| 189 | DYSIN | CDR-H1 |
| 190 | WINTETREPAYAYDFRG | CDR-H2 |
| 191 | DYSYAMDY | CDR-H3 |
| 192 | RASESVTILGSHLIH | CDR-L1 |
| 193 | LASNVQT | CDR-L2 |
| 194 | LQSRTIPRT | CDR-L3 |
| 195 | GYTFTDY | CDR-H1 |
| 196 | NTETRE | CDR-H2 |
| 197 | DYSYAMDY | CDR-H3 |
| 198 | RASESVTILGSHLIH | CDR-L1 |
| 199 | LASNVQT | CDR-L2 |
| 200 | LQSRTIPRT | CDR-L3 |
| 201 | GYTFTDYSIN | CDR-H1 |
| 202 | WINTETREPA | CDR-H2 |
| 203 | DYSYAMDY | CDR-H3 |
| 204 | RASESVTILGSHLIH | CDR-L1 |
| 205 | LASNVQT | CDR-L2 |
| 206 | LQSRTIPRT | CDR-L3 |
| 207 | GYTFTDYS | CDR-H1 |
| 208 | INTETREP | CDR-H2 |
| 209 | ALDYSYAMDY | CDR-H3 |
| 210 | ESVTILGSHL | CDR-L1 |
| 211 | LA | CDR-L2 |
| 212 | LQSRTIPRT | CDR-L3 |
| 213 | DIVLTQSPPSLAMSLGKRATISCRASESVTILGSHLIHWYQQKPGQP | scFv |
| | PTLLIQLASNVQTGVPARFSGSGSRTDFTLTIDPVEEDDVAVYYCL | |
| | QSRTIPRTFGGGTKLEIKGSTSGSGKPGSGEGSTKGQIQLVQSGPEL | |
| | KKPGETVKISCKASGYTFTDYSINWVKRAPGKGLKWMGWINTET | |
| | REPAYAYDERGRFAFSLETSASTAYLQINNLKYEDTATYFCALDYS | |
| | YAMDYWGQGTSVTVSS | |
| 214 | atggcactccccgtcaccgcccttctcttgcccctcgccctgctgctgcatgctgccaggcccg | BCMA CAR (nt) |
| | acattgtgctcactcagtcacctcccagcctggccatgagcctgggaaaaagggccaccatctcc | |
| | tgtagagccagtgagtccgtcacaatcttggggagccatcttattcactggtatcagcagaagcc | |
| | cgggcagcctccaacccttcttattcagctcgcgtcaaacgccagacgtgtacctgccagatttt | |
| | ctggtagcgggtcccgcactgattttacactgaccatagatccagtggaagaagacgatgtggcc | |
| | gtgtattattgtctgcagagcagaacgattcctcgcacatttggtgggggtactaagagattaag | |
| | ggaagcacgtccggctcagggaagccgggctccggcgagggaagcacgaaggggcaaattcagct | |
| | ggtccagagcggacctgagctgaaaaaacccggcgagactgttaagatagttaagcatctggcta | |
| | taccttcaccgactacagcataaattgggtgaaacgggcccctggaaagggcctcaaatggatgg | |
| | gttggatcaataccgaaactagggagcctgcttatgcatatgacttccgcgggagattcgccttt | |
| | tcactcgagacatctgcctctactgcttacctccaaataaacaacctcaagtatgaagatacagc | |
| | cacttacttttgcgccctcgactatagttacgccatggactactggggacagggaacctccgtta | |
| | ccgtcagttccgcggccgcaaccacaacacctgctccaaggccccccacacccgctccaactata | |
| | gccagccaaccattgagcctcagacctgaagcttgcaggcccgcagcaggaggcgccgtccatac | |
| | gcgaggcctggacttcgcgtgatattgggcccctttggccggaacatgtggggtgttgcttctct | |
| | cccttgtgatcactctcgcgggagaaagaagctcctgtacatcttcaagcagccttttatgcgac | |
| | ctgtgcaaaccactcaggaagaagatgggtgttcatgccgcttccccgaggaaggagggtgtgaa | |
| | ctgagggtgaaattttctagaagcgccgatgctcccgcatatcagcagggtcagaatcagctcta | |
| | caatgaattgaatctcggcaggcgagaagagtacgatgttctggacaagagacggggcagggatc | |
| | ccgagatggggsaaagccccggagaaaaaatcctcaggaggggttgtacaatgagctgcagaagg | |
| | acaagatggctgaagcctatagcgagatcggaatgaaaggcgaaagacgcagaggcaaggggcat | |
| | gacggtctgtaccagggtctctctacagccaccaaggacacttatgatgcgttgcatatgcaagc | |
| | cttgccaccccgctaatga | |
| 215 | MLQMAGQCSQNEYFDSLLHACIPCQLRCSSNTPPLTCQRYCNASV | Human BCMA |
| | TNSVKGTNAILWTCLGLSLIISLAVFVLMFLLRKINSEPLKDEFKNT | |
| | GSGLLGMANIDLEKSRTGDEIILPRGLEYTVEECTCEDCIKSKPKVD | |
| | SDHCFPLPAMEEGATILVTTKTNDYCKSLPAALSATEIEKSISAR | |
| 216 | EVQLLESGGGLVQPGGSLRLSCAASGFTFSSYAMSWVRQAPGKGL | Anti-BCMA scFv |
| | EWVSAISGSGGSTYYADSVKGRFTISRDNSKNTLYLQMNSLRAED | |
| | TAVYYCARAEMGAVFDIWGQGTMVTVSSGSTSGSGKPGSGEGST | |
| | KGEIVLTQSPATLSLSPGERATLSCRASQSVSRYLAWYQQKPGQAP | |
| | RLLIYDASNRATGIPARFSGSGSGTDFTLTISSLEPEDFAVYYCQQRI | |
| | SWPFTFGGGTKVEIK | |
| 217 | EIVLTQSPATLSLSPGERATLSCRASQSVSRYLAWYQQKPGQAPRL | Anti-BCMA scFv |
| | LIYDASNRATGIPARFSGSGSGTDFTLTISSLEPEDFAVYYCQQRIS | |
| | WPFTFGGGTKVEIKRGSTSGSGKPGSGEGSTKGEVQLLESGGGLV | |
| | QPGGSLRLSCAASGFTFSSYAMSWVRQAPGKGLEWVSAISGSGGS | |
| | TYYADSVKGRFTISRDNSKNTLYLQMNSLRAEDTAVYYCARAEM | |
| | GAVFDIWGQGTMVTVSS | |
| 218 | QVQLVESGGGVVQPGRSLRLSCAASGFTFSSYGMHWVRQAPGKG | Anti-BCMA scFv |
| | LEWVAVISYDGSNKYYADSVKGRFTISRDNSKNTLYLQMNSLRAE | |
| | DTAVYYCARDGTYLGGLWYFDLWGRGTLVTVSSGSTSGSGKPGS | |
| | GEGSTKGDIVMTQSPLSLPVTPGEPASISCRSSQSLLHSNGYNYLD | |
| | WYLQKPGQSPQLLIYLGSNRASGVPDRFSGSGSGTDFTLKISRVEA | |
| | EDVGVYYCMQGLGLPLTFGGGTKVEIK | |
| 219 | DIVMTQSPLSLPVTPGEPASISCRSSQSLLHSNGYNYLDWYLQKPG | Anti-BCMA scFv |
| | QSPQLLIYLGSNRASGVPDRFSGSGSGTDFTLKISRVEAEDVGVYY | |
| | CMQGLGLPLTFGGGTKVEIKRGSTSGSGKPGSGEGSTKGQVQLVE | |
| | SGGGVVQPGRSLRLSCAASGFTFSSYGMHWVRQAPGKGLEWVAV | |
| | ISYDGSNKYYADSVKGRFTISRDNSKNTLYLQMNSLRAEDTAVYY | |
| | CARDGTYLGGLWYFDLWGRGTLVTVSS | |
| 220 | QVQLVQSGAEVKKPGASVKVSCKASGYTFTSYYMHWVRQAPGQ | Anti-BCMA scFv |
| | GLEWMGIINPGGGSTSYAQKFQGRVTMTRDTSTSTVYMELSSLRS | |
| | EDTAVYYCARESWPMDVWGQGTTVTVSSGSTSGSGKPGSGEGST | |
| | KGEIVMTQSPATLSVSPGERATLSCRASQSVSSNLAWYQQKPGQA | |
| | PRLLIYGASTRATGIPARFSGSGSGTEFTLTISSLQSEDFAVYYCQQY | |
| | AAYPTFGGGTKVEIK | |
| 221 | EIVMTQSPATLSVSPGERATLSCRASQSVSSNLAWYQQKPGQAPRL | Anti-BCMA scFv |
| | LIYGASTRATGIPARFSGSGSGTEFTLTISSLQSEDFAVYYCQQYAA | |
| | YPTFGGGTKVEIKRGSTSGSGKPGSGEGSTKGQVQLVQSGAEVKK | |
| | PGASVKVSCKASGYTFTSYYMHWVRQAPGQGLEWMGIINPGGGS | |
| | TSYAQKFQGRVTMTRDTSTSTVYMELSSLRSEDTAVYYCARESWP | |
| | MDVWGQGTTVTVSS | |
| 222 | QLQLQESGPGLVKPSETLSLTCTVSGGSISSSSYYWGWIRQPPGKG | Anti-BCMA scFv |
| | LEWIGSISYSGSTYYNPSLKSRVTISVDTSKNQFSLKLSSVTAADTA | |
| | VYYCARGRGYATSLAFDIWGQGTMVTVSSGSTSGSGKPGSGEGST | |
| | KGEIVLTQSPATLSLSPGERATLSCRASQSVSSYLAWYQQKPGQAP | |
| | RLLIYDASNRATGIPARFSGSGSGTDFTLTISSLEPEDFAVYYCQQR | |
| | HVWPPTFGGGTKVEIK | |
| 223 | EIVLTQSPATLSLSPGERATLSCRASQSVSSYLAWYQQKPGQAPRL | Anti-BCMA scFv |
| | LIYDASNRATGIPARFSGSGSGTDFTLTISSLEPEDFAVYYCQQRHV | |
| | WPPTFGGGTKVEIKRGSTSGSGKPGSGEGSTKGQLQLQESGPGLVK | |
| | PSETLSLTCTVSGGSISSSSYYWGWIRQPPGKGLEWIGSISYSGSTY | |
| | YNPSLKSRVTISVDTSKNQFSLKLSSVTAADTAVYYCARGRGYATS | |
| | LAFDIWGQGTMVTVSS | |
| 224 | EVQLVESGGGLVQPGGSLRLSCAASGFTFSSYSMNWVRQAPGKGL | Anti-BCMA scFv |
| | EWVSTISSSSSTIYYADSVKGRFTISRDNAKNSLYLQMNSLRAEDT | |
| | AVYYCARGSQEHLIFDYWGQGTLVTVSSGSTSGSGKPGSGEGSTK | |
| | GEIVLTQSPATLSLSPGERATLSCRASQSVSRYLAWYQQKPGQAPR | |
| | LLIYDASNRATGIPARFSGSGSGTDFTLTISSLEPEDFAVYYCQQRF | |
| | YYPWTFGGGTKVEIK | |
| 225 | EIVLTQSPATLSLSPGERATLSCRASQSVSRYLAWYQQKPGQAPRL | Anti-BCMA scFv |
| | LIYDASNRATGIPARFSGSGSGTDFTLTISSLEPEDFAVYYCQQRFY | |
| | YPWTFGGGTKVEIKRGSTSGSGKPGSGEGSTKGEVQLVESGGGLV | |
| | QPGGSLRLSCAASGFTFSSYSMNWVRQAPGKGLEWVSTISSSSSTI | |
| | YYADSVKGRFTISRDNAKNSLYLQMNSLRAEDTAVYYCARGSQE | |
| | HLIFDYWGQGTLVTVSS | |
| 226 | QVQLVESGGGVVQPGRSLRLSCAASGFTFSSYGMHWVRQAPGKG | Anti-BCMA scFv |
| | LEWVAVISYDGSNKYYADSVKGRFTISRDNSKNTLYLQMNSLRAE | |
| | DTAVYYCARTDFWSGSPPGLDYWGQGTLVTVSSGSTSGSGKPGSG | |
| | EGSTKGDIQLTQSPSSVSASVGDRVTITCRASQGISSWLAWYQQKP | |
| | GKAPKLLIYGASSLQSGVPSRFSGSGSGTDFTLTISSLQPEDFATYY | |
| | CQQIYTFPFTFGGGTKVEIK | |
| 227 | DIQLTQSPSSVSASVGDRVTITCRASQGISSWLAWYQQKPGKAPKL | Anti-BCMA scFv |
| | LIYGASSLQSGVPSRFSGSGSGTDFTLTISSLQPEDFATYYCQQIYTF | |
| | PFTFGGGTKVEIKRGSTSGSGKPGSGEGSTKGQVQLVESGGGVVQP | |
| | GRSLRLSCAASGFTFSSYGMHWVRQAPGKGLEWVAVISYDGSNK | |
| | YYADSVKGRFTISRDNSKNTLYLQMNSLRAEDTAVYYCARTDFW | |
| | SGSPPGLDYWGQGTLVTVSS | |
| 228 | QVQLVQSGAEVKKPGSSVKVSCKASGGTFSSYAISWVRQAPGQGL | Anti-BCMA scFv |
| | EWMGGIIPIFGTANYAQKFQGRVTITADESTSTAYMELSSLRSEDT | |
| | AVYYCARTPEYSSSIWHYYYGMDVWGQGTTVTVSSGSTSGSGKP | |
| | GSGEGSTKGDIVMTQSPDSLAVSLGERATINCKSSQSVLYSSNNKN | |
| | YLAWYQQKPGQPPKLLIYWASTRESGVPDRFSGSGSGTDFTLTISS | |
| | LQAEDVAVYYCQQFAHTPFTFGGGTKVEIK | |
| 229 | DIVMTQSPDSLAVSLGERATINCKSSQSVLYSSNNKNYLAWYQQK | Anti-BCMA scFv |
| | PGQPPKLLIYWASTRESGVPDRFSGSGSGTDFTLTISSLQAEDVAVY | |
| | YCQQFAHTPFTFGGGTKVEIKRGSTSGSGKPGSGEGSTKGQVQLV | |
| | QSGAEVKKPGSSVKVSCKASGGTFSSYAISWVRQAPGQGLEWMG | |
| | GIIPIFGTANYAQKFQGRVTITADESTSTAYMELSSLRSEDTAVYYC | |
| | ARTPEYSSSIWHYYYGMDVWGQGTTVTVSS | |
| 230 | QVQLVESGGGVVQPGRSLRLSCAASGFTFSSYGMHWVRQAPGKG | Anti-BCMA scFv |
| | LEWVAVISYDGSNKYYADSVKGRFTISRDNSKNTLYLQMNSLRAE | |
| | DTAVYYCVKGPLQEPPYDYGMDVWGQGTTVTVSSGSTSGSGKPG | |
| | SGEGSTKGEIVMTQSPATLSVSPGERATLSCRASQSVSSNLAWYQQ | |
| | KPGQAPRLLIYSASTRATGIPARFSGSGSGTEFTLTISSLQSEDFAVY | |
| | YCQQHHVWPLTFGGGTKVEIK | |
| 231 | EIVMTQSPATLSVSPGERATLSCRASQSVSSNLAWYQQKPGQAPRL | Anti-BCMA scFv |
| | LIYSASTRATGIPARFSGSGSGTEFTLTISSLQSEDFAVYYCQQHHV | |
| | WPLTFGGGTKVEIKRGSTSGSGKPGSGEGSTKGQVQLVESGGGVV | |
| | QPGRSLRLSCAASGFTFSSYGMHWVRQAPGKGLEWVAVISYDGS | |
| | NKYYADSVKGRFTISRDNSKNTLYLQMNSLRAEDTAVYYCVKGP | |
| | LQEPPYDYGMDVWGQGTTVTVSS | |
| 232 | DIVLTQSPASLAVSLGERATINCRASESVSVIGAHLIHWYQQKPGQP | Anti-BCMA scFv |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSS | |
| 233 | DIVLTQSPASLAVSLGERATINCRASESVSVIGAHLIHWYQQKPGQP | Anti-BCMA scFv |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSS | |
| 234 | DIVLTQSPASLAVSLGERATINCRASESVSVIGAHLIHWYQQKPGQP | Anti-BCMA scFv |
| | PKLLIYLASNLETGVPARFSGSGSGTDFTLTISSLQAEDAAIYYCLQ | |
| | SRIFPRTFGQGTKLEIKGSTSGSGKPGSGEGSTKGQVQLVQSGSELK | |
| | KPGASVKVSCKASGYTFTDYSINWVRQAPGQGLEWMGWINTETR | |
| | EPAYAYDFRGRFVFSLDTSVSTAYLQISSLKAEDTAVYYCARDYSY | |
| | AMDYWGQGTLVTVSS | |
| 235 | EVQLVESGGGLVQPGGSLRLSCAVSGFALSNHGMSWVRRAPGKG | Anti-BCMA scFv |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSDIQ | |
| | LTQSPSSLSASVGDRVTITCRASQSISSYLNWYQQKPGKAPKLLIYA | |
| | ASSLQSGVPSRFSGSGSGTDFTLTISSLQPEDFATYYCQQSYSTPYTF | |
| | GQGTKVEIK | |
| 236 | QVQLVESGGGLVQPGRSLRLSCAASGFTFSNYAMSWVRQAPGKG | Anti-BCMA scFv |
| | LGWVSGISRSGENTYYADSVKGRFTISRDNSKNTLYLQMNSLRDE | |
| | DTAVYYCARSPAHYYGGMDVWGQGTTVTVSSASGGGGSGGRAS | |
| | GGGGSDIVLTQSPGTLSLSPGERATLSCRASQSISSSFLAWYQQKPG | |
| | QAPRLLIYGASRRATGIPDRFSGSGSGTDFTLTISRLEPEDSAVYYC | |
| | QQYHSSPSWTFGQGTKLEIK | |
| 237 | QVQLVESGGGLVQPGGSLRLSCAVSGFALSNHGMSWVRRAPGKG | Anti-BCMA scFv |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSDIR | |
| | LTQSPSPLSASVGDRVTITCQASEDINKFLNWYHQTPGKAPKLLIY | |
| | DASTLQTGVPSRFSGSGSGTDFTLTINSLQPEDIGTYYCQQYESLPL | |
| | TFGGGTKVEIK | |
| 238 | EVQLVESGGGLVQPGGSLRLSCAVSGFALSNHGMSWVRRAPGKG | Anti-BCMA scFv |
| | LEWVSGIVYSGSTYYAASVKGRFTISRDNSRNTLYLQMNSLRPEDT | |
| | AIYYCSAHGGESDVWGQGTTVTVSSASGGGGSGGRASGGGGSEIV | |
| | LTQSPGTLSLSPGERATLSCRASQSIGSSSLAWYQQKPGQAPRLLM | |
| | YGASSRASGIPDRFSGSGSGTDFTLTISRLEPEDFAVYYCQQYAGSP | |
| | PFTFGQGTKVEIK | |
| 239 | QIQLVQSGPELKKPGETVKISCKASGYTFTDYSINWVKRAPGKGLK | Anti-BCMA scFv |
| | WMGWINTETREPAYAYDFRGRFAFSLETSASTAYLQINNLKYEDT | |
| | ATYFCALDYSYAMDYWGQGTSVTVSSGGGGSGGGGSGGGGSDIV | |
| | LTQSPASLAMSLGKRATISCRASESVSVIGAHLIHWYQQKPGQPPK | |
| | LLIYLASNLETGVPARFSGSGSGTDFTLTIDPVEEDDVAIYSCLQSRI | |
| | FPRTFGGGTKLEIK | |
| 240 | QIQLVQSGPELKKPGETVKISCKASGYTFRHYSMNWVKQAPGKGL | Anti-BCMA scFv |
| | KWMGRINTESGVPIYADDFKGRFAFSVETSASTAYLVINNLKDEDT | |
| | ASYFCSNDYLYSLDFWGQGTALTVSSGGGGSGGGGSGGGGSDIVL | |
| | TQSPPSLAMSLGKRATISCRASESVTILGSHLIYWYQQKPGQPPTLL | |
| | IQLASNVQTGVPARFSGSGSRTDFTLTIDPVEEDDVAVYYCLQSRTI | |
| | PRTFGGGTKLEIK | |
| 241 | QIQLVQSGPELKKPGETVKISCKASGYTFTHYSMNWVKQAPGKGL | Anti-BCMA scFv |
| | KWMGRINTETGEPLYADDFKGRFAFSLETSASTAYLVINNLKNED | |
| | TATFFCSNDYLYSCDYWGQGTTLTVSSGGGGSGGGGSGGGGSDIV | |
| | LTQSPPSLAMSLGKRATISCRASESVTILGSHLIYWYQQKPGQPPTL | |
| | LIQLASNVQTGVPARFSGSGSRTDFTLTIDPVEEDDVAVYYCLQSR | |
| | TIPRTFGGGTKLEIK | |
| 242 | QVQLVQSGAEVKKPGASVKVSCKASGYSFPDYYINWVRQAPGQG | Anti-BCMA scFv |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIK | |
| 243 | QVQLVQSGAEVKKPGASVKVSCKASGYSFPDYYINWVRQAPGQG | Anti-BCMA scFv |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIK | |
| 244 | QVQLVQSGAEVKKPGASVKVSCKASGYSFPDYYINWVRQAPGQG | Anti-BCMA scFv |
| | LEWMGWIYFASGNSEYNQKFTGRVTMTRDTSINTAYMELSSLTSE | |
| | DTAVYFCASLYDYDWYFDVWGQGTMVTVSSGGGGSGGGGSGGG | |
| | GSDIVMTQTPLSLSVTPGQPASISCKSSQSLVHSNGNTYLHWYLQK | |
| | PGQSPQLLIYKVSNRFSGVPDRFSGSGSGTDFTLKISRVEAEDVGIY | |
| | YCSQSSIYPWTFGQGTKLEIK | |
| 245 | QVQLVQSGAEVKKPGASVKVSCKASGYSFPDYYINWVRQAPGQG | Anti-BCMA scFv |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIK | |
| 246 | QVQLVQSGAEVKKPGASVKVSCKASGYSFPDYYINWVRQAPGQG | Anti-BCMA scFv |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIK | |
| 247 | QVQLVQSGAEVKKPGASVKVSCKASGYSFPDYYINWVRQAPGQG | Anti-BCMA scFv |
| | LEWMGWIYFASGNSEYNQKFTGRVTMTRDTSSSTAYMELSSLRSE | |
| | DTAVYFCASLYDYDWYFDVWGQGTMVTVSSGGGGSGGGGSGGG | |
| | GSDIVMTQTPLSLSVTPGEPASISCKSSQSLVHSNGNTYLHWYLQK | |
| | PGQSPQLLIYKVSNRFSGVPDRFSGSGSGADFTLKISRVEAEDVGV | |
| | YYCAETSHVPWTFGQGTKLEIK | |
| 248 | RASQDISKYLN | CDR L1 |
| 249 | SRLHSGV | CDR L2 |
| 250 | GNTLPYTFG | CDR L3 |
| 251 | DYGVS | CDR H1 |
| 252 | VIWGSETTYYNSALKS | CDR H2 |
| 253 | YAMDYWG | CDR H3 |
| 254 | EVKLQESGPGLVAPSQSLSVTCTVSGVSLPDYGVSWIRQPPRKGLE | VH |
| | WLGVIWGSETTYYNSALKSRLTIIKDNSKSQVFLKMNSLQTDDTAI | |
| | YYCAKHYYYGGSYAMDYWGQGTSVTVSS | |
| 255 | DIQMTQTTSSLSASLGDRVTISCRASQDISKYLNWYQQKPDGTVKL | VL |
| | LIYHTSRLHSGVPSRFSGSGSGTDYSLTISNLEQEDIATYFCQQGNT | |
| | LPYTFGGGTKLEIT | |
| 256 | DIQMTQTTSSLSASLGDRVTISCRASQDISKYLNWYQQKPDGTVKL | scFv |
| | LIYHTSRLHSGVPSRFSGSGSGTDYSLTISNLEQEDIATYFCQQGNT | |
| | LPYTFGGGTKLEITGSTSGSGKPGSGEGSTKGEVKLQESGPGLVAP | |
| | SQSLSVTCTVSGVSLPDYGVSWIRQPPRKGLEWLGVIWGSETTYY | |
| | NSALKSRLTIIKDNSKSQVFLKMNSLQTDDTAIYYCAKHYYYGGS | |
| | YAMDYWGQGTSVTVSS | |
| 257 | KASQNVGTNVA | CDR L1 |
| 258 | SATYRNS | CDR L2 |
| 259 | QQYNRYPYT | CDR L3 |
| 260 | SYWMN | CDR H1 |
| 261 | QIYPGDGDTNYNGKFKG | CDR H2 |
| 262 | KTISSVVDFYFDY | CDR H3 |
| 263 | EVKLQQSGAELVRPGSSVKISCKASGYAFSSYWMNWVKQRPGQG | VH |
| | LEWIGQIYPGDGDTNYNGKFKGQATLTADKSSSTAYMQLSGLTSE | |
| | DSAVYFCARKTISSVVDFYFDYWGQGTTVTVSS | |
| 264 | DIELTQSPKFMSTSVGDRVSVTCKASQNVGTNVAWYQQKPGQSPK | VL |
| | PLIYSATYRNSGVPDRFTGSGSGTDFTLTITNVQSKDLADYFCQQY | |
| | NRYPYTSGGGTKLEIKR | |
| 265 | EVKLQQSGAELVRPGSSVKISCKASGYAFSSYWMNWVKQRPGQG | scFv |
| | LEWIGQIYPGDGDTNYNGKFKGQATLTADKSSSTAYMQLSGLTSE | |
| | DSAVYFCARKTISSVVDFYFDYWGQGTTVTVSSGGGGSGGGGSG | |
| | GGGSDIELTQSPKFMSTSVGDRVSVTCKASQNVGTNVAWYQQKP | |
| | GQSPKPLIYSATYRNSGVPDRFTGSGSGTDFTLTITNVQSKDLADYF | |
| | CQQYNRYPYTSGGGTKLEIKR | |
| 266 | HYYYGGSYAMDY | HC-CDR3 |
| 267 | HTSRLHS | LC-CDR2 |
| 268 | QQGNTLPYT | LC-CDR3 |
| 269 | gacatccagatgacccagaccacctccagcctgagcgccagcctgggcgaccgggtgaccatcag | Sequence |
| | ctgccgggccagccaggacatcagcaagtacctgaactggtatcagcagaagcccgacggcaccg | encoding scFv |
| | tcaagctgctgatctaccacaccagccggctgcacagcggcgtgcccagccggtttagcggcagc | |
| | ggctccggcaccgactacagcctgaccatctccaacctggaacaggaagatatcgccacctactt | |
| | ttgccagcagggcaacacactgccctacacctttggcggcggaacaaagctggaaatcaccggca | |
| | gcacctccggcagcggcaagcctggcagcggcgagggcagcaccaagggcgaggtgaagctgcag | |
| | gaaagcggccctggcctggtggcccccagccagagcctgagcgtgacctgcaccgtgagcggcgt | |
| | gagcctgcccgactacggcgtgagctggatccggcagccccccaggaagggcctggaatggctgg | |
| | gcgtgatctggggcagcgagaccacctactacaacagcgccctgaagagccggctgaccatcatc | |
| | aaggacaacagcaagagccaggtgttcctgaagatgaacagcctgcagaccgacgacaccgccat | |
| | ctactactgcgccaagcactactactacggcggcagctacgccatggactactggggccagggca | |
| | ccagcgtgaccgtgagcagc | |
| 270 | SFAMS | CDR-H1 |
| 271 | AISGSGGGTYYADSVKG | CDR-H2 |
| 272 | DKILWFGEPVFDY | CDR-H3 |
| 273 | RASQSVSSYLA | CDR-L1 |
| 274 | DASNRAT | CDR-L2 |
| 275 | FGQGTKVEIKRTV | CDR-L3 |
| 276 | EVQLLESGGGLVQPGGSLRLSCAVSGFTFNSFAMSWVRQAPGKGL | VH |
| | EWVSA | |
| | ISGSGGGTYYADSVKGRFTISRDNSKNTLYLQMNSLRAEDTAVYF | |
| | CAKDKILWFGEPVFDYWGQGTLVTVSS | |
| 277 | EIVLTQSPATLSLSPGERATLSCRASQSVSSYLAWYQQKPGQAPRL | VL |
| | LIYD | |
| | ASNRATGIPARFSGSGSGTDFTLTISSLEPEDFAVYYCQQRSNWPPT | |
| | FGQGTKVEIKRTV | |
| 278 | DYWMQ | CDR-H1 |
| 279 | TIYPGDGDTGYAQKFQG | CDR-H2 |
| 280 | GDYYGSNSLDY | CDR-H3 |
| 281 | KASQDVSTVVA | CDR-L1 |
| 282 | SASYRYI | CDR-L2 |
| 283 | QQHYSPPYT | CDR-L3 |
| 284 | QVQLVQSGAEVAKPGTSVKLSCKASGYTFTDYWMQWVKQRPGQ | VH |
| | GLEWIG | |
| | TIYPGDGDTGYAQKFQGKATLTADKSSKTVYMHLSSLASEDSAVY | |
| | YCARGDYYGSNSLDYWGQGTSVTVSS | |
| 285 | DIVMTQSHLSMSTSLGDPVSITCKASQDVSTVVAWYQQKPGQSPR | VL |
| | RLIY | |
| | SASYRYIGVPDRFTGSGAGTDFTFTISSVQAEDLAVYYCQQHYSPP | |
| | YTFGGGTKLEIKRT | |

What is claimed:

1. A method of selecting cells for manufacture of a cell therapy, comprising:
(1) determining the percentage of CD28+ T cells in a first biological sample obtained from a subject, wherein the first biological sample comprises T cells; and
(2) selecting the subject for manufacturing a cell therapy from a second biological sample obtained from the subject if the percentage of CD28+ T cells in the first biological sample is above a threshold value, wherein the second biological sample comprises T cells.

2. The method of claim 1, further comprising genetically engineering cells of the second biological sample to express a recombinant receptor, thereby generating a cell therapy comprising the genetically engineered cells.

3. A method of manufacturing a cell therapy, comprising:
(1) selecting a subject for manufacturing a cell therapy if the percentage of CD28+ T cells in a first biological sample obtained from the subject is above a threshold value, wherein the first biological sample comprises T cells; and
(2) genetically engineering cells of a second biological sample obtained from the subject to express a recombinant receptor, thereby generating a cell therapy comprising the genetically engineered cells, wherein the second biological sample comprises T cells.

4. The method of claim 2 or claim 3, further comprising administering a dose of the cell therapy to a subject.

5. A method of treating a subject with a cell therapy, comprising administering a dose of a cell therapy comprising genetically engineered cells to a subject, wherein:
(1) the percentage of CD28+ T cells in a first biological sample obtained from a subject is determined to be above a threshold value, wherein the first biological sample comprises T cells;
(2) the subject is selected for manufacture of the cell therapy based on the determining in step (1); and
(3) cells of a second biological sample obtained from the subject are genetically engineered to express a recombinant receptor, thereby generating the cell therapy, wherein the second biological sample comprises T cells.

6. The method of claim 5, further comprising selecting the subject for manufacturing the cell therapy from the second biological sample obtained from the subject if the percentage of CD28+ T cells in the first biological sample is above a threshold value.

7. The method of any of claims 3-6, further comprising determining the percentage of CD28+ T cells in the first biological sample.

8. The method of any of claims 1-7, wherein the first biological sample and the second biological sample are the same sample.

9. The method of any of claims 1-8, wherein the first biological sample and the second biological sample are the same sample, which is an apheresis sample or a leukapheresis sample.

10. A method of selecting cells for manufacture of a cell therapy, comprising:
(1) determining the percentage of CD28+ T cells in a first biological sample obtained from a subject; and
(2) selecting the subject for manufacturing a cell therapy from a second biological sample obtained from the subject if the percentage of CD28+ T cells in the first biological sample is above a threshold value,
wherein the first biological sample and the second biological sample are the same sample, which is an apheresis sample.

11. The method of claim 10, further comprising genetically engineering cells of the second biological sample to express a recombinant receptor, thereby generating a cell therapy comprising the genetically engineered cells.

12. A method of manufacturing a cell therapy, comprising:
(1) selecting a subject for manufacturing a cell therapy if the percentage of CD28+ T cells in a first biological sample obtained from the subject is above a threshold value; and
(2) genetically engineering cells of a second biological sample obtained from the subject to express a recombinant receptor, thereby generating a cell therapy comprising the genetically engineered cells,
wherein the first biological sample and the second biological sample are the same sample, which is an apheresis sample.

13. The method of claim 11 or claim 12, further comprising administering a dose of the cell therapy to a subject.

14. A method of treating a subject with a cell therapy, comprising administering a dose of a cell therapy comprising genetically engineered cells to a subject, wherein:
(1) the percentage of CD28+ T cells in a first biological sample obtained from a subject is determined to be above a threshold value;
(2) the subject is selected for manufacture of the cell therapy based on the determining in step (1); and
(3) cells of a second biological sample obtained from the subject are engineered to express a recombinant receptor, thereby generating the cell therapy,
wherein the first biological sample and the second biological sample are the same sample, which is an apheresis sample.

15. The method of claim 14, further comprising selecting the subject for manufacturing the cell therapy from the second biological sample obtained from the subject if the percentage of CD28+ T cells in the first biological sample is above a threshold value.

16. The method of any of claims 12-15, further comprising determining the percentage of CD28+ T cells in the first biological sample.

17. The method of any of claims 1-16, wherein the threshold value is between about 30% and about 50% CD28+ T cells, or between about 35% and about 45% CD28+ T cells.

18. The method of any of claims 1-17, wherein the threshold value is about 40% CD28+ T cells.

19. The method of any of claims 1-7, 17, and 18, wherein the first biological sample and the second biological sample are different samples.

20. The method of claim 19, wherein the first biological sample is a whole blood sample, an apheresis sample, or a leukapheresis sample.

21. The method of claim 19 or claim 20, wherein the second biological sample is a whole blood sample, an apheresis sample, or a leukapheresis sample.

22. The method of any of claims 1-7 and 17-21, wherein the first biological sample is a whole blood sample or an apheresis sample, and the second biological sample is an apheresis sample or a leukapheresis sample.

23. The method of any of claims 1-7 and 17-22, wherein the first biological sample is a whole blood sample and the second biological sample is a leukapheresis sample.

24. The method of any of claims 4-9 and 11-18, wherein the first biological sample and the second biological sample are the same sample, which is obtained from the subject between about six weeks and about one week prior to administration of the cell therapy to a subject.

25. The method of any of claims 4-9, 11-19, and 24, wherein the first biological sample and the second biological sample are the same sample, which is obtained from the subject about three weeks prior to administration of the cell therapy to a subject.

26. The method of any of claims 4-7 and 17-23, wherein:
(a) the first biological sample and the second biological sample are different samples;
(b) the first biological sample is obtained from the subject between about eight weeks prior and about four weeks prior to administration of the cell therapy to a subject; and
(c) the second biological sample is obtained from the subject between about four weeks and about two weeks prior to administration of the cell therapy to a subject.

27. The method of any of claims 4-7, 17-23, and 26, wherein the first biological sample and the second biological sample are obtained from the subject between about two weeks apart and about six weeks apart.

28. The method of any of claims 4-7, 17-23, 26, and 27, wherein the first biological sample and the second biological sample are obtained from the subject about three weeks apart.

29. The method of any of claims 26-28, wherein the second biological sample is obtained from the subject about three weeks prior to administration of the cell therapy to a subject.

30. The method of any of claims 2-9 and 11-29, wherein, prior to genetic engineering, the cells of the second biological sample are incubated under stimulating conditions.

31. A method of enriching for CD28+ cells, the method comprising:
(a) performing a first selection, the first selection comprising enriching for either of CD28+ or CD3+ cells from a biological sample comprising peripheral blood mononuclear cells (PBMCs) obtained from a subject, thereby generating an enriched cell population; and
(b) performing a second selection on the cells of the enriched cell population, thereby generating a CD28+ enriched population, wherein (i) the first selection comprises enriching for CD28+ cells and the second selection comprises enriching for CD3+ cells from the enriched population; or (ii) the first selection comprises enriching for CD3+ cells and the second selection comprises enriching for CD28+ cells from the enriched cell population,
wherein the CD28+ enriched population has a higher percentage of CD28+ cells than the biological sample and is enriched for CD3+ cells.

32. A method of enriching for CD28+ cells, the method comprising:
(a) performing a first selection, the first selection comprising enriching for CD28+ cells from a biological sample comprising peripheral blood mononuclear cells (PBMCs) obtained from a subject, thereby generating a first enriched population, the first enriched population having a higher percentage of CD28+ cells than the biological sample;
(b) performing a second selection on the cells from the first enriched population, the second selection comprising enriching for one of (i) CD4+ cells and (ii) CD8+ cells from the first enriched population, the enrichment thereby generating a second enriched population enriched for the one of (i) CD4+ cells and (ii) CD8+ cells and a non-selected population; and
(c) performing a third selection, the third selection comprising enriching for the other of (i) CD4+ cells and (ii) CD8+ cells from the non-selected population, the enrichment thereby generating a third enriched population enriched for the other of the (i) CD4+ cells and (ii) CD8+ cells.

33. The method of claim 32, further comprising combining the second enriched population and the third enriched population, optionally at a ratio of between about 1:3 and about 3:1, optionally about 1:1, thereby generating a CD28+ enriched population comprising the second enriched population and the third enriched population.

34. The method of claim 32 or claim 33, wherein the second selection comprises enriching for CD8+ cells.

35. The method of any of claims 31-34 wherein the biological sample is a whole blood sample, an apheresis sample, or a leukapheresis sample.

36. The method of any of claims 31 and 33-35, wherein the CD28+ enriched population comprises:
(i) less than about 5% CD28− T cells;
(ii) CD4+ T cells, wherein at least about 95% of the CD4+ T cells are CD28+; or
(iii) CD8+ T cells, wherein at least about 95% of the CD8+ T cells are CD28+.

37. The method of any of claims 31 and 33-36, wherein at least about 95% of the CD4+ T cells of the CD28+ enriched population comprises CD28+CD4+ T cells.

38. The method of any of claims 31 and 33-37, wherein at least about 95% of the CD8+ T cells of the CD28+ enriched population comprises CD28+CD8+ T cells.

39. The method of any of claims 31 and 33-38, wherein at least about 95% of the CD4+ T cells and at least about 95% of the CD8+ T cells of the CD28+ enriched population comprises CD28+CD4+ T cells and CD28+CD8+ T cells, respectively.

40. The method of any of claims 31 and 33-39, wherein at least about 95% of the CD3+ T cells of the CD28+ enriched population comprises CD28+CD3+ T cells.

41. The method of any of claims 31 and 33-40, wherein the percentage of the CD28− cells in the CD28+ enriched population is less than about or about 35%, 30%, 20%, 10%, 5%, 1% or 0.1% of the percentage of CD28− cells in the biological sample.

42. The method of any of claims 31 and 33-41, wherein CD28+ enriched population comprises less than about 3%, less than about 2%, less than about 1%, less than about 0.1% or less than about 0.01% CD28− cells.

43. The method of any of claims 31 and 33-42, wherein the CD28+ enriched population is free or is essentially free of CD28− cells.

44. The method of any of claims 31 and 33-43, wherein the percentage of naïve-like T cells in the CD28+ enriched population is at least about 10%, 20%, 30%, 40% or 50% greater than the percentage of naïve-like T cells in the biological sample.

45. The method of claim 44, wherein the naïve-like T cells are surface positive for one or more of markers selected from CD45RA, CD27, and CCR7.

46. The method of any of claims 31-45, wherein the enriching for CD28+ cells comprises immunoaffinity-based selection.

47. The method of claim 46, wherein the immunoaffinity-based selection comprises contacting cells with an antibody capable of specifically binding to CD28 and recovering cells bound to the antibody.

48. The method of claim 47, wherein the antibody is immobilized on a solid surface, optionally wherein the solid surface is a magnetic particle.

49. The method of claim 47 and claim 48, wherein the antibody is immobilized on or attached to an affinity chromatography matrix.

50. The method of any of claims 31 and 33-49, wherein the cells of the CD28+ enriched population are genetically engineered to express a recombinant receptor.

51. The method of claim 50, wherein, prior to genetic engineering, the cells of the CD28+ enriched population are incubated under stimulating conditions.

52. The method of any of claims 30 and 51, wherein the stimulating conditions comprise the presence of a stimulatory reagent capable of activating an intracellular signaling domains of a component of a T cell receptor (TCR) complex and an intracellular signaling domain of a costimulatory molecule.

53. The method of claim 52, wherein the stimulatory reagent comprises (i) a primary agent that binds to a member of a TCR complex, optionally that binds to CD3; and (ii) a secondary agent that binds to a T cell costimulatory molecule, optionally wherein the costimulatory molecule is selected from CD28, CD137 (4-1BB), OX40 and ICOS.

54. The method of claim 53, wherein the primary agent and/or the secondary agent comprises an antibody or an antigen-binding fragment thereof.

55. The method of claim 53 or claim 54, wherein the primary agent is an anti-CD3 antibody or an antigen-binding fragment thereof, and the secondary agent is an anti-CD28 antibody or an antigen-binding fragment thereof.

56. The method of any of claims 30 and 51-55, wherein the stimulating conditions comprise the presence of a recombinant cytokine.

57. The method of claim 56, wherein the recombinant cytokine comprises IL-2, IL-7, IL-15, or a combination thereof.

58. The method of any of claims 2-9, 11-30, and 50-57, wherein, following genetic engineering, the genetically engineered cells are cultivated under conditions to allow for expansion or proliferation of the engineered cells.

59. The method of claim 58, wherein the cultivation results in at least about a 2-fold, 3-fold, 4-fold, 5-fold increase in the number of viable genetically engineered cells, compared to at the initiation of cultivation.

60. The method of any one of claims 4-9, 13-30, and 52-59, wherein the cell therapy is an allogeneic cell therapy, and the subject from whom the first and second biological samples are obtained is different than the subject to whom the cell therapy is administered.

61. The method of any one of claims 4-9, 13-30, and 52-59, wherein the cell therapy is an autologous cell therapy, and the subject from whom the first and second biological samples are obtained is the same subject to whom the cell therapy is administered.

62. The method of any of claims 4-9, 13-30, and 52-61, wherein the subject administered the cell therapy has a disease or condition.

63. The method of claim 62, wherein the disease or condition is an infectious disease or disorder, an autoimmune disease, an inflammatory disease, or a cancer.

64. The method of claim 62 or claim 63, wherein the disease or condition is a cancer.

65. The method of claim 64, wherein the cancer is a leukemia, a lymphoma, or a myeloma.

66. The method of claim 64 or claim 65, wherein the cancer is a multiple myeloma (MM), optionally a relapsed/refractory MM.

67. The method of any of claims 62-66, wherein the recombinant receptor binds to an antigen expressed by cells of the disease or condition.

68. The method of any of claims 2-9, 11-30, and 50-67, wherein the recombinant receptor is a T cell receptor (TCR) is a chimeric antigen receptor (CAR).

69. The method of any of claims 2-9, 11-30, and 50-68, wherein the recombinant receptor is a CAR.

70. The method of claim 68 or claim 69, wherein the CAR comprises an extracellular antigen binding domain that binds to the antigen, a transmembrane domain, and an intracellular signaling region.

71. The method of claim 70, wherein the intracellular signaling region comprises an intracellular signaling domain of a CD3-zeta (CD3ζ) chain and a costimulatory signaling region.

72. The method of claim 71, wherein the costimulatory signaling region comprises an intracellular signaling domain of CD28, 4-1BB, or ICOS.

73. The method of claim 71 or claim 72, wherein the costimulatory signaling region comprises an intracellular signaling domain of 4-1BB.

74. The method of any one of claims 70-73, wherein the transmembrane domain is or comprises a transmembrane domain from CD28 or CD8, optionally human CD28 or CD8.

75. The method of any one of claims 70-74, wherein the CAR further comprises an extracellular spacer between the extracellular antigen binding domain and the transmembrane domain.

76. The method of claim 75, wherein the spacer is from CD8, optionally wherein the spacer is a CD8-alpha hinge.

77. The method of claim 75 or claim 76, wherein the transmembrane domain and the spacer are from CD8.

78. The method of any of claims 70-77, wherein the extracellular antigen binding domain binds to B cell maturation antigen (BCMA).

79. The method of any of claims 70-78, wherein the extracellular antigen-binding domain comprises a variable heavy chain (VH) region and, optionally, a variable light chain (VL) region.

80. The method of claim 79, wherein:
the VH region comprises a CDR-H1, a CDR-H2, and a CDR-H3 comprising the amino acid sequences set forth in SEQ ID NOS: 189, 190, and 191, respectively; and the VL region comprises a CDR-L1, a CDR-L2, and a CDR-L3 comprising the amino acid sequences set forth in SEQ ID NOS: 192, 193, and 194, respectively; or
the VH region comprises a CDR-H1, a CDR-H2, and a CDR-H3 comprising the amino acid sequences set forth in SEQ ID NOS: 173, 174 and 175, respectively; and the VL region comprises a CDR-L1, a CDR-L2, and a CDR-L3 comprising the amino acid sequences set forth in SEQ ID NOS: 183, 184 and 185, respectively.

81. The method of claim 79 or claim 80, wherein:
the VH region comprises an amino acid sequence set forth in SEQ ID NO: 18 and the VL region comprises the amino acid sequence set forth in SEQ ID NO: 19;
or the VH region comprises an amino acid sequence set forth in SEQ ID NO: 24, and the VL region comprises the amino acid sequence set forth in SEQ ID NO: 25.

82. The method of any one of claims 70-81, wherein the extracellular antigen-binding domain is a single chain variable fragment (scFv).

83. The method of claim 82, wherein the scFv comprises the amino acid sequence set forth in SEQ ID NO: 213 or SEQ ID NO: 188.

84. The method of any one of claims 68-83, wherein the CAR comprises the amino acid sequence set forth in SEQ ID NO: 116 or SEQ ID NO: 124.

85. The method of any one of claims 68-84, wherein the CAR is encoded by the polynucleotide sequence set forth in SEQ ID NO: 214.

86. The method of any one of claims 4-9, 13-30, and 52-85, wherein the dose of the cell therapy comprises T cells expressing a chimeric antigen receptor (CAR) as present in: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells.

87. The method of any one of claims 4-9, 13-30, and 52-86, wherein the dose of the cell therapy comprises T cells expressing a chimeric antigen receptor (CAR) as present in idecabtagene vicleucel cells.

88. The method of any of claims 70-77, wherein the extracellular antigen binding domain binds to CD19.

89. The method of any one of claims 1-30 and 52-88, wherein the cell therapy comprises CD4+ T cells and/or CD8+ T cells.

90. The method of claim 89, wherein the dose of the cell therapy comprises a defined ratio of CD4+ T cells to CD8+ T cells, which is between about 1:3 and about 3:1 or is about 1:1.

91. The method of any one of claims 4-9, 13-30, and 52-90, wherein the dose of the cell therapy comprises between about 0.5×10⁶ and about 6×10⁸ CAR-positive T cells.

92. The method of any one of claims 4-9, 13-30, and 52-91, wherein the dose of the cell therapy comprises between about 1×10⁸ and about 6×10⁸ CAR-positive T cells.

93. The method of any one of claims 4-9, 13-30, and 52-92, wherein the dose of the cell therapy comprises between about 1.5×10⁸ and about 4.5×10⁹ CAR-positive T cells.

94. The method of any one of claims 4-9, 13-30, and 52-93, wherein the dose of the cell therapy comprises about 1.5×10⁸, 3×10⁸, or about 4.5×10⁸ CAR-positive T cells.

95. The method of any one of claims 4-9, 13-30, and 52-91, wherein the dose of the cell therapy comprises between about 0.5×10⁶ and about 10×10⁶ CAR-positive T cells.

96. The method of any one of claims 1-30 and 52-95, wherein the percentage of CD28+ T cells is the percentage of total T cells that are CD28+.

97. A composition of cells produced by the method of any of claims 1-4, 7-13, and 16-96.

98. A method of treating a subject having or suspected of having a disease or condition, comprising administering to the subject a dose of the cell therapy produced by the method of any of claims 2-4, 7-13, 16-30, and 52-96.

99. Use of a dose of the cell therapy produced by the method of any of claims 2-4, 7-13, 16-30, and 53-96 or the composition of claim 97 for the treatment of a disease or condition in a subject.

100. A dose of the cell therapy produced by the method of any of claims 2-4, 7-13, 16-30, and 53-96 or the composition of claim 97 for use in treating a disease or condition in a subject.

101. A dose of the cell therapy produced by the method of any of claims 2-4, 7-13, 16-30, and 53-96 or the composition of claim 97 for use in the manufacture of a medicament for treating a disease or condition in a subject.

102. A method of treating a disease or condition in a human subject with a T cell therapy, the method comprising administering to a human subject having a disease or condition a therapeutically effective amount of a T cell therapy, wherein:
(a) at least about 40% of T cells in the subject are CD28+; and
(b) manufacture of the T cell therapy relies on CD28-mediated expansion of the T cells of the T cell therapy.

103. The method of claim 102, wherein the disease or condition is a multiple myeloma.

104. A method of treating multiple myeloma in a human subject, the method comprising administering to a human subject having a multiple myeloma a therapeutically effective amount of a T cell therapy, wherein at least about 40% of T cells in the subject are CD28+.

105. The method of claim 104, wherein manufacture of the T cell therapy relies on CD28− mediated expansion of the T cells of the T cell therapy.

106. The method of any one of claims 102-105, wherein, prior to administration of the T cell therapy to the subject, it has been determined that at least about 40% of T cells, e.g., peripheral T cells, in the subject are CD28+.

107. The method of any one of claims 102-106, wherein the T cell therapy targets B cell maturation antigen (BCMA).

108. The method of any one of claims 102-107, wherein the T cell therapy is an autologous T cell therapy.

109. The method of any one of claims 102-108, wherein the T cell therapy is a chimeric antigen receptor (CAR) T cell therapy.

110. The method of claim 109, wherein the CAR T cell therapy targets BCMA.

111. The method of any one of claims 102-110, wherein at least about 44%, at least about 45%, or at least about 50% of T cells, e.g., peripheral T cells, in the subject are CD28+.

112. The method of any one of claims 103-111, wherein the multiple myeloma is a relapsed/refractory multiple myeloma.

113. A method of increasing proliferation of T cells comprising incubating a population of T cells under stimulating conditions, wherein the population of T cells comprises a percentage of CD28+ T cells above a threshold value.

114. A method of increasing proliferation of T cells comprising:
(1) selecting a biological sample comprising a population of T cells in which the percentage of CD28+ T cells in the population of T cells is above a threshold value; and
(2) incubating the selected population of T cells under stimulating conditions.

115. The method of claim 113, wherein the population of T cells is obtained from a biological sample.

116. The method of claim 114 or claim 115, wherein the biological sample is obtained from a subject.

117. The method of claim 116, wherein the subject is human.

118. The method of any of claims 114-117, wherein the biological sample is a whole blood sample, an apheresis sample, or a leukapheresis sample.

119. The method of any of claims 113-118, wherein the stimulating conditions comprise the presence of a stimulatory reagent capable of activating an intracellular signaling domains of a component of a T cell receptor (TCR) complex and an intracellular signaling domain of a costimulatory molecule.

120. The method of claim 114, wherein the stimulatory reagent comprises (i) a primary agent that binds to a member of a TCR complex, optionally that binds to CD3; and (ii) a secondary agent that binds to a T cell costimulatory molecule, optionally wherein the costimulatory molecule is selected from CD28, CD137 (4-1BB), OX40 and ICOS.

121. The method of claim 115, wherein the primary agent and/or the secondary agent comprises an antibody or an antigen-binding fragment thereof.

122. The method of claim 115 or claim 116, wherein the primary agent is an anti-CD3 antibody or an antigen-binding fragment thereof, and the secondary agent is an anti-CD28 antibody or an antigen-binding fragment thereof.

123. The method of any of claims 113-117, wherein the stimulating conditions comprise the presence of a recombinant cytokine.

124. The method of claim 118, wherein the recombinant cytokine comprises IL-2, IL-7, IL-15, or a combination thereof.

125. The method of any of claims 113-124, wherein the method results in increased proliferation compared to a population of T cells comprising a percentage of CD28+ T cells that is not at or above the threshold value.

126. The method of any of claims 113-125, wherein the method results in increased proliferation compared to a population of CD28+ T cells comprising a percentage of CD28+ T cells that is below the threshold value.

127. The method of any of claims 113-126, wherein the threshold value is between about 30% and about 50% CD28+ T cells, or between about 35% and about 45% CD28+ T cells.

128. The method of any of claims 113-127, wherein the threshold value is about 40% CD28+ T cells.

129. The method of any of claims 113-128, wherein the increase in proliferation occurs on day 1, 2, 3, 4, or 5 after incubation.

130. The method of any of claims 1-129, wherein determining the percentage of CD28+ T cells comprises measuring CD28 protein.

131. The method of any of claims 1-129, wherein determining the percentage of CD28+ T cells comprises measuring CD28 RNA.

132. A method of manufacturing a cell therapy, comprising:
(1) selecting a subject for manufacturing a cell therapy if the percentage of any of CD28+, CD45RA+, CD45RO+, CD27+, CD197+, CD4+, CD57+, CD8+, CD25+, PD1+, LAG3+, CD3+ and/or TIM3+ T cells in a first biological sample obtained from the subject is above a threshold value, wherein the first biological sample comprises T cells; and
(2) genetically engineering cells of a second biological sample obtained from the subject to express a recombinant receptor, thereby generating a cell therapy comprising the genetically engineered cells, wherein the second biological sample comprises T cells.

133. The method of claim 132, further comprising determining the percentage of any of CD28+, CD45RA+, CD45RO+, CD27+, CD197+, CD4+, CD57+, CD8+, CD25+, PD1+, LAG3+, CD3+ and/or TIM3+ T cells in the first biological sample.

134. The method of claim 132 or claim 133, wherein the threshold value is calculated as the percentage of T cells that are CD28+, CD45RA+, CD45RO+, CD27+, CD197+, CD4+, CD57+, CD8+, CD25+, PD1+, LAG3+, CD3+ and/or TIM3+ in the first biological sample.

135. The method of any of claims 132-134, wherein the threshold value is between about 20% and about 40% CD28+ T cells, between about 30% and about 50% CD28+ T cells, or between about 35% and about 45% CD28+ T cells.

136. The method of any of claims 132-135, wherein the threshold value is about 40% CD28+ T cells.

137. The method of any of claims 132-134, wherein the threshold value is between about 20% and about 40% CD45RA+ T cells, between about 30% and about 50% CD45RA+ T cells, or between about 35% and about 45% CD45RA+ T cells.

138. The method of any of claims 132-134 or 137, wherein the threshold value is about 40% CD45RA+ T cells.

139. The method of any of claims 132-134, wherein the threshold value is between about 20% and about 40% CD45RO+ T cells, between about 30% and about 50% CD45RO+ T cells, or between about 35% and about 45% CD45RO+ T cells.

140. The method of any of claims 132-134 or 139, wherein the threshold value is about 40% CD45RO+ T cells.

141. The method of any of claims 132-134, wherein the threshold value is between about 20% and about 40% CD27+ T cells, between about 30% and about 50% CD27+ T cells, or between about 35% and about 45% CD27+ T cells.

142. The method of any of claims 132-134 or 141, wherein the threshold value is about 40% CD27+ T cells.

143. The method of any of claims 132-134, wherein the threshold value is between about 20% and about 40% CD197+ T cells, between about 30% and about 50% CD197+ T cells, or between about 35% and about 45% CD197+ T cells.

144. The method of any of claims 132-134 or 143, wherein the threshold value is about 40% CD197+ T cells.

145. The method of any of claims 132-144, further comprising administering a dose of the cell therapy to the subject.

146. A composition of cells produced by the method of any of claims 132-145.


SEQUENCE LISTING (285 sequences)

SEQ ID NO: 1 - protein, 12 aa, Homo sapiens
  REGION 1..12: note = spacer (IgG4hinge)
ESKYGPPCPPCP

SEQ ID NO: 2 - protein, 36 aa, Homo sapiens
  REGION 1..36: note = spacer (IgG4hinge)
GAATCTAAGTACGGACCGCCCTGCCCCCCTTGCCCT

SEQ ID NO: 3 - protein, 119 aa, Homo sapiens
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, synthetic construct
  REGION 1..24: note = T2A
LEGGGEGRGSLLTCGDVEENPGPR

SEQ ID NO: 7 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  REGION 1..27: note = CD28
FWVLVVVGGVLACYSLLVTVAFIIFWV

SEQ ID NO: 9 - protein, 66 aa, Homo sapiens
  REGION 1..66: note = CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  REGION 1..41: note = CD28
RSKRSRLLHSDYMNMTPRRPGPTRKHYQPYAPPRDFAAYRS

SEQ ID NO: 11 - protein, 41 aa, Homo sapiens
  REGION 1..41: note = CD28
RSKRSRGGHSDYMNMTPRRPGPTRKHYQPYAPPRDFAAYRS

SEQ ID NO: 12 - protein, 42 aa, Homo sapiens
  REGION 1..42: note = 4-1BB
KRGRKKLLYIFKQPFMRPVQTTQEEDGCSCRFPEEEEGGCEL

SEQ ID NO: 13 - protein, 112 aa, Homo sapiens
  REGION 1..112: note = CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 10 aa, synthetic construct
  REGION 1..10: note = linker
  REPEAT 5..9: note = SGGGG is repeated 5 times
PGGGSGGGGP

SEQ ID NO: 17 - protein, 17 aa, synthetic construct
  REGION 1..17: note = linker
GSADDAKKDAAKKDGKS

SEQ ID NO: 18 - protein, 117 aa, synthetic construct
  REGION 1..117: note = Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 19 - protein, 111 aa, synthetic construct
  REGION 1..111: note = Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 20 - protein, 122 aa, synthetic construct
  REGION 1..122: note = Variable heavy (VH) Anti-BCMA
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 21 - protein, 107 aa, synthetic construct
  REGION 1..107: note = Variable light (VL) Anti-BCMA
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 22 - protein, 116 aa, synthetic construct
  REGION 1..116: note = Variable heavy (VH) Anti-BCMA
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 23 - protein, 111 aa, synthetic construct
  REGION 1..111: note = Variable light (VL) Anti-BCMA
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 24 - protein, 118 aa, synthetic construct
  REGION 1..118: note = Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 25 - protein, 105 aa, synthetic construct
  REGION 1..105: note = Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 26 - protein, 5 aa, synthetic construct
  REGION 1..5: note = Linker
GGGGS

SEQ ID NO: 27 - protein, 4 aa, synthetic construct
  REGION 1..4: note = Linker
GGGS

SEQ ID NO: 28 - protein, 15 aa, synthetic construct
  REGION 1..15: note = Linker
GGGGSGGGGSGGGGS

SEQ ID NO: 29 - protein, 18 aa, synthetic construct
  REGION 1..18: note = Linker
GSTSGSGKPGSGEGSTKG

SEQ ID NO: 30 - protein, 21 aa, synthetic construct
  REGION 1..21: note = Linker
SRGGGGSGGGGSGGGGSLEMA

SEQ ID NO: 31 - protein, 228 aa, Homo sapiens
  REGION 1..228: note = Hinge-CH2-CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 122 aa, synthetic construct
  REGION 1..122: note = Variable heavy (VH) Anti-BCMA
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 33 - protein, 111 aa, synthetic construct
  REGION 1..111: note = Variable light (VL) Anti-BCMA
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 34 - protein, 120 aa, synthetic construct
  REGION 1..120: note = Variable heavy (VH) Anti-BCMA
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 35 - protein, 112 aa, synthetic construct
  REGION 1..112: note = Variable light (VL) Anti-BCMA
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 36 - protein, 118 aa, synthetic construct
  REGION 1..118: note = Variable heavy (VH) Anti-BCMA
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 37 - protein, 112 aa, synthetic construct
  REGION 1..112: note = Variable light (VL) Anti-BCMA
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 38 - protein, 17 aa, synthetic construct
  REGION 1..17: note = Linker
ASGGGGSGGRASGGGGS

SEQ ID NO: 39 - protein, 21 aa, synthetic construct
  REGION 1..21: note = CD8a signal peptide
MALPVTALLLPLALLLHAARP

SEQ ID NO: 40 - protein, 20 aa, synthetic construct
  REGION 1..20: note = signal peptide
METDTLLLWVLLLWVPGSTG

SEQ ID NO: 41 - protein, 118 aa, synthetic construct
  REGION 1..118: note = Variable heavy (VH) Anti-BCMA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 42 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 43 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 44 - protein, 112 aa, synthetic construct
  REGION 1..112: note = Variable light (VL) Anti-BCMA
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 45 - protein, 116 aa, synthetic construct
  REGION 1..116: note = Variable heavy (VH) Anti-BCMA
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 46 - protein, 106 aa, synthetic construct
  REGION 1..106: note = Variable light (VL) Anti-BCMA
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 47 - protein, 122 aa, synthetic construct
  REGION 1..122: note = Variable heavy (VH) Anti-BCMA
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 48 - protein, 107 aa, synthetic construct
  REGION 1..107: note = Variable light (VL) Anti-BCMA
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 49 - protein, 119 aa, synthetic construct
  REGION 1..119: note = Variable heavy (VH) Anti-BCMA
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 50 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 51 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 52 - protein, 107 aa, synthetic construct
  REGION 1..107: note = Variable light (VL) Anti-BCMA
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 53 - protein, 126 aa, synthetic construct
  REGION 1..126: note = Variable heavy (VH) Anti-BCMA
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 54 - protein, 113 aa, synthetic construct
  REGION 1..113: note = Variable light (VL) Anti-BCMA
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 55 - protein, 123 aa, synthetic construct
  REGION 1..123: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 56 - protein, 107 aa, synthetic construct
  REGION 1..107: note = Variable light (VL) Anti-BCMA
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 57 - protein, 121 aa, synthetic construct
  REGION 1..121: note = Variable heavy (VH) Anti-BCMA
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 58 - protein, 112 aa, synthetic construct
  REGION 1..112: note = Variable light (VL) Anti-BCMA
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 59 - protein, 117 aa, synthetic construct
  REGION 1..117: note = Variable heavy (VH) Anti-BCMA
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 60 - protein, 111 aa, synthetic construct
  REGION 1..111: note = Variable light (VL) Anti-BCMA
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 61 - protein, 115 aa, synthetic construct
  REGION 1..115: note = Variable heavy (VH) Anti-BCMA
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 62 - protein, 107 aa, synthetic construct
  REGION 1..107: note = Variable light (VL) Anti-BCMA
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 63 - protein, 120 aa, synthetic construct
  REGION 1..120: note = Variable heavy (VH) Anti-BCMA
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 64 - protein, 109 aa, synthetic construct
  REGION 1..109: note = Variable light (VL) Anti-BCMA
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 65 - protein, 115 aa, synthetic construct
  REGION 1..115: note = Variable heavy (VH) Anti-BCMA
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 66 - protein, 107 aa, synthetic construct
  REGION 1..107: note = Variable light (VL) Anti-BCMA
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 67 - protein, 115 aa, synthetic construct; identical to SEQ ID NO: 61
  REGION 1..115: note = Variable heavy (VH) Anti-BCMA

SEQ ID NO: 68 - protein, 109 aa, synthetic construct
  REGION 1..109: note = Variable light (VL) Anti-BCMA
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 69 - protein, 117 aa, synthetic construct
  REGION 1..117: note = Variable heavy (VH) Anti-BCMA
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 70 - protein, 111 aa, synthetic construct
  REGION 1..111: note = Variable light (VL) Anti-BCMA
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 71 - protein, 117 aa, synthetic construct
  REGION 1..117: note = Variable heavy (VH) Anti-BCMA
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 72 - protein, 111 aa, synthetic construct
  REGION 1..111: note = Variable light (VL) Anti-BCMA
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 73 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 74 - protein, 112 aa, synthetic construct
  REGION 1..112: note = Variable light (VL) Anti-BCMA
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 75 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 76 - protein, 112 aa, synthetic construct
  REGION 1..112: note = Variable light (VL) Anti-BCMA
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 77 - protein, 122 aa, synthetic construct
  REGION 1..122: note = Anti-BCMA sdAb
QVQLVESGGGLVQPGGSLRLSCEASGFTLDYYAIGWFRQAPGKEREGVICISRSDGSTYYADSVKGRFTISRDNAKKTVYLQMISLKPEDTAAYYCAAGADCSGYLRDYEFRGQGTQVTVSS

SEQ ID NO: 78 - protein, 39 aa, synthetic construct
  REGION 1..39: note = CD28 spacer
IEVMYPPPYLDNEKSNGTIIHVKGKHLCPSPLFPGPSKP

SEQ ID NO: 79 - protein, 25 aa, synthetic construct
  REGION 1..25: note = CD8a TM
IYIWAPLAGTCGVLLLSLVITLYCN

SEQ ID NO: 80 - protein, 30 aa, synthetic construct
  REGION 1..30: note = CD28 spacer (truncated)
LDNEKSNGTIIHVKGKHLCPSPLFPGPSKP

SEQ ID NO: 81 - protein, 46 aa, synthetic construct
  REGION 1..46: note = CD8a hinge
PTTTPAPRPPTPAPTIASQPLSLRPEACRPAAGGAVHTRGLDFACD

SEQ ID NO: 82 - protein, 45 aa, synthetic construct
  REGION 1..45: note = CD8a hinge
TTTPAPRPPTPAPTIASQPLSLRPEACRPAAGGAVHTRGLDFACD

SEQ ID NO: 83 - protein, 55 aa, synthetic construct
  REGION 1..55: note = CD8a hinge
FVPVFLPAKPTTTPAPRPPTPAPTIASQPLSLRPEACRPAAGGAVHTRGLDFACD

SEQ ID NO: 84 - protein, 39 aa, synthetic construct
  REGION 1..39: note = CTLA4 hinge
DTGLYICKVELMYPPPYYLGIGNGTQIYVIDPEPCPDSD

SEQ ID NO: 85 - protein, 24 aa, synthetic construct
  REGION 1..24: note = CTLA4 TM
FLLWILAAVSSGLFFYSFLLTAVS

SEQ ID NO: 86 - protein, 38 aa, synthetic construct
  REGION 1..38: note = PD-1 hinge
QIKESLRAELRVTERRAEVPTAHPSPSPRPAGQFQTLV

SEQ ID NO: 87 - protein, 21 aa, synthetic construct
  REGION 1..21: note = PD-1 TM
VGVVGGLLGSLVLLVWVLAVI

SEQ ID NO: 88 - protein, 16 aa, synthetic construct
  REGION 1..16: note = Fc(gamma)RIIIa hinge
GLAVSTISSFFPPGYQ

SEQ ID NO: 89 - protein, 231 aa, synthetic construct
  REGION 1..231: note = IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 457 aa, synthetic construct
  REGION 1..457: note = anti-BCMA CAR
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 457 aa, synthetic construct
  REGION 1..457: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 466 aa, synthetic construct
  REGION 1..466: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 466 aa, synthetic construct
  REGION 1..466: note = anti-BCMA CAR
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 454 aa, synthetic construct
  REGION 1..454: note = anti-BCMA CAR
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 454 aa, synthetic construct
  REGION 1..454: note = anti-BCMA CAR
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 461 aa, synthetic construct
  REGION 1..461: note = anti-BCMA CAR
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 461 aa, synthetic construct
  REGION 1..461: note = anti-BCMA CAR
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 458 aa, synthetic construct
  REGION 1..458: note = anti-BCMA CAR
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 458 aa, synthetic construct
  REGION 1..458: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 461 aa, synthetic construct
  REGION 1..461: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 461 aa, synthetic construct
  REGION 1..461: note = anti-BCMA CAR
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 471 aa, synthetic construct
  REGION 1..471: note = anti-BCMA CAR
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 471 aa, synthetic construct
  REGION 1..471: note = anti-BCMA CAR
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 462 aa, synthetic construct
  REGION 1..462: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 462 aa, synthetic construct
  REGION 1..462: note = anti-BCMA CAR
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 466 aa, synthetic construct
  REGION 1..466: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 473 aa, synthetic construct
  REGION 1..473: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 470 aa, synthetic construct
  REGION 1..470: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 476 aa, synthetic construct
  REGION 1..476: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 475 aa, synthetic construct
  REGION 1..475: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 482 aa, synthetic construct
  REGION 1..482: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 476 aa, synthetic construct
  REGION 1..476: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 481 aa, synthetic construct
  REGION 1..481: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 479 aa, synthetic construct
  REGION 1..479: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 472 aa, synthetic construct
  REGION 1..472: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 472 aa, synthetic construct
  REGION 1..472: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 472 aa, synthetic construct
  REGION 1..472: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 466 aa, synthetic construct
  REGION 1..466: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAADTGLYICKVELMYPPPYYLGIGNGTQIYVIDPEPCPDSDFLLWILAAVSSGLFFYSFLLTAVSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 464 aa, synthetic construct
  REGION 1..464: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAAQIKESLRAELRVTERRAEVPTAHPSPSPRPAGQFQTLVVGVVGGLLGSLVLLVWVLAVICSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 653 aa, synthetic construct
  REGION 1..653: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 650 aa, synthetic construct
  REGION 1..650: note = anti-BCMA CAR
EVQLVQSGGGLVQPGRSLRLSCTASGFTFGDYAMSWFKQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 651 aa, synthetic construct
  REGION 1..651: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 658 aa, synthetic construct
  REGION 1..658: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 654 aa, synthetic construct
  REGION 1..654: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 653 aa, synthetic construct
  REGION 1..653: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 462 aa, synthetic construct
  REGION 1..462: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 469 aa, synthetic construct
  REGION 1..469: note = anti-BCMA CAR
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 462 aa, synthetic construct
  REGION 1..462: note = anti-BCMA CAR
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 464 aa, synthetic construct
  REGION 1..464: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 467 aa, synthetic construct
  REGION 1..467: note = anti-BCMA CAR
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 472 aa, synthetic construct
  REGION 1..472: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 466 aa, synthetic construct
  REGION 1..466: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 466 aa, synthetic construct
  REGION 1..466: note = anti-BCMA CAR
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 466 aa, synthetic construct
  REGION 1..466: note = anti-BCMA CAR
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 482 aa, synthetic construct
  REGION 1..482: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 28 aa, synthetic construct
  REGION 1..28: note = CD8a TM
IYIWAPLAGTCGVLLLSLVITLYCNHRN

SEQ ID NO: 143 - protein, 21 aa, synthetic construct
  REGION 1..21: note = CD8a TM
IYIWAPLAGTCGVLLLSLVIT

SEQ ID NO: 144 - protein, 4 aa, synthetic construct
  REGION 1..4: note = linking peptide
RAAA

SEQ ID NO: 145 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSS

SEQ ID NO: 146 - protein, 110 aa, synthetic construct
  REGION 1..110: note = Variable light (VL) Anti-BCMA
QSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 147 - protein, 118 aa, synthetic construct
  REGION 1..118: note = Variable heavy (VH) Anti-BCMA
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSS

SEQ ID NO: 148 - protein, 107 aa, synthetic construct
  REGION 1..107: note = Variable light (VL) Anti-BCMA
DIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 149 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSS

SEQ ID NO: 150 - protein, 108 aa, synthetic construct
  REGION 1..108: note = Variable light (VL) Anti-BCMA
SYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 151 - protein, 122 aa, synthetic construct; identical to SEQ ID NO: 32
  REGION 1..122: note = Variable heavy (VH) Anti-BCMA

SEQ ID NO: 152 - protein, 111 aa, synthetic construct; identical to SEQ ID NO: 33
  REGION 1..111: note = Variable light (VL) Anti-BCMA

SEQ ID NO: 153 - protein, 16 aa, synthetic construct
  REGION 1..16: note = CD33 Signal peptide
MPLLLLLPLLWAGALA

SEQ ID NO: 154 - protein, 18 aa, synthetic construct
  REGION 1..18: note = CD8 alpha signal peptide
MALPVTALLLPLALLLHA

SEQ ID NO: 155 - DNA, 66 bp, synthetic construct
  misc_feature 1..66: note = GMCSFR alpha chain signal sequence
  source 1..66: mol_type = other DNA
atgcttctcctggtgacaagccttctgctctgtgagttaccacacccagcattcctcctgatccca

SEQ ID NO: 156 - protein, 22 aa, synthetic construct
  REGION 1..22: note = GMCSFR alpha chain signal sequence
MLLLVTSLLLCELPHPAFLLIP

SEQ ID NO: 157 - protein, 14 aa, synthetic construct
  REGION 1..14: note = Exemplary IgG Hinge
EVVVKYGPPCPPCP

SEQ ID NO: 158 - skipped

SEQ ID NO: 159 - protein, 15 aa, synthetic construct
  REGION 1..15: note = Exemplary IgG Hinge
EPKSCDKTHTCPPCP

SEQ ID NO: 160 - protein, 12 aa, synthetic construct
  REGION 1..12: note = Exemplary IgG Hinge
ERKCCVECPPCP

SEQ ID NO: 161 - protein, 61 aa, synthetic construct
  REGION 1..61: note = Exemplary IgG Hinge
ELKTPLGDTHTCPRCPEPKSCDTPPPCPRCPEPKSCDTPPPCPRCPEPKSCDTPPPCPRCP

SEQ ID NO: 162 - protein, 12 aa, synthetic construct
  REGION 1..12: note = Exemplary IgG Hinge
ESKYGPPCPSCP

SEQ ID NO: 163 - protein, 12 aa, synthetic construct
  REGION 1..12: note = Exemplary IgG Hinge
ESKYGPPCPPCP

SEQ ID NO: 164 - protein, 9 aa, synthetic construct
  REGION 1..9: note = Exemplary IgG Hinge
YGPPCPPCP

SEQ ID NO: 165 - protein, 10 aa, synthetic construct
  REGION 1..10: note = Exemplary IgG Hinge
KYGPPCPPCP

SEQ ID NO: 166 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 167 - protein, 18 aa, synthetic construct
  REGION 1..18: note = T2A
EGRGSLLTCGDVEENPGP

SEQ ID NO: 168 - protein, 22 aa, synthetic construct
  REGION 1..22: note = P2A
GSGATNFSLLKQAGDVEENPGP

SEQ ID NO: 169 - protein, 19 aa, synthetic construct
  REGION 1..19: note = P2A
ATNFSLLKQAGDVEENPGP

SEQ ID NO: 170 - protein, 20 aa, synthetic construct
  REGION 1..20: note = E2A
QCTNYALLKLAGDVESNPGP

SEQ ID NO: 171 - protein, 22 aa, synthetic construct
  REGION 1..22: note = F2A
VKQTLNFDLLKLAGDVESNPGP

SEQ ID NO: 172 - protein, 290 aa, synthetic construct
  REGION 1..290: note = BCMA-Fc fusion polypeptide
MLQMAGQCSQNEYFDSLLHACIPCQLRCSSNTPPLTCQRYCNASVTNSVKGTNAGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 5 aa, synthetic construct
  REGION 1..5: note = CDR-H1
DYYVY

SEQ ID NO: 174 - protein, 17 aa, synthetic construct
  REGION 1..17: note = CDR-H2
WINPNSGGTNYAQKFQG

SEQ ID NO: 175 - protein, 9 aa, synthetic construct
  REGION 1..9: note = CDR-H3
SQRDGYMDY

SEQ ID NO: 176 - protein, 7 aa, synthetic construct
  REGION 1..7: note = CDR-H1
GYTFIDY

SEQ ID NO: 177 - protein, 6 aa, synthetic construct
  REGION 1..6: note = CDR-H2
NPNSGG

SEQ ID NO: 178 - protein, 10 aa, synthetic construct
  REGION 1..10: note = CDR-H1
GYTFIDYYVY

SEQ ID NO: 179 - protein, 10 aa, synthetic construct
  REGION 1..10: note = CDR-H2
WINPNSGGTN

SEQ ID NO: 180 - protein, 8 aa, synthetic construct
  REGION 1..8: note = CDR-H1
GYTFIDYY

SEQ ID NO: 181 - protein, 8 aa, synthetic construct
  REGION 1..8: note = CDR-H2
INPNSGGT

SEQ ID NO: 182 - protein, 11 aa, synthetic construct
  REGION 1..11: note = CDR-H3
ARSQRDGYMDY

SEQ ID NO: 183 - protein, 8 aa, synthetic construct
  REGION 1..8: note = CDR-L1
TGTSSDVG

SEQ ID NO: 184 - protein, 7 aa, synthetic construct
  REGION 1..7: note = CDR-L2
EDSKRPS

SEQ ID NO: 185 - protein, 10 aa, synthetic construct
  REGION 1..10: note = CDR-L3
SSNTRSSTLV

SEQ ID NO: 186 - protein, 9 aa, synthetic construct
  REGION 1..9: note = CDR-L1
ISCTGTSSD

SEQ ID NO: 187 - skipped

SEQ ID NO: 188 - protein, 244 aa, synthetic construct
  REGION 1..244: note = anti-BCMA scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 189 - protein, 5 aa, synthetic construct
  REGION 1..5: note = CDR-H1
DYSIN

SEQ ID NO: 190 - protein, 17 aa, synthetic construct
  REGION 1..17: note = CDR-H2
WINTETREPAYAYDFRG

SEQ ID NO: 191 - protein, 8 aa, synthetic construct
  REGION 1..8: note = CDR-H3
DYSYAMDY

SEQ ID NO: 192 - protein, 15 aa, synthetic construct
  REGION 1..15: note = CDR-L1
RASESVTILGSHLIH

SEQ ID NO: 193 - protein, 7 aa, synthetic construct
  REGION 1..7: note = CDR-L2
LASNVQT

SEQ ID NO: 194 - protein, 9 aa, synthetic construct
  REGION 1..9: note = CDR-L3
LQSRTIPRT

SEQ ID NO: 195 - protein, 7 aa, synthetic construct
  REGION 1..7: note = CDR-H1
GYTFTDY

SEQ ID NO: 196 - protein, 6 aa, synthetic construct
  REGION 1..6: note = CDR-H2
NTETRE

SEQ ID NO: 197 - protein, 8 aa, synthetic construct
  REGION 1..8: note = CDR-H3
DYSYAMDY

SEQ ID NO: 198 - protein, 15 aa, synthetic construct
  REGION 1..15: note = CDR-L1
RASESVTILGSHLIH

SEQ ID NO: 199 - protein, 7 aa, synthetic construct
  REGION 1..7: note = CDR-L2
LASNVQT

SEQ ID NO: 200 - protein, 9 aa, synthetic construct
  REGION 1..9: note = CDR-L3
LQSRTIPRT

SEQ ID NO: 201 - protein, 10 aa, synthetic construct
  REGION 1..10: note = CDR-H1
GYTFTDYSIN

SEQ ID NO: 202 - protein, 10 aa, synthetic construct
  REGION 1..10: note = CDR-H2
WINTETREPA

SEQ ID NO: 203 - protein, 8 aa, synthetic construct
  REGION 1..8: note = CDR-H3
DYSYAMDY

SEQ ID NO: 204 - protein, 15 aa, synthetic construct
  REGION 1..15: note = CDR-L1
RASESVTILGSHLIH

SEQ ID NO: 205 - protein, 7 aa, synthetic construct
  REGION 1..7: note = CDR-L2
LASNVQT

SEQ ID NO: 206 - protein, 9 aa, synthetic construct
  REGION 1..9: note = CDR-L3
LQSRTIPRT

SEQ ID NO: 207 - protein, 8 aa, synthetic construct
  REGION 1..8: note = CDR-H1
GYTFTDYS

SEQ ID NO: 208 - protein, 8 aa, synthetic construct
  REGION 1..8: note = CDR-H2
INTETREP

SEQ ID NO: 209 - protein, 10 aa, synthetic construct
  REGION 1..10: note = CDR-H3
ALDYSYAMDY

SEQ ID NO: 210 - protein, 10 aa, synthetic construct
  REGION 1..10: note = CDR-L1
ESVTILGSHL

SEQ ID NO: 211 - skipped

SEQ ID NO: 212 - protein, 9 aa, synthetic construct
  REGION 1..9: note = CDR-L3
LQSRTIPRT

SEQ ID NO: 213 - protein, 246 aa, synthetic construct
  REGION 1..246: note = scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 214 - DNA, 1485 bp, synthetic construct
  misc_feature 1..1485: note = BCMA CAR (nt)
  source 1..1485: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

SEQ ID NO: 215 - protein, 184 aa, Homo sapiens
  REGION 1..184: note = Human BCMA
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 216 - protein, 243 aa, synthetic construct
  REGION 1..243: note = Anti-BCMA scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 217 - protein, 244 aa, synthetic construct
  REGION 1..244: note = Anti-BCMA scFv
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 218 - protein, 252 aa, synthetic construct
  REGION 1..252: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 219 - protein, 253 aa, synthetic construct
  REGION 1..253: note = Anti-BCMA scFv
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 220 - protein, 240 aa, synthetic construct
  REGION 1..240: note = Anti-BCMA scFv
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 221 - protein, 241 aa, synthetic construct
  REGION 1..241: note = Anti-BCMA scFv
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 222 - protein, 247 aa, synthetic construct
  REGION 1..247: note = Anti-BCMA scFv
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 223 - protein, 248 aa, synthetic construct
  REGION 1..248: note = Anti-BCMA scFv
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 224 - protein, 244 aa, synthetic construct
  REGION 1..244: note = Anti-BCMA scFv
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 225 - protein, 245 aa, synthetic construct
  REGION 1..245: note = Anti-BCMA scFv
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 226 - protein, 247 aa, synthetic construct
  REGION 1..247: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 227 - protein, 248 aa, synthetic construct
  REGION 1..248: note = Anti-BCMA scFv
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 228 - protein, 257 aa, synthetic construct
  REGION 1..257: note = Anti-BCMA scFv
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 229 - protein, 258 aa, synthetic construct
  REGION 1..258: note = Anti-BCMA scFv
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 230 - protein, 248 aa, synthetic construct
  REGION 1..248: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 231 - protein, 249 aa, synthetic construct
  REGION 1..249: note = Anti-BCMA scFv
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 232 - protein, 246 aa, synthetic construct
  REGION 1..246: note = Anti-BCMA scFv
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 233 - protein, 246 aa, synthetic construct; identical to SEQ ID NO: 232
  REGION 1..246: note = Anti-BCMA scFv

SEQ ID NO: 234 - protein, 246 aa, synthetic construct; identical to SEQ ID NO: 232
  REGION 1..246: note = Anti-BCMA scFv

SEQ ID NO: 235 - protein, 239 aa, synthetic construct
  REGION 1..239: note = Anti-BCMA scFv
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 236 - protein, 246 aa, synthetic construct
  REGION 1..246: note = Anti-BCMA scFv
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 237 - protein, 239 aa, synthetic construct
  REGION 1..239: note = Anti-BCMA scFv
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 238 - protein, 241 aa, synthetic construct
  REGION 1..241: note = Anti-BCMA scFv
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 239 - protein, 243 aa, synthetic construct
  REGION 1..243: note = Anti-BCMA scFv
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 240 - protein, 243 aa, synthetic construct
  REGION 1..243: note = Anti-BCMA scFv
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 241 - protein, 243 aa, synthetic construct
  REGION 1..243: note = Anti-BCMA scFv
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 242 - protein, 246 aa, synthetic construct
  REGION 1..246: note = Anti-BCMA scFv
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 243 - protein, 246 aa, synthetic construct; identical to SEQ ID NO: 242
  REGION 1..246: note = Anti-BCMA scFv

SEQ ID NO: 244 - protein, 246 aa, synthetic construct; identical to SEQ ID NO: 242
  REGION 1..246: note = Anti-BCMA scFv

SEQ ID NO: 245 - protein, 246 aa, synthetic construct
  REGION 1..246: note = Anti-BCMA scFv
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 246 - protein, 246 aa, synthetic construct; identical to SEQ ID NO: 245
  REGION 1..246: note = Anti-BCMA scFv

SEQ ID NO: 247 - protein, 246 aa, synthetic construct; identical to SEQ ID NO: 245
  REGION 1..246: note = Anti-BCMA scFv

SEQ ID NO: 248 - protein, 11 aa, synthetic construct
  UNSURE 1..11: note = CDR L1
RASQDISKYLN

SEQ ID NO: 249 - protein, 7 aa, synthetic construct
  UNSURE 1..7: note = CDR L2
SRLHSGV

SEQ ID NO: 250 - protein, 9 aa, synthetic construct
  UNSURE 1..9: note = CDR L3
GNTLPYTFG

SEQ ID NO: 251 - protein, 5 aa, synthetic construct
  UNSURE 1..5: note = CDR H1
DYGVS

SEQ ID NO: 252 - protein, 16 aa, synthetic construct
  UNSURE 1..16: note = CDR H2
VIWGSETTYYNSALKS

SEQ ID NO: 253 - protein, 7 aa, synthetic construct
YAMDYWG

SEQ ID NO: 254 - protein, 120 aa, synthetic construct
  UNSURE 1..120: note = VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 255 - protein, 107 aa, synthetic construct
  UNSURE 1..107: note = VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 256 - protein, 245 aa, synthetic construct
  UNSURE 1..245: note = scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 257 - protein, 11 aa, synthetic construct
  UNSURE 1..11: note = CDR L1
KASQNVGTNVA

SEQ ID NO: 258 - protein, 7 aa, synthetic construct
  UNSURE 1..7: note = CDR L2
SATYRNS

SEQ ID NO: 259 - protein, 9 aa, synthetic construct
  UNSURE 1..9: note = CDR L3
QQYNRYPYT

SEQ ID NO: 260 - protein, 5 aa, synthetic construct
  UNSURE 1..5: note = CDR H1
SYWMN

SEQ ID NO: 261 - protein, 17 aa, synthetic construct
  UNSURE 1..17: note = CDR H2
QIYPGDGDTNYNGKFKG

SEQ ID NO: 262 - protein, 13 aa, synthetic construct
  UNSURE 1..13: note = CDR H3
KTISSVVDFYFDY

SEQ ID NO: 263 - protein, 122 aa, synthetic construct
  UNSURE 1..122: note = VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 264 - protein, 108 aa, synthetic construct
  UNSURE 1..108: note = VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 265 - protein, 245 aa, synthetic construct
  UNSURE 1..245: note = scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 266 - protein, 12 aa, synthetic construct
  UNSURE 1..12: note = HC-CDR3
HYYYGGSYAMDY

SEQ ID NO: 267 - protein, 7 aa, synthetic construct
  UNSURE 1..7: note = LC-CDR2
HTSRLHS

SEQ ID NO: 268 - protein, 9 aa, synthetic construct
  UNSURE 1..9: note = LC-CDR3
QQGNTLPYT

SEQ ID NO: 269 - DNA, 735 bp, synthetic construct
  source 1..735: mol_type = other DNA
  unsure 1..735: note = Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 270 - protein, 5 aa, synthetic construct
  UNSURE 1..5: note = CDR-H1
SFAMS

SEQ ID NO: 271 - protein, 17 aa, synthetic construct
  UNSURE 1..17: note = CDR-H2
AISGSGGGTYYADSVKG

SEQ ID NO: 272 - protein, 13 aa, synthetic construct
  UNSURE 1..13: note = CDR-H3
DKILWFGEPVFDY

SEQ ID NO: 273 - protein, 11 aa, synthetic construct
  UNSURE 1..11: note = CDR-L1
RASQSVSSYLA

SEQ ID NO: 274 - protein, 7 aa, synthetic construct
  UNSURE 1..7: note = CDR-L2
DASNRAT

SEQ ID NO: 275 - protein, 13 aa, synthetic construct
  UNSURE 1..13: note = CDR-L3
FGQGTKVEIKRTV

SEQ ID NO: 276 - protein, 122 aa, synthetic construct [sequence omitted]

SEQ ID NO: 277 - protein, 110 aa, synthetic construct
  UNSURE 1..110: note = VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTV

SEQ ID NO: 278 - protein, 5 aa, synthetic construct
  UNSURE 1..5: note = CDR-H1
DYWMQ

SEQ ID NO: 279 - protein, 17 aa, synthetic construct
  UNSURE 1..17: note = CDR-H2
TIYPGDGDTGYAQKFQG

SEQ ID NO: 280 - protein, 11 aa, synthetic construct
  UNSURE 1..11: note = CDR-H3
GDYYGSNSLDY

SEQ ID NO: 281 - protein, 11 aa, synthetic construct
  UNSURE 1..11: note = CDR-L1
KASQDVSTVVA

SEQ ID NO: 282 - protein, 7 aa, synthetic construct
  UNSURE 1..7: note = CDR-L2
SASYRYI

SEQ ID NO: 283 - protein, 9 aa, synthetic construct
  UNSURE 1..9: note = CDR-L3
QQHYSPPYT

SEQ ID NO: 284 - protein, 120 aa, synthetic construct [sequence omitted]

SEQ ID NO: 285 - protein, 109 aa, synthetic construct
  UNSURE 1..109: note = VL
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRT